(19) United States
(12) Patent Application Publication
(10) Pub. No.: US 2022/0132852 A1
(43) Pub. Date: May 5, 2022

(54) PESTICIDALLY ACTIVE HETEROCYCLIC DERIVATIVES WITH SULFUR CONTAINING SUBSTITUENTS

(71) Applicant: SYNGENTA CROP PROTECTION AG, Basel (CH)

(72) Inventors: Amandine KOLLETH KRIEGER, Stein (CH); Michel MUEHLEBACH, Stein (CH); André STOLLER, Stein (CH); Daniel EMERY, Stein (CH); Sebastian RENDLER, Basel (CH); Anke BUCHHOLZ, Stein (CH); Vikas SIKERVAR, Goa (IN); Indira SEN, Goa (IN)

(73) Assignee: SYNGENTA CROP PROTECTION AG, Basel (CH)

(21) Appl. No.: 17/436,868

(22) PCT Filed: Mar. 4, 2020
(86) PCT No.: PCT/EP2020/055724

§ 371 Date: Sep. 7, 2021

(30) Foreign Application Priority Data
Mar. 8, 2019 (IN) 201911009149
Jun. 19, 2019 (EP) 19181169.4

(51) Int. Cl. A01N 43/90; C07D 471/04; A01P 7/02; A01P 7/04

(52) U.S. Cl. CPC A01N 43/90; A01P 7/04; A01P 7/02; C07D 471/04

(57) ABSTRACT

Compounds of the formula (I) wherein the substituents are as defined in claim 1. Furthermore, the present invention relates to agrochemical compositions which comprise compounds of formula (I), to preparation of these compositions, and to the use of the compounds or compositions in agriculture or horticulture for combating, preventing or controlling animal pests, including arthropods and in particular insects, nematodes, molluscs or representatives of the order Acarina.

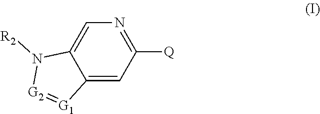

The present invention relates to pesticidally active, in particular insecticidally active heterocyclic derivatives containing sulfur substituents, to processes for their preparation, to compositions comprising those compounds, and to their use for controlling animal pests, including arthropods and in particular insects or representatives of the order Acarina.

Heterocyclic compounds with pesticidal action are known and described, for example, in WO2013191112.

It has now surprisingly been found that certain novel pesticidally active derivatives with sulfur containing substitutents have favourable properties as pesticides.

The present invention therefore provides compounds of formula I,

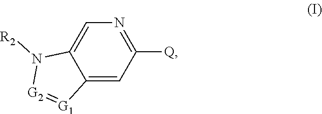

wherein
G₁ and G₂ are, independently from each other, CH or N;
R₂ is C₁-C₆haloalkyl;
Q is a radical selected from the group consisting of formula Qa and Qb

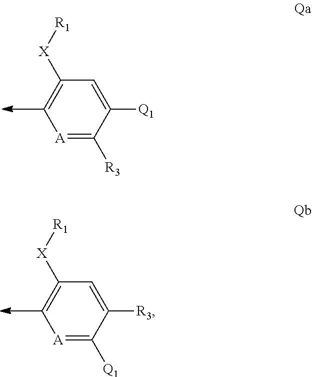

wherein the arrow denotes the point of attachment to the carbon atom of the bicyclic ring;
and wherein A represents CH or N;

### X is S, SO, or SO₂;

R₁ is C₁-C₄alkyl or C₃-C₆cycloalkyl-C₁-C₄alkyl;
Q₁ is hydrogen, halogen, C₁-C₆haloalkyl, C₃-C₆cycloalkyl, C₃-C₆cycloalkyl monosubstituted by cyano, C₁-C₆cyanoalkyl, C₁-C₆cyanoalkoxy, C₁-C₆haloalkoxy, —N(R₄)₂, —N(R₄)COR₅, —N(R₄)CON(R₄)₂, (oxazolidin-2-one)-3-yl or 2-pyridyloxy; or
Q₁ is a five- to six-membered aromatic ring system linked via a ring carbon atom to the ring which contains the substituent A, said ring system is unsubstituted or is mono- or polysubstituted by substituents selected from the group consisting of halogen, cyano, C₁-C₄alkyl, C₁-C₄haloalkyl, C₁-C₄alkoxy, C₁-C₄haloalkoxy, C₁-C₄alkylsulfanyl, C₁-C₄alkylsulfinyl and C₁-C₄alkylsulfonyl; and said ring system can contain 1, 2 or 3 ring heteroatoms selected from the group consisting of nitrogen, oxygen and sulphur, where said ring system may not contain more than one ring oxygen atom and not more than one ring sulfur atom; or
Q₁ is a five-membered aromatic ring system linked via a ring nitrogen atom to the ring which contains the substituent A, said ring system is unsubstituted or is mono- or polysubstituted by substituents selected from the group consisting of halogen, cyano, C₁-C₄alkyl, C₁-C₄haloalkyl, C₁-C₄alkoxy, C₁-C₄haloalkoxy, C₁-C₄alkylsulfanyl, C₁-C₄alkylsulfinyl and C₁-C₄alkylsulfonyl; and said ring system contains 1, 2 or 3 ring heteroatoms selected from the group consisting of nitrogen, oxygen and sulphur, where said ring system contains at least one ring nitrogen atom and may not contain more than one ring oxygen atom and not more than one ring sulfur atom;
R₃ is hydrogen, halogen or C₁-C₄alkyl;
Each R₄ is independently hydrogen, C₁-C₄alkyl or C₃-C₆cycloalkyl; and
R₅ is C₁-C₆alkyl, C₁-C₆haloalkyl or C₃-C₆cycloalkyl.

The present invention also provides agrochemically acceptable salts, stereoisomers, enantiomers, tautomers and N-oxides of the compounds of formula I.

Compounds of formula I which have at least one basic centre can form, for example, acid addition salts, for example with strong inorganic acids such as mineral acids, for example perchloric acid, sulfuric acid, nitric acid, nitrous acid, a phosphorus acid or a hydrohalic acid, with strong organic carboxylic acids, such as C₁-C₄alkanecarboxylic acids which are unsubstituted or substituted, for example by halogen, for example acetic acid, such as saturated or unsaturated dicarboxylic acids, for example oxalic acid, malonic acid, succinic acid, maleic acid, fumaric acid or phthalic acid, such as hydroxycarboxylic acids, for example ascorbic acid, lactic acid, malic acid, tartaric acid or citric acid, or such as benzoic acid, or with organic sulfonic acids, such as C₁-C₄alkane- or arylsulfonic acids which are unsubstituted or substituted, for example by halogen, for example methane- or p-toluenesulfonic acid. Compounds of formula I which have at least one acidic group can form, for example, salts with bases, for example mineral salts such as alkali metal or alkaline earth metal salts, for example sodium, potassium or magnesium salts, or salts with ammonia or an organic amine, such as mor-pholine, piperidine, pyrrolidine, a mono-, di- or tri-lower-alkylamine, for example ethyl-, diethyl-, triethyl- or dimethylpropylamine, or a mono-, di- or trihydroxy-lower-alkylamine, for example mono-, di- or triethanolamine.

In each case, the compounds of formula (I) according to the invention are in free form, in oxidized form as a N-oxide or in salt form, e.g. an agronomically usable salt form.

N-oxides are oxidized forms of tertiary amines or oxidized forms of nitrogen containing heteroaromatic compounds. They are described for instance in the book “Heterocyclic N-oxides” by A. Albini and S. Pietra, CRC Press, Boca Raton 1991.

The compounds of formula I according to the invention also include hydrates which may be formed during the salt formation.

Where substituents are indicated as being itself further substituted, this means that they carry one or more identical or different substituents, e.g. one to four substituents. Normally not more than three such optional substituents are present at the same time. Preferably not more than two such substituents are present at the same time (i.e. the group is substituted by one or two of the substituents indicated). Where the additional substituent group is a larger group, such as cycloalkyl or phenyl, it is most preferred that only one such optional substituent is present. Where a group is indicated as being substituted, e.g. alkyl, this includes those groups that are part of other groups, e.g. the alkyl in alkylthio.

The term “C₁-Cₙalkyl” as used herein refers to a saturated straight-chain or branched hydrocarbon radical attached via any of the carbon atoms having 1 to n carbon atoms, for example, any one of the radicals methyl, ethyl, n-propyl, 1-methylbutyl, 2-methylbutyl, 3-methylbutyl, 2, 2-dimethylpropyl, 1-ethylpropyl, n-hexyl, n-pentyl, 1, 1-dimethylpropyl, 1, 2-dimethylpropyl, 1-methylpentyl, 2-methylpentyl, 3-methylpentyl, 4-methylpentyl, 1, 1-dimethylbutyl, 1,2-dimethylbutyl, 1, 3-dimethylbutyl, 2, 2-dimethylbutyl, 2, 3-dimethylbutyl, 3, 3-dimethylbutyl, 1-ethylbutyl, 2-ethylbutyl, 1,1, 2-trimethylpropyl, 1,2, 2-trimethylpropyl, 1-ethyl-1-methylpropyl, or 1-ethyl-2-methylpropyl.

The term “C₁-Cₙhaloalkyl” as used herein refers to a straight-chain or branched saturated alkyl radical attached via any of the carbon atoms having 1 to n carbon atoms (as mentioned above), where some or all of the hydrogen atoms in these radicals may be replaced by fluorine, chlorine, bromine and/or iodine, i.e., for example, any one of chloromethyl, dichloromethyl, trichloromethyl, fluoromethyl, difluoromethyl, trifluoromethyl, chlorofluoromethyl, dichlorofluoromethyl, chlorodifluoromethyl, 2-fluoroethyl, 2-chloroethyl, 2-bromoethyl, 2-iodoethyl, 2, 2-difluoroethyl, 2,2, 2-trifluoroethyl, 2-chloro-2-fluoroethyl, 2-chloro-2, 2-difluoroethyl, 2, 2-dichloro-2-fluoroethyl, 2,2, 2-trichloroethyl, pentafluoroethyl, 2-fluoropropyl, 3-fluoropropyl, 2,2-difluoropropyl, 2, 3-difluoropropyl, 2-chloropropyl, 3-chloropropyl, 2, 3-dichloropropyl, 2-bromopropyl, 3-bromopropyl, 3,3, 3-trifluoropropyl, 3,3, 3-trichloropropyl, 2,2, 3,3, 3-pentafluoropropyl, heptafluoropropyl, 1-(fluoromethyl)-2-fluoroethyl, 1-(chloromethyl)-2-chloroethyl, 1-(bromomethyl)-2-bromoethyl, 4-fluorobutyl, 4-chlorobutyl, 4-bromobutyl or nonafluorobutyl. According a term “C₁-C₂-fluoroalkyl” would refer to a C₁-C₂-alkyl radical which carries 1, 2, 3, 4, or 5 fluorine atoms, for example, any one of difluoromethyl, trifluoromethyl, 1-fluoroethyl, 2-fluoroethyl, 2, 2-difluoroethyl, 2,2, 2-trifluoroethyl, 1,1, 2, 2-tetrafluoroethyl or penta-fluoroethyl.

The term “C₁-Cₙalkoxy” as used herein refers to a straight-chain or branched saturated alkyl radical having 1 to n carbon atoms (as mentioned above) which is attached via an oxygen atom, i.e., for example, any one of methoxy, ethoxy, n-propoxy, 1-methylethoxy, n-butoxy, 1-methylpropoxy, 2-methylpropoxy or 1, 1-dimethylethoxy.

The term “C₁-Cₙhaloalkoxy” as used herein refers to a C₁-Cₙalkoxy radical as mentioned above which is partially or fully substituted by fluorine, chlorine, bromine and/or iodine, i.e., for example, any one of chloromethoxy, dichloromethoxy, trichloromethoxy, fluoromethoxy, difluoromethoxy, trifluoromethoxy, chlorofluoromethoxy, dichlorofluoromethoxy, chlorodifluoromethoxy, 2-fluoroethoxy, 2-chloroethoxy, 2-bromoethoxy, 2-iodoethoxy, 2, 2-difluoroethoxy, 2,2, 2-trifluoroethoxy, 2-chloro-2-fluoroethoxy, 2-chloro-2, 2-difluoroethoxy, 2, 2-dichloro-2-fluoroethoxy, 2,2, 2-trichloroethoxy, pentafluoroeth-oxy, 2-fluoropropoxy, 3-fluoropropoxy, 2, 2-difluoropropoxy, 2, 3-difluoropropoxy, 2-chloropropoxy, 3-chloropropoxy, 2, 3-dichloropropoxy, 2-bromopropoxy, 3-bromopropoxy, 3,3, 3-trifluoropropoxy, 3,3, 3-trichloropropoxy, 2,2, 3,3, 3-pentafluoropropoxy, heptafluoropropoxy, 1-(fluoromethyl)-2-fluoroethoxy, 1-(chloromethyl)-2-chloroethoxy, 1-(bromomethyl)-2-bromoethoxy, 4-fluorobutoxy, 4-chlorobutoxy, or 4-bromobutoxy.

The term “C₁-Cₙ-alkylsulfanyl” as used herein refers to a straight chain or branched saturated alkyl radical having 1 to n carbon atoms (as mentioned above) which is attached via a sulfur atom, i.e., for example, any one of methylthio, ethylthio, n-propylthio, 1-methylethylthio, butylthio, 1-methylpropylthio, 2-methylpropylthio or 1, 1-dimethylethylthio.

The term “C₁-Cₙalkylsulfinyl” as used herein refers to a straight chain or branched saturated alkyl radical having 1 to n carbon atoms (as mentioned above) which is attached via the sulfur atom of the sulfinyl group, i.e., for example, any one of methylsulfinyl, ethylsulfinyl, n-propylsulfinyl, 1-methylethyl-sulfinyl, n-butylsulfinyl, 1-methylpropylsulfinyl, 2-methylpropylsulfinyl, 1, 1-dimethyl-ethylsulfinyl, n-pentylsulfinyl, 1-methylbutylsulfinyl, 2-methylbutylsulfinyl, 3-methyl-butylsulfinyl, 1, 1-dimethylpropylsulfinyl, 1, 2-dimethylpropylsulfinyl, 2,2-dimethylpropylsulfinyl or 1-ethylpropylsulfinyl.

The term “C₁-Cₙalkylsulfonyl” as used herein refers to a straight chain or branched saturated alkyl radical having 1 to n carbon atoms (as mentioned above) which is attached via the sulfur atom of the sulfonyl group, i.e., for example, any one of methylsulfonyl, ethylsulfonyl, n-propylsulfonyl, isopropylsulfonyl, n-butylsulfonyl, 1-methylpropylsulfonyl, 2-methylpropylsulfonyl ort-butylsulphonyl.

The term “C₁-Cₙhaloalkylsulfanyl” as used herein refers to a C₁-Cₙalkylthio radical as mentioned above which is partially or fully substituted by fluorine, chlorine, bromine and/or iodine, i.e., for example, any one of fluoromethylthio, difluoromethylthio, trifluoromethylthio, chlorodifluoromethylthio, bromodifluoromethylthio, 2-fluoroethylthio, 2-chloroethylthio, 2-bromoethylthio, 2-iodoethylthio, 2, 2-difluoroethylthio, 2,2,2-trifluoroethylthio, 2,2, 2-trichloroethylthio, 2-chloro-2-fluoroethylthio, 2-chloro-2,2-difluoroethylthio, 2, 2-dichloro-2-fluoroethylthio, pentafluoroethylthio, 2-fluoropropylthio, 3-fluoropropylthio, 2-chloropropylthio, 3-chloropropylthio, 2-bromopropylthio, 3-bromopropylthio, 2,2-difluoropropylthio, 2,3-difluoropropylthio, 2, 3-dichloropropylthio, 3,3, 3-trifluoropropylthio, 3,3, 3-trichloropropylthio, 2,2, 3,3, 3-pentafluoropropylthio, heptafluoropropylthio, 1-(fluoromethyl)-2-fluoroethylthio, 1-(chloromethyl)-2-chloroethylthio, 1-(bromomethyl)-2-bromoethylthio, 4-fluorobutylthio, 4-chlorobutylthio, or 4-bromobutylthio.

The term “C₁-Cₙhaloalkylsulfinyl” and “C₁-Cₙhaloalkylsulfonyl” refers to the groups above but with the sulfur in oxidations state 1 or 2 respectively.

The term “C₁-Cₙcyanoalkyl” as used herein refers to a straight chain or branched saturated alkyl radicals having 1 to n carbon atoms (as mentioned above) which is substituted by a cyano group, for example cyanomethylene, cyanoethylene, 1,1-dimethylcyanomethyl, cyanomethyl, cyanoethyl, and 1-dimethylcyanomethyl.

The term “C₁-Cₙcyanoalkoxy” refers to the groups above but which is attached via an oxygen atom.

The suffix “—C₁-Cₙalkyl” after terms such as “C₃-Cₙcycloalkyl”, wherein n is an integer from 1-6, as used herein refers to a straight chain or branched saturated alkyl radicals which is substituted by C₃-Cₙcycloalkyl. An example of C₃-Cₙcycloalkyl-C₁-Cₙalkyl is for example, cyclopropylmethyl.

The term “C₃-C₆cycloalkyl” as used herein refers to 3-6 membered cycloylkyl groups such as cyclopropane, cyclobutane, cyclopropane, cyclopentane and cyclohexane.

Halogen is generally fluorine, chlorine, bromine or iodine. This also applies, correspondingly, to halogen in combination with other meanings, such as haloalkyl.

In the context of this invention “mono- or polysubstituted” in the definition of the Q₁ substituents, means typically, depending on the chemical structure of the substituents, monosubstituted to five-times substituted, more preferably mono-, double- or triple-substituted.

In the context of the this invention, the phrase “Q₁ is a five- to six-membered aromatic ring system, linked via a ring carbon atom . . . ” and the phrase “Q₁ is a five-membered aromatic ring system linked via a ring nitrogen atom . . . ”, as the case may be, refer to the manner of attachment of particular embodiments of the substituent Q₁ to the radical Q as represented by either formula Qa or formula Qb, which can be pyridyl or phenyl when A represents N or CH, respectively, as the case may be.

In the context of this invention, examples of “Q₁ is a five- to six-membered aromatic ring system, linked via a ring carbon atom . . . ; and said ring system can contain 1, 2 or 3 heteroatoms . . . ” are, but not limited to, phenyl, pyrazolyl, triazolyl, pyridinyl and pyrimidinyl; preferably phenyl, 2-pyridyl, 3-pyridyl, 4-pyridyl, pyrimidin-2-yl, pyrimidin-4-yl, and pyrimidin-5-yl.

In the context of this invention, examples of “Q₁ is a five-membered aromatic ring system linked via a ring nitrogen atom . . . ; and said ring system contains 1, 2 or 3 heteroatoms . . . ;” are, but not limited to, pyrazolyl, pyrrolyl, imidazolyl and triazolyl; preferably pyrrol-1-yl, pyrazol-1-yl, triazol-2-yl, 1,2,4-triazol-1-yl, triazol-1-yl, and imidazol-1-yl.

Certain embodiments according to the invention are provided as set out below.

Embodiment 1 provides compounds of formula I, or an agrochemically acceptable salt, stereoisomer, enantiomer, tautomer or N-oxide thereof, as defined above.

Embodiment 2 provides compounds, or an agrochemically acceptable salt, stereoisomer, enantiomer, tautomer or N-oxide thereof, according to embodiment 1 wherein Q is Qa and having preferred values of R₂, A, X, R₁, Q₁, R₄, R₅, G₁, G₂ and R₃ as set out below.

Embodiment 3 provides compounds, or an agrochemically acceptable salt, stereoisomer, enantiomer, tautomer or N-oxide thereof, according to embodiment 1 wherein Q is Qb and having preferred values of R₂, A, X, R₁, Q₁, R₄, R₅, G₁, G₂ and R₃ as set out below.

With respect to embodiments 1-3, preferred values of R₂, A, X, R₁, Q₁, R₄, R₅, G₁, G₂ and R₃ are, in any combination thereof, as set out below:

Preferably R₂ is C₁-C₆haloalkyl.

More preferably R₂ is C₁-C₆fluoroalkyl.

Most preferably R₂ is —CH₂CF₂CHF₂ or —CH₂CF₂CF₃.

Preferably A is N.

Preferably X is S or SO₂.

Most preferably X is SO₂.

Preferably R₁ is C₁-C₄alkyl or cyclopropyl-C₁-C₄alkyl.

More preferably R₁ is ethyl or cyclopropylmethyl.

Most preferably R₁ is ethyl.

Preferably Q₁ is hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂, —N(R₄)COR₅, or —N(R₄)CON(R₄)₂, (oxazolidin-2-one)-3-yl or 2-pyridyloxy; or

Q₁ is a five- to six-membered aromatic ring system linked via a ring carbon atom to the ring which contains the substituent A, said ring system is unsubstituted or is mono-substituted by a substituent selected from the group consisting of halogen, cyano and C₁-C₄haloalkyl; and said ring system can contain 1 or 2 ring nitrogen atoms; or
Q₁ is a five-membered aromatic ring system linked via a ring nitrogen atom to the ring which contains the substituent A, said ring system is unsubstituted or is mono-substituted by a substituent selected from the group consisting of halogen, cyano and C₁-C₄haloalkyl; and said ring system contains 2 or 3 ring nitrogen atoms.

More preferably Q₁ is hydrogen, halogen, trifluoromethyl, cyclopropyl, cyanocyclopropyl, cyanoisopropyl, trifluoroethoxy, —N(R₄)₂, —N(R₄)COR₅, or —N(R₄)CON(R₄)₂, in each of which R₄ is independently either hydrogen or methyl and R₅ is either methyl or cyclopropyl, or Q₁ is (oxazolidin-2-one)-3-yl, 2-pyridyloxy, N-linked pyrazolyl which is unsubstituted or is mono-substituted by chloro, cyano or trifluoromethyl; or Q₁ is N-linked triazolyl or C-linked pyrimidinyl.

Most preferably Q₁ is hydrogen, chlorine, bromine, trifluoromethyl, cyclopropyl, 1-cyanocyclopropyl, 1-cyano-1-methyl-ethyl, 2,2,2-trifluoroethoxy, —NH(CH₃), —N(CH₃)COCH₃, —N(CH₃)CO(cyclopropyl), —N(H)CONH(CH₃), —N(CH₃)CONH(CH₃), (oxazolidin-2-one)-3-yl, 2-pyridyloxy, pyrazol-1-yl, 3-chloro-pyrazol-1-yl, 3-cyano-pyrazol-1-yl, 3-trifluoromethyl-pyrazol-1-yl, 1,2,4-triazol-1-yl or pyrimidin-2-yl.

Preferably each R₄ is independently hydrogen or C₁-C₄alkyl.

Most preferably each R₄ is independently hydrogen or methyl.

Preferably R₅ is C₁-C₆alkyl or C₃-C₆cycloalkyl.

More preferably R₅ is methyl or cyclopropyl.

Most preferably R₅ is methyl.

Preferably either one of G₁ and G₂ is N.

Also preferred is when both G₁ and G₂ are CH.

Most preferably G₁ is CH and G₂ is N.

Preferably R₃ is hydrogen or C₁-C₄alkyl.

More preferably R₃ is hydrogen or methyl.

Most preferably R₃ is hydrogen.

One group of compounds according to the invention are those of formula I-1

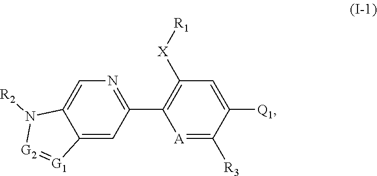

wherein A, X, R₁, R₂, G₁ and G₂ are as defined for compounds of formula I (above), or an agrochemically acceptable salt, stereoisomer, enantiomer, tautomer or N-oxide thereof, and wherein Q₁ is preferably hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂, —N(R₄)COR₅, or —N(R₄)CON(R₄)₂, (oxazolidin-2-one)-3-yl or 2-pyridyloxy; or
Q₁ is a five- to six-membered aromatic ring system linked via a ring carbon atom to the ring which contains the substituent A, said ring system is unsubstituted or is mono-substituted by a substituent selected from the group consisting of halogen, cyano and C₁-C₄haloalkyl; and said ring system can contain 1 or 2 ring nitrogen atoms; or
Q₁ is a five-membered aromatic ring system linked via a ring nitrogen atom to the ring which contains the substituent A, said ring system is unsubstituted or is mono-substituted by a substituent selected from the group consisting of halogen, cyano and C₁-C₄haloalkyl; and said ring system contains 2 or 3 ring nitrogen atoms;
R₃ is preferably hydrogen or C₁-C₄alkyl;
Preferably each R₄ is independently hydrogen or C₁-C₄alkyl; and
R₅ is preferably C₁-C₆alkyl or C₃-C₆cycloalkyl.

Preferred definitions of A, X, R₁, R₂, G₁ and G₂ are as defined for compounds of formula I (above), and more preferably Q₁ is hydrogen, halogen, trifluoromethyl, cyclopropyl, cyanocyclopropyl, cyanoisopropyl, trifluoroethoxy, —N(R₄)₂, —N(R₄)COR₅, or —N(R₄)CON(R₄)₂, in which each R₄ is independently either hydrogen or methyl and R₅ is either methyl or cyclopropyl, or Q₁ is (oxazolidin-2-one)-3-yl, 2-pyridyloxy, N-linked pyrazolyl which is unsubstituted or is mono-substituted by chloro, cyano or trifluoromethyl; or Q₁ is N-linked triazolyl or C-linked pyrimidinyl; and R₃ is hydrogen or methyl, preferably hydrogen.

One group of compounds according to this embodiment are compounds of formula (I-1a) which are compounds of formula (I-1) wherein A is N.

Another group of compounds according to this embodiment are compounds of formula (I-1 b) which are compounds of formula (I-1) wherein A is CH.

One group of compounds according to this embodiment are compounds of formula (I-1c) which are compounds of formula (I-1) wherein G₂ is N and G₁ is CH.

Another group of compounds according to this embodiment are compounds of formula (I-1d) which are compounds of formula (I-1) wherein G₂ and G₁ are both CH.

Another group of compounds according to this embodiment are compounds of formula (I-1e) which are compounds of formula (I-1) wherein G₂ is CH and G₁ is N.

Another group of compounds according to the invention are those of formula I-2

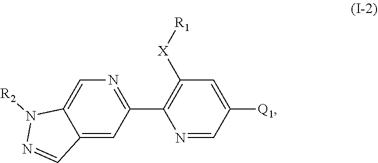

wherein X, R₁ and R₂ are as defined for compounds of formula I (above), or an agrochemically acceptable salt, stereoisomer, enantiomer, tautomer or N-oxide thereof, and wherein Q₁ is preferably hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂, —N(R₄)COR₅, or —N(R₄)CON(R₄)₂, (oxazolidin-2-one)-3-yl or 2-pyridyloxy; or
Q₁ is a five- to six-membered aromatic ring system linked via a ring carbon atom to the pyridyl ring substituted by X—R₁, said ring system is unsubstituted or is mono-substituted by a substituent selected from the group consisting of halogen, cyano and C₁-C₄haloalkyl; and said ring system can contain 1 or 2 ring nitrogen atoms; or
Q₁ is a five-membered aromatic ring system linked via a ring nitrogen atom to the pyridyl ring substituted by X—R₁, said ring system is unsubstituted or is mono-substituted by a substituent selected from the group consisting of halogen, cyano and C₁-C₄haloalkyl; and said ring system contains 2 or 3 ring nitrogen atoms;
Preferably each R₄ is independently hydrogen or C₁-C₄alkyl; and
R₅ is preferably C₁-C₆alkyl or C₃-C₆cycloalkyl.

Preferred definitions of X, R₁ and R₂ are as defined for compounds of formula I (above), and more preferably Q₁ is hydrogen, halogen, trifluoromethyl, cyclopropyl, cyanocyclopropyl, cyanoisopropyl, trifluoroethoxy, —N(R₄)₂, —N(R₄)COR₅, or —N(R₄)CON(R₄)₂, in which each R₄ is independently either hydrogen or methyl and R₅ is either methyl or cyclopropyl, or Q₁ is (oxazolidin-2-one)-3-yl, 2-pyridyloxy, N-linked pyrazolyl which is unsubstituted or is mono-substituted by chloro, cyano or trifluoromethyl; or Q₁ is N-linked triazolyl or C-linked pyrimidinyl; even more preferably Q₁ is hydrogen, halogen, trifluoromethyl, cyclopropyl, cyanocyclopropyl, cyanoisopropyl, —N(R₄)₂, —N(R₄)COR₅, or —N(R₄)CON(R₄)₂, in which each R₄ is independently either hydrogen or methyl and R₅ is either methyl or cyclopropyl, or Q₁ is (oxazolidin-2-one)-3-yl, 2-pyridyloxy, N-linked pyrazolyl which is mono-substituted by chloro or trifluoromethyl; or Q₁ is N-linked triazolyl or C-linked pyrimidinyl.

One group of compounds according to this embodiment are compounds of formula (I-2a) which are compounds of formula (I-2) wherein X is S or SO₂, preferably SO₂.

Another group of compounds according to this embodiment are compounds of formula (I-2b-1) which are compounds of formula (I-2) wherein R₁ is C₁-C₄alkyl or cyclopropyl-C₁-C₄alkyl, preferably ethyl or cyclopropylmethyl.

Another group of compounds according to this embodiment are compounds of formula (I-2b-2) which are compounds of formula (I-2) wherein R₁ is C₁-C₄alkyl, preferably ethyl.

Another group of compounds according to this embodiment are compounds of formula (I-2c) which are compounds of formula (I-2) wherein R₂ is C₁-C₆haloalkyl, more preferably C₁-C₆fluoroalkyl, most preferably —CH₂CF₂CHF₂ or —CH₂CF₂CF₃.

Another group of compounds according to the invention are those of formula I-3

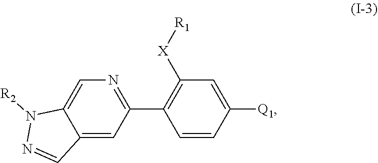

wherein X, R₁ and R₂ are as defined for compounds of formula I (above), or an agrochemically acceptable salt, stereoisomer, enantiomer, tautomer or N-oxide thereof, and wherein Q₁ is preferably hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂, —N(R₄)COR₅, or —N(R₄)CON(R₄)₂, (oxazolidin-2-one)-3-yl or 2-pyridyloxy; or
Q₁ is a five- to six-membered aromatic ring system linked via a ring carbon atom to the phenyl ring substituted by X—R₁, said ring system is unsubstituted or is mono-substituted by a substituent selected from the group consisting of halogen, cyano and C₁-C₄haloalkyl; and said ring system can contain 1 or 2 ring nitrogen atoms; or
Q₁ is a five-membered aromatic ring system linked via a ring nitrogen atom to the phenyl ring substituted by X—R₁, said ring system is unsubstituted or is mono-substituted by a substituent selected from the group consisting of halogen, cyano and C₁-C₄haloalkyl; and said ring system contains 2 or 3 ring nitrogen atoms;
Preferably each R₄ is independently hydrogen or C₁-C₄alkyl; and
R₅ is preferably C₁-C₆alkyl or C₃-C₆cycloalkyl.

Preferred definitions of X, R₁ and R₂ are as defined for compounds of formula I (above), and more preferably Q₁ is hydrogen, halogen, trifluoromethyl, cyclopropyl, cyanocyclopropyl, cyanoisopropyl, trifluoroethoxy, —N(R₄)₂, —N(R₄)COR₅, or —N(R₄)CON(R₄)₂, in which each R₄ is independently either hydrogen or methyl and R₅ is either methyl or cyclopropyl, or Q₁ is (oxazolidin-2-one)-3-yl, 2-pyridyloxy, N-linked pyrazolyl which is unsubstituted or is mono-substituted by chloro, cyano or trifluoromethyl; or Q₁ is N-linked triazolyl or C-linked pyrimidinyl; even more preferably Q₁ is hydrogen, halogen, trifluoromethyl, cyclopropyl, cyanocyclopropyl, cyanoisopropyl, —N(R₄)₂, —N(R₄)COR₅, or —N(R₄)CON(R₄)₂, in which each R₄ is independently either hydrogen or methyl and R₅ is either methyl or cyclopropyl, or Q₁ is (oxazolidin-2-one)-3-yl, 2-pyridyloxy, N-linked pyrazolyl which is mono-substituted by chloro or trifluoromethyl; or Q₁ is N-linked triazolyl or C-linked pyrimidinyl.

One group of compounds according to this embodiment are compounds of formula (I-3a) which are compounds of formula (I-3) wherein X is S or SO₂, preferably SO₂.

Another group of compounds according to this embodiment are compounds of formula (I-3b-1) which are compounds of formula (I-3) wherein R₁ is C₁-C₄alkyl or cyclopropyl-C₁-C₄alkyl, preferably ethyl or cyclopropylmethyl.

Another group of compounds according to this embodiment are compounds of formula (I-3b-2) which are compounds of formula (I-3) wherein R₁ is C₁-C₄alkyl, preferably ethyl.

Another group of compounds according to this embodiment are compounds of formula (I-3c) which are compounds of formula (I-3) wherein R₂ is C₁-C₆haloalkyl, more preferably C₁-C₆fluoroalkyl, most preferably —CH₂CF₂CHF₂ or —CH₂CF₂CF₃.

Another group of compounds according to the invention are those of formula I-4

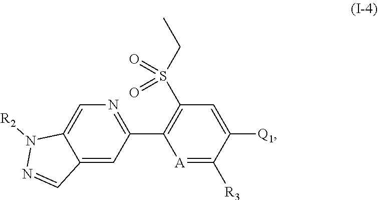

wherein
A is CH or N, preferably N;
R₂ is C₁-C₆haloalkyl, preferably —CH₂CF₂CHF₂ or —CH₂CF₂CF₃;
R₃ is hydrogen or C₁-C₄alkyl, preferably hydrogen or methyl;
Q₁ is hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂, —N(R₄)COR₅, or —N(R₄)CON(R₄)₂, (oxazolidin-2-one)-3-yl or 2-pyridyloxy; or
Q₁ is a five- to six-membered aromatic ring system linked via a ring carbon atom to the ring which contains the substituent A, said ring system is unsubstituted or is mono-substituted by a substituent selected from the group consisting of halogen, cyano and C₁-C₄haloalkyl; and said ring system can contain 1 or 2 ring nitrogen atoms; or
Q₁ is a five-membered aromatic ring system linked via a ring nitrogen atom to the ring which contains the substituent A, said ring system is unsubstituted or is mono-substituted by a substituent selected from the group consisting of halogen, cyano and C₁-C₄haloalkyl; and said ring system contains 2 or 3 ring nitrogen atoms;
Each R₄ is independently hydrogen or C₁-C₄alkyl, preferably hydrogen or methyl; and
R₅ is C₁-C₆alkyl or C₃-C₆cycloalkyl, preferably methyl or cyclopropyl; or
an agrochemically acceptable salt, stereoisomer, enantiomer, tautomer or N-oxide thereof.

One further preferred group of compounds according to this embodiment are compounds of formula (I-4a) which are compounds of formula (I-4) wherein A is N.

Another preferred group of compounds according to this embodiment are compounds of formula (I-4b) which are compounds of formula (I-4) wherein A is CH.

One further preferred group of compounds according to this embodiment are compounds of formula (I-4c) which are compounds of formula (I-4) wherein R₃ is hydrogen.

Another preferred group of compounds according to this embodiment are compounds of formula (I-4d) which are compounds of formula (I-4) wherein R₃ is C₁-C₄alkyl, preferably methyl.

One further preferred group of compounds according to this embodiment are compounds of formula (I-4e) which are compounds of formula (I-4) wherein A is N and R₃ is hydrogen.

One further preferred group of compounds according to this embodiment are compounds of formula (I-4f) which are compounds of formula (I-4) wherein Q₁ is hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂ or —N(R₄)COR₅, both in which R₄ is independently hydrogen or C₁-C₄alkyl (preferably hydrogen or methyl) and R₅ is C₁-C₆alkyl (preferably methyl), or Q₁ is 2-pyridyloxy; preferably Q₁ is hydrogen, halogen, trifluoromethyl, cyclopropyl, cyanocyclopropyl, cyanoisopropyl, trifluoroethoxy, —N(R₄)₂ or —N(R₄)COCH₃, both in which R₄ is independently hydrogen or methyl, or Q₁ is 2-pyridyloxy; or

Q₁ is a five-membered aromatic ring system linked via a ring nitrogen atom to the ring which contains the substituent A, said ring system is unsubstituted or is mono-substituted by a substituent selected from the group consisting of halogen, cyano and C₁-C₄haloalkyl; and said ring system contains 2 ring nitrogen atoms; preferably Q₁ is N-linked pyrazolyl which is unsubstituted or is mono-substituted by chloro, cyano or trifluoromethyl.

One further preferred group of compounds according to this embodiment are compounds of formula (I-4g) which are compounds of formula (I-4) wherein Q₁ is hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂ or —N(R₄)COR₅, both in which R₄ is independently hydrogen or C₁-C₄alkyl (preferably hydrogen or methyl) and R₅ is C₁-C₆alkyl (preferably methyl), 2-pyridyloxy or N-linked pyrazolyl which is unsubstituted or is mono-substituted by chloro, cyano or trifluoromethyl; preferably Q₁ is hydrogen, chlorine, bromine, trifluoromethyl, cyclopropyl, 1-cyanocyclopropyl, 1-cyano-1-methyl-ethyl, 2,2,2-trifluoroethoxy, —NH₂, —NH(CH₃), —N(CH₃)₂, —NHCOCH₃, —N(CH₃)COCH₃, 2-pyridyloxy, pyrazol-1-yl, 3-chloro-pyrazol-1-yl, 3-cyano-pyrazol-1-yl or 3-trifluoromethyl-pyrazol-1-yl.

One further preferred group of compounds according to this embodiment are compounds of formula (I-4g-1) which are compounds of formula (I-4) wherein Q₁ is hydrogen, C₁-C₆haloalkyl, C₃-C₆cycloalkyl, C₃-C₆cycloalkyl monosubstituted by cyano, C₁-C₆cyanoalkyl, —N(R₄)₂ or —N(R₄)COR₅, both in which R₄ is independently hydrogen or C₁-C₄alkyl (preferably hydrogen or methyl) and R₅ is C₁-C₆alkyl (preferably methyl), 2-pyridyloxy or N-linked pyrazolyl which is mono-substituted by chloro or trifluoromethyl; preferably Q₁ is hydrogen, trifluoromethyl, cyclopropyl, 1-cyanocyclopropyl, 1-cyano-1-methyl-ethyl, —NH(CH₃), —N(CH₃)COCH₃, 2-pyridyloxy, 3-chloro-pyrazol-1-yl or 3-trifluoromethyl-pyrazol-1-yl.

One further preferred group of compounds according to this embodiment are compounds of formula (I-4h) which are compounds of formula (I-4) wherein

### A is N;

R₂ is C₁-C₆haloalkyl, preferably —CH₂CF₂CHF₂ or —CH₂CF₂CF₃;
R₃ is hydrogen or C₁-C₄alkyl, preferably hydrogen or methyl; and
Q₁ is hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂ or —N(R₄)COR₅, both in which R₄ is independently hydrogen or C₁-C₄alkyl (preferably hydrogen or methyl) and R₅ is C₁-C₆alkyl (preferably methyl), or Q₁ is 2-pyridyloxy; preferably Q₁ is hydrogen, halogen, trifluoromethyl, cyclopropyl, cyanocyclopropyl, cyanoisopropyl, trifluoroethoxy, —N(R₄)₂ or —N(R₄)COCH₃, both in which R₄ is independently hydrogen or methyl, or Q₁ is 2-pyridyloxy; or
Q₁ is a five-membered aromatic ring system linked via a ring nitrogen atom to the ring which contains the substituent A, said ring system is unsubstituted or is mono-substituted by a substituent selected from the group consisting of halogen, cyano and C₁-C₄haloalkyl; and said ring system contains 2 ring nitrogen atoms; preferably Q₁ is N-linked pyrazolyl which is unsubstituted or is mono-substituted by chloro, cyano or trifluoromethyl.

Another preferred group of compounds according to this embodiment are compounds of formula (I-4i) which are compounds of formula (I-4) wherein

### A is N;

R₂ is C₁-C₆haloalkyl, preferably —CH₂CF₂CHF₂ or —CH₂CF₂CF₃;
R₃ is hydrogen; and
Q₁ is hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂ or —N(R₄)COR₅, both in which R₄ is independently hydrogen or C₁-C₄alkyl (preferably hydrogen or methyl) and R₅ is C₁-C₆alkyl (preferably methyl), 2-pyridyloxy or N-linked pyrazolyl which is unsubstituted or is mono-substituted by chloro, cyano or trifluoromethyl;
preferably Q₁ is hydrogen, chlorine, bromine, trifluoromethyl, cyclopropyl, 1-cyanocyclopropyl, 1-cyano-1-methyl-ethyl, 2,2,2-trifluoroethoxy, —NH₂, —NH(CH₃), —N(CH₃)₂, —NHCOCH₃, —N(CH₃)COCH₃, 2-pyridyloxy, pyrazol-1-yl, 3-chloro-pyrazol-1-yl, 3-cyano-pyrazol-1-yl or 3-trifluoromethyl-pyrazol-1-yl.

Another preferred group of compounds according to this embodiment are compounds of formula (I-4i-1) which are compounds of formula (I-4) wherein

### A is N;

R₂ is C₁-C₆haloalkyl, preferably —CH₂CF₂CHF₂ or —CH₂CF₂CF₃, more preferably —CH₂CF₂CF₃;
R₃ is hydrogen; and
Q₁ is hydrogen, C₁-C₆haloalkyl, C₃-C₆cycloalkyl, C₃-C₆cycloalkyl monosubstituted by cyano, C₁-C₆cyanoalkyl, —N(R₄)₂ or —N(R₄)COR₅, both in which R₄ is independently hydrogen or C₁-C₄alkyl (preferably hydrogen or methyl) and R₅ is C₁-C₆alkyl (preferably methyl), 2-pyridyloxy or N-linked pyrazolyl which is mono-substituted by chloro or trifluoromethyl; preferably Q₁ is hydrogen, trifluoromethyl, cyclopropyl, 1-cyanocyclopropyl, 1-cyano-1-methyl-ethyl, —NH(CH₃), —N(CH₃)COCH₃, 2-pyridyloxy, 3-chloro-pyrazol-1-yl or 3-trifluoromethyl-pyrazol-1-yl.

Another preferred group of compounds according to this embodiment are compounds of formula (I-4j) which are compounds of formula (I-4) wherein

### A is N;

R₂ is C₁-C₆haloalkyl, preferably —CH₂CF₂CHF₂ or —CH₂CF₂CF₃;
R₃ is hydrogen; and
Q₁ is hydrogen, halogen, C₃-C₆cycloalkyl, C₁-C₆cyanoalkyl, or —N(R₄)COR₅ in which R₄ is hydrogen or C₁-C₄alkyl (preferably hydrogen or methyl) and R₅ is C₁-C₆alkyl (preferably methyl); preferably Q₁ is hydrogen, chlorine, bromine, cyclopropyl, 1-cyano-1-methyl-ethyl, or —N(CH₃)COCH₃.

Another preferred group of compounds according to this embodiment are compounds of formula (I-4k) which are compounds of formula (I-4) wherein

### A is N;

R₂ is C₁-C₆haloalkyl, preferably —CH₂CF₂CHF₂ or —CH₂CF₂CF₃;
R₃ is C₁-C₄alkyl, preferably methyl; and
Q₁ is hydrogen, halogen, C₃-C₆cycloalkyl, C₁-C₆cyanoalkyl, or —N(R₄)COR₅ in which R₄ is hydrogen or C₁-C₄alkyl (preferably hydrogen or methyl) and R₅ is C₁-C₆alkyl (preferably methyl); preferably Q₁ is hydrogen, chlorine, bromine, cyclopropyl, 1-cyano-1-methyl-ethyl, or —N(CH₃)COCH₃.

One group of compounds according to the invention are those of formula I-5

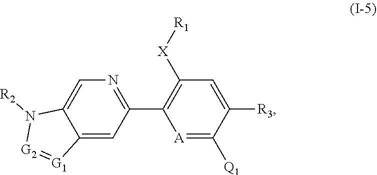

wherein A, X, R₁, R₂, G₁ and G₂ are as defined for compounds of formula I (above), or an agrochemically acceptable salt, stereoisomer, enantiomer, tautomer or N-oxide thereof, and wherein Q₁ is preferably hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂, —N(R₄)COR₅, or —N(R₄)CON(R₄)₂, (oxazolidin-2-one)-3-yl or 2-pyridyloxy; or
Q₁ is a five- to six-membered aromatic ring system linked via a ring carbon atom to the ring which contains the substituent A, said ring system is unsubstituted or is mono-substituted by a substituent selected from the group consisting of halogen, cyano and C₁-C₄haloalkyl; and said ring system can contain 1 or 2 ring nitrogen atoms; or
Q₁ is a five-membered aromatic ring system linked via a ring nitrogen atom to the ring which contains the substituent A, said ring system is unsubstituted or is mono-substituted by a substituent selected from the group consisting of halogen, cyano and C₁-C₄haloalkyl; and said ring system contains 2 or 3 ring nitrogen atoms;
R₃ is preferably hydrogen or C₁-C₄alkyl;
Preferably each R₄ is independently hydrogen or C₁-C₄alkyl; and
R₅ is preferably C₁-C₆alkyl or C₃-C₆cycloalkyl.

Preferred definitions of A, X, R₁, R₂, G₁ and G₂ are as defined for compounds of formula I (above), and more preferably Q₁ is hydrogen, halogen, trifluoromethyl, cyclopropyl, cyanocyclopropyl, cyanoisopropyl, trifluoroethoxy, —N(R₄)₂, —N(R₄)COR₅, or —N(R₄)CON(R₄)₂, in which each R₄ is independently either hydrogen or methyl and R₅ is either methyl or cyclopropyl, or Q₁ is (oxazolidin-2-one)-3-yl, 2-pyridyloxy, N-linked pyrazolyl which is unsubstituted or is mono-substituted by chloro, cyano or trifluoromethyl; or Q₁ is N-linked triazolyl or C-linked pyrimidinyl; and R₃ is hydrogen or methyl, preferably hydrogen.

One group of compounds according to this embodiment are compounds of formula (I-5a) which are compounds of formula (I-5) wherein A is N.

Another group of compounds according to this embodiment are compounds of formula (I-5b) which are compounds of formula (I-5) wherein A is CH.

One group of compounds according to this embodiment are compounds of formula (I-5c) which are compounds of formula (I-5) wherein G₂ is N and G₁ is CH.

Another group of compounds according to this embodiment are compounds of formula (I-5d) which are compounds of formula (I-5) wherein G₂ and G₁ are both CH.

Another group of compounds according to this embodiment are compounds of formula (I-5e) which are compounds of formula (I-5) wherein G₂ is CH and G₁ is N.

Another group of compounds according to the invention are those of formula I-6

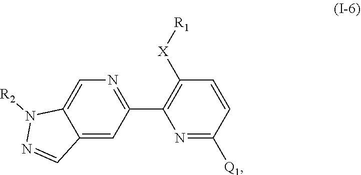

wherein X, R₁ and R₂ are as defined for compounds of formula I (above), or an agrochemically acceptable salt, stereoisomer, enantiomer, tautomer or N-oxide thereof, and wherein Q₁ is preferably hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂, —N(R₄)COR₅, or —N(R₄)CON(R₄)₂, (oxazolidin-2-one)-3-yl or 2-pyridyloxy; or
Q₁ is a five- to six-membered aromatic ring system linked via a ring carbon atom to the pyridyl ring substituted by X—R₁, said ring system is unsubstituted or is mono-substituted by a substituent selected from the group consisting of halogen, cyano and C₁-C₄haloalkyl; and said ring system can contain 1 or 2 ring nitrogen atoms; or
Q₁ is a five-membered aromatic ring system linked via a ring nitrogen atom to the pyridyl ring substituted by X—R₁, said ring system is unsubstituted or is mono-substituted by a substituent selected from the group consisting of halogen, cyano and C₁-C₄haloalkyl; and said ring system contains 2 or 3 ring nitrogen atoms;
Preferably each R₄ is independently hydrogen or C₁-C₄alkyl; and
R₅ is preferably C₁-C₆alkyl or C₃-C₆cycloalkyl.

Preferred definitions of X, R₁ and R₂ are as defined for compounds of formula I (above), and more preferably Q₁ is hydrogen, halogen, trifluoromethyl, cyclopropyl, cyanocyclopropyl, cyanoisopropyl, trifluoroethoxy, —N(R₄)₂, —N(R₄)COR₅, or —N(R₄)CON(R₄)₂, in which each R₄ is independently either hydrogen or methyl and R₅ is either methyl or cyclopropyl, or Q₁ is (oxazolidin-2-one)-3-yl, 2-pyridyloxy, N-linked pyrazolyl which is unsubstituted or is mono-substituted by chloro, cyano or trifluoromethyl; or Q₁ is N-linked triazolyl or C-linked pyrimidinyl; even more preferably Q₁ is hydrogen, halogen, trifluoromethyl, cyclopropyl, cyanocyclopropyl, cyanoisopropyl, —N(R₄)₂, —N(R₄)COR₅, or —N(R₄)CON(R₄)₂, in which each R₄ is independently either hydrogen or methyl and R₅ is either methyl or cyclopropyl, or Q₁ is (oxazolidin-2-one)-3-yl, 2-pyridyloxy, N-linked pyrazolyl which is mono-substituted by chloro or trifluoromethyl; or Q₁ is N-linked triazolyl or C-linked pyrimidinyl.

One group of compounds according to this embodiment are compounds of formula (I-6a) which are compounds of formula (I-6) wherein X is S or SO₂, preferably SO₂.

Another group of compounds according to this embodiment are compounds of formula (I-6b-1) which are compounds of formula (I-6) wherein R₁ is C₁-C₄alkyl or cyclopropyl-C₁-C₄alkyl, preferably ethyl or cyclopropylmethyl.

Another group of compounds according to this embodiment are compounds of formula (I-6b-2) which are compounds of formula (I-6) wherein R₁ is C₁-C₄alkyl, preferably ethyl.

Another group of compounds according to this embodiment are compounds of formula (I-6c) which are compounds of formula (I-6) wherein R₂ is C₁-C₆haloalkyl, more preferably C₁-C₆fluoroalkyl, most preferably —CH₂CF₂CHF₂ or —CH₂CF₂CF₃.

Another group of compounds according to the invention are those of formula I-7

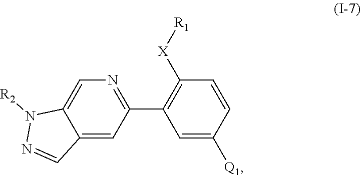

wherein X, R₁ and R₂ are as defined for compounds of formula I (above), or an agrochemically acceptable salt, stereoisomer, enantiomer, tautomer or N-oxide thereof, and wherein Q₁ is preferably hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂, —N(R₄)COR₅, or —N(R₄)CON(R₄)₂, (oxazolidin-2-one)-3-yl or 2-pyridyloxy; or
Q₁ is a five- to six-membered aromatic ring system linked via a ring carbon atom to the phenyl ring substituted by X—R₁, said ring system is unsubstituted or is mono-substituted by a substituent selected from the group consisting of halogen, cyano and C₁-C₄haloalkyl; and said ring system can contain 1 or 2 ring nitrogen atoms; or
Q₁ is a five-membered aromatic ring system linked via a ring nitrogen atom to the phenyl ring substituted by X—R₁, said ring system is unsubstituted or is mono-substituted by a substituent selected from the group consisting of halogen, cyano and C₁-C₄haloalkyl; and said ring system contains 2 or 3 ring nitrogen atoms;
Preferably each R₄ is independently hydrogen or C₁-C₄alkyl; and
R₅ is preferably C₁-C₆alkyl or C₃-C₆cycloalkyl.

Preferred definitions of X, R₁ and R₂ are as defined for compounds of formula I (above), and more preferably Q₁ is hydrogen, halogen, trifluoromethyl, cyclopropyl, cyanocyclopropyl, cyanoisopropyl, trifluoroethoxy, —N(R₄)₂, —N(R₄)COR₅, or —N(R₄)CON(R₄)₂, in which each R₄ is independently either hydrogen or methyl and R₅ is either methyl or cyclopropyl, or Q₁ is (oxazolidin-2-one)-3-yl, 2-pyridyloxy, N-linked pyrazolyl which is unsubstituted or is mono-substituted by chloro, cyano or trifluoromethyl; or Q₁ is N-linked triazolyl or C-linked pyrimidinyl; even more preferably Q₁ is hydrogen, halogen, trifluoromethyl, cyclopropyl, cyanocyclopropyl, cyanoisopropyl, —N(R₄)₂, —N(R₄)COR₅, or —N(R₄)CON(R₄)₂, in which each R₄ is independently either hydrogen or methyl and R₅ is either methyl or cyclopropyl, or Q₁ is (oxazolidin-2-one)-3-yl, 2-pyridyloxy, N-linked pyrazolyl which is mono-substituted by chloro or trifluoromethyl; or Q₁ is N-linked triazolyl or C-linked pyrimidinyl.

One group of compounds according to this embodiment are compounds of formula (I-7a) which are compounds of formula (I-7) wherein X is S or SO₂, preferably SO₂.

Another group of compounds according to this embodiment are compounds of formula (I-7b-1) which are compounds of formula (I-7) wherein R₁ is C₁-C₄alkyl or cyclopropyl-C₁-C₄alkyl, preferably ethyl or cyclopropylmethyl.

Another group of compounds according to this embodiment are compounds of formula (I-7b-2) which are compounds of formula (I-7) wherein R₁ is C₁-C₄alkyl, preferably ethyl.

Another group of compounds according to this embodiment are compounds of formula (I-7c) which are compounds of formula (I-7) wherein R₂ is C₁-C₆haloalkyl, more preferably C₁-C₆fluoroalkyl, most preferably —CH₂CF₂CHF₂ or —CH₂CF₂CF₃.

Another group of compounds according to the invention are those of formula I-8

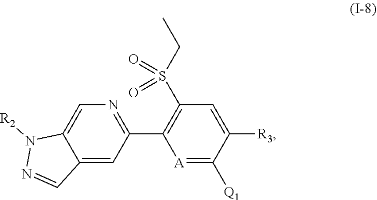

wherein
A is CH or N, preferably N;
R₂ is C₁-C₆haloalkyl, preferably —CH₂CF₂CHF₂ or —CH₂CF₂CF₃;
R₃ is hydrogen or C₁-C₄alkyl, preferably hydrogen or methyl;
Q₁ is hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂, —N(R₄)COR₅, or —N(R₄)CON(R₄)₂, (oxazolidin-2-one)-3-yl or 2-pyridyloxy; or
Q₁ is a five- to six-membered aromatic ring system linked via a ring carbon atom to the ring which contains the substituent A, said ring system is unsubstituted or is mono-substituted by a substituent selected from the group consisting of halogen, cyano and C₁-C₄haloalkyl; and said ring system can contain 1 or 2 ring nitrogen atoms; or
Q₁ is a five-membered aromatic ring system linked via a ring nitrogen atom to the ring which contains the substituent A, said ring system is unsubstituted or is mono-substituted by a substituent selected from the group consisting of halogen, cyano and C₁-C₄haloalkyl; and said ring system contains 2 or 3 ring nitrogen atoms;
Each R₄ is independently hydrogen or C₁-C₄alkyl, preferably hydrogen or methyl; and
R₅ is C₁-C₆alkyl or C₃-C₆cycloalkyl, preferably methyl or cyclopropyl; or
an agrochemically acceptable salt, stereoisomer, enantiomer, tautomer or N-oxide thereof.

One further preferred group of compounds according to this embodiment are compounds of formula (I-8a) which are compounds of formula (I-8) wherein A is N.

Another preferred group of compounds according to this embodiment are compounds of formula (I-8b) which are compounds of formula (I-8) wherein A is CH.

One further preferred group of compounds according to this embodiment are compounds of formula (I-8c) which are compounds of formula (I-8) wherein R₃ is hydrogen.

Another preferred group of compounds according to this embodiment are compounds of formula (I-8d) which are compounds of formula (I-8) wherein R₃ is C₁-C₄alkyl, preferably methyl.

One further preferred group of compounds according to this embodiment are compounds of formula (I-8e) which are compounds of formula (I-8) wherein A is N and R₃ is hydrogen.

One further preferred group of compounds according to this embodiment are compounds of formula (I-8f) which are compounds of formula (I-8) wherein Q₁ is hydrogen, halogen, C₃-C₆cycloalkyl, —N(R₄)₂, —N(R₄)COR₅ or —N(R₄)CON(R₄)₂, in each of which R₄ is independently either hydrogen or C₁-C₄alkyl (preferably hydrogen or methyl) and R₅ is C₁-C₆alkyl or C₃-C₆cycloalkyl (preferably methyl or cyclopropyl), or Q₁ is (oxazolidin-2-one)-3-yl; preferably Q₁ is hydrogen, halogen, cyclopropyl, —N(R₄)₂, —N(R₄)COR₅ or —N(R₄)CON(R₄)₂, in each of which R₄ is independently either hydrogen or methyl and R₅ is methyl or cyclopropyl, or Q₁ is (oxazolidin-2-one)-3-yl; or

Q₁ is a six-membered aromatic ring system linked via a ring carbon atom to the ring which contains the substituent A, said ring system is unsubstituted or is mono-substituted by a substituent selected from the group consisting of halogen, cyano and C₁-C₄haloalkyl; and said ring system contains 2 ring nitrogen atoms; preferably Q₁ is pyrimidinyl which is unsubstituted or is mono-substituted by chloro, cyano or trifluoromethyl; or
Q₁ is a five-membered aromatic ring system linked via a ring nitrogen atom to the ring which contains the substituent A, said ring system is unsubstituted or is mono-substituted by a substituent selected from the group consisting of halogen, cyano and C₁-C₄haloalkyl; and said ring system contains 3 ring nitrogen atoms; preferably Q₁ is N-linked triazolyl which is unsubstituted or is mono-substituted by chloro, cyano or trifluoromethyl.

One further preferred group of compounds according to this embodiment are compounds of formula (I-8g) which are compounds of formula (I-8) wherein Q₁ is hydrogen, halogen, C₃-C₆cycloalkyl, —N(R₄)₂, —N(R₄)COR₅ or —N(R₄)CON(R₄)₂, in each of which R₄ is independently either hydrogen or C₁-C₄alkyl (preferably hydrogen or methyl) and R₅ is C₁-C₆alkyl or C₃-C₆cycloalkyl (preferably methyl or cyclopropyl); or Q₁ is (oxazolidin-2-one)-3-yl, C-linked pyrimidinyl or N-linked triazolyl; preferably Q₁ is hydrogen, chlorine, bromine, cyclopropyl, —NH₂, —NH(CH₃), —NHCOCH₃, —N(CH₃)COCH₃, —NHCO(cyclopropyl), —N(CH₃)CO(cyclopropyl), —N(H)CONH₂, —N(H)CONH(CH₃), —N(H)CON(CH₃)₂, —N(CH₃)CONH₂, —N(CH₃)CONH(CH₃), —N(CH₃)CON(CH₃)₂, (oxazolidin-2-one)-3-yl, pyrimidin-2-yl or 1,2,4-triazol-1-yl; more preferably Q₁ is hydrogen, chlorine, cyclopropyl, —NH(CH₃), —N(CH₃)COCH₃, —N(CH₃)CO(cyclopropyl), —N(CH₃)CONH(CH₃), (oxazolidin-2-one)-3-yl, pyrimidin-2-yl or 1,2,4-triazol-1-yl.

One further preferred group of compounds according to this embodiment are compounds of formula (I-8h) which are compounds of formula (I-8) wherein

### A is N

R₂ is C₁-C₆haloalkyl, preferably —CH₂CF₂CHF₂ or —CH₂CF₂CF₃;
R₃ is hydrogen or C₁-C₄alkyl, preferably hydrogen or methyl; and
Q₁ is hydrogen, halogen, C₃-C₆cycloalkyl, —N(R₄)₂, —N(R₄)COR₅ or —N(R₄)CON(R₄)₂, in each of which R₄ is independently either hydrogen or C₁-C₄alkyl (preferably hydrogen or methyl) and R₅ is C₁-C₆alkyl or C₃-C₆cycloalkyl (preferably methyl or cyclopropyl), or Q₁ is (oxazolidin-2-one)-3-yl; preferably Q₁ is hydrogen, halogen, cyclopropyl, —N(R₄)₂, —N(R₄)COR₅ or —N(R₄)CON(R₄)₂, in each of which R₄ is independently either hydrogen or methyl and R₅ is methyl or cyclopropyl, or Q₁ is (oxazolidin-2-one)-3-yl; or
Q₁ is a six-membered aromatic ring system linked via a ring carbon atom to the ring which contains the substituent A, said ring system is unsubstituted or is mono-substituted by a substituent selected from the group consisting of halogen, cyano and C₁-C₄haloalkyl; and said ring system contains 2 ring nitrogen atoms; preferably Q₁ is pyrimidinyl which is unsubstituted or is mono-substituted by chloro, cyano or trifluoromethyl; or
Q₁ is a five-membered aromatic ring system linked via a ring nitrogen atom to the ring which contains the substituent A, said ring system is unsubstituted or is mono-substituted by a substituent selected from the group consisting of halogen, cyano and C₁-C₄haloalkyl; and said ring system contains 3 ring nitrogen atoms; preferably Q₁ is N-linked triazolyl which is unsubstituted or is mono-substituted by chloro, cyano or trifluoromethyl.

Another preferred group of compounds according to this embodiment are compounds of formula (I-8i) which are compounds of formula (I-8) wherein

### A is N;

R₂ is C₁-C₆haloalkyl, preferably —CH₂CF₂CHF₂ or —CH₂CF₂CF₃;
R₃ is hydrogen; and
Q₁ is hydrogen, halogen, C₃-C₆cycloalkyl, —N(R₄)₂, —N(R₄)COR₅ or —N(R₄)CON(R₄)₂, in each of which R₄ is independently either hydrogen or C₁-C₄alkyl (preferably hydrogen or methyl) and R₅ is C₁-C₆alkyl or C₃-C₆cycloalkyl (preferably methyl or cyclopropyl); or Q₁ is (oxazolidin-2-one)-3-yl, C-linked pyrimidinyl or N-linked triazolyl; preferably Q₁ is hydrogen, chlorine, bromine, cyclopropyl, —NH₂, —NH(CH₃), —NHCOCH₃, —N(CH₃)COCH₃, —NHCO(cyclopropyl), —N(CH₃)CO(cyclopropyl), —N(H)CONH₂, —N(H)CONH(CH₃), —N(H)CON(CH₃)₂, —N(CH₃)CONH₂, —N(CH₃)CONH(CH₃), —N(CH₃)CON(CH₃)₂, (oxazolidin-2-one)-3-yl, pyrimidin-2-yl or 1,2,4-triazol-1-yl; more preferably Q₁ is hydrogen, chlorine, cyclopropyl, —NH(CH₃), —N(CH₃)COCH₃, —N(CH₃)CO(cyclopropyl), —N(CH₃)CONH(CH₃), (oxazolidin-2-one)-3-yl, pyrimidin-2-yl or 1,2,4-triazol-1-yl.

Another preferred group of compounds according to this embodiment are compounds of formula (I-8j) which are compounds of formula (I-8) wherein

### A is N;

R₂ is C₁-C₆haloalkyl, preferably —CH₂CF₂CHF₂ or —CH₂CF₂CF₃;
R₃ is hydrogen; and
Q₁ is hydrogen, halogen, —N(R₄)CON(R₄)₂, in which R₄ is independently either hydrogen or C₁-C₄alkyl (preferably hydrogen or methyl) or 1,2,4-triazol-1-yl; preferably Q₁ is hydrogen, chlorine, bromine, —N(H)CONH(CH₃), —N(CH₃)CONH(CH₃) or 1,2,4-triazol-1-yl.

Another group of compounds according to the invention are those of formula I-9

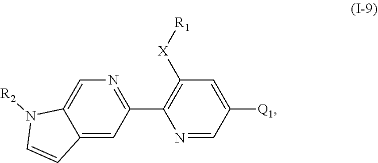

wherein X, R₁ and R₂ are as defined for compounds of formula I (above), or an agrochemically acceptable salt, stereoisomer, enantiomer, tautomer or N-oxide thereof, and wherein Q₁ is preferably hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂, —N(R₄)COR₅, or —N(R₄)CON(R₄)₂, (oxazolidin-2-one)-3-yl or 2-pyridyloxy; or
Q₁ is a five- to six-membered aromatic ring system linked via a ring carbon atom to the pyridyl ring substituted by X—R₁, said ring system is unsubstituted or is mono-substituted by a substituent selected from the group consisting of halogen, cyano and C₁-C₄haloalkyl; and said ring system can contain 1 or 2 ring nitrogen atoms; or
Q₁ is a five-membered aromatic ring system linked via a ring nitrogen atom to the pyridyl ring substituted by X—R₁, said ring system is unsubstituted or is mono-substituted by a substituent selected from the group consisting of halogen, cyano and C₁-C₄haloalkyl; and said ring system contains 2 or 3 ring nitrogen atoms;
Preferably each R₄ is independently hydrogen or C₁-C₄alkyl; and
R₅ is preferably C₁-C₆alkyl or C₃-C₆cycloalkyl.

Preferred definitions of X, R₁ and R₂ are as defined for compounds of formula I (above), and more preferably Q₁ is hydrogen, halogen, trifluoromethyl, cyclopropyl, cyanocyclopropyl, cyanoisopropyl, trifluoroethoxy, —N(R₄)₂, —N(R₄)COR₅, or —N(R₄)CON(R₄)₂, in which each R₄ is independently either hydrogen or methyl and R₅ is either methyl or cyclopropyl, or Q₁ is (oxazolidin-2-one)-3-yl, 2-pyridyloxy, N-linked pyrazolyl which is unsubstituted or is mono-substituted by chloro, cyano or trifluoromethyl; or Q₁ is N-linked triazolyl or C-linked pyrimidinyl; even more preferably Q₁ is hydrogen, halogen, trifluoromethyl, cyclopropyl, cyanocyclopropyl, cyanoisopropyl, —N(R₄)₂, —N(R₄)COR₅, or —N(R₄)CON(R₄)₂, in which each R₄ is independently either hydrogen or methyl and R₅ is either methyl or cyclopropyl, or Q₁ is (oxazolidin-2-one)-3-yl, 2-pyridyloxy, N-linked pyrazolyl which is mono-substituted by chloro or trifluoromethyl; or Q₁ is N-linked triazolyl or C-linked pyrimidinyl.

One group of compounds according to this embodiment are compounds of formula (I-9a) which are compounds of formula (I-9) wherein X is S or SO₂, preferably SO₂.

Another group of compounds according to this embodiment are compounds of formula (I-9b-1) which are compounds of formula (I-9) wherein R₁ is C₁-C₄alkyl or cyclopropyl-C₁-C₄alkyl, preferably ethyl or cyclopropylmethyl.

Another group of compounds according to this embodiment are compounds of formula (I-9b-2) which are compounds of formula (I-9) wherein R₁ is C₁-C₄alkyl, preferably ethyl.

Another group of compounds according to this embodiment are compounds of formula (I-9c) which are compounds of formula (I-9) wherein R₂ is C₁-C₆haloalkyl, more preferably C₁-C₆fluoroalkyl, most preferably —CH₂CF₂CHF₂ or —CH₂CF₂CF₃.

Another group of compounds according to the invention are those of formula I-10

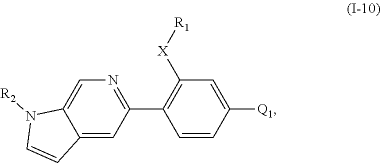

wherein X, R₁ and R₂ are as defined for compounds of formula I (above), or an agrochemically acceptable salt, stereoisomer, enantiomer, tautomer or N-oxide thereof, and wherein Q₁ is preferably hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂, —N(R₄)COR₅, or —N(R₄)CON(R₄)₂, (oxazolidin-2-one)-3-yl or 2-pyridyloxy; or
Q₁ is a five- to six-membered aromatic ring system linked via a ring carbon atom to the phenyl ring, said ring system is unsubstituted or is mono-substituted by a substituent selected from the group consisting of halogen, cyano and C₁-C₄haloalkyl; and said ring system can contain 1 or 2 ring nitrogen atoms; or
Q₁ is a five-membered aromatic ring system linked via a ring nitrogen atom to the phenyl ring substituted by X—R₁, said ring system is unsubstituted or is mono-substituted by a substituent selected from the group consisting of halogen, cyano and C₁-C₄haloalkyl; and said ring system contains 2 or 3 ring nitrogen atoms;
Preferably each R₄ is independently hydrogen or C₁-C₄alkyl; and
R₅ is preferably C₁-C₆alkyl or C₃-C₆cycloalkyl.

Preferred definitions of X, R₁ and R₂ are as defined for compounds of formula I (above), and more preferably Q₁ is hydrogen, halogen, trifluoromethyl, cyclopropyl, cyanocyclopropyl, cyanoisopropyl, trifluoroethoxy, —N(R₄)₂, —N(R₄)COR₅, or —N(R₄)CON(R₄)₂, in which each R₄ is independently either hydrogen or methyl and R₅ is either methyl or cyclopropyl, or Q₁ is (oxazolidin-2-one)-3-yl, 2-pyridyloxy, N-linked pyrazolyl which is unsubstituted or is mono-substituted by chloro, cyano or trifluoromethyl; or Q₁ is N-linked triazolyl or C-linked pyrimidinyl; even more preferably Q₁ is hydrogen, halogen, trifluoromethyl, cyclopropyl, cyanocyclopropyl, cyanoisopropyl, —N(R₄)₂, —N(R₄)COR₅, or —N(R₄)CON(R₄)₂, in which each R₄ is independently either hydrogen or methyl and R₅ is either methyl or cyclopropyl, or Q₁ is (oxazolidin-2-one)-3-yl, 2-pyridyloxy, N-linked pyrazolyl which is mono-substituted by chloro or trifluoromethyl; or Q₁ is N-linked triazolyl or C-linked pyrimidinyl.

One group of compounds according to this embodiment are compounds of formula (I-10a) which are compounds of formula (I-10) wherein X is S or SO₂, preferably SO₂.

Another group of compounds according to this embodiment are compounds of formula (I-10b-1) which are compounds of formula (I-10) wherein R₁ is C₁-C₄alkyl or cyclopropyl-C₁-C₄alkyl, preferably ethyl or cyclopropylmethyl.

Another group of compounds according to this embodiment are compounds of formula (I-10b-2) which are compounds of formula (I-10) wherein R₁ is C₁-C₄alkyl, preferably ethyl.

Another group of compounds according to this embodiment are compounds of formula (I-10c) which are compounds of formula (I-10) wherein R₂ is C₁-C₆haloalkyl, more preferably C₁-C₆fluoroalkyl, most preferably —CH₂CF₂CHF₂ or —CH₂CF₂CF₃.

Another group of compounds according to the invention are those of formula I-11

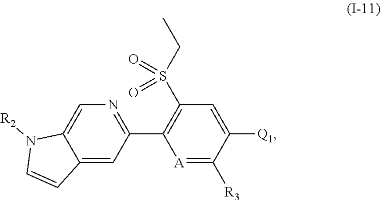

wherein
A is CH or N, preferably N;
R₂ is C₁-C₆haloalkyl, preferably —CH₂CF₂CHF₂ or —CH₂CF₂CF₃;
R₃ is hydrogen or C₁-C₄alkyl, preferably hydrogen or methyl;
Q₁ is hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂, —N(R₄)COR₅, or —N(R₄)CON(R₄)₂, (oxazolidin-2-one)-3-yl or 2-pyridyloxy; or
Q₁ is a five- to six-membered aromatic ring system linked via a ring carbon atom to the ring which contains the substituent A, said ring system is unsubstituted or is mono-substituted by a substituent selected from the group consisting of halogen, cyano and C₁-C₄haloalkyl; and said ring system can contain 1 or 2 ring nitrogen atoms; or
Q₁ is a five-membered aromatic ring system linked via a ring nitrogen atom to the ring which contains the substituent A, said ring system is unsubstituted or is mono-substituted by a substituent selected from the group consisting of halogen, cyano and C₁-C₄haloalkyl; and said ring system contains 2 or 3 ring nitrogen atoms;
Each R₄ is independently hydrogen or C₁-C₄alkyl, preferably hydrogen or methyl; and
R₅ is C₁-C₆alkyl or C₃-C₆cycloalkyl, preferably methyl or cyclopropyl; or an agrochemically acceptable salt, stereoisomer, enantiomer, tautomer or N-oxide thereof.

One further preferred group of compounds according to this embodiment are compounds of formula (I-11a) which are compounds of formula (I-11) wherein A is N.

Another preferred group of compounds according to this embodiment are compounds of formula (I-11b) which are compounds of formula (I-11) wherein A is CH.

One further preferred group of compounds according to this embodiment are compounds of formula (I-11c) which are compounds of formula (I-11) wherein R₃ is hydrogen.

Another preferred group of compounds according to this embodiment are compounds of formula (I-11d) which are compounds of formula (I-11) wherein R₃ is C₁-C₄alkyl, preferably methyl.

One further preferred group of compounds according to this embodiment are compounds of formula (I-11e) which are compounds of formula (I-11) wherein A is N and R₃ is hydrogen.

One further preferred group of compounds according to this embodiment are compounds of formula (I-11f) which are compounds of formula (I-11) wherein Q₁ is hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂ or —N(R₄)COR₅, both in which R₄ is independently hydrogen or C₁-C₄alkyl (preferably hydrogen or methyl) and R₅ is C₁-C₆alkyl (preferably methyl), or Q₁ is 2-pyridyloxy; preferably Q₁ is hydrogen, halogen, trifluoromethyl, cyclopropyl, cyanocyclopropyl, cyanoisopropyl, trifluoroethoxy, —N(R₄)₂ or —N(R₄)COCH₃, both in which R₄ is independently hydrogen or methyl, or Q₁ is 2-pyridyloxy; or

Q₁ is a five-membered aromatic ring system linked via a ring nitrogen atom to the ring which contains the substituent A, said ring system is unsubstituted or is mono-substituted by a substituent selected from the group consisting of halogen, cyano and C₁-C₄haloalkyl; and said ring system contains 2 ring nitrogen atoms; preferably Q₁ is N-linked pyrazolyl which is unsubstituted or is mono-substituted by chloro, cyano or trifluoromethyl.

One further preferred group of compounds according to this embodiment are compounds of formula (I-11g) which are compounds of formula (I-11) wherein Q₁ is hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂ or —N(R₄)COR₅, both in which R₄ is independently hydrogen or C₁-C₄alkyl (preferably hydrogen or methyl) and R₅ is C₁-C₆alkyl (preferably methyl), 2-pyridyloxy or N-linked pyrazolyl which is unsubstituted or is mono-substituted by chloro, cyano or trifluoromethyl; preferably Q₁ is hydrogen, chlorine, bromine, trifluoromethyl, cyclopropyl, 1-cyanocyclopropyl, 1-cyano-1-methyl-ethyl, 2,2,2-trifluoroethoxy, —NH₂, —NH(CH₃), —N(CH₃)₂, —NHCOCH₃, —N(CH₃)COCH₃, 2-pyridyloxy, pyrazol-1-yl, 3-chloro-pyrazol-1-yl, 3-cyano-pyrazol-1-yl or 3-trifluoromethyl-pyrazol-1-yl.

One further preferred group of compounds according to this embodiment are compounds of formula (I-11g-1) which are compounds of formula (I-11) wherein Q₁ is hydrogen, C₁-C₆haloalkyl, C₃-C₆cycloalkyl, C₃-C₆cycloalkyl monosubstituted by cyano, C₁-C₆cyanoalkyl, —N(R₄)₂ or —N(R₄)COR₅, both in which R₄ is independently hydrogen or C₁-C₄alkyl (preferably hydrogen or methyl) and R₅ is C₁-C₆alkyl (preferably methyl), 2-pyridyloxy or N-linked pyrazolyl which is mono-substituted by chloro or trifluoromethyl; preferably Q₁ is hydrogen, trifluoromethyl, cyclopropyl, 1-cyanocyclopropyl, 1-cyano-1-methyl-ethyl, —NH(CH₃), —N(CH₃)COCH₃, 2-pyridyloxy, 3-chloro-pyrazol-1-yl or 3-trifluoromethyl-pyrazol-1-yl.

One further preferred group of compounds according to this embodiment are compounds of formula (I-11g-2) which are compounds of formula (I-11) wherein Q₁ is hydrogen, C₃-C₆cycloalkyl, C₃-C₆cycloalkyl monosubstituted by cyano, C₁-C₆cyanoalkyl, —N(R₄)₂ or —N(R₄)COR₅, both in which R₄ is independently hydrogen or C₁-C₄alkyl (preferably hydrogen or methyl) and R₅ is C₁-C₆alkyl (preferably methyl), or 2-pyridyloxy; preferably Q₁ is hydrogen, cyclopropyl, 1-cyanocyclopropyl, 1-cyano-1-methyl-ethyl, —NH(CH₃), —N(CH₃)COCH₃, or 2-pyridyloxy.

One further preferred group of compounds according to this embodiment are compounds of formula (I-11h) which are compounds of formula (I-11) wherein

### A is N;

R₂ is C₁-C₆haloalkyl, preferably —CH₂CF₂CHF₂ or —CH₂CF₂CF₃;
R₃ is hydrogen or C₁-C₄alkyl, preferably hydrogen or methyl; and
Q₁ is hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂ or —N(R₄)COR₅, both in which R₄ is independently hydrogen or C₁-C₄alkyl (preferably hydrogen or methyl) and R₅ is C₁-C₆alkyl (preferably methyl), or Q₁ is 2-pyridyloxy; preferably Q₁ is hydrogen, halogen, trifluoromethyl, cyclopropyl, cyanocyclopropyl, cyanoisopropyl, trifluoroethoxy, —N(R₄)₂ or —N(R₄)COCH₃, both in which R₄ is independently hydrogen or methyl, or Q₁ is 2-pyridyloxy; or
Q₁ is a five-membered aromatic ring system linked via a ring nitrogen atom to the ring which contains the substituent A, said ring system is unsubstituted or is mono-substituted by a substituent selected from the group consisting of halogen, cyano and C₁-C₄haloalkyl; and said ring system contains 2 ring nitrogen atoms; preferably Q₁ is N-linked pyrazolyl which is unsubstituted or is mono-substituted by chloro, cyano or trifluoromethyl.

Another preferred group of compounds according to this embodiment are compounds of formula (I-11i) which are compounds of formula (I-11) wherein

### A is N;

R₂ is C₁-C₆haloalkyl, preferably —CH₂CF₂CHF₂ or —CH₂CF₂CF₃;
R₃ is hydrogen; and
Q₁ is hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂ or —N(R₄)COR₅, both in which R₄ is independently hydrogen or C₁-C₄alkyl (preferably hydrogen or methyl) and R₅ is C₁-C₆alkyl (preferably methyl), 2-pyridyloxy or N-linked pyrazolyl which is unsubstituted or is mono-substituted by chloro, cyano or trifluoromethyl; preferably Q₁ is hydrogen, chlorine, bromine, trifluoromethyl, cyclopropyl, 1-cyanocyclopropyl, 1-cyano-1-methyl-ethyl, 2,2,2-trifluoroethoxy, —NH₂, —NH(CH₃), —N(CH₃)₂, —NHCOCH₃, —N(CH₃)COCH₃, 2-pyridyloxy, pyrazol-1-yl, 3-chloro-pyrazol-1-yl, 3-cyano-pyrazol-1-yl or 3-trifluoromethyl-pyrazol-1-yl.

Another preferred group of compounds according to this embodiment are compounds of formula (I-11i-1) which are compounds of formula (I-11) wherein

### A is N;

R₂ is C₁-C₆haloalkyl, preferably —CH₂CF₂CHF₂ or —CH₂CF₂CF₃;
R₃ is hydrogen; and
Q₁ is hydrogen, C₁-C₆haloalkyl, C₃-C₆cycloalkyl, C₃-C₆cycloalkyl monosubstituted by cyano, C₁-C₆cyanoalkyl, —N(R₄)₂ or —N(R₄)COR₅, both in which R₄ is independently hydrogen or C₁-C₄alkyl (preferably hydrogen or methyl) and R₅ is C₁-C₆alkyl (preferably methyl), 2-pyridyloxy or N-linked pyrazolyl which is mono-substituted by chloro or trifluoromethyl; preferably Q₁ is hydrogen, trifluoromethyl, cyclopropyl, 1-cyanocyclopropyl, 1-cyano-1-methyl-ethyl, —NH(CH₃), —N(CH₃)COCH₃, 2-pyridyloxy, 3-chloro-pyrazol-1-yl or 3-trifluoromethyl-pyrazol-1-yl.

Another preferred group of compounds according to this embodiment are compounds of formula (I-11i-2) which are compounds of formula (I-11) wherein

### A is N;

R₂ is C₁-C₆haloalkyl, preferably —CH₂CF₂CHF₂ or —CH₂CF₂CF₃;
R₃ is hydrogen; and
Q₁ is hydrogen, C₃-C₆cycloalkyl, C₃-C₆cycloalkyl monosubstituted by cyano, C₁-C₆cyanoalkyl, —N(R₄)₂ or —N(R₄)COR₅, both in which R₄ is independently hydrogen or C₁-C₄alkyl (preferably hydrogen or methyl) and R₅ is C₁-C₆alkyl (preferably methyl), or 2-pyridyloxy; preferably Q₁ is hydrogen, cyclopropyl, 1-cyanocyclopropyl, 1-cyano-1-methyl-ethyl, —NH(CH₃), —N(CH₃)COCH₃, or 2-pyridyloxy.

Another preferred group of compounds according to this embodiment are compounds of formula (I-11j) which are compounds of formula (I-11) wherein

### A is N;

R₂ is C₁-C₆haloalkyl, preferably —CH₂CF₂CHF₂ or —CH₂CF₂CF₃;
R₃ is hydrogen; and
Q₁ is hydrogen, halogen, C₃-C₆cycloalkyl, C₁-C₆cyanoalkyl, or —N(R₄)COR₅ in which R₄ is hydrogen or C₁-C₄alkyl (preferably hydrogen or methyl) and R₅ is C₁-C₆alkyl (preferably methyl); preferably Q₁ is hydrogen, chlorine, bromine, cyclopropyl, 1-cyano-1-methyl-ethyl, or —N(CH₃)COCH₃.

Another preferred group of compounds according to this embodiment are compounds of formula (I-11k) which are compounds of formula (I-11) wherein

### A is N;

R₂ is C₁-C₆haloalkyl, preferably —CH₂CF₂CHF₂ or —CH₂CF₂CF₃;
R₃ is C₁-C₄alkyl, preferably methyl; and
Q₁ is hydrogen, halogen, C₃-C₆cycloalkyl, C₁-C₆cyanoalkyl, or —N(R₄)COR₅ in which R₄ is hydrogen or C₁-C₄alkyl (preferably hydrogen or methyl) and R₅ is C₁-C₆alkyl (preferably methyl); preferably Q₁ is hydrogen, chlorine, bromine, cyclopropyl, 1-cyano-1-methyl-ethyl, or —N(CH₃)COCH₃.

Another group of compounds according to the invention are those of formula I-12

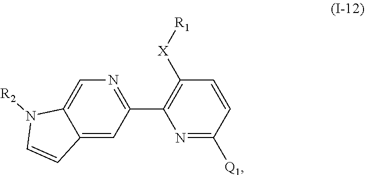

wherein X, R₁ and R₂ are as defined for compounds of formula I (above), or an agrochemically acceptable salt, stereoisomer, enantiomer, tautomer or N-oxide thereof, and wherein Q₁ is preferably hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂, —N(R₄)COR₅, or —N(R₄)CON(R₄)₂, (oxazolidin-2-one)-3-yl or 2-pyridyloxy; or
Q₁ is a five- to six-membered aromatic ring system linked via a ring carbon atom to the pyridyl ring substituted by X—R₁, said ring system is unsubstituted or is mono-substituted by a substituent selected from the group consisting of halogen, cyano and C₁-C₄haloalkyl; and said ring system can contain 1 or 2 ring nitrogen atoms; or
Q₁ is a five-membered aromatic ring system linked via a ring nitrogen atom to the pyridyl ring substituted by X—R₁, said ring system is unsubstituted or is mono-substituted by a substituent selected from the group consisting of halogen, cyano or C₁-C₄haloalkyl; and said ring system contains 2 or 3 nitrogen atoms;
Preferably each R₄ is independently hydrogen or C₁-C₄alkyl; and
R₅ is preferably C₁-C₆alkyl or C₃-C₆cycloalkyl.

Preferred definitions of X, R₁ and R₂ are as defined for compounds of formula I (above), and more preferably Q₁ is hydrogen, halogen, trifluoromethyl, cyclopropyl, cyanocyclopropyl, cyanoisopropyl, trifluoroethoxy, —N(R₄)₂, —N(R₄)COR₅, or —N(R₄)CON(R₄)₂, in which each R₄ is independently either hydrogen or methyl and R₅ is either methyl or cyclopropyl, or Q₁ is (oxazolidin-2-one)-3-yl, 2-pyridyloxy, N-linked pyrazolyl which is unsubstituted or is mono-substituted by chloro, cyano or trifluoromethyl; or Q₁ is N-linked triazolyl or C-linked pyrimidinyl; even more preferably Q₁ is hydrogen, halogen, trifluoromethyl, cyclopropyl, cyanocyclopropyl, cyanoisopropyl, —N(R₄)₂, —N(R₄)COR₅, or —N(R₄)CON(R₄)₂, in which each R₄ is independently either hydrogen or methyl and R₅ is either methyl or cyclopropyl, or Q₁ is (oxazolidin-2-one)-3-yl, 2-pyridyloxy, N-linked pyrazolyl which is mono-substituted by chloro or trifluoromethyl; or Q₁ is N-linked triazolyl or C-linked pyrimidinyl.

One group of compounds according to this embodiment are compounds of formula (I-12a) which are compounds of formula (I-12) wherein X is S or SO₂, preferably SO₂.

Another group of compounds according to this embodiment are compounds of formula (I-12b-1) which are compounds of formula (I-12) wherein R₁ is C₁-C₄alkyl or cyclopropyl-C₁-C₄alkyl, preferably ethyl or cyclopropylmethyl.

Another group of compounds according to this embodiment are compounds of formula (I-12b-2) which are compounds of formula (I-12) wherein R₁ is C₁-C₄alkyl, preferably ethyl.

Another group of compounds according to this embodiment are compounds of formula (I-12c) which are compounds of formula (I-12) wherein R₂ is C₁-C₆haloalkyl, more preferably C₁-C₆fluoroalkyl, most preferably —CH₂CF₂CHF₂ or —CH₂CF₂CF₃.

Another group of compounds according to the invention are those of formula I-13

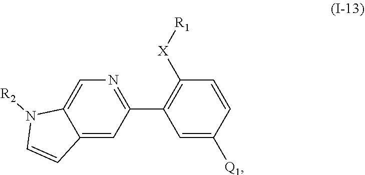

wherein X, R₁ and R₂ are as defined for compounds of formula I (above), or an agrochemically acceptable salt, stereoisomer, enantiomer, tautomer or N-oxide thereof, and wherein Q₁ is preferably hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂, —N(R₄)COR₅, or —N(R₄)CON(R₄)₂, (oxazolidin-2-one)-3-yl or 2-pyridyloxy; or
Q₁ is a five- to six-membered aromatic ring system linked via a ring carbon atom to the phenyl ring substituted by X—R₁, said ring system is unsubstituted or is mono-substituted by a substituent selected from the group consisting of halogen, cyano and C₁-C₄haloalkyl; and said ring system can contain 1 or 2 ring nitrogen atoms; or
Q₁ is a five-membered aromatic ring system linked via a ring nitrogen atom to the phenyl ring substituted by X—R₁, said ring system is unsubstituted or is mono-substituted by a substituent selected from the group consisting of halogen, cyano and C₁-C₄haloalkyl; and said ring system contains 2 or 3 ring nitrogen atoms;
Preferably each R₄ is independently hydrogen or C₁-C₄alkyl; and
R₅ is preferably C₁-C₆alkyl or C₃-C₆cycloalkyl.

Preferred definitions of X, R₁ and R₂ are as defined for compounds of formula I (above), and more preferably Q₁ is hydrogen, halogen, trifluoromethyl, cyclopropyl, cyanocyclopropyl, cyanoisopropyl, trifluoroethoxy, —N(R₄)₂, —N(R₄)COR₅, or —N(R₄)CON(R₄)₂, in which each R₄ is independently either hydrogen or methyl and R₅ is either methyl or cyclopropyl, or Q₁ is (oxazolidin-2-one)-3-yl, 2-pyridyloxy, N-linked pyrazolyl which is unsubstituted or is mono-substituted by chloro, cyano or trifluoromethyl; or Q₁ is N-linked triazolyl or C-linked pyrimidinyl; even more preferably Q₁ is hydrogen, halogen, trifluoromethyl, cyclopropyl, cyanocyclopropyl, cyanoisopropyl, —N(R₄)₂, —N(R₄)COR₅, or —N(R₄)CON(R₄)₂, in which each R₄ is independently either hydrogen or methyl and R₅ is either methyl or cyclopropyl, or Q₁ is (oxazolidin-2-one)-3-yl, 2-pyridyloxy, N-linked pyrazolyl which is mono-substituted by chloro or trifluoromethyl; or Q₁ is N-linked triazolyl or C-linked pyrimidinyl.

One group of compounds according to this embodiment are compounds of formula (I-13a) which are compounds of formula (I-13) wherein X is S or SO₂, preferably SO₂.

Another group of compounds according to this embodiment are compounds of formula (I-13b-1) which are compounds of formula (I-13) wherein R₁ is C₁-C₄alkyl or cyclopropyl-C₁-C₄alkyl, preferably ethyl or cyclopropylmethyl.

Another group of compounds according to this embodiment are compounds of formula (I-13b-2) which are compounds of formula (I-13) wherein R₁ is C₁-C₄alkyl, preferably ethyl.

Another group of compounds according to this embodiment are compounds of formula (I-13c) which are compounds of formula (I-13) wherein R₂ is C₁-C₆haloalkyl, more preferably C₁-C₆fluoroalkyl, most preferably —CH₂CF₂CHF₂ or —CH₂CF₂CF₃.

Another group of compounds according to the invention are those of formula I-14

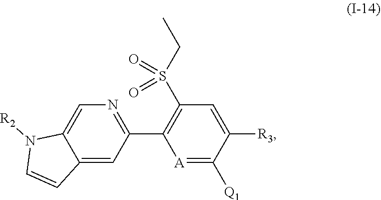

wherein
A is CH or N, preferably N;
R₂ is C₁-C₆haloalkyl, preferably —CH₂CF₂CHF₂ or —CH₂CF₂CF₃;
R₃ is hydrogen or C₁-C₄alkyl, preferably hydrogen or methyl;
Q₁ is hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂, —N(R₄)COR₅, or —N(R₄)CON(R₄)₂, (oxazolidin-2-one)-3-yl or 2-pyridyloxy; or
Q₁ is a five- to six-membered aromatic ring system linked via a ring carbon atom to the ring which contains the substituent A, said ring system is unsubstituted or is mono-substituted by a substituent selected from the group consisting of halogen, cyano and C₁-C₄haloalkyl; and said ring system can contain 1 or 2 ring nitrogen atoms; or
Q₁ is a five-membered aromatic ring system linked via a ring nitrogen atom to the ring which contains the substituent A, said ring system is unsubstituted or is mono-substituted by a substituent selected from the group consisting of halogen, cyano and C₁-C₄haloalkyl; and said ring system contains 2 or 3 ring nitrogen atoms;
Each R₄ is independently hydrogen or C₁-C₄alkyl, preferably hydrogen or methyl; and
R₅ is C₁-C₆alkyl or C₃-C₆cycloalkyl, preferably methyl or cyclopropyl; or
an agrochemically acceptable salt, stereoisomer, enantiomer, tautomer or N-oxide thereof.

One further preferred group of compounds according to this embodiment are compounds of formula (I-14a) which are compounds of formula (I-14) wherein A is N.

Another preferred group of compounds according to this embodiment are compounds of formula (I-14b) which are compounds of formula (I-14) wherein A is CH.

One further preferred group of compounds according to this embodiment are compounds of formula (I-14c) which are compounds of formula (I-14) wherein R₃ is hydrogen.

Another preferred group of compounds according to this embodiment are compounds of formula (I-14d) which are compounds of formula (I-14) wherein R₃ is C₁-C₄alkyl, preferably methyl.

One further preferred group of compounds according to this embodiment are compounds of formula (I-14e) which are compounds of formula (I-14) wherein A is N and R₃ is hydrogen.

One further preferred group of compounds according to this embodiment are compounds of formula (I-14f) which are compounds of formula (I-14) wherein Q₁ is hydrogen, halogen, C₃-C₆cycloalkyl, —N(R₄)₂, —N(R₄)COR₅ or —N(R₄)CON(R₄)₂, in each of which R₄ is independently either hydrogen or C₁-C₄alkyl (preferably hydrogen or methyl) and R₅ is C₁-C₆alkyl or C₃-C₆cycloalkyl (preferably methyl or cyclopropyl), or Q₁ is (oxazolidin-2-one)-3-yl; preferably Q₁ is hydrogen, halogen, cyclopropyl, —N(R₄)₂, —N(R₄)COR₅ or —N(R₄)CON(R₄)₂, in each of which R₄ is independently either hydrogen or methyl and R₅ is methyl or cyclopropyl, or Q₁ is (oxazolidin-2-one)-3-yl; or

Q₁ is a six-membered aromatic ring system linked via a ring carbon atom to the ring which contains the substituent A, said ring system is unsubstituted or is mono-substituted by a substituent selected from the group consisting of halogen, cyano and C₁-C₄haloalkyl; and said ring system contains 2 ring nitrogen atoms; preferably Q₁ is pyrimidinyl which is unsubstituted or is mono-substituted by chloro, cyano or trifluoromethyl; or
Q₁ is a five-membered aromatic ring system linked via a ring nitrogen atom to the ring which contains the substituent A, said ring system is unsubstituted or is mono-substituted by a substituent selected from the group consisting of halogen, cyano and C₁-C₄haloalkyl; and said ring system contains 3 ring nitrogen atoms; preferably Q₁ is N-linked triazolyl which is unsubstituted or is mono-substituted by chloro, cyano or trifluoromethyl.

One further preferred group of compounds according to this embodiment are compounds of formula (I-14g) which are compounds of formula (I-14) wherein Q₁ is hydrogen, halogen, C₃-C₆cycloalkyl, —N(R₄)₂, —N(R₄)COR₅ or —N(R₄)CON(R₄)₂, in each of which R₄ is independently either hydrogen or C₁-C₄alkyl (preferably hydrogen or methyl) and R₅ is C₁-C₆alkyl or C₃-C₆cycloalkyl (preferably methyl or cyclopropyl); or Q₁ is (oxazolidin-2-one)-3-yl, C-linked pyrimidinyl or N-linked triazolyl; preferably Q₁ is hydrogen, chlorine, bromine, cyclopropyl, —NH₂, —NH(CH₃), —NHCOCH₃, —N(CH₃)COCH₃, —NHCO(cyclopropyl), —N(CH₃)CO(cyclopropyl), —N(H)CONH₂, —N(H)CONH(CH₃), —N(H)CON(CH₃)₂, —N(CH₃)CONH₂, —N(CH₃)CONH(CH₃), —N(CH₃)CON(CH₃)₂, (oxazolidin-2-one)-3-yl, pyrimidin-2-yl or 1,2,4-triazol-1-yl.

One further preferred group of compounds according to this embodiment are compounds of formula (I-14g-1) which are compounds of formula (I-14) wherein Q₁ is hydrogen, halogen, —N(R₄)₂, or —N(R₄)CON(R₄)₂, in each of which R₄ is independently either hydrogen or C₁-C₄alkyl (preferably hydrogen or methyl), or N-linked triazolyl; preferably Q₁ is hydrogen, chlorine, —NH(CH₃), —N(CH₃)CONH(CH₃), or 1,2,4-triazol-1-yl.

One further preferred group of compounds according to this embodiment are compounds of formula (I-14h) which are compounds of formula (I-14) wherein

### A is N;

R₂ is C₁-C₆haloalkyl, preferably —CH₂CF₂CHF₂ or —CH₂CF₂CF₃;
R₃ is hydrogen or C₁-C₄alkyl, preferably hydrogen or methyl; and
Q₁ is hydrogen, halogen, C₃-C₆cycloalkyl, —N(R₄)₂, —N(R₄)COR₅ or —N(R₄)CON(R₄)₂, in each of which R₄ is independently either hydrogen or C₁-C₄alkyl (preferably hydrogen or methyl) and R₅ is C₁-C₆alkyl or C₃-C₆cycloalkyl (preferably methyl or cyclopropyl), or Q₁ is (oxazolidin-2-one)-3-yl; preferably Q₁ is hydrogen, halogen, cyclopropyl, —N(R₄)₂, —N(R₄)COR₅ or —N(R₄)CON(R₄)₂, in each of which R₄ is independently either hydrogen or methyl and R₅ is methyl or cyclopropyl, or Q₁ is (oxazolidin-2-one)-3-yl; or
Q₁ is a six-membered aromatic ring system linked via a ring carbon atom to the ring which contains the substituent A, said ring system is unsubstituted or is mono-substituted by a substituent selected from the group consisting of halogen, cyano and C₁-C₄haloalkyl; and said ring system contains 2 ring nitrogen atoms; preferably Q₁ is pyrimidinyl which is unsubstituted or is mono-substituted by chloro, cyano or trifluoromethyl; or
Q₁ is a five-membered aromatic ring system linked via a ring nitrogen atom to the ring which contains the substituent A, said ring system is unsubstituted or is mono-substituted by a substituent selected from the group consisting of halogen, cyano and C₁-C₄haloalkyl; and said ring system contains 3 ring nitrogen atoms; preferably Q₁ is N-linked triazolyl which is unsubstituted or is mono-substituted by chloro, cyano or trifluoromethyl.

Another preferred group of compounds according to this embodiment are compounds of formula (I-14i) which are compounds of formula (I-14) wherein

### A is N;

R₂ is C₁-C₆haloalkyl, preferably —CH₂CF₂CHF₂ or —CH₂CF₂CF₃;
R₃ is hydrogen; and
Q₁ is hydrogen, halogen, C₃-C₆cycloalkyl, —N(R₄)₂, —N(R₄)COR₅ or —N(R₄)CON(R₄)₂, in each of which R₄ is independently either hydrogen or C₁-C₄alkyl (preferably hydrogen or methyl) and R₅ is C₁-C₆alkyl or C₃-C₆cycloalkyl (preferably methyl or cyclopropyl); or Q₁ is (oxazolidin-2-one)-3-yl, C-linked pyrimidinyl or N-linked triazolyl; preferably Q₁ is hydrogen, chlorine, bromine, cyclopropyl, —NH₂, —NH(CH₃), —NHCOCH₃, —N(CH₃)COCH₃, —NHCO(cyclopropyl), —N(CH₃)CO(cyclopropyl), —N(H)CONH₂, —N(H)CONH(CH₃), —N(H)CON(CH₃)₂, —N(CH₃)CONH₂, —N(CH₃)CONH(CH₃), —N(CH₃)CON(CH₃)₂, (oxazolidin-2-one)-3-yl, pyrimidin-2-yl or 1,2,4-triazol-1-yl.

Another preferred group of compounds according to this embodiment are compounds of formula (I-14i-1) which are compounds of formula (I-14) wherein

### A is N;

R₂ is C₁-C₆haloalkyl, preferably —CH₂CF₂CHF₂ or —CH₂CF₂CF₃;
R₃ is hydrogen; and
Q₁ is hydrogen, halogen, —N(R₄)₂, or —N(R₄)CON(R₄)₂, in each of which R₄ is independently either hydrogen or C₁-C₄alkyl (preferably hydrogen or methyl), or N-linked triazolyl; preferably Q₁ is hydrogen, chlorine, —NH(CH₃), —N(CH₃)CONH(CH₃), or 1,2,4-triazol-1-yl.

Another preferred group of compounds according to this embodiment are compounds of formula (I-14j) which are compounds of formula (I-14) wherein

### A is N;

R₂ is C₁-C₆haloalkyl, preferably —CH₂CF₂CHF₂ or —CH₂CF₂CF₃;
R₃ is hydrogen; and
Q₁ is hydrogen, halogen, —N(R₄)CON(R₄)₂, in which each R₄ is independently either hydrogen or C₁-C₄alkyl (preferably hydrogen or methyl) or 1,2,4-triazol-1-yl; preferably Q₁ is hydrogen, chlorine, bromine, —N(H)CONH(CH₃), —N(CH₃)CONH(CH₃) or 1,2,4-triazol-1-yl.

Another group of compounds according to the invention are those of formula I-15

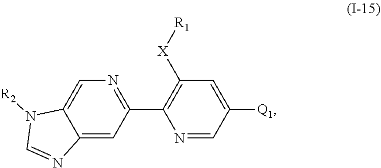

wherein X, R₁ and R₂ are as defined for compounds of formula I (above), or an agrochemically acceptable salt, stereoisomer, enantiomer, tautomer or N-oxide thereof, and wherein Q₁ is preferably hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂, —N(R₄)COR₅, or —N(R₄)CON(R₄)₂, (oxazolidin-2-one)-3-yl or 2-pyridyloxy; or
Q₁ is a five- to six-membered aromatic ring system linked via a ring carbon atom to the pyridyl ring substituted by X—R₁, said ring system is unsubstituted or is mono-substituted by a substituent selected from the group consisting of halogen, cyano and C₁-C₄haloalkyl; and said ring system can contain 1 or 2 ring nitrogen atoms; or
Q₁ is a five-membered aromatic ring system linked via a ring nitrogen atom to the pyridyl ring substituted by X—R₁, said ring system is unsubstituted or is mono-substituted by a substituent selected from the group consisting of halogen, cyano and C₁-C₄haloalkyl; and said ring system contains 2 or 3 ring nitrogen atoms;
Preferably each R₄ is independently hydrogen or C₁-C₄alkyl; and
R₅ is preferably C₁-C₆alkyl or C₃-C₆cycloalkyl.

Preferred definitions of X, R₁ and R₂ are as defined for compounds of formula I (above), and more preferably Q₁ is hydrogen, halogen, trifluoromethyl, cyclopropyl, cyanocyclopropyl, cyanoisopropyl, trifluoroethoxy, —N(R₄)₂, —N(R₄)COR₅, or —N(R₄)CON(R₄)₂, in which each R₄ is independently either hydrogen or methyl and R₅ is either methyl or cyclopropyl, or Q₁ is (oxazolidin-2-one)-3-yl, 2-pyridyloxy, N-linked pyrazolyl which is unsubstituted or is mono-substituted by chloro, cyano or trifluoromethyl; or Q₁ is N-linked triazolyl or C-linked pyrimidinyl; even more preferably Q₁ is hydrogen, halogen, trifluoromethyl, cyclopropyl, cyanocyclopropyl, cyanoisopropyl, —N(R₄)₂, —N(R₄)COR₅, or —N(R₄)CON(R₄)₂, in which each R₄ is independently either hydrogen or methyl and R₅ is either methyl or cyclopropyl, or Q₁ is (oxazolidin-2-one)-3-yl, 2-pyridyloxy, N-linked pyrazolyl which is mono-substituted by chloro or trifluoromethyl; or Q₁ is N-linked triazolyl or C-linked pyrimidinyl.

One group of compounds according to this embodiment are compounds of formula (I-15a) which are compounds of formula (I-15) wherein X is S or SO₂, preferably SO₂.

Another group of compounds according to this embodiment are compounds of formula (I-15b-1) which are compounds of formula (I-15) wherein R₁ is C₁-C₄alkyl or cyclopropyl-C₁-C₄alkyl, preferably ethyl or cyclopropylmethyl.

Another group of compounds according to this embodiment are compounds of formula (I-15b-2) which are compounds of formula (I-15) wherein R₁ is C₁-C₄alkyl, preferably ethyl.

Another group of compounds according to this embodiment are compounds of formula (I-15c) which are compounds of formula (I-15) wherein R₂ is C₁-C₆haloalkyl, more preferably C₁-C₆fluoroalkyl, most preferably —CH₂CF₂CHF₂ or —CH₂CF₂CF₃.

Another group of compounds according to the invention are those of formula I-16

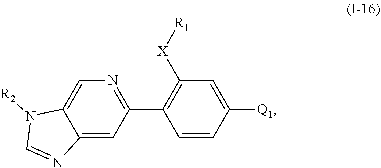

wherein X, R₁ and R₂ are as defined for compounds of formula I (above), or an agrochemically acceptable salt, stereoisomer, enantiomer, tautomer or N-oxide thereof, and wherein Q₁ is preferably hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂, —N(R₄)COR₅, or —N(R₄)CON(R₄)₂, (oxazolidin-2-one)-3-yl or 2-pyridyloxy; or
Q₁ is a five- to six-membered aromatic ring system linked via a ring carbon atom to the phenyl ring substituted by X—R₁, said ring system is unsubstituted or is mono-substituted by a substituent selected from the group consisting of halogen, cyano and C₁-C₄haloalkyl; and said ring system can contain 1 or 2 ring nitrogen atoms; or
Q₁ is a five-membered aromatic ring system linked via a ring nitrogen atom to the phenyl ring substituted by X—R₁, said ring system is unsubstituted or is mono-substituted by a substituent selected from the group consisting of halogen, cyano and C₁-C₄haloalkyl; and said ring system contains 2 or 3 ring nitrogen atoms;
Preferably each R₄ is independently hydrogen or C₁-C₄alkyl; and
R₅ is preferably C₁-C₆alkyl or C₃-C₆cycloalkyl.

Preferred definitions of X, R₁ and R₂ are as defined for compounds of formula I (above), and more preferably Q₁ is hydrogen, halogen, trifluoromethyl, cyclopropyl, cyanocyclopropyl, cyanoisopropyl, trifluoroethoxy, —N(R₄)₂, —N(R₄)COR₅, or —N(R₄)CON(R₄)₂, in which each R₄ is independently either hydrogen or methyl and R₅ is either methyl or cyclopropyl, or Q₁ is (oxazolidin-2-one)-3-yl, 2-pyridyloxy, N-linked pyrazolyl which is unsubstituted or is mono-substituted by chloro, cyano or trifluoromethyl; or Q₁ is N-linked triazolyl or C-linked pyrimidinyl; even more preferably Q₁ is hydrogen, halogen, trifluoromethyl, cyclopropyl, cyanocyclopropyl, cyanoisopropyl, —N(R₄)₂, —N(R₄)COR₅, or —N(R₄)CON(R₄)₂, in which each R₄ is independently either hydrogen or methyl and R₅ is either methyl or cyclopropyl, or Q₁ is (oxazolidin-2-one)-3-yl, 2-pyridyloxy, N-linked pyrazolyl which is mono-substituted by chloro or trifluoromethyl; or Q₁ is N-linked triazolyl or C-linked pyrimidinyl.

One group of compounds according to this embodiment are compounds of formula (I-16a) which are compounds of formula (I-16) wherein X is S or SO₂, preferably SO₂.

Another group of compounds according to this embodiment are compounds of formula (I-16b-1) which are compounds of formula (I-16) wherein R₁ is C₁-C₄alkyl or cyclopropyl-C₁-C₄alkyl, preferably ethyl or cyclopropylmethyl.

Another group of compounds according to this embodiment are compounds of formula (I-16b-2) which are compounds of formula (I-16) wherein R₁ is C₁-C₄alkyl, preferably ethyl.

Another group of compounds according to this embodiment are compounds of formula (I-16c) which are compounds of formula (I-16) wherein R₂ is C₁-C₆haloalkyl, more preferably C₁-C₆fluoroalkyl, most preferably —CH₂CF₂CHF₂ or —CH₂CF₂CF₃.

Another group of compounds according to the invention are those of formula I-17

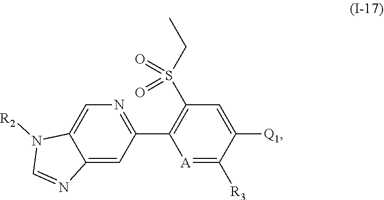

wherein
A is CH or N, preferably N;
R₂ is C₁-C₆haloalkyl, preferably —CH₂CF₂CHF₂ or —CH₂CF₂CF₃;
R₃ is hydrogen or C₁-C₄alkyl, preferably hydrogen or methyl;
Q₁ is hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂, —N(R₄)COR₅, or —N(R₄)CON(R₄)₂, (oxazolidin-2-one)-3-yl or 2-pyridyloxy; or
Q₁ is a five- to six-membered aromatic ring system linked via a ring carbon atom to the ring which contains the substituent A, said ring system is unsubstituted or is mono-substituted by a substituent selected from the group consisting of halogen, cyano and C₁-C₄haloalkyl; and said ring system can contain 1 or 2 ring nitrogen atoms; or
Q₁ is a five-membered aromatic ring system linked via a ring nitrogen atom to the ring which contains the substituent A, said ring system is unsubstituted or is mono-substituted by a substituent selected from the group consisting of halogen, cyano and C₁-C₄haloalkyl; and said ring system contains 2 or 3 ring nitrogen atoms;
Each R₄ is independently hydrogen or C₁-C₄alkyl, preferably hydrogen or methyl; and
R₅ is C₁-C₆alkyl or C₃-C₆cycloalkyl, preferably methyl or cyclopropyl; or
an agrochemically acceptable salt, stereoisomer, enantiomer, tautomer or N-oxide thereof.

One further preferred group of compounds according to this embodiment are compounds of formula (I-17a) which are compounds of formula (I-17) wherein A is N.

Another preferred group of compounds according to this embodiment are compounds of formula (I-17b) which are compounds of formula (I-17) wherein A is CH.

One further preferred group of compounds according to this embodiment are compounds of formula (I-17c) which are compounds of formula (I-17) wherein R₃ is hydrogen.

Another preferred group of compounds according to this embodiment are compounds of formula (I-17d) which are compounds of formula (I-17) wherein R₃ is C₁-C₄alkyl, preferably methyl.

One further preferred group of compounds according to this embodiment are compounds of formula (I-17e) which are compounds of formula (I-17) wherein A is N and R₃ is hydrogen.

One further preferred group of compounds according to this embodiment are compounds of formula (I-17f) which are compounds of formula (I-17) wherein Q₁ is hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂ or —N(R₄)COR₅, both in which R₄ is independently hydrogen or C₁-C₄alkyl (preferably hydrogen or methyl) and R₅ is C₁-C₆alkyl (preferably methyl), or Q₁ is 2-pyridyloxy; preferably Q₁ is hydrogen, halogen, trifluoromethyl, cyclopropyl, cyanocyclopropyl, cyanoisopropyl, trifluoroethoxy, —N(R₄)₂ or —N(R₄)COCH₃, both in which R₄ is independently hydrogen or methyl, or Q₁ is 2-pyridyloxy; or

Q₁ is a five-membered aromatic ring system linked via a ring nitrogen atom to the ring which contains the substituent A, said ring system is unsubstituted or is mono-substituted by a substituent selected from the group consisting of halogen, cyano and C₁-C₄haloalkyl; and said ring system contains 2 ring nitrogen atoms; preferably Q₁ is N-linked pyrazolyl which is unsubstituted or is mono-substituted by chloro, cyano or trifluoromethyl.

One further preferred group of compounds according to this embodiment are compounds of formula (I-17g) which are compounds of formula (I-17) wherein Q₁ is hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂ or —N(R₄)COR₅, both in which R₄ is independently hydrogen or C₁-C₄alkyl (preferably hydrogen or methyl) and R₅ is C₁-C₆alkyl (preferably methyl), 2-pyridyloxy or N-linked pyrazolyl which is unsubstituted or is mono-substituted by chloro, cyano or trifluoromethyl; preferably Q₁ is hydrogen, chlorine, bromine, trifluoromethyl, cyclopropyl, 1-cyanocyclopropyl, 1-cyano-1-methyl-ethyl, 2,2,2-trifluoroethoxy, —NH₂, —NH(CH₃), —N(CH₃)₂, —NHCOCH₃, —N(CH₃)COCH₃, 2-pyridyloxy, pyrazol-1-yl, 3-chloro-pyrazol-1-yl, 3-cyano-pyrazol-1-yl or 3-trifluoromethyl-pyrazol-1-yl.

One further preferred group of compounds according to this embodiment are compounds of formula (I-17g-1) which are compounds of formula (I-17) wherein Q₁ is hydrogen, C₁-C₆haloalkyl, C₃-C₆cycloalkyl, C₃-C₆cycloalkyl monosubstituted by cyano, C₁-C₆cyanoalkyl, —N(R₄)₂ or —N(R₄)COR₅, both in which R₄ is independently hydrogen or C₁-C₄alkyl (preferably hydrogen or methyl) and R₅ is C₁-C₆alkyl (preferably methyl), 2-pyridyloxy or N-linked pyrazolyl which is mono-substituted by chloro or trifluoromethyl; preferably Q₁ is hydrogen, trifluoromethyl, cyclopropyl, 1-cyanocyclopropyl, 1-cyano-1-methyl-ethyl, —NH(CH₃), —N(CH₃)COCH₃, 2-pyridyloxy, 3-chloro-pyrazol-1-yl or 3-trifluoromethyl-pyrazol-1-yl.

One further preferred group of compounds according to this embodiment are compounds of formula (I-17g-2) which are compounds of formula (I-17) wherein Q₁ is hydrogen, C₃-C₆cycloalkyl, C₃-C₆cycloalkyl monosubstituted by cyano, or C₁-C₆cyanoalkyl; preferably Q₁ is hydrogen, cyclopropyl, 1-cyanocyclopropyl, or 1-cyano-1-methyl-ethyl.

One further preferred group of compounds according to this embodiment are compounds of formula (I-17h) which are compounds of formula (I-17) wherein

### A is N;

R₂ is C₁-C₆haloalkyl, preferably —CH₂CF₂CHF₂ or —CH₂CF₂CF₃;
R₃ is hydrogen or C₁-C₄alkyl, preferably hydrogen or methyl; and
Q₁ is hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂ or —N(R₄)COR₅, both in which R₄ is independently hydrogen or C₁-C₄alkyl (preferably hydrogen or methyl) and R₅ is C₁-C₆alkyl (preferably methyl), or Q₁ is 2-pyridyloxy; preferably Q₁ is hydrogen, halogen, trifluoromethyl, cyclopropyl, cyanocyclopropyl, cyanoisopropyl, trifluoroethoxy, —N(R₄)₂ or —N(R₄)COCH₃, both in which R₄ is independently hydrogen or methyl, or Q₁ is 2-pyridyloxy; or
Q₁ is a five-membered aromatic ring system linked via a ring nitrogen atom to the ring which contains the substituent A, said ring system is unsubstituted or is mono-substituted by a substituent selected from the group consisting of halogen, cyano and C₁-C₄haloalkyl; and said ring system contains 2 ring nitrogen atoms; preferably Q₁ is N-linked pyrazolyl which is unsubstituted or is mono-substituted by chloro, cyano or trifluoromethyl.

Another preferred group of compounds according to this embodiment are compounds of formula (I-17i) which are compounds of formula (I-17) wherein

### A is N;

R₂ is C₁-C₆haloalkyl, preferably —CH₂CF₂CHF₂ or —CH₂CF₂CF₃;
R₃ is hydrogen; and
Q₁ is hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂ or —N(R₄)COR₅, both in which R₄ is independently hydrogen or C₁-C₄alkyl (preferably hydrogen or methyl) and R₅ is C₁-C₆alkyl (preferably methyl), 2-pyridyloxy or N-linked pyrazolyl which is unsubstituted or is mono-substituted by chloro, cyano or trifluoromethyl; preferably Q₁ is hydrogen, chlorine, bromine, trifluoromethyl, cyclopropyl, 1-cyanocyclopropyl, 1-cyano-1-methyl-ethyl, 2,2,2-trifluoroethoxy, —NH₂, —NH(CH₃), —N(CH₃)₂, —NHCOCH₃, —N(CH₃)COCH₃, 2-pyridyloxy, pyrazol-1-yl, 3-chloro-pyrazol-1-yl, 3-cyano-pyrazol-1-yl or 3-trifluoromethyl-pyrazol-1-yl.

Another preferred group of compounds according to this embodiment are compounds of formula (I-17i-1) which are compounds of formula (I-17) wherein

### A is N;

R₂ is C₁-C₆haloalkyl, preferably —CH₂CF₂CHF₂ or —CH₂CF₂CF₃;
R₃ is hydrogen; and
Q₁ is hydrogen, C₁-C₆haloalkyl, C₃-C₆cycloalkyl, C₃-C₆cycloalkyl monosubstituted by cyano, C₁-C₆cyanoalkyl, —N(R₄)₂ or —N(R₄)COR₅, both in which R₄ is independently hydrogen or C₁-C₄alkyl (preferably hydrogen or methyl) and R₅ is C₁-C₆alkyl (preferably methyl), 2-pyridyloxy or N-linked pyrazolyl which is mono-substituted by chloro or trifluoromethyl; preferably Q₁ is hydrogen, trifluoromethyl, cyclopropyl, 1-cyanocyclopropyl, 1-cyano-1-methyl-ethyl, —NH(CH₃), —N(CH₃)COCH₃, 2-pyridyloxy, 3-chloro-pyrazol-1-yl or 3-trifluoromethyl-pyrazol-1-yl.

Another preferred group of compounds according to this embodiment are compounds of formula (I-17i-2) which are compounds of formula (I-17) wherein

### A is N;

R₂ is C₁-C₆haloalkyl, preferably —CH₂CF₂CHF₂ or —CH₂CF₂CF₃;
R₃ is hydrogen; and
Q₁ is hydrogen, C₃-C₆cycloalkyl, C₃-C₆cycloalkyl monosubstituted by cyano, or C₁-C₆cyanoalkyl; preferably Q₁ is hydrogen, cyclopropyl, 1-cyanocyclopropyl, or 1-cyano-1-methyl-ethyl.

Another preferred group of compounds according to this embodiment are compounds of formula (I-17j) which are compounds of formula (I-17) wherein

### A is N;

R₂ is C₁-C₆haloalkyl, preferably —CH₂CF₂CHF₂ or —CH₂CF₂CF₃;
R₃ is hydrogen; and
Q₁ is hydrogen, halogen, C₃-C₆cycloalkyl, C₁-C₆cyanoalkyl, or —N(R₄)COR₅ in which R₄ is hydrogen or C₁-C₄alkyl (preferably hydrogen or methyl) and R₅ is C₁-C₆alkyl (preferably methyl); preferably Q₁ is hydrogen, chlorine, bromine, cyclopropyl, 1-cyano-1-methyl-ethyl, or —N(CH₃)COCH₃.

Another preferred group of compounds according to this embodiment are compounds of formula (I-17k) which are compounds of formula (I-17) wherein

### A is N;

R₂ is C₁-C₆haloalkyl, preferably —CH₂CF₂CHF₂ or —CH₂CF₂CF₃;
R₃ is C₁-C₄alkyl, preferably methyl; and
Q₁ is hydrogen, halogen, C₃-C₆cycloalkyl, C₁-C₆cyanoalkyl, or —N(R₄)COR₅ in which R₄ is hydrogen or C₁-C₄alkyl (preferably hydrogen or methyl) and R₅ is C₁-C₆alkyl (preferably methyl); preferably Q₁ is hydrogen, chlorine, bromine, cyclopropyl, 1-cyano-1-methyl-ethyl, or —N(CH₃)COCH₃.

Another group of compounds according to the invention are those of formula I-18

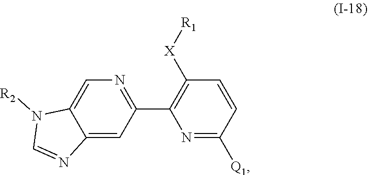

wherein X, R₁ and R₂ are as defined for compounds of formula I (above), or an agrochemically acceptable salt, stereoisomer, enantiomer, tautomer or N-oxide thereof, and wherein Q₁ is preferably hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂, —N(R₄)COR₅, or —N(R₄)CON(R₄)₂, (oxazolidin-2-one)-3-yl or 2-pyridyloxy; or
Q₁ is a five- to six-membered aromatic ring system linked via a ring carbon atom to the pyridyl ring substituted by X—R₁, said ring system is unsubstituted or is mono-substituted by a substituent selected from the group consisting of halogen, cyano and C₁-C₄haloalkyl; and said ring system can contain 1 or 2 ring nitrogen atoms; or
Q₁ is a five-membered aromatic ring system linked via a ring nitrogen atom to the pyridyl ring substituted by X—R₁, said ring system is unsubstituted or is mono-substituted by a substituent selected from the group consisting of halogen, cyano and C₁-C₄haloalkyl; and said ring system contains 2 or 3 ring nitrogen atoms;
Preferably each R₄ is independently hydrogen or C₁-C₄alkyl; and
R₅ is preferably C₁-C₆alkyl or C₃-C₆cycloalkyl.

Preferred definitions of X, R₁ and R₂ are as defined for compounds of formula I (above), and more preferably Q₁ is hydrogen, halogen, trifluoromethyl, cyclopropyl, cyanocyclopropyl, cyanoisopropyl, trifluoroethoxy, —N(R₄)₂, —N(R₄)COR₅, or —N(R₄)CON(R₄)₂, in which each R₄ is independently either hydrogen or methyl and R₅ is either methyl or cyclopropyl, or Q₁ is (oxazolidin-2-one)-3-yl, 2-pyridyloxy, N-linked pyrazolyl which is unsubstituted or is mono-substituted by chloro, cyano or trifluoromethyl; or Q₁ is N-linked triazolyl or C-linked pyrimidinyl; even more preferably Q₁ is hydrogen, halogen, trifluoromethyl, cyclopropyl, cyanocyclopropyl, cyanoisopropyl, —N(R₄)₂, —N(R₄)COR₅, or —N(R₄)CON(R₄)₂, in which each R₄ is independently either hydrogen or methyl and R₅ is either methyl or cyclopropyl, or Q₁ is (oxazolidin-2-one)-3-yl, 2-pyridyloxy, N-linked pyrazolyl which is mono-substituted by chloro or trifluoromethyl; or Q₁ is N-linked triazolyl or C-linked pyrimidinyl.

One group of compounds according to this embodiment are compounds of formula (I-18a) which are compounds of formula (I-18) wherein X is S or SO₂, preferably SO₂.

Another group of compounds according to this embodiment are compounds of formula (I-18b-1) which are compounds of formula (I-18) wherein R₁ is C₁-C₄alkyl or cyclopropyl-C₁-C₄alkyl, preferably ethyl or cyclopropylmethyl.

Another group of compounds according to this embodiment are compounds of formula (I-18b-2) which are compounds of formula (I-18) wherein R₁ is C₁-C₄alkyl, preferably ethyl.

Another group of compounds according to this embodiment are compounds of formula (I-18c) which are compounds of formula (I-18) wherein R₂ is C₁-C₆haloalkyl, more preferably C₁-C₆fluoroalkyl, most preferably —CH₂CF₂CHF₂ or —CH₂CF₂CF₃.

Another group of compounds according to the invention are those of formula I-19

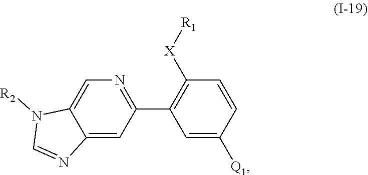

wherein X, R₁ and R₂ are as defined for compounds of formula I (above), or an agrochemically acceptable salt, stereoisomer, enantiomer, tautomer or N-oxide thereof, and wherein Q₁ is preferably hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂, —N(R₄)COR₅, or —N(R₄)CON(R₄)₂, (oxazolidin-2-one)-3-yl or 2-pyridyloxy; or
Q₁ is a five- to six-membered aromatic ring system linked via a ring carbon atom to the phenyl ring substituted by X—R₁, said ring system is unsubstituted or is mono-substituted by a substituent selected from the group consisting of halogen, cyano and C₁-C₄haloalkyl; and said ring system can contain 1 or 2 ring nitrogen atoms; or
Q₁ is a five-membered aromatic ring system linked via a ring nitrogen atom to the phenyl ring substituted by X—R₁, said ring system is unsubstituted or is mono-substituted by a substituent selected from the group consisting of halogen, cyano and C₁-C₄haloalkyl; and said ring system contains 2 or 3 ring nitrogen atoms;
Preferably each R₄ is independently hydrogen or C₁-C₄alkyl; and
R₅ is preferably C₁-C₆alkyl or C₃-C₆cycloalkyl.

Preferred definitions of X, R₁ and R₂ are as defined for compounds of formula I (above), and more preferably Q₁ is hydrogen, halogen, trifluoromethyl, cyclopropyl, cyanocyclopropyl, cyanoisopropyl, trifluoroethoxy, —N(R₄)₂, —N(R₄)COR₅, or —N(R₄)CON(R₄)₂, in which each R₄ is independently either hydrogen or methyl and R₅ is either methyl or cyclopropyl, or Q₁ is (oxazolidin-2-one)-3-yl, 2-pyridyloxy, N-linked pyrazolyl which is unsubstituted or is mono-substituted by chloro, cyano or trifluoromethyl; or Q₁ is N-linked triazolyl or C-linked pyrimidinyl; even more preferably Q₁ is hydrogen, halogen, trifluoromethyl, cyclopropyl, cyanocyclopropyl, cyanoisopropyl, —N(R₄)₂, —N(R₄)COR₅, or —N(R₄)CON(R₄)₂, in which each R₄ is independently either hydrogen or methyl and R₅ is either methyl or cyclopropyl, or Q₁ is (oxazolidin-2-one)-3-yl, 2-pyridyloxy, N-linked pyrazolyl which is mono-substituted by chloro or trifluoromethyl; or Q₁ is N-linked triazolyl or C-linked pyrimidinyl.

One group of compounds according to this embodiment are compounds of formula (I-19a) which are compounds of formula (I-19) wherein X is S or SO₂, preferably SO₂.

Another group of compounds according to this embodiment are compounds of formula (I-19b-1) which are compounds of formula (I-19) wherein R₁ is C₁-C₄alkyl or cyclopropyl-C₁-C₄alkyl, preferably ethyl or cyclopropylmethyl.

Another group of compounds according to this embodiment are compounds of formula (I-19b-2) which are compounds of formula (I-19) wherein R₁ is C₁-C₄alkyl, preferably ethyl.

Another group of compounds according to this embodiment are compounds of formula (I-19c) which are compounds of formula (I-19) wherein R₂ is C₁-C₆haloalkyl, more preferably C₁-C₆fluoroalkyl, most preferably —CH₂CF₂CHF₂ or —CH₂CF₂CF₃.

Another group of compounds according to the invention are those of formula I-20

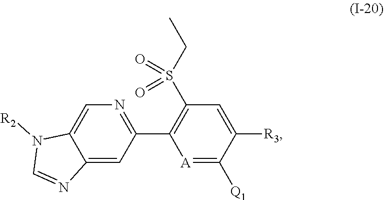

wherein
A is CH or N, preferably N;
R₂ is C₁-C₆haloalkyl, preferably —CH₂CF₂CHF₂ or —CH₂CF₂CF₃;
R₃ is hydrogen or C₁-C₄alkyl, preferably hydrogen or methyl;
Q₁ is hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂, —N(R₄)COR₅, or —N(R₄)CON(R₄)₂, (oxazolidin-2-one)-3-yl or 2-pyridyloxy; or
Q₁ is a five- to six-membered aromatic ring system linked via a ring carbon atom to the ring which contains the substituent A, said ring system is unsubstituted or is mono-substituted by a substituent selected from the group consisting of halogen, cyano and C₁-C₄haloalkyl; and said ring system can contain 1 or 2 ring nitrogen atoms; or
Q₁ is a five-membered aromatic ring system linked via a ring nitrogen atom to the ring which contains the substituent A, said ring system is unsubstituted or is mono-substituted by a substituent selected from the group consisting of halogen, cyano and C₁-C₄haloalkyl; and said ring system contains 2 or 3 ring nitrogen atoms;
Each R₄ is independently hydrogen or C₁-C₄alkyl, preferably hydrogen or methyl; and
R₅ is C₁-C₆alkyl or C₃-C₆cycloalkyl, preferably methyl or cyclopropyl; or
an agrochemically acceptable salt, stereoisomer, enantiomer, tautomer or N-oxide thereof.

One further preferred group of compounds according to this embodiment are compounds of formula (I-20a) which are compounds of formula (I-20) wherein A is N.

Another preferred group of compounds according to this embodiment are compounds of formula (I-20b) which are compounds of formula (I-20) wherein A is CH.

One further preferred group of compounds according to this embodiment are compounds of formula (I-20c) which are compounds of formula (I-20) wherein R₃ is hydrogen.

Another preferred group of compounds according to this embodiment are compounds of formula (I-20d) which are compounds of formula (I-20) wherein R₃ is C₁-C₄alkyl, preferably methyl.

One further preferred group of compounds according to this embodiment are compounds of formula (I-20e) which are compounds of formula (I-20) wherein A is N and R₃ is hydrogen.

One further preferred group of compounds according to this embodiment are compounds of formula (I-20f) which are compounds of formula (I-20) wherein Q₁ is hydrogen, halogen, C₃-C₆cycloalkyl, —N(R₄)₂, —N(R₄)COR₅ or —N(R₄)CON(R₄)₂, in each of which R₄ is independently either hydrogen or C₁-C₄alkyl (preferably hydrogen or methyl) and R₅ is C₁-C₆alkyl or C₃-C₆cycloalkyl (preferably methyl or cyclopropyl), or Q₁ is (oxazolidin-2-one)-3-yl; preferably Q₁ is hydrogen, halogen, cyclopropyl, —N(R₄)₂, —N(R₄)COR₅ or —N(R₄)CON(R₄)₂, in each of which R₄ is independently either hydrogen or methyl and R₅ is methyl or cyclopropyl, or Q₁ is (oxazolidin-2-one)-3-yl; or

Q₁ is a six-membered aromatic ring system linked via a ring carbon atom to the ring which contains the substituent A, said ring system is unsubstituted or is mono-substituted by a substituent selected from the group consisting of halogen, cyano and C₁-C₄haloalkyl; and said ring system contains 2 ring nitrogen atoms; preferably Q₁ is pyrimidinyl which is unsubstituted or is mono-substituted by chloro, cyano or trifluoromethyl; or
Q₁ is a five-membered aromatic ring system linked via a ring nitrogen atom to the ring which contains the substituent A, said ring system is unsubstituted or is mono-substituted by a substituent selected from the group consisting of halogen, cyano and C₁-C₄haloalkyl; and said ring system contains 3 ring nitrogen atoms; preferably Q₁ is N-linked triazolyl which is unsubstituted or is mono-substituted by chloro, cyano or trifluoromethyl.

One further preferred group of compounds according to this embodiment are compounds of formula (I-20g) which are compounds of formula (I-20) wherein Q₁ is hydrogen, halogen, C₃-C₆cycloalkyl, —N(R₄)₂, —N(R₄)COR₅ or —N(R₄)CON(R₄)₂, in each of which R₄ is independently either hydrogen or C₁-C₄alkyl (preferably hydrogen or methyl) and R₅ is C₁-C₆alkyl or C₃-C₆cycloalkyl (preferably methyl or cyclopropyl); or Q₁ is (oxazolidin-2-one)-3-yl, C-linked pyrimidinyl or N-linked triazolyl; preferably Q₁ is hydrogen, chlorine, bromine, cyclopropyl, —NH₂, —NH(CH₃), —NHCOCH₃, —N(CH₃)COCH₃, —NHCO(cyclopropyl), —N(CH₃)CO(cyclopropyl), —N(H)CONH₂, —N(H)CONH(CH₃), —N(H)CON(CH₃)₂, —N(CH₃)CONH₂, —N(CH₃)CONH(CH₃), —N(CH₃)CON(CH₃)₂, (oxazolidin-2-one)-3-yl, pyrimidin-2-yl or 1,2,4-triazol-1-yl.

One further preferred group of compounds according to this embodiment are compounds of formula (I-20g-1) which are compounds of formula (I-20) wherein Q₁ is hydrogen, halogen, or N-linked triazolyl; preferably Q₁ is hydrogen, chlorine or 1,2,4-triazol-1-yl.

One further preferred group of compounds according to this embodiment are compounds of formula (I-20h) which are compounds of formula (I-20) wherein

### A is N;

R₂ is C₁-C₆haloalkyl, preferably —CH₂CF₂CHF₂ or —CH₂CF₂CF₃;
R₃ is hydrogen or C₁-C₄alkyl, preferably hydrogen or methyl; and
Q₁ is hydrogen, halogen, C₃-C₆cycloalkyl, —N(R₄)₂, —N(R₄)COR₅ or —N(R₄)CON(R₄)₂, in each of which R₄ is independently either hydrogen or C₁-C₄alkyl (preferably hydrogen or methyl) and R₅ is C₁-C₆alkyl or C₃-C₆cycloalkyl (preferably methyl or cyclopropyl), or Q₁ is (oxazolidin-2-one)-3-yl; preferably Q₁ is hydrogen, halogen, cyclopropyl, —N(R₄)₂, —N(R₄)COR₅ or —N(R₄)CON(R₄)₂, in each of which R₄ is independently either hydrogen or methyl and R₅ is methyl or cyclopropyl, or Q₁ is (oxazolidin-2-one)-3-yl; or
Q₁ is a six-membered aromatic ring system linked via a ring carbon atom to the ring which contains the substituent A, said ring system is unsubstituted or is mono-substituted by a substituent selected from the group consisting of halogen, cyano and C₁-C₄haloalkyl; and said ring system contains 2 ring nitrogen atoms; preferably Q₁ is pyrimidinyl which is unsubstituted or is mono-substituted by chloro, cyano or trifluoromethyl; or
Q₁ is a five-membered aromatic ring system linked via a ring nitrogen atom to the ring which contains the substituent A, said ring system is unsubstituted or is mono-substituted by a substituent selected from the group consisting of halogen, cyano and C₁-C₄haloalkyl; and said ring system contains 3 ring nitrogen atoms; preferably Q₁ is N-linked triazolyl which is unsubstituted or is mono-substituted by chloro, cyano or trifluoromethyl.

Another preferred group of compounds according to this embodiment are compounds of formula (I-20i) which are compounds of formula (I-20) wherein

### A is N

R₂ is C₁-C₆haloalkyl, preferably —CH₂CF₂CHF₂ or —CH₂CF₂CF₃;
R₃ is hydrogen; and
Q₁ is hydrogen, halogen, C₃-C₆cycloalkyl, —N(R₄)₂, —N(R₄)COR₅ or —N(R₄)CON(R₄)₂, in each of which R₄ is independently either hydrogen or C₁-C₄alkyl (preferably hydrogen or methyl) and R₅ is C₁-C₆alkyl or C₃-C₆cycloalkyl (preferably methyl or cyclopropyl); or Q₁ is (oxazolidin-2-one)-3-yl, C-linked pyrimidinyl or N-linked triazolyl; preferably Q₁ is hydrogen, chlorine, bromine, cyclopropyl, —NH₂, —NH(CH₃), —NHCOCH₃, —N(CH₃)COCH₃, —NHCO(cyclopropyl), —N(CH₃)CO(cyclopropyl), —N(H)CONH₂, —N(H)CONH(CH₃), —N(H)CON(CH₃)₂, —N(CH₃)CONH₂, —N(CH₃)CONH(CH₃), —N(CH₃)CON(CH₃)₂, (oxazolidin-2-one)-3-yl, pyrimidin-2-yl or 1,2,4-triazol-1-yl.

Another preferred group of compounds according to this embodiment are compounds of formula (I-20i-1) which are compounds of formula (I-20) wherein

### A is N;

R₂ is C₁-C₆haloalkyl, preferably —CH₂CF₂CHF₂ or —CH₂CF₂CF₃;
R₃ is hydrogen; and
Q₁ is hydrogen, halogen, or N-linked triazolyl; preferably Q₁ is hydrogen, chlorine or 1,2,4-triazol-1-yl.

Another preferred group of compounds according to this embodiment are compounds of formula (I-20j) which are compounds of formula (I-20) wherein

### A is N;

R₂ is C₁-C₆haloalkyl, preferably —CH₂CF₂CHF₂ or —CH₂CF₂CF₃;
R₃ is hydrogen; and
Q₁ is hydrogen, halogen, —N(R₄)CON(R₄)₂, in which R₄ is independently either hydrogen or C₁-C₄alkyl (preferably hydrogen or methyl) or 1,2,4-triazol-1-yl; preferably Q₁ is hydrogen, chlorine, bromine, —N(H)CONH(CH₃), —N(CH₃)CONH(CH₃) or 1,2,4-triazol-1-yl.

An outstanding group of compounds according to the invention are those of formula I-21

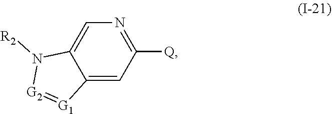

wherein G₁ and G₂ are as defined for compounds of formula I (above), and wherein
R₂ is C₁-C₆haloalkyl, preferably —CH₂CF₂CHF₂ or —CH₂CF₂CF₃;
Q is a radical selected from the group consisting of formula Qa1 and Qb1

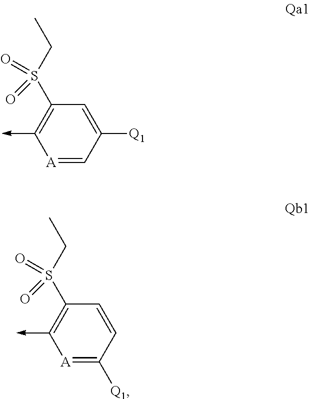

wherein the arrow denotes the point of attachment to the carbon atom of the bicyclic ring;
and wherein
A is CH or N, preferably N; and
Q₁ is hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂, —N(R₄)COR₅ or —N(R₄)CON(R₄)₂, in each of which R₄ is independently either hydrogen or C₁-C₄alkyl (preferably hydrogen or methyl) and R₅ is C₁-C₆alkyl or C₃-C₆cycloalkyl (preferably methyl or cyclopropyl); or Q₁ is (oxazolidin-2-one)-3-yl, 2-pyridyloxy, N-linked pyrazolyl which is unsubstituted or is mono-substituted by chloro, cyano or trifluoromethyl; or Q₁ is C-linked pyrimidinyl or N-linked triazolyl; preferably Q₁ is hydrogen, chlorine, bromine, trifluoromethyl, cyclopropyl, 1-cyanocyclopropyl, 1-cyano-1-methyl-ethyl, 2,2,2-trifluoroethoxy, —NH₂, —NH(CH₃), —N(CH₃)₂, —NHCOCH₃, —N(CH₃)COCH₃, —NHCO(cyclopropyl), —N(CH₃)CO(cyclopropyl), —N(H)CONH₂, —N(H)CONH(CH₃), —N(H)CON(CH₃)₂, —N(CH₃)CONH₂, —N(CH₃)CONH(CH₃), —N(CH₃)CON(CH₃)₂, (oxazolidin-2-one)-3-yl, 2-pyridyloxy, pyrazol-1-yl, 3-chloro-pyrazol-1-yl, 3-cyano-pyrazol-1-yl, 3-trifluoromethyl-pyrazol-1-yl, pyrimidin-2-yl or 1,2,4-triazol-1-yl.

One group of compounds according to this embodiment are compounds of formula (I-21a) which are compounds of formula (I-21) wherein G₂ is N and G₁ is CH.

Another group of compounds according to this embodiment are compounds of formula (I-21 b) which are compounds of formula (I-21) wherein G₂ and G₁ are both CH.

Another group of compounds according to this embodiment are compounds of formula (I-21c) which are compounds of formula (I-21) wherein G₂ is CH and G₁ is N.

One further outstanding group of compounds according to this embodiment are compounds of formula (I-21-1) which are compounds of formula (I-21) wherein G₁ and G₂ are as defined for compounds of formula I (above), and wherein

R₂ is C₁-C₆haloalkyl, preferably —CH₂CF₂CHF₂ or —CH₂CF₂CF₃;
Q is a radical selected from the group consisting of formula Qa1 and Qb1, wherein

### A is N; and

Q₁ is hydrogen, halogen, C₃-C₆cycloalkyl, C₁-C₆cyanoalkyl, —N(R₄)COR₅ or —N(R₄)CON(R₄)₂, both in which R₄ is independently either hydrogen or C₁-C₄alkyl (preferably hydrogen or methyl) and R₅ is C₁-C₆alkyl (preferably methyl) or 1,2,4-triazol-1-yl; preferably Q₁ is hydrogen, chlorine, bromine, cyclopropyl, 1-cyano-1-methyl-ethyl, —N(CH₃)COCH₃, —N(H)CONH(CH₃), —N(CH₃)CONH(CH₃) or 1,2,4-triazol-1-yl.

One group of compounds according to this embodiment are compounds of formula (I-21-1a) which are compounds of formula (I-21-1) wherein G₂ is N and G₁ is CH.

Another group of compounds according to this embodiment are compounds of formula (I-21-1 b) which are compounds of formula (I-21-1) wherein G₂ and G₁ are both CH.

Another group of compounds according to this embodiment are compounds of formula (I-21-1c) which are compounds of formula (I-21-1) wherein G₂ is CH and G₁ is N.

One further outstanding group of compounds according to this embodiment are compounds of formula (I-21-2) which are compounds of formula (I-21) wherein G₁ and G₂ are as defined for compounds of formula I (above), and wherein

R₂ is C₁-C₆haloalkyl, preferably —CH₂CF₂CHF₂ or —CH₂CF₂CF₃;
Q is a radical selected from the group consisting of formula Qa1 and Qb1, wherein

### A is N; and

Q₁ is hydrogen, chlorine, bromine, cyclopropyl, 1-cyano-1-methyl-ethyl, —N(CH₃)COCH₃, —N(H)CONH(CH₃), —N(CH₃)CONH(CH₃) or 1,2,4-triazol-1-yl.

One group of compounds according to this embodiment are compounds of formula (I-21-2a) which are compounds of formula (I-21-2) wherein G₂ is N and G₁ is CH.

Another group of compounds according to this embodiment are compounds of formula (I-21-2b) which are compounds of formula (I-21-2) wherein G₂ and G₁ are both CH.

Another group of compounds according to this embodiment are compounds of formula (I-21-2c) which are compounds of formula (I-21-2) wherein G₂ is CH and G₁ is N.

One further outstanding group of compounds according to this embodiment are compounds of formula (I-21-3) which are compounds of formula (I-21) wherein G₁ and G₂ are as defined for compounds of formula I (above), and wherein

R₂ is C₁-C₆haloalkyl, preferably —CH₂CF₂CHF₂ or —CH₂CF₂CF₃;
Q is a radical selected from the group consisting of formula Qa1 and Qb1, wherein

### A is N; and

Q₁ is hydrogen, bromine, cyclopropyl, 1-cyano-1-methyl-ethyl or —N(CH₃)COCH₃ when Q is Qa1; or
Q₁ is hydrogen, chlorine, —N(H)CONH(CH₃), —N(CH₃)CONH(CH₃) or 1,2,4-triazol-1-yl, when Q is Qb1.

One group of compounds according to this embodiment are compounds of formula (I-21-3a) which are compounds of formula (I-21-3) wherein G₂ is N and G₁ is CH.

Another group of compounds according to this embodiment are compounds of formula (I-21-3b) which are compounds of formula (I-21-3) wherein G₂ and G₁ are both CH.

Another group of compounds according to this embodiment are compounds of formula (I-21-3c) which are compounds of formula (I-21-3) wherein G₂ is CH and G₁ is N.

One further outstanding group of compounds according to this embodiment are compounds of formula (I-21-4) which are compounds of formula (I-21) wherein G₁ and G₂ are as defined for compounds of formula I (above), and wherein

R₂ is C₁-C₆haloalkyl, preferably —CH₂CF₂CHF₂ or —CH₂CF₂CF₃;
Q is a radical selected from the group consisting of formula Qa1 and Qb1, wherein

### A is N; and

Q₁ is hydrogen, halogen, C₁-C₆haloalkyl, C₃-C₆cycloalkyl, C₃-C₆cycloalkyl monosubstituted by cyano, C₁-C₆cyanoalkyl, —N(R₄)₂, —N(R₄)COR₅ or —N(R₄)CON(R₄)₂, in each of which R₄ is independently either hydrogen or C₁-C₄alkyl (preferably hydrogen or methyl) and R₅ is C₁-C₆alkyl or C₃-C₆cycloalkyl (preferably methyl or cyclopropyl); or Q₁ is (oxazolidin-2-one)-3-yl, 2-pyridyloxy, N-linked pyrazolyl which is mono-substituted by chloro or trifluoromethyl; or Q₁ is C-linked pyrimidinyl or N-linked triazolyl; preferably Q₁ is hydrogen, chlorine, trifluoromethyl, cyclopropyl, 1-cyanocyclopropyl, 1-cyano-1-methyl-ethyl, —NH(CH₃), —N(CH₃)COCH₃, —N(CH₃)CO(cyclopropyl), —N(CH₃)CONH(CH₃), (oxazolidin-2-one)-3-yl, 2-pyridyloxy, 3-chloro-pyrazol-1-yl, 3-trifluoromethyl-pyrazol-1-yl, pyrimidin-2-yl or 1,2,4-triazol-1-yl.

One group of compounds according to this embodiment are compounds of formula (I-21-4a) which are compounds of formula (I-21-4) wherein G₂ is N and G₁ is CH.

Another group of compounds according to this embodiment are compounds of formula (I-21-4b) which are compounds of formula (I-21-4) wherein G₂ and G₁ are both CH.

Another group of compounds according to this embodiment are compounds of formula (I-21-4c) which are compounds of formula (I-21-4) wherein G₂ is CH and G₁ is N.

One further outstanding group of compounds according to this embodiment are compounds of formula (I-21-5) which are compounds of formula (I-21) wherein G₁ and G₂ are as defined for compounds of formula I (above), and wherein

R₂ is C₁-C₆haloalkyl, preferably —CH₂CF₂CHF₂ or —CH₂CF₂CF₃, more preferably —CH₂CF₂CF₃;
Q is a radical selected from the group consisting of formula Qa1 and Qb1, wherein

### A is N; and

Q₁ is hydrogen, C₁-C₆haloalkyl, C₃-C₆cycloalkyl, C₃-C₆cycloalkyl monosubstituted by cyano, C₁-C₆cyanoalkyl, —N(R₄)₂, or —N(R₄)COR₅, in each of which R₄ is independently either hydrogen or C₁-C₄alkyl (preferably hydrogen or methyl) and R₅ is C₁-C₆alkyl (preferably methyl), 2-pyridyloxy, or N-linked pyrazolyl which is mono-substituted by chloro or trifluoromethyl; preferably Q₁ is hydrogen, trifluoromethyl, cyclopropyl, 1-cyanocyclopropyl, 1-cyano-1-methyl-ethyl, —NH(CH₃), —N(CH₃)COCH₃, 2-pyridyloxy, 3-chloro-pyrazol-1-yl, or 3-trifluoromethyl-pyrazol-1-yl when Q is Qa1; or
Q₁ is hydrogen, halogen, C₃-C₆cycloalkyl, —N(R₄)₂, —N(R₄)COR₅ or —N(R₄)CON(R₄)₂, in each of which R₄ is independently either hydrogen or C₁-C₄alkyl (preferably hydrogen or methyl) and R₅ is C₁-C₆alkyl or C₃-C₆cycloalkyl (preferably methyl or cyclopropyl); or Q₁ is, (oxazolidin-2-one)-3-yl, C-linked pyrimidinyl or N-linked triazolyl; preferably Q₁ is hydrogen, chlorine, cyclopropyl, —NH(CH₃), —N(CH₃)COCH₃, —N(CH₃)CO(cyclopropyl), —N(CH₃)CONH(CH₃), (oxazolidin-2-one)-3-yl, pyrimidin-2-yl or 1,2,4-triazol-1-yl when Q is Qb1.

One group of compounds according to this embodiment are compounds of formula (I-21-4a) which are compounds of formula (I-21-5) wherein G₂ is N and G₁ is CH.

Another group of compounds according to this embodiment are compounds of formula (I-21-4b) which are compounds of formula (I-21-5) wherein G₂ and G₁ are both CH.

Another group of compounds according to this embodiment are compounds of formula (I-21-4c) which are compounds of formula (I-21-5) wherein G₂ is CH and G₁ is N.

Compounds according to the invention may possess any number of benefits including, inter alia, advantageous levels of biological activity for protecting plants against insects or superior properties for use as agrochemical active ingredients (for example, greater biological activity, an advantageous spectrum of activity, an increased safety profile, improved physico-chemical properties, or increased biodegradability or environmental profile). In particular, it has been surprisingly found that certain compounds of formula (I) may show an advantageous safety profile with respect to non-target arthropods, in particular pollinators such as honey bees, solitary bees, and bumble bees. Most particularly, Apis mellifera.

In another aspect the present invention provides a composition comprising an insecticidally, acaricidally, nematicidally or molluscicidally effective amount of a compound of formula (I), or an agrochemically acceptable salt, stereoisomer, enantiomer, tautomer or N-oxide thereof, as defined in any of the embodiments under compounds of formula (I-1), (I-2), (I-3), (I-4), (I-5), (I-6), (I-7), (I-8), (I-9), (I-10), (I-11), (I-12), (I-13), (I-14), (I-15), (I-16), (I-17), (I-18), (I-19), (I-20) and (I-21) (above), and, optionally, an auxiliary or diluent.

In a further aspect the present invention provides a method of combating and controlling insects, acarines, nematodes or molluscs which comprises applying to a pest, to a locus of a pest, or to a plant susceptible to attack by a pest an insecticidally, acaricidally, nematicidally or molluscicidally effective amount of a compound of formula (I), or an agrochemically acceptable salt, stereoisomer, enantiomer, tautomer or N-oxide thereof, as defined in any of the embodiments under compounds of formula (I-1), (I-2), (I-3), (I-4), (I-5), (I-6), (I-7), (I-8), (I-9), (I-10), (I-11), (I-12), (I-13), (I-14), (I-15), (I-16), (I-17), (I-18), (I-19), (I-20) and (I-21) (above) or a composition as defined above.

In a yet further aspect the present invention provides a method for the protection of plant propagation material from the attack by insects, acarines, nematodes or molluscs, which comprises treating the propagation material or the site, where the propagation material is planted, with a composition as defined above.

The process according to the invention for preparing compounds of formula I is carried out in principle by methods known to those skilled in the art. More specifically, the subgroup of compounds of formula I, wherein X is SO (sulfoxide) and/or SO₂ (sulfone), may be obtained by means of an oxidation reaction of the corresponding sulfide compounds of formula I, wherein X is S, involving reagents such as, for example, m-chloroperoxybenzoic acid (mCPBA), hydrogen peroxide, oxone, sodium periodate, sodium hypochlorite or tert-butyl hypochlorite amongst other oxidants. The oxidation reaction is generally conducted in the presence of a solvent. Examples of the solvent to be used in the reaction include aliphatic halogenated hydrocarbons such as dichloromethane and chloroform; alcohols such as methanol and ethanol; acetic acid; water; and mixtures thereof. The amount of the oxidant to be used in the reaction is generally 1 to 3 moles, preferably 1 to 1.2 moles, relative to 1 mole of the sulfide compounds I to produce the sulfoxide compounds I, and preferably 2 to 2.2 moles of oxidant, relative to 1 mole of the sulfide compounds I to produce the sulfone compounds I. Such oxidation reactions are disclosed, for example, in WO 2013/018928.

The subgroup of compounds of formula I, wherein G₁, G₂ and R₂ are as defined in formula I and wherein Q is defined as Qa, in which Q₁, R₃, X and R₁ are as defined in formula I and wherein A is N, may be defined as compounds of formula I-Qa (scheme 1).

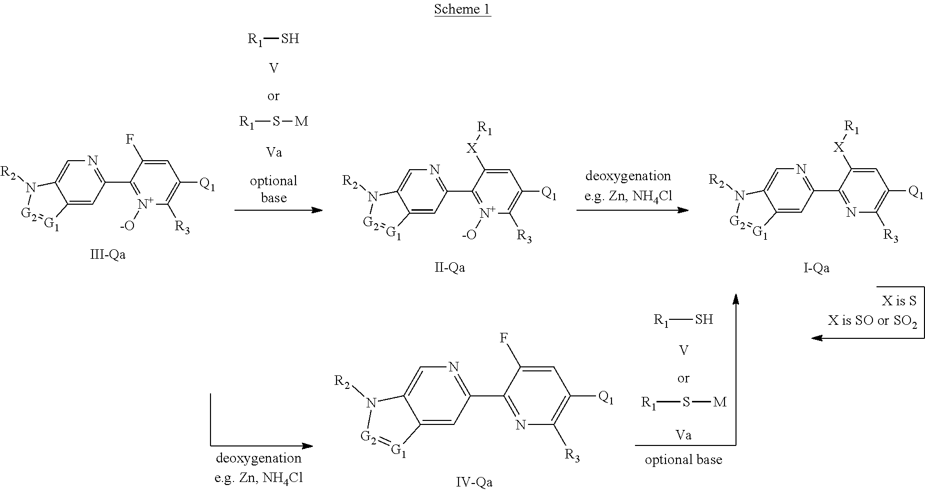

Compounds of formula I-Qa, wherein X is S, and in which G₁, G₂, R₂, Q₁, R₃ and R₁ are as defined in formula I, can be prepared by deoxygenation of compounds of formula II-Qa, wherein X is S, and in which G₁, G₂, R₂, Q₁, R₃ and R₁ are as defined in formula I, using reagents such as zinc powder and ammonium chloride, preferably an aqueous saturated ammonium chloride solution, in ether solvents such as tetrahydrofuran or dioxane, at temperatures between 0° C. and refluxing conditions. Alternatively, such a reduction may also be achieved under conditions known to a person skilled in the art, for example by involving iron powder in acetic acid, or using molecular hydrogen (H₂), optionally under pressure, usually in the presence of a catalyst such as for example Raney-Nickel, or using transfer hydrogenation conditions (for example, ammonium formiate and 5-10% palladium on charcoal in tetrahydrofuran around room temperature), or using phosphorus based reagents such as phosphorus trichloride, triethyl phosphite or triphenyl phosphine.

Compounds of formula II-Qa, wherein X is S, and in which G₁, G₂, R₂, Q₁, R₃ and R₁ are as defined in formula I, can be prepared by reacting compounds of formula III-Qa, wherein G₁, G₂, R₂, Q₁ and R₃ are as defined in formula I, with a reagent of the formula V

R₁—SH (V),

or a salt thereof, wherein R₁ is as defined in formula I, optionally in the presence of a suitable base, such as alkali metal carbonates, for example sodium carbonate and potassium carbonate, or alkali metal hydrides such as sodium hydride, or alkali metal hydroxides such as sodium hydroxide and potassium hydroxide, or sodium or potassium tert-butoxide, in an inert solvent at temperatures preferably between 25-120° C. Examples of solvent to be used include ethers such as tetrahydrofuran THF, ethylene glycol dimethyl ether, tert-butylmethyl ether, and 1,4-dioxane, aromatic hydrocarbons such as toluene and xylene, nitriles such as acetonitrile or polar aprotic solvents such as N,N-dimethylformamide, N,N-dimethylacetamide, N-methyl-2-pyrrolidone NMP or dimethyl sulfoxide.

Examples of salts of the compound of formula V include compounds of the formula Va

R₁—S-M (Va),

wherein R₁ is as defined above and wherein M is, for example, sodium or potassium. Such a process to prepare compounds of formula V can be found, for example, in WO16/091731.

Alternatively, this reaction to form II-Qa can be carried out in the presence of a palladium catalyst, such as tris(dibenzylideneacetone)dipalladium(0), in the presence of a phosphine ligand, such as xanthphos, in an inert solvent, for example, xylene at temperatures between 100-160° C., preferably 140° C., as described in Tetrahedron 2005, 61, 5253-5259.

Such compounds of formula II-Qa

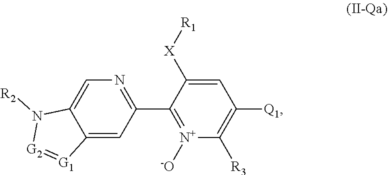

wherein
X is S, and in which G₁, G₂, R₂, Q₁, R₃ and R₁ are as defined in formula I,
are novel, especially developed for the preparation of the compounds of formula I according to the invention and therefore represent a further object of the invention. The preferences and preferred embodiments of the substituents of the compounds of formula I are also valid for the compounds of formula II-Qa.

Alternatively, compounds of formula I-Qa, wherein X is S, may be prepared from compounds of formula III-Qa, by involving the same chemistry as described above, but by changing the order of the steps (i.e. by running the sequence III-Qa to IV-Qa via deoxygenation/reduction, followed by reaction of IV-Qa with V or Va to form I-Qa, wherein all substituent definitions mentioned previously remain valid).

Compounds of formula IV-Qa

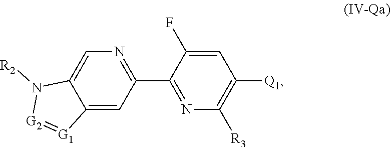

wherein
G₁, G₂, R₂, Q₁ and R₃ are as defined in formula I,
are novel, especially developed for the preparation of the compounds of formula I according to the invention and therefore represent a further object of the invention. The preferences and preferred embodiments of the substituents of the compounds of formula I are also valid for the compounds of formula IV-Qa.

Oxidation of any compounds of formula II-Qa and I-Qa in scheme 1, wherein the substituents are as defined above, and in which X is S (sulfide), with a suitable oxidizing agent, into corresponding compounds wherein X is SO (sulfoxide) or SO₂ (sulfone) may be achieved under conditions already described above.

Compounds of formula V, wherein R₁ is as defined in formula I, and compounds of formula Va, wherein R₁ is as defined above and wherein M is, for example, sodium or potassium, are either known, commercially available or may be prepared by methods known to a person skilled in the art.

Compounds of formula III-Qa, wherein G₁, G₂, R₂, Q₁ and R₃ are as defined in formula I,

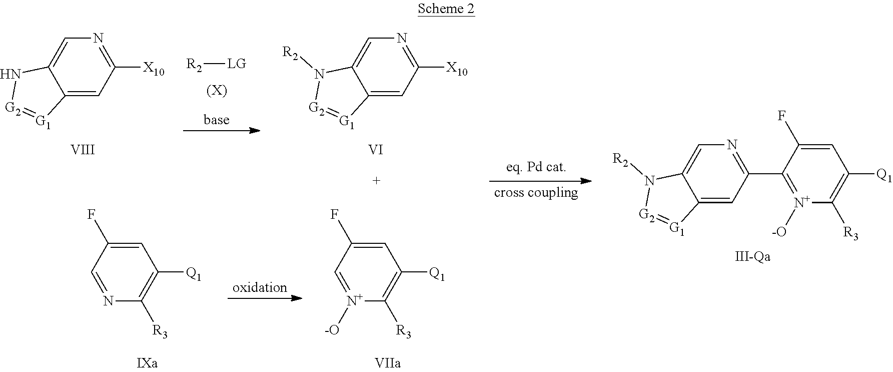

can be prepared (scheme 2) by a cross-coupling reaction between compounds of formula VI, wherein G₁, G₂ and R₂ are as defined in formula I, and in which X₁₀ is a halogen (or a pseudo-halogen leaving group, such as a triflate), preferably bromine or chlorine, and compounds of formula Vila, wherein Q₁ and R₃ are as defined in formula I, under metal catalysis (preferably palladium catalysis) conditions, for example involving [1,1′bis(diphenylphosphino)ferrocene]dichloropalladium (PdCl₂(dppf), optionally as a complex with dichloromethane (preferably a 1:1 complex), in presence of a base such as 2,2,6,6-tetramethylpiperidide zinc chloride lithium chloride (TMPZnCl.LiCl; commercial or prepared according to Org. Lett. 2009, 11, 1837-1840), preferably in form of a solution of the tetramethylpiperidinyl zinc chloride lithium chloride complex in tetrahydrofuran, in ether solvents such as tetrahydrofuran, dioxane or 1,2-dimethoxyethane, preferably tetrahydrofuran, at temperatures between 0° C. and refluxing conditions, preferably between room temperature and 80° C., preferably under inert atmosphere, and optionally microwave irradiation. Such cross-coupling conditions have been described in, for example, Org. Lett. 2012, 14, 862-865.

Alternatively, this cross-coupling step may also perform under Fagnou-type conditions (described by Fagnou et al. in, for example, Org. Lett. 2011, 13, 2310-13 and J. Am. Chem. Soc. 2009, 131, 3291-3306) involving palladium acetate and a phosphine ligand such as tri-tert-butylphosphonium tetrafluoroborate (PtBu₃—HBF₄), in the presence of a base such as potassium carbonate or cesium carbonate, in solvents such as tetrahydrofuran, dioxane, acetonitrile, N,N-dimethylformamide or toluene, at temperatures between 0° C. and 150° C., preferably between room temperature and 120° C., preferably under inert atmosphere, and optionally microwave irradiation.

Such compounds of formula III-Qa

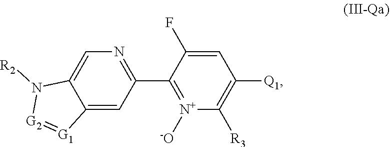

wherein
G₁, G₂, R₂, Q₁ and R₃ are as defined in formula I,
are novel, especially developed for the preparation of the compounds of formula I according to the invention and therefore represent a further object of the invention. The preferences and preferred embodiments of the substituents of the compounds of formula I are also valid for the compounds of formula III-Qa.

Compounds of formula VI, wherein G₁, G₂ and R₂ are as defined in formula I, and in which X₁₀ is a halogen (or a pseudo-halogen leaving group, such as a triflate), preferably bromine or chlorine, can be prepared by reacting compounds of formula VIII, wherein G₁ and G₂ are as defined in formula I, and in which X₁₀ is a halogen (or a pseudo-halogen leaving group, such as a triflate), with reagents of the formula R₂-LG (X), wherein R₂ is as defined in formula I, and in which LG is a halogen, preferably iodine, bromine or chlorine (or a pseudo-halogen leaving group, such as a (halo)alkyl or phenyl sulfonate ester, e.g. triflate), in the presence of a base, such as sodium hydride or an alkaline earth metal hydride, carbonate (e.g. sodium carbonate, potassium carbonate or cesium carbonate) or hydroxide, in an inert solvent such as tetrahydrofuran, dioxane, N,N-dimethylformamide DMF, N,N-dimethylacetamide or acetonitrile and the like, at temperatures between 0 and 120° C., by procedures well known to those skilled in the art.

Such compounds of formula VI

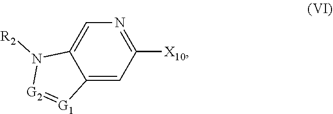

wherein
G₁, G₂ and R₂ are as defined in formula I; and
X₁₀ is a halogen or a pseudo-halogen leaving group, such as a triflate,
are novel, especially developed for the preparation of the compounds of formula I according to the invention and therefore represent a further object of the invention. The preferences and preferred embodiments of the substituents of the compounds of formula I are also valid for the compounds of formula VI. Preferably, X₁₀ is bromo or chloro; even more preferably X₁₀ is bromo.

Compounds of formula VIIa, wherein Q₁ and R₃ are as defined in formula I, can be prepared by oxidation of compounds of formula IXa, wherein Q₁ and R₃ are as defined in formula I, under conditions known to those skilled in the art, involving for example, meta-chloro perbenzoic acid in an inert solvent such as ethyl acetate, chloroform or methylene chloride, at temperatures between 0° C. and 80° C., preferably 10 to 70° C. Alternatively, other suitable oxidizing agents may be used, such as for example methyltrioxorhenium and hydrogen peroxide (either aqueous or as a urea complex), hydrogen peroxide in acetic acid, or the H₂O₂/urea adduct in the presence of an acid anhydride, e.g. trifluoroacetic anhydride. Such oxidations are known from the literature, for example from J. Med. Chem. 1989, 32, 2561 or WO 00/15615.

Compounds of formula IXa, wherein Q₁ and R₃ are as defined in formula I; and

compounds of formula VIII, wherein G₁ and G₂ are as defined in formula I, and in which X₁₀ is a halogen (or a pseudo-halogen leaving group, such as a triflate); and
reagents of the formula R₂-LG (X), wherein R₂ is as defined in formula I, and in which LG is a halogen, preferably iodine, bromine or chlorine (or a pseudo-halogen leaving group, such as a (halo)alkyl or phenyl sulfonate ester, e.g. triflate);
are either known, commercially available or may be prepared by methods known to a person skilled in the art.

The subgroup of compounds of formula I, wherein G₁, G₂ and R₂ are as defined in formula I and wherein Q is defined as Qb, in which Q₁, R₃, X and R₁ are as defined in formula I and wherein A is N, may be defined as compounds of formula I-Qb (scheme 3).

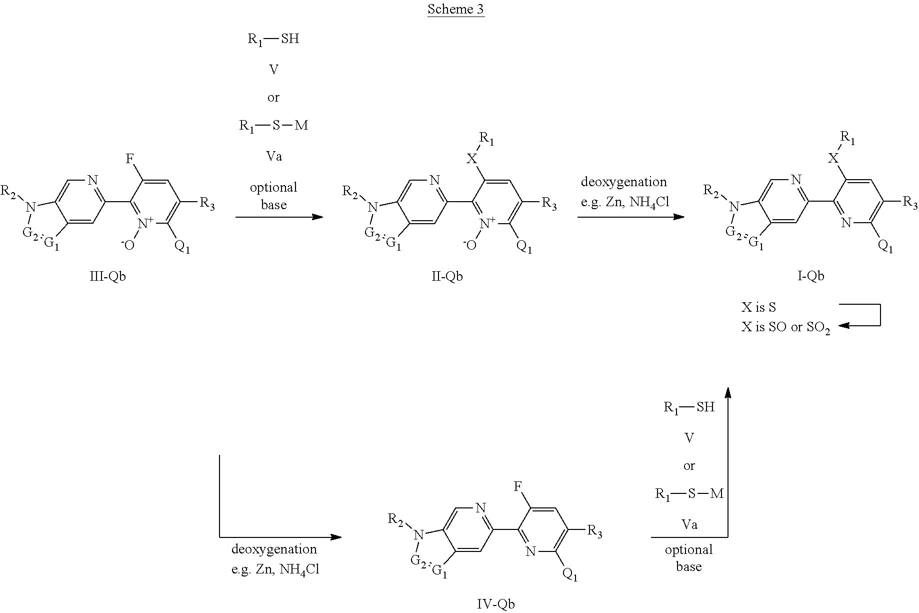

The chemistry described previously in scheme 1 to access compounds of formula I-Qa from compounds of formula III-Qa and reagents of formula V/Va (including the alternative involving IV-Qa), can be applied analogously (scheme 3) for the preparation of compounds of formula I-Qb from compounds of formula III-Qb (including the alternative involving IV-Qb), wherein all substituent definitions mentioned previously remain valid.

The chemistry described previously in scheme 2 to access compounds of formula III-Qa from compounds of formula VI and compounds of formula VIIa can be applied analogously (scheme 4) for the preparation of compounds of formula III-Qb from compounds of formula VI and compounds of formula VIIb, wherein all substituent definitions mentioned previously remain valid.

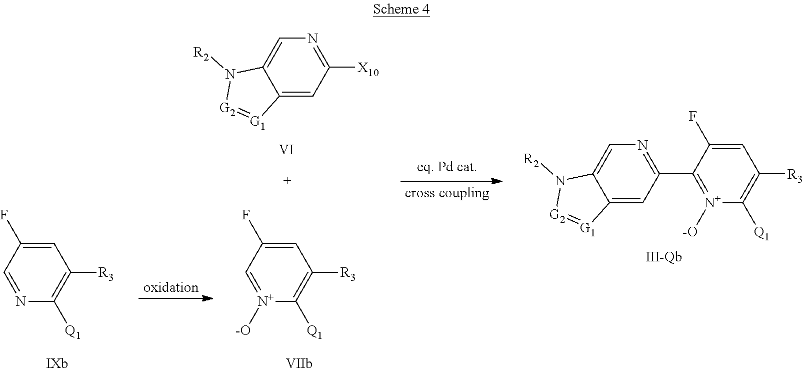

Compounds of formula IXb, wherein Q₁ and R₃ are as defined in formula I, are oxidized by methods described above (scheme 2) into compounds of formula VIIb, wherein Q₁ and R₃ are as defined in formula I.

Compounds of formula IXb, wherein Q₁ and R₃ are as defined in formula I, are either known, commercially available or may be prepared by methods known to a person skilled in the art.

In the particular situation when Q₁ is an optionally substituted triazole linked via a ring nitrogen atom to the ring which contains the group A, then compounds of formula I-Qb, wherein X is S and in which G₁, G₂, R₂, Q₁, R₃ and R₁ are as defined in formula I,

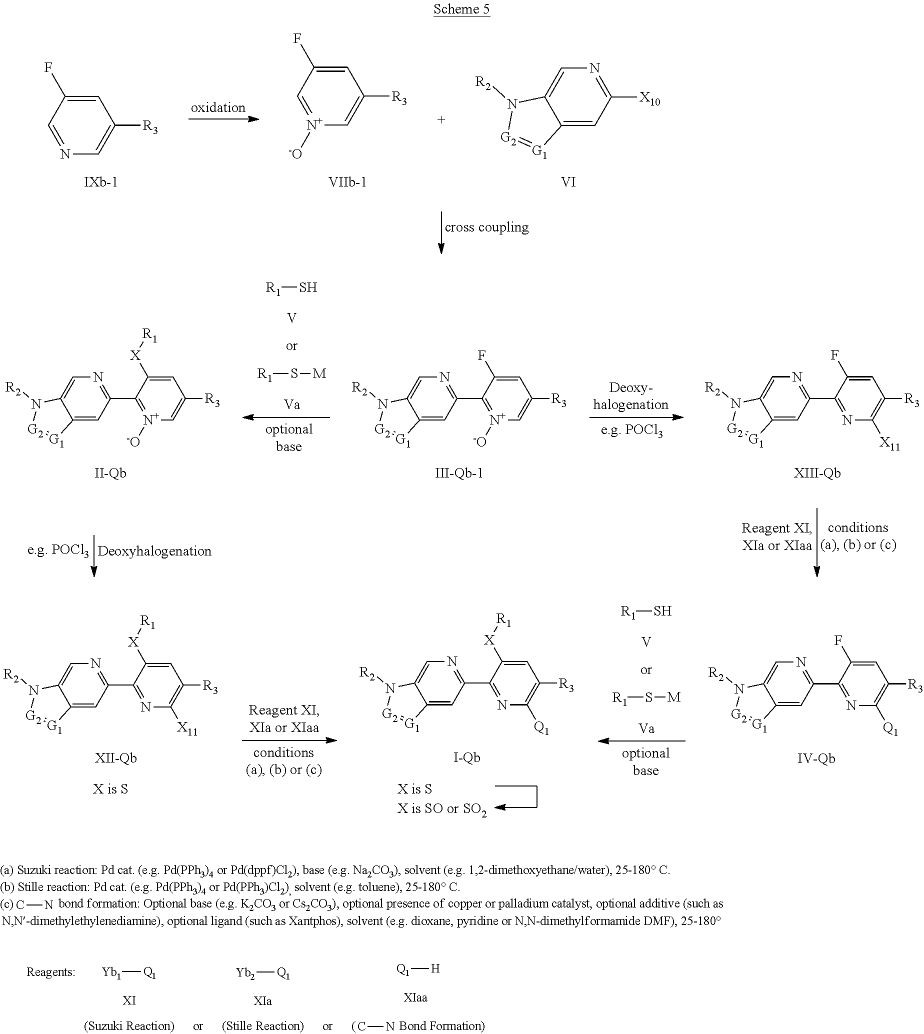

may alternatively be prepared (scheme 5) from compounds of formula XII-Qb, wherein X is S and in which G₁, G₂, R₂, R₃ and R₁ are as defined in formula I, and wherein X₁₁ is a leaving group like, for example, chlorine, bromine or iodine (preferably chlorine or bromine), or an aryl- or alkylsulfonate such as trifluoromethanesulfonate, by reaction (C—N bond formation) with an optionally substituted triazole Q₁-H (which contains an appropriate NH functionality) (XIaa), wherein Q₁ is N-linked triazolyl, in solvents such as alcohols (eg. methanol, ethanol, isopropanol, or higher boiling linear or branched alcohols), pyridine or acetic acid, optionally in the presence of an additional base, such as potassium carbonate K₂CO₃ or cesium carbonate Cs₂CO₃, optionally in the presence of a copper catalyst, for example copper(I) iodide, at temperatures between 30-180° C., optionally under microwave irradiation.

In the particular situation within scheme 5 when Q₁ is —N(R₄)COR₅, or —N(R₄)CON(R₄)₂, wherein R₄ and R₅ are as defined in formula I, then compounds of formula I-Qb, wherein X is S, may be prepared from compounds of formula XII-Qb, wherein X is S and in which G₁, G₂, R₂, R₃ and R₁ are as defined in formula I, and wherein X₁₁ is a leaving group like, for example, chlorine, bromine or iodine (preferably chlorine or bromine), or an aryl- or alkylsulfonate such as trifluoromethanesulfonate, by reaction (C—N bond formation) with a reagent Q₁-H (XIaa) equivalent to HN(R₄)COR₅, or HN(R₄)CON(R₄)₂, wherein R₄ and R₅ are as defined in formula I. Such a reaction is performed in the presence of a base, such as potassium carbonate, cesium carbonate, sodium hydroxide, in an inert solvent, such as toluene, dimethylformamide DMF, N-methyl pyrrolidine NMP, dimethyl sulfoxide DMSO, dioxane, tetrahydrofuran THF, and the like, optionally in the presence of a catalyst, for example palladium(II)acetate, bis(dibenzylideneacetone)palladium(0) (Pd(dba)₂) or tris(dibenzylideneacetone) dipalladium(0) (Pd₂(dba)₃, optionally in form of a chloroform adduct), or a palladium pre-catalyst such as for example tert-BuBrettPhos Pd G3 [(2-Di-tert-butylphosphino-3,6-dimethoxy-2′,4′,6′-triisopropyl-1,1′-biphenyl)-2-(2′-amino-1,1′-biphenyl)]palladium(II) methanesulfonate or BrettPhos Pd G3 [(2-di-cyclohexylphosphino-3,6-dimethoxy-2′,4′,6′-triisopropyl-1,1′-biphenyl)-2-(2′-amino-1,1′-biphenyl)]palladium(II) methanesulfonate, and optionally in the presence of a ligand, for example SPhos, t-BuBrettPhos or Xantphos, at temperatures between 60-120° C., optionally under microwave irradiation.

In the particular situation within scheme 5 when Q₁ is —N(R₄)₂, wherein R₄ is as defined in formula I, then compounds of formula I-Qb, wherein X is S, may be prepared from compounds of formula XII-Qb, wherein X is S and in which G₁, G₂, R₂, R₃ and R₁ are as defined in formula I, and wherein X₁₁ is a leaving group like, for example, chlorine, bromine or iodine (preferably chlorine or bromine), or an aryl- or alkylsulfonate such as trifluoromethanesulfonate, by reaction (C—N bond formation) with a reagent Q₁-H (XIaa) equivalent to HN(R₄)₂, or a salt thereof (such as a hydrohalide salt, preferably a hydrochloride or a hydrobromide salt, or a trifluoroacetic acid salt, or any other equivalent salt), wherein R₄ is as defined in formula I. Such a reaction is commonly performed in an inert solvent such as alcohols, amides, esters, ethers, nitriles and water, particularly preferred are methanol, ethanol, 2,2,2-trifluoroethanol, propanol, isopropanol, N,N-dimethylformamide, N,N-dimethylacetamide, dioxane, tetrahydrofuran, dimethoxyethane, acetonitrile, ethyl acetate, toluene, water or mixtures thereof, at temperatures between 0-150° C., optionally under microwave irradiation or pressurized conditions using an autoclave, optionally in the presence of a copper catalyst, such as copper powder, copper(I) iodide or copper sulfate (optionally in form of a hydrate), or mixtures thereof, optionally in presence a ligand, for example diamine ligands (e.g. N,N′-dimethylethylenediamine or trans-cyclohexyldiamine) or dibenzylideneacetone (dba), or 1,10-phenanthroline, and optionally in presence of a base such as potassium phosphate.

Reagents HN(R₄)₂, HN(R₄)COR₅, or HN(R₄)CON(R₄)₂, wherein R₄ and R₅ are as defined in formula I, are either known, commercially available or may be prepared by methods known to a person skilled in the art.

Alternatively, compounds of formula I-Qb, wherein X is S, may be prepared by a Suzuki reaction (scheme 5), which involves for example, reacting compounds of formula XII-Qb, wherein X is S and in which G₁, G₂, R₂, R₃ and R₁ are as defined in formula I, and wherein X₁₁ is a leaving group like, for example, chlorine, bromine or iodine (preferably chlorine or bromine), or an aryl- or alkylsulfonate such as trifluoromethanesulfonate, with compounds of formula XI, wherein Q₁ is as defined in formula I, and wherein Y_{b1} can be a boron-derived functional group, such as for example B(OH)₂ or B(OR_{b1})₂ wherein R_{b1} can be a C₁-C₄alkyl group or the two groups OR_{b1} can form together with the boron atom a five membered ring, as for example a pinacol boronic ester. The reaction may be catalyzed by a palladium based catalyst, for example tetrakis(triphenyl-phosphine)palladium(0), (1,1′bis(diphenylphosphino) ferrocene)dichloro-palladium-dichloromethane (1:1 complex) or chloro(2-dicyclohexylphosphino-2′,4′, 6′-triisopropyl-1,1′-biphenyl)[2-(2′-amino-1,1′-biphenyl)]palladium(II) (XPhos palladacycle), in presence of a base, like sodium carbonate, tripotassium phosphate or cesium fluoride, in a solvent or a solvent mixture, like, for example dioxane, acetonitrile, N,N-dimethylformamide, a mixture of 1,2-dimethoxyethane and water or of dioxane/water, or of toluene/water, preferably under inert atmosphere. The reaction temperature can preferentially range from room temperature to the boiling point of the reaction mixture, or the reaction may be performed under microwave irradiation. Such Suzuki reactions are well known to those skilled in the art and have been reviewed, for example, in J. Orgmet. Chem. 576, 1999, 147-168.

Alternatively compounds of formula I-Qb, wherein X is S, may be prepared by a Stille reaction between compounds of formula XIa, wherein Q₁ is as defined above, and wherein Y_{b2} is a trialkyl tin derivative, preferably tri-n-butyl tin or tri-methyl-tin, and compounds of formula XII-Qb, wherein X is S and in which G₁, G₂, R₂, R₃ and R₁ are as defined in formula I, and wherein X₁₁ is a leaving group like, for example, chlorine, bromine or iodine (preferably chlorine or bromine), or an aryl- or alkylsulfonate such as trifluoromethanesulfonate. Such Stille reactions are usually carried out in the presence of a palladium catalyst, for example tetrakis(triphenylphosphine)palladium(0), or bis(triphenylphosphine) palladium(II) dichloride, in an inert solvent such as N,N-dimethylformamide, acetonitrile, toluene or dioxane, optionally in the presence of an additive, such as cesium fluoride, or lithium chloride, and optionally in the presence of a further catalyst, for example copper(I)iodide. Such Stille couplings are also well known to those skilled in the art, and have been described in for example J. Org. Chem., 2005, 70, 8601-8604, J. Org. Chem., 2009, 74, 5599-5602, and Angew. Chem. Int. Ed., 2004, 43, 1132-1136.

When Q₁ is a five-membered aromatic ring system linked via a ring nitrogen atom to the ring which contains the substituent A, then compounds of formula I-Qb, wherein X is S, may be prepared from compounds of formula XII-Qb, wherein X is S and in which G₁, G₂, R₂, R₃ and R₁ are as defined in formula I, and wherein X₁₁ is a leaving group like, for example, chlorine, bromine or iodine (preferably chlorine or bromine), or an aryl- or alkylsulfonate such as trifluoromethanesulfonate, by reaction with a heterocycle Q₁-H (which contains an appropriate NH functionality) (XIaa), wherein Q₁ is as defined above, in the presence of a base, such as potassium carbonate K₂CO₃ or cesium carbonate Cs₂CO₃, optionally in the presence of a copper catalyst, for example copper(I) iodide, with or without an additive such as L-proline, N,N′-dimethylcyclohexane-1,2-diamine or N,N′-dimethyl-ethylene-diamine, in an inert solvent such as N-methylpyrrolidone NMP or N,N-dimethylformamide DMF at temperatures between 30-150° C., optionally under microwave irradiation.

A large number of compounds of the formula (XI), (XIa) and (XIaa) are commercially available or can be prepared by those skilled in the art.

Alternatively, compounds of formula I-Qb, wherein X is SO or SO₂, may be prepared from compounds of formula XII-Qb, wherein X is SO or SO₂ and in which G₁, G₂, R₂, R₃ and R₁ are as defined in formula I, and wherein X₁₁ is a leaving group like, for example, chlorine, bromine or iodine (preferably chlorine or bromine), or an aryl- or alkylsulfonate such as trifluoromethanesulfonate, by involving the same chemistry as described above, but by changing the order of the steps (i.e. by running an oxidation step on XII-Qb, wherein X is S, to form XII-Qb, wherein X is SO or SO₂, followed by the sequence XII-Qb (X is SO or SO₂) to I-Qb (X is SO or SO₂) via Suzuki, Stille or C—N bond formation).

Oxidation of compounds of formula XII-Qb, wherein X is S and in which G₁, G₂, R₂, R₃ and R₁ are as defined in formula I, and wherein X₁₁ is a leaving group like, for example, chlorine, bromine or iodine (preferably chlorine or bromine), or an aryl- or alkylsulfonate such as trifluoromethanesulfonate, with a suitable oxidizing agent, into compounds of formula XII-Qb, wherein X is SO or SO₂ may be achieved under conditions already described above.

Compounds of formula XII-Qb, wherein X is S and in which G₁, G₂, R₂, R₃ and R₁ are as defined in formula I, and wherein X₁₁ is a leaving group like, for example, chlorine, bromine or iodine (preferably chlorine or bromine), or an aryl- or alkylsulfonate such as trifluoromethanesulfonate, may be prepared (scheme 5) by reacting compounds of formula II-Qb-1, wherein X is S and in which G₁, G₂, R₂, R₃ and R₁ are as defined in formula I, with a halogenating agent such as phosphorus oxychloride POCl₃ or phosphorus oxybromide, neat or in an appropriate solvent, such as chloroform or toluene, optionally in the presence of a base, such as triethylamine or pyridine, at temperatures between room temperature and refluxing conditions. Such deoxyhalogenation have been described in, for example, WO16/116338.

Compounds of formula II-Qb-1, wherein X is S and in which G₁, G₂, R₂, R₃ and R₁ are as defined in formula I, may be prepared by reacting compounds of formula III-Qb-1, wherein G₁, G₂, R₂ and R₃ are as defined in formula I, with reagents of formula V or Va, wherein R₁ is as defined in formula I, under conditions already described above (see text schemes 1 and 3).

Compounds of formula III-Qb-1, wherein G₁, G₂, R₂ and R₃ are as defined in formula I, may be prepared by cross-coupling compounds of formula VI, wherein G₁, G₂ and R₂ are as defined in formula I, and in which X₁₀ is a halogen (or a pseudo-halogen leaving group, such as a triflate), preferably bromine or chlorine, with compounds of formula VIIb-1, wherein R₃ is as defined in formula I, under conditions already described above (see text schemes 2 and 4).

Compounds of formula IXb-1, wherein R₃ is as defined in formula I, are oxidized by methods described above (see text schemes 2 and 4) into compounds of formula VIIb-1, wherein R₃ is as defined in formula I.

Compounds of formula IXb-1, wherein R₃ is as defined in formula I, are either known, commercially available or may be prepared by methods known to a person skilled in the art.

Alternatively, compounds of formula I-Qb, wherein X is S, SO or SO₂, may be prepared (scheme 5) from compounds of formula III-Qb-1, by involving the same chemistry as described above, but by changing the order of the steps (i.e. by running the sequence III-Qb-1 to XIII-Qb, XIII-Qb to IV-Qb which was described previously, and IV-Qb to I-Qb, followed by oxidation, and wherein all substituent definitions mentioned previously remain valid).

In the particular situation when R₃ is C₁-C₄alkyl, then compounds of formula I-Qa, wherein X is S and in which G₁, G₂, R₂, Q₁ and R₁ are as defined in formula I,

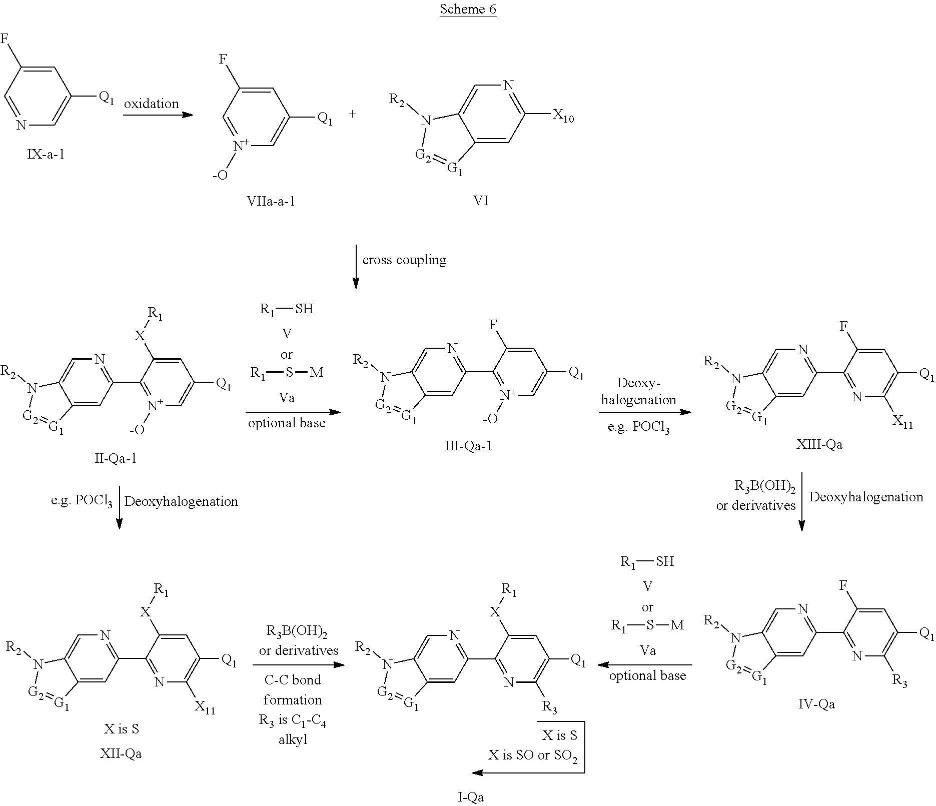

may alternatively be prepared (scheme 6) from compounds of formula XII-Qa, wherein X is S and in which G₁, G₂, R₂, Q₁ and R₁ are as defined in formula I, and wherein X₁₁ is a leaving group like, for example, chlorine, bromine or iodine (preferably chlorine or bromine), or an aryl- or alkylsulfonate such as trifluoromethanesulfonate, by means of a C—C bond formation reaction typically under palladium-catalyzed (alternatively nickel-catalyzed) cross-coupling conditions. Such Suzuki-Miyaura cross-coupling reactions between compounds of formula XII-Qa and C₁-C₄alkyl boronic acids of the formula R₃B(OH)₂, wherein R₃ is C₁-C₄alkyl, or the corresponding C₁-C₄alkyl boronate ester derivatives, or the corresponding 6-membered tri(C₁-C₄alkyl) boroxine derivatives of the formula (R₃BO)₃, wherein R₃ is C₁-C₄alkyl, are well known to a person skilled in the art. In the particular situation where R₃ is methyl, compounds of formula XII-Qa can be reacted, for example, with trimethylboroxine (also known as 2,4,6-trimethyl-1,3,5,2,4,6-trioxatriborinane) in the presence of palladium catalyst, such as tetrakis(triphenylphosphine)-palladium(0) or [1,1′-bis(diphenylphosphino)ferrocene]palladium(II) dichloride dichloromethane complex, and a base, such as sodium or potassium carbonate, in a solvent, such as N,N-dimethylformamide, dioxane or dioxane-water mixtures, at temperatures between room temperature and 160° C., optionally under microwave heating conditions, and preferably under inert atmosphere. Such conditions are described, for example, in Tetrahedron Letters (2000), 41(32), 6237-6240.

Compounds of formula XII-Qa, wherein X is S and in which G₁, G₂, R₂, Q₁ and R₁ are as defined in formula I, and wherein X₁₁ is a leaving group like, for example, chlorine, bromine or iodine (preferably chlorine or bromine), or an aryl- or alkylsulfonate such as trifluoromethanesulfonate, may be prepared from compounds of formula IXa-1 (via compounds of formula III-Qa-1), wherein Q₁ is as defined in formula I, in a sequence and under conditions already described above (see text scheme 5), and wherein all substituent definitions mentioned previously remain valid).

Alternatively, compounds of formula I-Qa, wherein X is SO or SO₂, may be prepared from compounds of formula XII-Qa, wherein X is SO or SO₂ and in which G₁, G₂, R₂, Q₁ and R₁ are as defined in formula I, and wherein X₁₁ is a leaving group like, for example, chlorine, bromine or iodine (preferably chlorine or bromine), or an aryl- or alkylsulfonate such as trifluoromethanesulfonate, by involving the same chemistry as described above, but by changing the order of the steps (i.e. by running an oxidation step on XII-Qa, wherein X is S, to form XII-Qa, wherein X is SO or SO₂, followed by the sequence XII-Qa (X is SO or SO₂) to I-Qa (X is SO or SO₂) via C—C bond formation with R₃B(OH)₂, or equivalent).

Oxidation of compounds of formula XII-Qa, wherein X is S and in which G₁, G₂, R₂, Q₁ and R₁ are as defined in formula I, and wherein X₁₁ is a leaving group like, for example, chlorine, bromine or iodine (preferably chlorine or bromine), or an aryl- or alkylsulfonate such as trifluoromethanesulfonate, with a suitable oxidizing agent, into compounds of formula XII-Qa, wherein X is SO or SO₂ may be achieved under conditions already described above.

Alternatively, compounds of formula I-Qa, wherein X is S, SO or SO₂, may be prepared (scheme 6) from compounds of formula III-Qa-1, by involving the same chemistry as just described above, but by changing the order of the steps (i.e. by running the sequence III-Qa-1 to XIII-Qa, XIII-Qa to IV-Qa which was described previously, and IV-Qa to I-Qa, followed by oxidation, and wherein all substituent definitions mentioned previously remain valid).

In the particular situation when R₃ is hydrogen, then compounds of formula I-Qa, wherein X is S, SO or SO₂, and in which G₁, G₂, R₂, Q₁ and R₁ are as defined in formula I, may alternatively be prepared (scheme 6) from compounds of formula XII-Qa, wherein X is S, SO or SO₂, and in which G₁, G₂, R₂, Q₁ and R₁ are as defined in formula I, and wherein X₁₁ is a leaving group like, for example, chlorine, bromine or iodine (preferably chlorine or bromine), or an aryl- or alkylsulfonate such as trifluoro-methanesulfonate, by means of a reductive dehalogenation. Such a hydrodehalogenation can be achieved, for example, using zinc dust and acetic acid or trifluoroacetic acid, or mixtures thereof, at temperatures between 0° C. and 120° C., preferably between 50° C. and reflux temperature, as described, for example, in Journal of the Chemical Society, Perkin Transactions 1: Organic and Bio-Organic Chemistry (1972-1999), (10), 2501-6, 1983 or in US20100076027.

C₁-C₄alkyl boronic acids of the formula R₃B(OH)₂, wherein R₃ is C₁-C₄alkyl, or the corresponding C₁-C₄alkyl boronate ester derivatives, or the corresponding 6-membered tri(C₁-C₄alkyl) boroxine derivatives of the formula (R₃BO)₃, wherein R₃ is C₁-C₄alkyl, are either known, commercially available or may be prepared by methods known to a person skilled in the art.

The subgroup of compounds of formula I, wherein G₁, G₂ and R₂ are as defined in formula I and wherein Q is defined as Qa, in which A is N and R₃, X and R₁ are as defined in formula I, and wherein Q₁ is —N(R₄)COR₅, in which R₄ and R₅ are as defined in formula I, may be defined as compounds of formula I-Qa-1 (scheme 7).

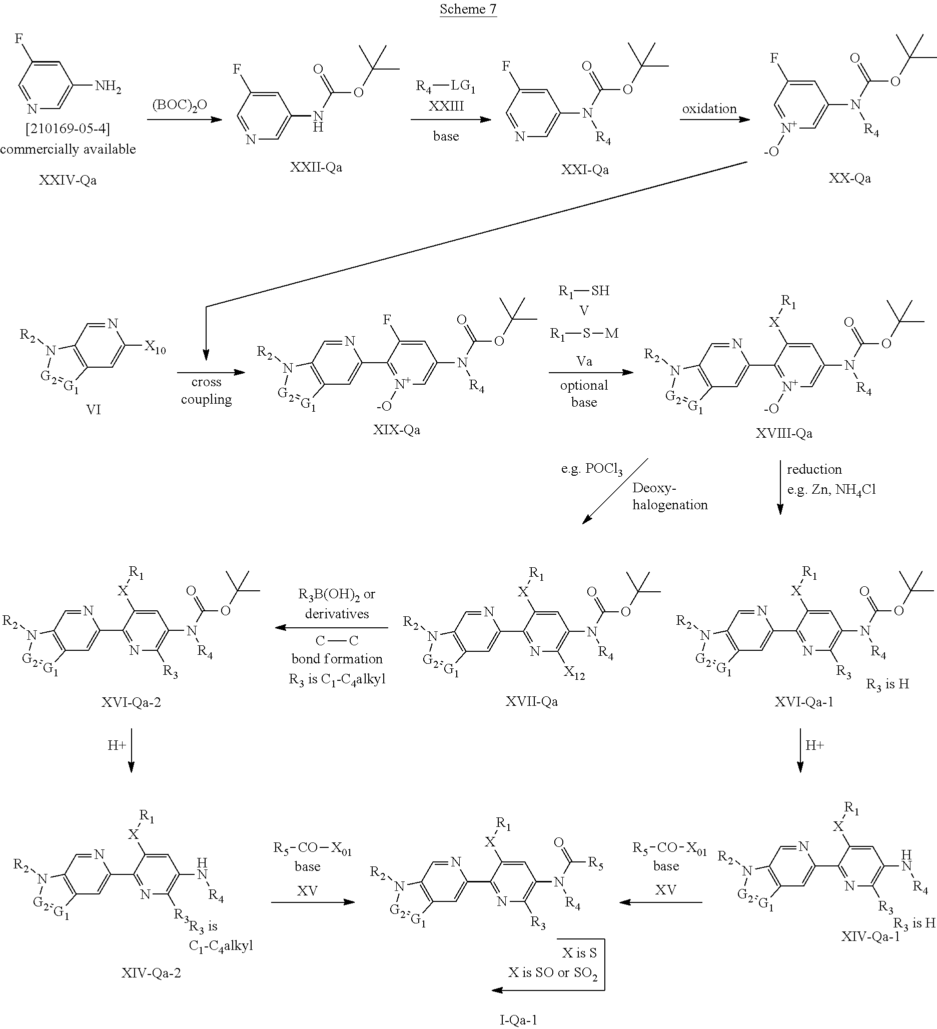

Such compounds of formula I-Qa-1, wherein X is S and R₃ is H, and in which G₁, G₂, R₂, R₁, R₄ and R₅ are as defined in formula I, can be prepared (scheme 7) by reacting compounds of formula XIV-Qa-1, wherein X is S and R₃ is H, and in which G₁, G₂, R₂, R₁ and R₄ are as defined in formula I, with compounds of formula XV, wherein R₅ is as defined in formula I, and X₀₁ is a halogen, preferably chlorine (alternatively, X₀₁ is the leaving group —O(CO)R₅), in the presence of a base, such as triethylamine, N,N-diisopropyl-ethylamine or pyridine, optionally in the presence of a catalyst (such as 4-dimethylaminopyridine DMAP), in an inert solvents such as dichloromethane, tetrahydrofuran, dioxane, N,N-dimethylformamide, N,N-dimethylacetamide, acetonitrile, ethyl acetate or toluene, at temperatures between 0 and 50° C. Certain bases, such as pyridine and triethylamine, may be employed successfully as both base and solvent.

Compounds of formula XIV-Qa-1, wherein X is S and R₃ is H, and in which G₁, G₂, R₂, R₁ and R₄ are as defined in formula I, can be prepared from compounds of formula XVI-Qa-1, wherein X is S and R₃ is H, and in which G₁, G₂, R₂, R₁ and R₄ are as defined in formula I, by treatment with organic acids, for example trifluoroacetic acid, acetic acid and the like, or mineral acids such as hydrochloric acid, in inert solvents, such as dichloromethane or tetrahydrofuran THF, optionally in the presence of water, at temperatures between 0 and 80° C., by methods well known to those skilled in the art.

Compounds of formula XVI-Qa-1, wherein X is S and R₃ is H, and in which G₁, G₂, R₂, R₁ and R₄ are as defined in formula I, can be prepared by deoxygenation/reduction of compounds of formula XVIII-Qa, wherein X is S, and in which G₁, G₂, R₂, R₁ and R₄ are as defined in formula I, under conditions already described above (see scheme 1, transformation of compounds II-Qa into I-Qa).

Alternatively, compounds of formula I-Qa-1, wherein X is S and R₃ is C₁-C₄alkyl, and in which G₁, G₂, R₂, R₁, R₄ and R₅ are as defined in formula I, may be prepared (scheme 7) by reacting compounds of formula XIV-Qa-2, wherein X is S and R₃ is C₁-C₄alkyl, and in which G₁, G₂, R₂, R₁ and R₄ are as defined in formula I, with compounds of formula XV, wherein R₅ is as defined in formula I, and X₀₁ is a halogen, preferably chlorine (alternatively, X₀₁ is the leaving group —O(CO)R₅), under conditions already described above (see scheme 7, transformation of compounds XIV-Qa-1 into I-Qa-1).

Compounds of formula XIV-Qa-2, wherein X is S and R₃ is C₁-C₄alkyl, and in which G₁, G₂, R₂, R₁ and R₄ are as defined in formula I, may be prepared by treating compounds of formula XVI-Qa-2, wherein X is S and R₃ is C₁-C₄alkyl, and in which G₁, G₂, R₂, R₁ and R₄ are as defined in formula I, with acids under conditions already described above (see scheme 7, transformation of compounds XVI-Qa-1 into XIV-Qa-1).

Compounds of formula XVI-Qa-2, wherein X is S and R₃ is C₁-C₄alkyl, and in which G₁, G₂, R₂, R₁ and R₄ are as defined in formula I, may be prepared by reacting compounds of formula XVII-Qa, wherein X is S, and in which G₁, G₂, R₂, R₁ and R₄ are as defined in formula I, and wherein X₁₂ is a leaving group like, for example, chlorine, bromine or iodine (preferably chlorine or bromine), or an aryl- or alkylsulfonate such as trifluoromethanesulfonate, with C₁-C₄alkyl boronic acids of the formula R₃B(OH)₂, wherein R₃ is C₁-C₄alkyl, or the corresponding C₁-C₄alkyl boronate ester derivatives, or the corresponding 6-membered tri(C₁-C₄alkyl) boroxine derivatives of the formula (R₃BO)₃, wherein R₃ is C₁-C₄alkyl, under conditions already described above (see scheme 6, transformation of compounds XII-Qa into I-Qa by means of a C—C bond formation reaction).

Compounds of formula XVII-Qa, wherein X is S, and in which G₁, G₂, R₂, R₁ and R₄ are as defined in formula I, and wherein X₁₂ is a leaving group like, for example, chlorine, bromine or iodine (preferably chlorine or bromine), or an aryl- or alkylsulfonate such as trifluoromethanesulfonate, may be prepared from compounds of formula XVIII-Qa, wherein X is S, and in which G₁, G₂, R₂, R₁ and R₄ are as defined in formula I, by means of a deoxyhalogenation under conditions already described above (see scheme 5, transformation of compounds II-Qb-1 into XII-Qb).

Compounds of formula XVIII-Qa, wherein X is S, and in which G₁, G₂, R₂, R₁ and R₄ are as defined in formula I, can be prepared by reacting compounds of formula XIX-Qa, wherein G₁, G₂, R₂ and R₄ are as defined in formula I, with reagents of formula V or Va, wherein R₁ is as defined in formula I, under conditions already described above (see text schemes 1 and 3).

Compounds of formula XIX-Qa, wherein G₁, G₂, R₂ and R₄ are as defined in formula I, can be prepared by cross-coupling compounds of formula VI, wherein G₁, G₂ and R₂ are as defined in formula I, and in which X₁₀ is a halogen (or a pseudo-halogen leaving group, such as a triflate), preferably bromine or chlorine, with compounds of formula XX-Qa, wherein R₄ is as defined in formula I, under conditions already described above (see text schemes 2 and 4).

Compounds of formula XX-Qa, wherein R₄ is as defined in formula I, can be prepared by oxidation of compounds of formula XXI-Qa, wherein R₄ is as defined in formula I, under conditions already described above (see text schemes 2 and 4).

Compounds of formula XXI-Qa, wherein R₄ is as defined in formula I, can be prepared by reacting the compound of formula XXII-Qa with compounds of formula R₄-LG₁ XXIII, wherein R₄ is as defined in formula I and in which LG₁ is a leaving group, such as halogen, preferably iodine, bromine or chlorine (for example is R₄-LG₁ XXIII is methyl iodide), in presence of a base, such as sodium carbonate, potassium carbonate or cesium carbonate, or sodium hydride NaH, in a appropriate solvent such as for example tetrahydrofuran THF, N,N-dimethylformamide, N,N-dimethylacetamide or acetonitrile, at temperatures between −20 and 100° C., preferably between 0° C. and 80° C.

The compound of formula XXII-Qa may be prepared by reacting the compound of formula XXIV-Qa with, for example, di-tert-butyl dicarbonate (BOC anhydride, BOC₂O), in presence of a base, such as triethylamine NEt₃, diisopropylethylamine or pyridine, optionally in presence of an acylation catalyst such as 4-dimethylaminopyridine DMAP, in an inert solvent such as dichloromethane DCM, tetrahydrofuran, dioxane or toluene, at temperatures between 0 and 100° C., preferably between 0° C. and 30° C. Such conditions are known to those skilled in the art, see for example Protective Groups in Organic Synthesis, 3nd Ed Greene T W, Wuts PGM, 1999, pp 518-525.

Oxidation of any compounds of formula XVIII-Qa, XVII-Qa, XVI-Qa-1, XIV-Qa-1, XVI-Qa-2, XIV-Qa-2 and I-Qa-1 in scheme 7, wherein the substituents are as defined above, and in which X is S (sulfide), with a suitable oxidizing agent, into corresponding compounds wherein X is SO (sulfoxide) or SO₂ (sulfone) may be achieved under conditions already described above.

Compounds of formula XV, wherein R₅ is as defined in formula I, and X₀₁ is a halogen, preferably chlorine (alternatively, X₀₁ is the leaving group —O(CO)R₅); and

compounds of formula R₄-LG₁ XXIII, wherein R₄ is as defined in formula I and in which LG₁ is a leaving group, such as halogen, preferably iodine, bromine or chlorine; and
the compound of formula XXII-Qa and the compound of formula XXIV-Qa;
are either known, commercially available or may be prepared by methods known to a person skilled in the art.

The subgroup of compounds of formula IXa-1, wherein Q₁ is defined as Q_{1SP}, in which Q_{1SP} is 1-cyanocyclopropyl, 1-cyano-1-methyl-ethyl or 2-pyridyloxy, may be defined as compounds of formula IXa-1-SP (scheme 8).

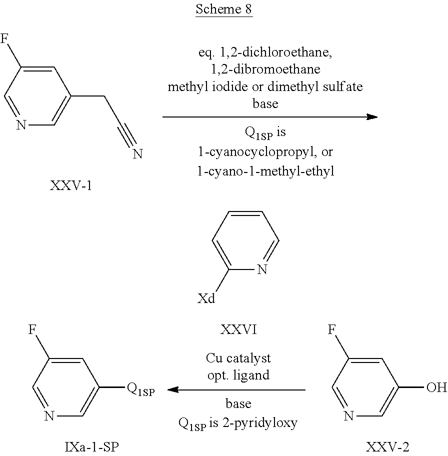

The compound of formula IXa-1-SP, wherein Q_{1SP} is 1-cyanocyclopropyl (compound 135, Table I) can be prepared from the commercial compound of formula XXV-1 by applying conditions described, for example, in WO17/089190.

The compound of formula IXa-1-SP, wherein Q_{1SP} is 1-cyano-1-methyl-ethyl (compound 134, Table I) can be prepared from the commercial compound of formula XXV-1 by applying conditions described, for example, in WO18/153778.

The compound of formula IXa-1-SP, wherein Q_{1SP} is 2-pyridyloxy (compound 136, Table I) can be prepared by reacting the commercial compound of formula XXV-2 with compounds of formula XXVI, wherein Xd is a leaving group such as, for example, chlorine, bromine or iodine (preferably bromine or iodine), or an aryl-, alkyl- or haloalkylsulfonate such as trifluoromethanesulfonate, in the presence of a base such as, for example, sodium hydride, potassium or cesium carbonate, in a suitable solvent such as dioxane, acetonitrile, N,N-dimethylformamide or N,N-dimethylacetamide, in the presence of a copper catalyst, for example copper(I) iodide, optionally in the presence of an additional ligand, at temperatures between 20° C. and 200° C., preferably at temperatures ranging from room temperature to the boiling point of the reaction mixture, optionally under microwave irradiation.

Compounds of formula XXV-1, XXV-2 and XXVI (substituents as defined above) are either known compounds, commercially available or can be prepared by known methods.

The compounds of formula IXa-1-SP (scheme 8)

wherein
Q_{1SP} is 1-cyanocyclopropyl, 1-cyano-1-methyl-ethyl or 2-pyridyloxy,
are novel, especially developed for the preparation of the compounds of formula I according to the invention and therefore represent a further object of the invention.

The subgroup of compounds of formula VIIa-1, wherein Q₁ is defined as Q_{1SP′}, in which Q_{1SP′} is cyclopropyl, 1-cyanocyclopropyl, 1-cyano-1-methyl-ethyl or 2-pyridyloxy, may be defined as compounds of formula VIIa-1-SP′ (scheme 9).

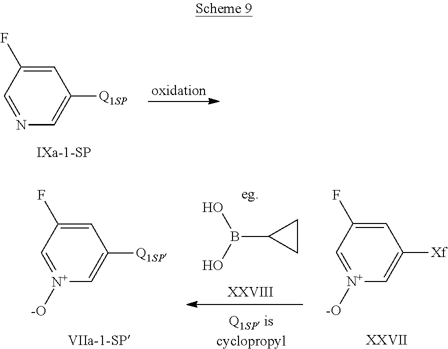

Compounds of formula VIIa-1-SP′, wherein Q_{1SP′} is 1-cyanocyclopropyl, 1-cyano-1-methyl-ethyl or 2-pyridyloxy (respectively compounds 126, 125 and 127, Table I) can be prepared by oxidation of compounds of formula IXa-1-SP, wherein Q_{1SP} is 1-cyanocyclopropyl, 1-cyano-1-methyl-ethyl or 2-pyridyloxy, under conditions already described above.

The compound of formula VIIa-1-SP′, wherein Q_{1SP′} is cyclopropyl (compound 124, Table I) can be prepared by reacting compounds of formula XXVII, wherein Xf is a leaving group like, for example, chlorine, bromine or iodine (preferably chlorine or bromine), or an aryl- or alkylsulfonate such as trifluoromethanesulfonate, with a compound of formula XXVIII (or a corresponding boronate ester derivative, such as a pinacol boronic ester; or a corresponding 6-membered boroxine derivative) by means of a C—C bond formation reaction typically under palladium-catalyzed (alternatively nickel-catalyzed) cross-coupling conditions. The reaction may be catalyzed by a palladium based catalyst, for example tetrakis(triphenyl-phosphine)palladium(0), (1,1′bis(diphenylphosphino) ferrocene)dichloro-palladium-dichloromethane (1:1 complex) or chloro(2-dicyclohexylphosphino-2′,4′,6′-triisopropyl-1,1′-biphenyl)[2-(2′-amino-1,1′-biphenyl)]palladium(II) (XPhos palladacycle), in presence of a base, like sodium carbonate, tripotassium phosphate or cesium fluoride, in a solvent or a solvent mixture, like, for example dioxane, acetonitrile, N,N-dimethylformamide, a mixture of 1,2-dimethoxyethane and water or of dioxane/water, or of toluene/water, preferably under inert atmosphere. The reaction temperature can preferentially range from room temperature to the boiling point of the reaction mixture, or the reaction may be performed under microwave irradiation. Such Suzuki-Miyaura reactions are well known to those skilled in the art and have been reviewed, for example, in J. Orgmet. Chem. 576, 1999, 147-168.

Compounds of formula XXVII and XXVIII (substituents as defined above) are either known compounds, commercially available or can be prepared by known methods.

The compounds of formula VIIa-1-SP′ (scheme 9)

wherein
Q_{1SP′} is cyclopropyl, 1-cyanocyclopropyl, 1-cyano-1-methyl-ethyl or 2-pyridyloxy,
are novel, especially developed for the preparation of the compounds of formula I according to the invention and therefore represent a further object of the invention.

The reactants can be reacted in the presence of a base. Examples of suitable bases are alkali metal or alkaline earth metal hydroxides, alkali metal or alkaline earth metal hydrides, alkali metal or alkaline earth metal amides, alkali metal or alkaline earth metal alkoxides, alkali metal or alkaline earth metal acetates, alkali metal or alkaline earth metal carbonates, alkali metal or alkaline earth metal dialkylamides or alkali metal or alkaline earth metal alkylsilylamides, alkylamines, alkylenediamines, free or N-alkylated saturated or unsaturated cycloalkylamines, basic heterocycles, ammonium hydroxides and carbocyclic amines. Examples which may be mentioned are sodium hydroxide, sodium hydride, sodium amide, sodium methoxide, sodium acetate, sodium carbonate, potassium tert-butoxide, potassium hydroxide, potassium carbonate, potassium hydride, lithium diisopropylamide, potassium bis(trimethylsilyl)amide, calcium hydride, triethylamine, diisopropylethylamine, triethylenediamine, cyclohexylamine, N-cyclohexyl-N,N-dimethylamine, N,N-diethylaniline, pyridine, 4-(N,N-dimethylamino)pyridine, quinuclidine, N-methylmorpholine, benzyltrimethylammonium hydroxide and 1,8-diazabicyclo[5.4.0]undec-7-ene (DBU).

The reactants can be reacted with each other as such, i.e. without adding a solvent or diluent. In most cases, however, it is advantageous to add an inert solvent or diluent or a mixture of these. If the reaction is carried out in the presence of a base, bases which are employed in excess, such as triethylamine, pyridine, N-methylmorpholine or N,N-diethylaniline, may also act as solvents or diluents.

The reactions are advantageously carried out in a temperature range from approximately −80° C. to approximately +140° C., preferably from approximately −30° C. to approximately +100° C., in many cases in the range between ambient temperature and approximately +80° C.

A compound of formula I can be converted in a manner known per se into another compound of formula I by replacing one or more substituents of the starting compound of formula I in the customary manner by (an)other substituent(s) according to the invention, and by post modification of compounds of with reactions such as oxidation, alkylation, reduction, acylation and other methods known by those skilled in the art.

Depending on the choice of the reaction conditions and starting materials which are suitable in each case, it is possible, for example, in one reaction step only to replace one substituent by another substituent according to the invention, or a plurality of substituents can be replaced by other substituents according to the invention in the same reaction step.

Salts of compounds of formula I can be prepared in a manner known per se. Thus, for example, acid addition salts of compounds of formula I are obtained by treatment with a suitable acid or a suitable ion exchanger reagent and salts with bases are obtained by treatment with a suitable base or with a suitable ion exchanger reagent.

Salts of compounds of formula I can be converted in the customary manner into the free compounds I, acid addition salts, for example, by treatment with a suitable basic compound or with a suitable ion exchanger reagent and salts with bases, for example, by treatment with a suitable acid or with a suitable ion exchanger reagent.

Salts of compounds of formula I can be converted in a manner known per se into other salts of compounds of formula I, acid addition salts, for example, into other acid addition salts, for example by treatment of a salt of inorganic acid such as hydrochloride with a suitable metal salt such as a sodium, barium or silver salt, of an acid, for example with silver acetate, in a suitable solvent in which an inorganic salt which forms, for example silver chloride, is insoluble and thus precipitates from the reaction mixture.

Depending on the procedure or the reaction conditions, the compounds of formula I, which have salt-forming properties can be obtained in free form or in the form of salts.

The compounds of formula I and, where appropriate, the tautomers thereof, in each case in free form or in salt form, can be present in the form of one of the isomers which are possible or as a mixture of these, for example in the form of pure isomers, such as antipodes and/or diastereomers, or as isomer mixtures, such as enantiomer mixtures, for example racemates, diastereomer mixtures or racemate mixtures, depending on the number, absolute and relative configuration of asymmetric carbon atoms which occur in the molecule and/or depending on the configuration of non-aromatic double bonds which occur in the molecule; the invention relates to the pure isomers and also to all isomer mixtures which are possible and is to be understood in each case in this sense hereinabove and hereinbelow, even when stereochemical details are not mentioned specifically in each case.

Diastereomer mixtures or racemate mixtures of compounds of formula I, in free form or in salt form, which can be obtained depending on which starting materials and procedures have been chosen can be separated in a known manner into the pure diasteromers or racemates on the basis of the physicochemical differences of the components, for example by fractional crystallization, distillation and/or chromatography.

Enantiomer mixtures, such as racemates, which can be obtained in a similar manner can be resolved into the optical antipodes by known methods, for example by recrystallization from an optically active solvent, by chromatography on chiral adsorbents, for example high-performance liquid chromatography (HPLC) on acetyl cellulose, with the aid of suitable microorganisms, by cleavage with specific, immobilized enzymes, via the formation of inclusion compounds, for example using chiral crown ethers, where only one enantiomer is complexed, or by conversion into diastereomeric salts, for example by reacting a basic end-product racemate with an optically active acid, such as a carboxylic acid, for example camphor, tartaric or malic acid, or sulfonic acid, for example camphorsulfonic acid, and separating the diastereomer mixture which can be obtained in this manner, for example by fractional crystallization based on their differing solubilities, to give the diastereomers, from which the desired enantiomer can be set free by the action of suitable agents, for example basic agents.

Pure diastereomers or enantiomers can be obtained according to the invention not only by separating suitable isomer mixtures, but also by generally known methods of diastereoselective or enantioselective synthesis, for example by carrying out the process according to the invention with starting materials of a suitable stereochemistry.

N-oxides can be prepared by reacting a compound of the formula I with a suitable oxidizing agent, for example the H₂O₂/urea adduct in the presence of an acid anhydride, e.g. trifluoroacetic anhydride. Such oxidations are known from the literature, for example from J. Med. Chem., 32 (12), 2561-73, 1989 or WO 2000/15615.

It is advantageous to isolate or synthesize in each case the biologically more effective isomer, for example enantiomer or diastereomer, or isomer mixture, for example enantiomer mixture or diastereomer mixture, if the individual components have a different biological activity.

The compounds of formula I and, where appropriate, the tautomers thereof, in each case in free form or in salt form, can, if appropriate, also be obtained in the form of hydrates and/or include other solvents, for example those which may have been used for the crystallization of compounds which are present in solid form.

The compounds according to the following Tables A-1 to A-81, Tables B-1 to B-54, Tables C-1 to C-81, Tables D-1 to D-54, Tables E-1 to E-81 and Tables F-1 to F-54 below can be prepared according to the methods described above. The examples which follow are intended to illustrate the invention and show preferred compounds of formula I.

The tables A-1 to A-81 below illustrate specific compounds of the invention.

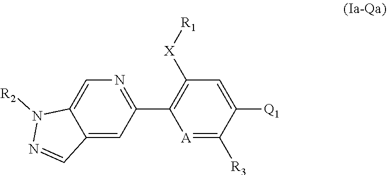

TABLE Y
Substituent definitions of Q₁

| Index | Q₁ | Index | Q₁ |
|---|---|---|---|
| 1 | H | 11 | —N(CH₃)COCH₃ |
| 2 | —N(CH₃)COCH₂CH₃ | 12 | 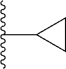 [SMILES: *C1CC1] |
| 3 | Cl | 13 | 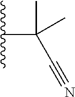 [SMILES: *C(C)(C)C#N] |
| 4 | Br | 14 | 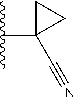 [SMILES: *C1(C#N)CC1] |
| 5 | —N(CH₃)COcycloC3 | 15 | —OCH₂CF₃ |
| 6 | CF₃ | 16 | 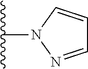 [SMILES: *n1cccn1] |
| 7 | —NH₂ | 17 | 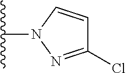 [SMILES: *n1ccc(Cl)n1] |
| 8 | —NH(CH₃) | 18 | 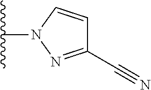 [SMILES: *n1ccc(C#N)n1] |
| 9 | —N(CH₃)₂ | 19 | 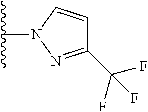 [SMILES: *n1ccc(C(F)(F)F)n1] |
| 10 | —NHCOCH₃ | 20 | 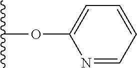 [SMILES: *Oc1ccccn1] |

In the table Y and in tables A, “cycloC3” represents cyclopropyl.

Table A-1 provides 20 compounds A-1.001 to A-1.020 of formula Ia-Qa wherein R₁ is CH₂CH₃, R₂ is CH₂CF₂CF₃, R₃ is H, X is S, A is N and Q₁ is as defined in table Y.

Table A-2 provides 20 compounds A-2.001 to A-2.020 of formula Ia-Qa wherein R₁ is CH₂CH₃, R₂ is CH₂CF₂CF₃, R₃ is H, X is SO, A is N and Q₁ is as defined in table Y.

Table A-3 provides 20 compounds A-3.001 to A-3.020 of formula Ia-Qa wherein R₁ is CH₂CH₃, R₂ is CH₂CF₂CF₃, R₃ is H, X is SO₂, A is N and Q₁ is as defined in table Y.

Table A-4 provides 20 compounds A-4.001 to A-4.020 of formula Ia-Qa wherein R₁ is CH₂CH₃, R₂ is CH₂CF₂CHF₂, R₃ is H, X is S, A is N and Q₁ is as defined in table Y.

Table A-5 provides 20 compounds A-5.001 to A-5.020 of formula Ia-Qa wherein R₁ is CH₂CH₃, R₂ is CH₂CF₂CHF₂, R₃ is H, X is SO, A is N and Q₁ is as defined in table Y.

Table A-6 provides 20 compounds A-6.001 to A-6.020 of formula Ia-Qa wherein R₁ is CH₂CH₃, R₂ is CH₂CF₂CHF₂, R₃ is H, X is SO₂, A is N and Q₁ is as defined in table Y.

Table A-7 provides 20 compounds A-7.001 to A-7.020 of formula Ia-Qa wherein R₁ is CH₂CH₃, R₂ is CH₂CHF₂, R₃ is H, X is S, A is N and Q₁ is as defined in table Y.

Table A-8 provides 20 compounds A-8.001 to A-8.020 of formula Ia-Qa wherein R₁ is CH₂CH₃, R₂ is CH₂CHF₂, R₃ is H, X is SO, A is N and Q₁ is as defined in table Y.

Table A-9 provides 20 compounds A-9.001 to A-9.020 of formula Ia-Qa wherein R₁ is CH₂CH₃, R₂ is CH₂CHF₂, R₃ is H, X is SO₂, A is N and Q₁ is as defined in table Y.

Table A-10 provides 20 compounds A-10.001 to A-10.020 of formula Ia-Qa wherein R₁ is CH₂CH₃, R₂ is CH₂CF₃, R₃ is H, X is S, A is N and Q₁ is as defined in table Y.

Table A-11 provides 20 compounds A-11.001 to A-11.020 of formula Ia-Qa wherein R₁ is CH₂CH₃, R₂ is CH₂CF₃, R₃ is H, X is SO, A is N and Q₁ is as defined in table Y.

Table A-12 provides 20 compounds A-12.001 to A-12.020 of formula Ia-Qa wherein R₁ is CH₂CH₃, R₂ is CH₂CF₃, R₃ is H, X is SO₂, A is N and Q₁ is as defined in table Y.

Table A-13 provides 20 compounds A-13.001 to A-13.020 of formula Ia-Qa wherein R₁ is CH₂CH₃, R₂ is CH₂CF₂CHFCF₃, R₃ is H, X is S, A is N and Q₁ is as defined in table Y.

Table A-14 provides 20 compounds A-14.001 to A-14.020 of formula Ia-Qa wherein R₁ is CH₂CH₃, R₂ is CH₂CF₂CHFCF₃, R₃ is H, X is SO, A is N and Q₁ is as defined in table Y.

Table A-15 provides 20 compounds A-15.001 to A-15.020 of formula Ia-Qa wherein R₁ is CH₂CH₃, R₂ is CH₂CF₂CHFCF₃, R₃ is H, X is SO₂, A is N and Q₁ is as defined in table Y.

Table A-16 provides 20 compounds A-16.001 to A-16.020 of formula Ia-Qa wherein R₁ is CH₂CH₃, R₂ is SO₂CF₃, R₃ is H, X is S, A is N and Q₁ is as defined in table Y.

Table A-17 provides 20 compounds A-17.001 to A-17.020 of formula Ia-Qa wherein R₁ is CH₂CH₃, R₂ is SO₂CF₃, R₃ is H, X is SO, A is N and Q₁ is as defined in table Y.

Table A-18 provides 20 compounds A-18.001 to A-18.020 of formula Ia-Qa wherein R₁ is CH₂CH₃, R₂ is SO₂CF₃, R₃ is H, X is SO₂, A is N and Q₁ is as defined in table Y.

Table A-19 provides 20 compounds A-19.001 to A-19.020 of formula Ia-Qa wherein R₁ is CH₂CH₃, R₂ is CH₂CF₂CF₃, R₃ is H, X is S, A is CH and Q₁ is as defined in table Y.

Table A-20 provides 20 compounds A-20.001 to A-20.020 of formula Ia-Qa wherein R₁ is CH₂CH₃, R₂ is CH₂CF₂CF₃, R₃ is H, X is SO, A is CH and Q₁ is as defined in table Y.

Table A-21 provides 20 compounds A-21.001 to A-21.020 of formula Ia-Qa wherein R₁ is CH₂CH₃, R₂ is CH₂CF₂CF₃, R₃ is H, X is SO₂, A is CH and Q₁ is as defined in table Y.

Table A-22 provides 20 compounds A-22.001 to A-22.020 of formula Ia-Qa wherein R₁ is CH₂CH₃, R₂ is CH₂CF₂CHF₂, R₃ is H, X is S, A is CH and Q₁ is as defined in table Y.

Table A-23 provides 20 compounds A-23.001 to A-23.020 of formula Ia-Qa wherein R₁ is CH₂CH₃, R₂ is CH₂CF₂CHF₂, R₃ is H, X is SO, A is CH and Q₁ is as defined in table Y.

Table A-24 provides 20 compounds A-24.001 to A-24.020 of formula Ia-Qa wherein R₁ is CH₂CH₃, R₂ is CH₂CF₂CHF₂, R₃ is H, X is SO₂, A is CH and Q₁ is as defined in table Y.

Table A-25 provides 20 compounds A-25.001 to A-25.020 of formula Ia-Qa wherein R₁ is CH₂CH₃, R₂ is CH₂CHF₂, R₃ is H, X is S, A is CH and Q₁ is as defined in table Y.

Table A-26 provides 20 compounds A-26.001 to A-26.020 of formula Ia-Qa wherein R₁ is CH₂CH₃, R₂ is CH₂CHF₂, R₃ is H, X is SO, A is CH and Q₁ is as defined in table Y.

Table A-27 provides 20 compounds A-27.001 to A-27.020 of formula Ia-Qa wherein R₁ is CH₂CH₃, R₂ is CH₂CHF₂, R₃ is H, X is SO₂, A is CH and Q₁ is as defined in table Y.

Table A-28 provides 20 compounds A-28.001 to A-28.020 of formula Ia-Qa wherein R₁ is CH₂CH₃, R₂ is CH₂CF₃, R₃ is H, X is S, A is CH and Q₁ is as defined in table Y.

Table A-29 provides 20 compounds A-29.001 to A-29.020 of formula Ia-Qa wherein R₁ is CH₂CH₃, R₂ is CH₂CF₃, R₃ is H, X is SO, A is CH and Q₁ is as defined in table Y.

Table A-30 provides 20 compounds A-30.001 to A-30.020 of formula Ia-Qa wherein R₁ is CH₂CH₃, R₂ is CH₂CF₃, R₃ is H, X is SO₂, A is CH and Q₁ is as defined in table Y.

Table A-31 provides 20 compounds A-31.001 to A-31.020 of formula Ia-Qa wherein R₁ is CH₂CH₃, R₂ is CH₂CF₂CHFCF₃, R₃ is H, X is S, A is CH and Q₁ is as defined in table Y.

Table A-32 provides 20 compounds A-32.001 to A-32.020 of formula Ia-Qa wherein R₁ is CH₂CH₃, R₂ is CH₂CF₂CHFCF₃, R₃ is H, X is SO, A is CH and Q₁ is as defined in table Y.

Table A-33 provides 20 compounds A-33.001 to A-33.020 of formula Ia-Qa wherein R₁ is CH₂CH₃, R₂ is CH₂CF₂CHFCF₃, R₃ is H, X is SO₂, A is CH and Q₁ is as defined in table Y.

Table A-34 provides 20 compounds A-34.001 to A-34.020 of formula Ia-Qa wherein R₁ is CH₂CH₃, R₂ is SO₂CF₃, R₃ is H, X is S, A is CH and Q₁ is as defined in table Y.

Table A-35 provides 20 compounds A-35.001 to A-35.020 of formula Ia-Qa wherein R₁ is CH₂CH₃, R₂ is SO₂CF₃, R₃ is H, X is SO, A is CH and Q₁ is as defined in table Y.

Table A-36 provides 20 compounds A-36.001 to A-36.020 of formula Ia-Qa wherein R₁ is CH₂CH₃, R₂ is SO₂CF₃, R₃ is H, X is SO₂, A is CH and Q₁ is as defined in table Y.

Table A-37 provides 20 compounds A-37.001 to A-37.020 of formula Ia-Qa wherein R₁ is CH₂CH₃, R₂ is CH₂CF₂CF₃, R₃ is CH₃, X is S, A is N and Q₁ is as defined in table Y.

Table A-38 provides 20 compounds A-38.001 to A-38.020 of formula Ia-Qa wherein R₁ is CH₂CH₃, R₂ is CH₂CF₂CF₃, R₃ is CH₃, X is SO, A is N and Q₁ is as defined in table Y.

Table A-39 provides 20 compounds A-39.001 to A-39.020 of formula Ia-Qa wherein R₁ is CH₂CH₃, R₂ is CH₂CF₂CF₃, R₃ is CH₃, X is SO₂, A is N and Q₁ is as defined in table Y.

Table A-40 provides 20 compounds A-40.001 to A-40.020 of formula Ia-Qa wherein R₁ is CH₂CH₃, R₂ is CH₂CF₂CHF₂, R₃ is CH₃, X is S, A is N and Q₁ is as defined in table Y.

Table A-41 provides 20 compounds A-41.001 to A-41.020 of formula Ia-Qa wherein R₁ is CH₂CH₃, R₂ is CH₂CF₂CHF₂, R₃ is CH₃, X is SO, A is N and Q₁ is as defined in table Y.

Table A-42 provides 20 compounds A-42.001 to A-42.020 of formula Ia-Qa wherein R₁ is CH₂CH₃, R₂ is CH₂CF₂CHF₂, R₃ is CH₃, X is SO₂, A is N and Q₁ is as defined in table Y.

Table A-43 provides 20 compounds A-43.001 to A-43.020 of formula Ia-Qa wherein R₁ is CH₂CH₃, R₂ is CH₂CHF₂, R₃ is CH₃, X is S, A is N and Q₁ is as defined in table Y.

Table A-44 provides 20 compounds A-44.001 to A-44.020 of formula Ia-Qa wherein R₁ is CH₂CH₃, R₂ is CH₂CHF₂, R₃ is CH₃, X is SO, A is N and Q₁ is as defined in table Y.

Table A-45 provides 20 compounds A-45.001 to A-45.020 of formula Ia-Qa wherein R₁ is CH₂CH₃, R₂ is CH₂CHF₂, R₃ is CH₃, X is SO₂, A is N and Q₁ is as defined in table Y.

Table A-46 provides 20 compounds A-46.001 to A-46.020 of formula Ia-Qa wherein R₁ is CH₂CH₃, R₂ is CH₂CF₃, R₃ is CH₃, X is S, A is N and Q₁ is as defined in table Y.

Table A-47 provides 20 compounds A-47.001 to A-47.020 of formula Ia-Qa wherein R₁ is CH₂CH₃, R₂ is CH₂CF₃, R₃ is CH₃, X is SO, A is N and Q₁ is as defined in table Y.

Table A-48 provides 20 compounds A-48.001 to A-48.020 of formula Ia-Qa wherein R₁ is CH₂CH₃, R₂ is CH₂CF₃, R₃ is CH₃, X is SO₂, A is N and Q₁ is as defined in table Y.

Table A-49 provides 20 compounds A-49.001 to A-49.020 of formula Ia-Qa wherein R₁ is CH₂CH₃, R₂ is CH₂CF₂CHFCF₃, R₃ is CH₃, X is S, A is N and Q₁ is as defined in table Y.

Table A-50 provides 20 compounds A-50.001 to A-50.020 of formula Ia-Qa wherein R₁ is CH₂CH₃, R₂ is CH₂CF₂CHFCF₃, R₃ is CH₃, X is SO, A is N and Q₁ is as defined in table Y.

Table A-51 provides 20 compounds A-51.001 to A-51.020 of formula Ia-Qa wherein R₁ is CH₂CH₃, R₂ is CH₂CF₂CHFCF₃, R₃ is CH₃, X is SO₂, A is N and Q₁ is as defined in table Y.

Table A-52 provides 20 compounds A-52.001 to A-52.020 of formula Ia-Qa wherein R₁ is CH₂CH₃, R₂ is SO₂CF₃, R₃ is CH₃, X is S, A is N and Q₁ is as defined in table Y.

Table A-53 provides 20 compounds A-53.001 to A-53.020 of formula Ia-Qa wherein R₁ is CH₂CH₃, R₂ is SO₂CF₃, R₃ is CH₃, X is SO, A is N and Q₁ is as defined in table Y.

Table A-54 provides 20 compounds A-54.001 to A-54.020 of formula Ia-Qa wherein R₁ is CH₂CH₃, R₂ is SO₂CF₃, R₃ is CH₃, X is SO₂, A is N and Q₁ is as defined in table Y.

Table A-55 provides 20 compounds A-55.001 to A-55.020 of formula Ia-Qa wherein R₁ is —CH₂cyclopropyl, R₂ is CH₂CF₂CF₃, R₃ is H, X is S, A is N and Q₁ is as defined in table Y.

Table A-56 provides 20 compounds A-56.001 to A-56.020 of formula Ia-Qa wherein R₁ is —CH₂cyclopropyl, R₂ is CH₂CF₂CF₃, R₃ is H, X is SO, A is N and Q₁ is as defined in table Y.

Table A-57 provides 20 compounds A-57.001 to A-57.020 of formula Ia-Qa wherein R₁ is —CH₂cyclopropyl, R₂ is CH₂CF₂CF₃, R₃ is H, X is SO₂, A is N and Q₁ is as defined in table Y.

Table A-58 provides 20 compounds A-58.001 to A-58.020 of formula Ia-Qa wherein R₁ is —CH₂cyclopropyl, R₂ is CH₂CF₂CHF₂, R₃ is H, X is S, A is N and Q₁ is as defined in table Y.

Table A-59 provides 20 compounds A-59.001 to A-59.020 of formula Ia-Qa wherein R₁ is —CH₂cyclopropyl, R₂ is CH₂CF₂CHF₂, R₃ is H, X is SO, A is N and Q₁ is as defined in table Y.

Table A-60 provides 20 compounds A-60.001 to A-60.020 of formula Ia-Qa wherein R₁ is —CH₂cyclopropyl, R₂ is CH₂CF₂CHF₂, R₃ is H, X is SO₂, A is N and Q₁ is as defined in table Y.

Table A-61 provides 20 compounds A-61.001 to A-61.020 of formula Ia-Qa wherein R₁ is —CH₂cyclopropyl, R₂ is CH₂CF₃, R₃ is H, X is S, A is N and Q₁ is as defined in table Y.

Table A-62 provides 20 compounds A-62.001 to A-62.020 of formula Ia-Qa wherein R₁ is —CH₂cyclopropyl, R₂ is CH₂CF₃, R₃ is H, X is SO, A is N and Q₁ is as defined in table Y.

Table A-63 provides 20 compounds A-63.001 to A-63.020 of formula Ia-Qa wherein R₁ is —CH₂cyclopropyl, R₂ is CH₂CF₃, R₃ is H, X is SO₂, A is N and Q₁ is as defined in table Y.

Table A-64 provides 20 compounds A-64.001 to A-64.020 of formula Ia-Qa wherein R₁ is —CH₂cyclopropyl, R₂ is CH₂CF₂CF₃, R₃ is H, X is S, A is CH and Q₁ is as defined in table Y.

Table A-65 provides 20 compounds A-65.001 to A-65.020 of formula Ia-Qa wherein R₁ is —CH₂cyclopropyl, R₂ is CH₂CF₂CF₃, R₃ is H, X is SO, A is CH and Q₁ is as defined in table Y.

Table A-66 provides 20 compounds A-66.001 to A-66.020 of formula Ia-Qa wherein R₁ is —CH₂cyclopropyl, R₂ is CH₂CF₂CF₃, R₃ is H, X is SO₂, A is CH and Q₁ is as defined in table Y.

Table A-67 provides 20 compounds A-67.001 to A-67.020 of formula Ia-Qa wherein R₁ is —CH₂cyclopropyl, R₂ is CH₂CF₂CHF₂, R₃ is H, X is S, A is CH and Q₁ is as defined in table Y.

Table A-68 provides 20 compounds A-68.001 to A-68.020 of formula Ia-Qa wherein R₁ is —CH₂cyclopropyl, R₂ is CH₂CF₂CHF₂, R₃ is H, X is SO, A is CH and Q₁ is as defined in table Y.

Table A-69 provides 20 compounds A-69.001 to A-69.020 of formula Ia-Qa wherein R₁ is —CH₂cyclopropyl, R₂ is CH₂CF₂CHF₂, R₃ is H, X is SO₂, A is CH and Q₁ is as defined in table Y.

Table A-70 provides 20 compounds A-70.001 to A-70.020 of formula Ia-Qa wherein R₁ is —CH₂cyclopropyl, R₂ is CH₂CF₃, R₃ is H, X is S, A is CH and Q₁ is as defined in table Y.

Table A-71 provides 20 compounds A-71.001 to A-71.020 of formula Ia-Qa wherein R₁ is —CH₂cyclopropyl, R₂ is CH₂CF₃, R₃ is H, X is SO, A is CH and Q₁ is as defined in table Y.

Table A-72 provides 20 compounds A-72.001 to A-72.020 of formula Ia-Qa wherein R₁ is —CH₂cyclopropyl, R₂ is CH₂CF₃, R₃ is H, X is SO₂, A is CH and Q₁ is as defined in table Y.

Table A-73 provides 20 compounds A-73.001 to A-73.020 of formula Ia-Qa wherein R₁ is —CH₂cyclopropyl, R₂ is CH₂CF₂CF₃, R₃ is CH₃, X is S, A is N and Q₁ is as defined in table Y.

Table A-74 provides 20 compounds A-74.001 to A-74.020 of formula Ia-Qa wherein R₁ is —CH₂cyclopropyl, R₂ is CH₂CF₂CF₃, R₃ is CH₃, X is SO, A is N and Q₁ is as defined in table Y.

Table A-75 provides 20 compounds A-75.001 to A-75.020 of formula Ia-Qa wherein R₁ is —CH₂cyclopropyl, R₂ is CH₂CF₂CF₃, R₃ is CH₃, X is SO₂, A is N and Q₁ is as defined in table Y.

Table A-76 provides 20 compounds A-76.001 to A-76.020 of formula Ia-Qa wherein R₁ is —CH₂cyclopropyl, R₂ is CH₂CF₂CHF₂, R₃ is CH₃, X is S, A is N and Q₁ is as defined in table Y.

Table A-77 provides 20 compounds A-77.001 to A-77.020 of formula Ia-Qa wherein R₁ is —CH₂cyclopropyl, R₂ is CH₂CF₂CHF₂, R₃ is CH₃, X is SO, A is N and Q₁ is as defined in table Y.

Table A-78 provides 20 compounds A-78.001 to A-78.020 of formula Ia-Qa wherein R₁ is —CH₂cyclopropyl, R₂ is CH₂CF₂CHF₂, R₃ is CH₃, X is SO₂, A is N and Q₁ is as defined in table Y.

Table A-79 provides 20 compounds A-79.001 to A-79.020 of formula Ia-Qa wherein R₁ is —CH₂cyclopropyl, R₂ is CH₂CF₃, R₃ is CH₃, X is S, A is N and Q₁ is as defined in table Y.

Table A-80 provides 20 compounds A-80.001 to A-80.020 of formula Ia-Qa wherein R₁ is —CH₂cyclopropyl, R₂ is CH₂CF₃, R₃ is CH₃, X is SO, A is N and Q₁ is as defined in table Y.

Table A-81 provides 20 compounds A-81.001 to A-81.020 of formula Ia-Qa wherein R₁ is —CH₂cyclopropyl, R₂ is CH₂CF₃, R₃ is CH₃, X is SO₂, A is N and Q₁ is as defined in table Y.

The tables B-1 to B-54 below illustrate further specific compounds of the invention.

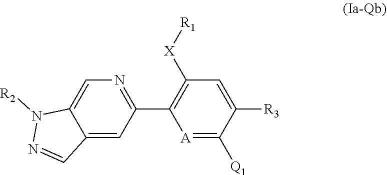

TABLE Z
Substituent definitions of Q₁

| Index | Q₁ | Index | Q₁ |
|---|---|---|---|
| 1 | H | 12 | 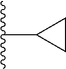 [SMILES: *C1CC1] |
| 2 | Cl | 13 | —N(H)CONH₂ |
| 3 | Br | 14 | —N(H)CONH(CH₃) |
| 4 | —N(CH₃)COCH₂CH₃ | 15 | —N(H)CON(CH₃)₂ |
| 5 | —NH₂ | 16 | —N(CH₃)CONH₂ |
| 6 | —NH(CH₃) | 17 | —N(CH₃)CONH(CH₃) |
| 7 | —N(CH₃)₂ | 18 | —N(CH₃)CON(CH₃)₂ |
| 8 | —NHCOCH₃ | 19 | 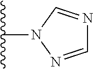 [SMILES: *n1cncn1] |
| 9 | —N(CH₃)COCH₃ | 20 | 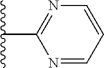 [SMILES: *c1ncccn1] |
| 10 | —NHCOcycloC3 | 21 | 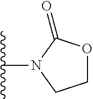 [SMILES: *N1CCOC1=O] |
| 11 | —N(CH₃)COcycloC3 | | |

In the table Z and in tables B, D and F, “cycloC3” represents cyclopropyl.

Table B-1 provides 21 compounds B-1.001 to B-1.021 of formula Ia-Qb wherein R₁ is CH₂CH₃, R₂ is CH₂CF₂CF₃, R₃ is H, X is S, A is N and Q₁ is as defined in table Z.

Table B-2 provides 21 compounds B-2.001 to B-2.021 of formula Ia-Qb wherein R₁ is CH₂CH₃, R₂ is CH₂CF₂CF₃, R₃ is H, X is SO, A is N and Q₁ is as defined in table Z.

Table B-3 provides 21 compounds B-3.001 to B-3.021 of formula Ia-Qb wherein R₁ is CH₂CH₃, R₂ is CH₂CF₂CF₃, R₃ is H, X is SO₂, A is N and Q₁ is as defined in table Z.

Table B-4 provides 21 compounds B-4.001 to B-4.021 of formula Ia-Qb wherein R₁ is CH₂CH₃, R₂ is CH₂CF₂CHF₂, R₃ is H, X is S, A is N and Q₁ is as defined in table Z.

Table B-5 provides 21 compounds B-5.001 to B-5.021 of formula Ia-Qb wherein R₁ is CH₂CH₃, R₂ is CH₂CF₂CHF₂, R₃ is H, X is SO, A is N and Q₁ is as defined in table Z.

Table B-6 provides 21 compounds B-6.001 to B-6.021 of formula Ia-Qb wherein R₁ is CH₂CH₃, R₂ is CH₂CF₂CHF₂, R₃ is H, X is SO₂, A is N and Q₁ is as defined in table Z.

Table B-7 provides 21 compounds B-7.001 to B-7.021 of formula Ia-Qb wherein R₁ is CH₂CH₃, R₂ is CH₂CHF₂, R₃ is H, X is S, A is N and Q₁ is as defined in table Z.

Table B-8 provides 21 compounds B-8.001 to B-8.021 of formula Ia-Qb wherein R₁ is CH₂CH₃, R₂ is CH₂CHF₂, R₃ is H, X is SO, A is N and Q₁ is as defined in table Z.

Table B-9 provides 21 compounds B-9.001 to B-9.021 of formula Ia-Qb wherein R₁ is CH₂CH₃, R₂ is CH₂CHF₂, R₃ is H, X is SO₂, A is N and Q₁ is as defined in table Z.

Table B-10 provides 21 compounds B-10.001 to B-10.021 of formula Ia-Qb wherein R₁ is CH₂CH₃, R₂ is CH₂CF₃, R₃ is H, X is S, A is N and Q₁ is as defined in table Z.

Table B-11 provides 21 compounds B-11.001 to B-11.021 of formula Ia-Qb wherein R₁ is CH₂CH₃, R₂ is CH₂CF₃, R₃ is H, X is SO, A is N and Q₁ is as defined in table Z.

Table B-12 provides 21 compounds B-12.001 to B-12.021 of formula Ia-Qb wherein R₁ is CH₂CH₃, R₂ is CH₂CF₃, R₃ is H, X is SO₂, A is N and Q₁ is as defined in table Z.

Table B-13 provides 21 compounds B-13.001 to B-13.021 of formula Ia-Qb wherein R₁ is CH₂CH₃, R₂ is CH₂CF₂CHFCF₃, R₃ is H, X is S, A is N and Q₁ is as defined in table Z.

Table B-14 provides 21 compounds B-14.001 to B-14.021 of formula Ia-Qb wherein R₁ is CH₂CH₃, R₂ is CH₂CF₂CHFCF₃, R₃ is H, X is SO, A is N and Q₁ is as defined in table Z.

Table B-15 provides 21 compounds B-15.001 to B-15.021 of formula Ia-Qb wherein R₁ is CH₂CH₃, R₂ is CH₂CF₂CHFCF₃, R₃ is H, X is SO₂, A is N and Q₁ is as defined in table Z.

Table B-16 provides 21 compounds B-16.001 to B-16.021 of formula Ia-Qb wherein R₁ is CH₂CH₃, R₂ is SO₂CF₃, R₃ is H, X is S, A is N and Q₁ is as defined in table Z.

Table B-17 provides 21 compounds B-17.001 to B-17.021 of formula Ia-Qb wherein R₁ is CH₂CH₃, R₂ is SO₂CF₃, R₃ is H, X is SO, A is N and Q₁ is as defined in table Z.

Table B-18 provides 21 compounds B-18.001 to B-18.021 of formula Ia-Qb wherein R₁ is CH₂CH₃, R₂ is SO₂CF₃, R₃ is H, X is SO₂, A is N and Q₁ is as defined in table Z.

Table B-19 provides 21 compounds B-19.001 to B-19.021 of formula Ia-Qb wherein R₁ is CH₂CH₃, R₂ is CH₂CF₂CF₃, R₃ is H, X is S, A is CH and Q₁ is as defined in table Z.

Table B-20 provides 21 compounds B-20.001 to B-20.021 of formula Ia-Qb wherein R₁ is CH₂CH₃, R₂ is CH₂CF₂CF₃, R₃ is H, X is SO, A is CH and Q₁ is as defined in table Z.

Table B-21 provides 21 compounds B-21.001 to B-21.021 of formula Ia-Qb wherein R₁ is CH₂CH₃, R₂ is CH₂CF₂CF₃, R₃ is H, X is SO₂, A is CH and Q₁ is as defined in table Z.

Table B-22 provides 21 compounds B-22.001 to B-22.021 of formula Ia-Qb wherein R₁ is CH₂CH₃, R₂ is CH₂CF₂CHF₂, R₃ is H, X is S, A is CH and Q₁ is as defined in table Z.

Table B-23 provides 21 compounds B-23.001 to B-23.021 of formula Ia-Qb wherein R₁ is CH₂CH₃, R₂ is CH₂CF₂CHF₂, R₃ is H, X is SO, A is CH and Q₁ is as defined in table Z.

Table B-24 provides 21 compounds B-24.001 to B-24.021 of formula Ia-Qb wherein R₁ is CH₂CH₃, R₂ is CH₂CF₂CHF₂, R₃ is H, X is SO₂, A is CH and Q₁ is as defined in table Z.

Table B-25 provides 21 compounds B-25.001 to B-25.021 of formula Ia-Qb wherein R₁ is CH₂CH₃, R₂ is CH₂CHF₂, R₃ is H, X is S, A is CH and Q₁ is as defined in table Z.

Table B-26 provides 21 compounds B-26.001 to B-26.021 of formula Ia-Qb wherein R₁ is CH₂CH₃, R₂ is CH₂CHF₂, R₃ is H, X is SO, A is CH and Q₁ is as defined in table Z.

Table B-27 provides 21 compounds B-27.001 to B-27.021 of formula Ia-Qb wherein R₁ is CH₂CH₃, R₂ is CH₂CHF₂, R₃ is H, X is SO₂, A is CH and Q₁ is as defined in table Z.

Table B-28 provides 21 compounds B-28.001 to B-28.021 of formula Ia-Qb wherein R₁ is CH₂CH₃, R₂ is CH₂CF₃, R₃ is H, X is S, A is CH and Q₁ is as defined in table Z.

Table B-29 provides 21 compounds B-29.001 to B-29.021 of formula Ia-Qb wherein R₁ is CH₂CH₃, R₂ is CH₂CF₃, R₃ is H, X is SO, A is CH and Q₁ is as defined in table Z.

Table B-30 provides 21 compounds B-30.001 to B-30.021 of formula Ia-Qb wherein R₁ is CH₂CH₃, R₂ is CH₂CF₃, R₃ is H, X is SO₂, A is CH and Q₁ is as defined in table Z.

Table B-31 provides 21 compounds B-31.001 to B-31.021 of formula Ia-Qb wherein R₁ is CH₂CH₃, R₂ is CH₂CF₂CHFCF₃, R₃ is H, X is S, A is CH and Q₁ is as defined in table Z.

Table B-32 provides 21 compounds B-32.001 to B-32.021 of formula Ia-Qb wherein R₁ is CH₂CH₃, R₂ is CH₂CF₂CHFCF₃, R₃ is H, X is SO, A is CH and Q₁ is as defined in table Z.

Table B-33 provides 21 compounds B-33.001 to B-33.021 of formula Ia-Qb wherein R₁ is CH₂CH₃, R₂ is CH₂CF₂CHFCF₃, R₃ is H, X is SO₂, A is CH and Q₁ is as defined in table Z.

Table B-34 provides 21 compounds B-34.001 to B-34.021 of formula Ia-Qb wherein R₁ is CH₂CH₃, R₂ is SO₂CF₃, R₃ is H, X is S, A is CH and Q₁ is as defined in table Z.

Table B-35 provides 21 compounds B-35.001 to B-35.021 of formula Ia-Qb wherein R₁ is CH₂CH₃, R₂ is SO₂CF₃, R₃ is H, X is SO, A is CH and Q₁ is as defined in table Z.

Table B-36 provides 21 compounds B-36.001 to B-36.021 of formula Ia-Qb wherein R₁ is CH₂CH₃, R₂ is SO₂CF₃, R₃ is H, X is SO₂, A is CH and Q₁ is as defined in table Z.

Table B-37 provides 21 compounds B-37.001 to B-37.021 of formula Ia-Qb wherein R₁ is —CH₂cyclopropyl, R₂ is CH₂CF₂CF₃, R₃ is H, X is S, A is N and Q₁ is as defined in table Z.

Table B-38 provides 21 compounds B-38.001 to B-38.021 of formula Ia-Qb wherein R₁ is —CH₂cyclopropyl, R₂ is CH₂CF₂CF₃, R₃ is H, X is SO, A is N and Q₁ is as defined in table Z.

Table B-39 provides 21 compounds B-39.001 to B-39.021 of formula Ia-Qb wherein R₁ is —CH₂cyclopropyl, R₂ is CH₂CF₂CF₃, R₃ is H, X is SO₂, A is N and Q₁ is as defined in table Z.

Table B-40 provides 21 compounds B-40.001 to B-40.021 of formula Ia-Qb wherein R₁ is —CH₂cyclopropyl, R₂ is CH₂CF₂CHF₂, R₃ is H, X is S, A is N and Q₁ is as defined in table Z.

Table B-41 provides 21 compounds B-41.001 to B-41.021 of formula Ia-Qb wherein R₁ is —CH₂cyclopropyl, R₂ is CH₂CF₂CHF₂, R₃ is H, X is SO, A is N and Q₁ is as defined in table Z.

Table B-42 provides 21 compounds B-42.001 to B-42.021 of formula Ia-Qb wherein R₁ is —CH₂cyclopropyl, R₂ is CH₂CF₂CHF₂, R₃ is H, X is SO₂, A is N and Q₁ is as defined in table Z.

Table B-43 provides 21 compounds B-43.001 to B-43.021 of formula Ia-Qb wherein R₁ is —CH₂cyclopropyl, R₂ is CH₂CF₃, R₃ is H, X is S, A is N and Q₁ is as defined in table Z.

Table B-44 provides 21 compounds B-44.001 to B-44.021 of formula Ia-Qb wherein R₁ is —CH₂cyclopropyl, R₂ is CH₂CF₃, R₃ is H, X is SO, A is N and Q₁ is as defined in table Z.

Table B-45 provides 21 compounds B-45.001 to B-45.021 of formula Ia-Qb wherein R₁ is —CH₂cyclopropyl, R₂ is CH₂CF₃, R₃ is H, X is SO₂, A is N and Q₁ is as defined in table Z.

Table B-46 provides 21 compounds B-46.001 to B-46.021 of formula Ia-Qb wherein R₁ is —CH₂cyclopropyl, R₂ is CH₂CF₂CF₃, R₃ is H, X is S, A is CH and Q₁ is as defined in table Z.

Table B-47 provides 21 compounds B-47.001 to B-47.021 of formula Ia-Qb wherein R₁ is —CH₂cyclopropyl, R₂ is CH₂CF₂CF₃, R₃ is H, X is SO, A is CH and Q₁ is as defined in table Z.

Table B-48 provides 21 compounds B-48.001 to B-48.021 of formula Ia-Qb wherein R₁ is —CH₂cyclopropyl, R₂ is CH₂CF₂CF₃, R₃ is H, X is SO₂, A is CH and Q₁ is as defined in table Z.

Table B-49 provides 21 compounds B-49.001 to B-49.021 of formula Ia-Qb wherein R₁ is —CH₂cyclopropyl, R₂ is CH₂CF₂CHF₂, R₃ is H, X is S, A is CH and Q₁ is as defined in table Z.

Table B-50 provides 21 compounds B-50.001 to B-50.021 of formula Ia-Qb wherein R₁ is —CH₂cyclopropyl, R₂ is CH₂CF₂CHF₂, R₃ is H, X is SO, A is CH and Q₁ is as defined in table Z.

Table B-51 provides 21 compounds B-51.001 to B-51.021 of formula Ia-Qb wherein R₁ is —CH₂cyclopropyl, R₂ is CH₂CF₂CHF₂, R₃ is H, X is SO₂, A is CH and Q₁ is as defined in table Z.

Table B-52 provides 21 compounds B-52.001 to B-52.021 of formula Ia-Qb wherein R₁ is —CH₂cyclopropyl, R₂ is CH₂CF₃, R₃ is H, X is S, A is CH and Q₁ is as defined in table Z.

Table B-53 provides 21 compounds B-53.001 to B-53.021 of formula Ia-Qb wherein R₁ is —CH₂cyclopropyl, R₂ is CH₂CF₃, R₃ is H, X is SO, A is CH and Q₁ is as defined in table Z.

Table B-54 provides 21 compounds B-54.001 to B-54.021 of formula Ia-Qb wherein R₁ is —CH₂cyclopropyl, R₂ is CH₂CF₃, R₃ is H, X is SO₂, A is CH and Q₁ is as defined in table Z.

The tables C-1 to C-81 below illustrate specific compounds of the invention.

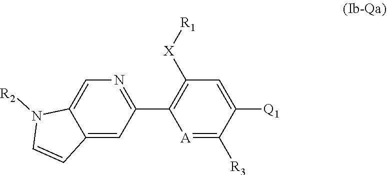

Table C-1 provides 20 compounds C-1.001 to C-1.020 of formula Ib-Qa wherein R₁ is CH₂CH₃, R₂ is CH₂CF₂CF₃, R₃ is H, X is S, A is N and Q₁ is as defined in table Y.

Table C-2 provides 20 compounds C-2.001 to C-2.020 of formula Ib-Qa wherein R₁ is CH₂CH₃, R₂ is CH₂CF₂CF₃, R₃ is H, X is SO, A is N and Q₁ is as defined in table Y.

Table C-3 provides 20 compounds C-3.001 to C-3.020 of formula Ib-Qa wherein R₁ is CH₂CH₃, R₂ is CH₂CF₂CF₃, R₃ is H, X is SO₂, A is N and Q₁ is as defined in table Y.

Table C-4 provides 20 compounds C-4.001 to C-4.020 of formula Ib-Qa wherein R₁ is CH₂CH₃, R₂ is CH₂CF₂CHF₂, R₃ is H, X is S, A is N and Q₁ is as defined in table Y.

Table C-5 provides 20 compounds C-5.001 to C-5.020 of formula Ib-Qa wherein R₁ is CH₂CH₃, R₂ is CH₂CF₂CHF₂, R₃ is H, X is SO, A is N and Q₁ is as defined in table Y.

Table C-6 provides 20 compounds C-6.001 to C-6.020 of formula Ib-Qa wherein R₁ is CH₂CH₃, R₂ is CH₂CF₂CHF₂, R₃ is H, X is SO₂, A is N and Q₁ is as defined in table Y.

Table C-7 provides 20 compounds C-7.001 to C-7.020 of formula Ib-Qa wherein R₁ is CH₂CH₃, R₂ is CH₂CHF₂, R₃ is H, X is S, A is N and Q₁ is as defined in table Y.

Table C-8 provides 20 compounds C-8.001 to C-8.020 of formula Ib-Qa wherein R₁ is CH₂CH₃, R₂ is CH₂CHF₂, R₃ is H, X is SO, A is N and Q₁ is as defined in table Y.

Table C-9 provides 20 compounds C-9.001 to C-9.020 of formula Ib-Qa wherein R₁ is CH₂CH₃, R₂ is CH₂CHF₂, R₃ is H, X is SO₂, A is N and Q₁ is as defined in table Y.

Table C-10 provides 20 compounds C-10.001 to C-10.020 of formula Ib-Qa wherein R₁ is CH₂CH₃, R₂ is CH₂CF₃, R₃ is H, X is S, A is N and Q₁ is as defined in table Y.

Table C-11 provides 20 compounds C-11.001 to C-11.020 of formula Ib-Qa wherein R₁ is CH₂CH₃, R₂ is CH₂CF₃, R₃ is H, X is SO, A is N and Q₁ is as defined in table Y.

Table C-12 provides 20 compounds C-12.001 to C-12.020 of formula Ib-Qa wherein R₁ is CH₂CH₃, R₂ is CH₂CF₃, R₃ is H, X is SO₂, A is N and Q₁ is as defined in table Y.

Table C-13 provides 20 compounds C-13.001 to C-13.020 of formula Ib-Qa wherein R₁ is CH₂CH₃, R₂ is CH₂CF₂CHFCF₃, R₃ is H, X is S, A is N and Q₁ is as defined in table Y.

Table C-14 provides 20 compounds C-14.001 to C-14.020 of formula Ib-Qa wherein R₁ is CH₂CH₃, R₂ is CH₂CF₂CHFCF₃, R₃ is H, X is SO, A is N and Q₁ is as defined in table Y.

Table C-15 provides 20 compounds C-15.001 to C-15.020 of formula Ib-Qa wherein R₁ is CH₂CH₃, R₂ is CH₂CF₂CHFCF₃, R₃ is H, X is SO₂, A is N and Q₁ is as defined in table Y.

Table C-16 provides 20 compounds C-16.001 to C-16.020 of formula Ib-Qa wherein R₁ is CH₂CH₃, R₂ is SO₂CF₃, R₃ is H, X is S, A is N and Q₁ is as defined in table Y.

Table C-17 provides 20 compounds C-17.001 to C-17.020 of formula Ib-Qa wherein R₁ is CH₂CH₃, R₂ is SO₂CF₃, R₃ is H, X is SO, A is N and Q₁ is as defined in table Y.

Table C-18 provides 20 compounds C-18.001 to C-18.020 of formula Ib-Qa wherein R₁ is CH₂CH₃, R₂ is SO₂CF₃, R₃ is H, X is SO₂, A is N and Q₁ is as defined in table Y.

Table C-19 provides 20 compounds C-19.001 to C-19.020 of formula Ib-Qa wherein R₁ is CH₂CH₃, R₂ is CH₂CF₂CF₃, R₃ is H, X is S, A is CH and Q₁ is as defined in table Y.

Table C-20 provides 20 compounds C-20.001 to C-20.020 of formula Ib-Qa wherein R₁ is CH₂CH₃, R₂ is CH₂CF₂CF₃, R₃ is H, X is SO, A is CH and Q₁ is as defined in table Y.

Table C-21 provides 20 compounds C-21.001 to C-21.020 of formula Ib-Qa wherein R₁ is CH₂CH₃, R₂ is CH₂CF₂CF₃, R₃ is H, X is SO₂, A is CH and Q₁ is as defined in table Y.

Table C-22 provides 20 compounds C-22.001 to C-22.020 of formula Ib-Qa wherein R₁ is CH₂CH₃, R₂ is CH₂CF₂CHF₂, R₃ is H, X is S, A is CH and Q₁ is as defined in table Y.

Table C-23 provides 20 compounds C-23.001 to C-23.020 of formula Ib-Qa wherein R₁ is CH₂CH₃, R₂ is CH₂CF₂CHF₂, R₃ is H, X is SO, A is CH and Q₁ is as defined in table Y.

Table C-24 provides 20 compounds C-24.001 to C-24.020 of formula Ib-Qa wherein R₁ is CH₂CH₃, R₂ is CH₂CF₂CHF₂, R₃ is H, X is SO₂, A is CH and Q₁ is as defined in table Y.

Table C-25 provides 20 compounds C-25.001 to C-25.020 of formula Ib-Qa wherein R₁ is CH₂CH₃, R₂ is CH₂CHF₂, R₃ is H, X is S, A is CH and Q₁ is as defined in table Y.

Table C-26 provides 20 compounds C-26.001 to C-26.020 of formula Ib-Qa wherein R₁ is CH₂CH₃, R₂ is CH₂CHF₂, R₃ is H, X is SO, A is CH and Q₁ is as defined in table Y.

Table C-27 provides 20 compounds C-27.001 to C-27.020 of formula Ib-Qa wherein R₁ is CH₂CH₃, R₂ is CH₂CHF₂, R₃ is H, X is SO₂, A is CH and Q₁ is as defined in table Y.

Table C-28 provides 20 compounds C-28.001 to C-28.020 of formula Ib-Qa wherein R₁ is CH₂CH₃, R₂ is CH₂CF₃, R₃ is H, X is S, A is CH and Q₁ is as defined in table Y.

Table C-29 provides 20 compounds C-29.001 to C-29.020 of formula Ib-Qa wherein R₁ is CH₂CH₃, R₂ is CH₂CF₃, R₃ is H, X is SO, A is CH and Q₁ is as defined in table Y.

Table C-30 provides 20 compounds C-30.001 to C-30.020 of formula Ib-Qa wherein R₁ is CH₂CH₃, R₂ is CH₂CF₃, R₃ is H, X is SO₂, A is CH and Q₁ is as defined in table Y.

Table C-31 provides 20 compounds C-31.001 to C-31.020 of formula Ib-Qa wherein R₁ is CH₂CH₃, R₂ is CH₂CF₂CHFCF₃, R₃ is H, X is S, A is CH and Q₁ is as defined in table Y.

Table C-32 provides 20 compounds C-32.001 to C-32.020 of formula Ib-Qa wherein R₁ is CH₂CH₃, R₂ is CH₂CF₂CHFCF₃, R₃ is H, X is SO, A is CH and Q₁ is as defined in table Y.

Table C-33 provides 20 compounds C-33.001 to C-33.020 of formula Ib-Qa wherein R₁ is CH₂CH₃, R₂ is CH₂CF₂CHFCF₃, R₃ is H, X is SO₂, A is CH and Q₁ is as defined in table Y.

Table C-34 provides 20 compounds C-34.001 to C-34.020 of formula Ib-Qa wherein R₁ is CH₂CH₃, R₂ is SO₂CF₃, R₃ is H, X is S, A is CH and Q₁ is as defined in table Y.

Table C-35 provides 20 compounds C-35.001 to C-35.020 of formula Ib-Qa wherein R₁ is CH₂CH₃, R₂ is SO₂CF₃, R₃ is H, X is SO, A is CH and Q₁ is as defined in table Y.

Table C-36 provides 20 compounds C-36.001 to C-36.020 of formula Ib-Qa wherein R₁ is CH₂CH₃, R₂ is SO₂CF₃, R₃ is H, X is SO₂, A is CH and Q₁ is as defined in table Y.

Table C-37 provides 20 compounds C-37.001 to C-37.020 of formula Ib-Qa wherein R₁ is CH₂CH₃, R₂ is CH₂CF₂CF₃, R₃ is CH₃, X is S, A is N and Q₁ is as defined in table Y.

Table C-38 provides 20 compounds C-38.001 to C-38.020 of formula Ib-Qa wherein R₁ is CH₂CH₃, R₂ is CH₂CF₂CF₃, R₃ is CH₃, X is SO, A is N and Q₁ is as defined in table Y.

Table C-39 provides 20 compounds C-39.001 to C-39.020 of formula Ib-Qa wherein R₁ is CH₂CH₃, R₂ is CH₂CF₂CF₃, R₃ is CH₃, X is SO₂, A is N and Q₁ is as defined in table Y.

Table C-40 provides 20 compounds C-40.001 to C-40.020 of formula Ib-Qa wherein R₁ is CH₂CH₃, R₂ is CH₂CF₂CHF₂, R₃ is CH₃, X is S, A is N and Q₁ is as defined in table Y.

Table C-41 provides 20 compounds C-41.001 to C-41.020 of formula Ib-Qa wherein R₁ is CH₂CH₃, R₂ is CH₂CF₂CHF₂, R₃ is CH₃, X is SO, A is N and Q₁ is as defined in table Y.

Table C-42 provides 20 compounds C-42.001 to C-42.020 of formula Ib-Qa wherein R₁ is CH₂CH₃, R₂ is CH₂CF₂CHF₂, R₃ is CH₃, X is SO₂, A is N and Q₁ is as defined in table Y.

Table C-43 provides 20 compounds C-43.001 to C-43.020 of formula Ib-Qa wherein R₁ is CH₂CH₃, R₂ is CH₂CHF₂, R₃ is CH₃, X is S, A is N and Q₁ is as defined in table Y.

Table C-44 provides 20 compounds C-44.001 to C-44.020 of formula Ib-Qa wherein R₁ is CH₂CH₃, R₂ is CH₂CHF₂, R₃ is CH₃, X is SO, A is N and Q₁ is as defined in table Y.

Table C-45 provides 20 compounds C-45.001 to C-45.020 of formula Ib-Qa wherein R₁ is CH₂CH₃, R₂ is CH₂CHF₂, R₃ is CH₃, X is SO₂, A is N and Q₁ is as defined in table Y.

Table C-46 provides 20 compounds C-46.001 to C-46.020 of formula Ib-Qa wherein R₁ is CH₂CH₃, R₂ is CH₂CF₃, R₃ is CH₃, X is S, A is N and Q₁ is as defined in table Y.

Table C-47 provides 20 compounds C-47.001 to C-47.020 of formula Ib-Qa wherein R₁ is CH₂CH₃, R₂ is CH₂CF₃, R₃ is CH₃, X is SO, A is N and Q₁ is as defined in table Y.

Table C-48 provides 20 compounds C-48.001 to C-48.020 of formula Ib-Qa wherein R₁ is CH₂CH₃, R₂ is CH₂CF₃, R₃ is CH₃, X is SO₂, A is N and Q₁ is as defined in table Y.

Table C-49 provides 20 compounds C-49.001 to C-49.020 of formula Ib-Qa wherein R₁ is CH₂CH₃, R₂ is CH₂CF₂CHFCF₃, R₃ is CH₃, X is S, A is N and Q₁ is as defined in table Y.

Table C-50 provides 20 compounds C-50.001 to C-50.020 of formula Ib-Qa wherein R₁ is CH₂CH₃, R₂ is CH₂CF₂CHFCF₃, R₃ is CH₃, X is SO, A is N and Q₁ is as defined in table Y.

Table C-51 provides 20 compounds C-51.001 to C-51.020 of formula Ib-Qa wherein R₁ is CH₂CH₃, R₂ is CH₂CF₂CHFCF₃, R₃ is CH₃, X is SO₂, A is N and Q₁ is as defined in table Y.

Table C-52 provides 20 compounds C-52.001 to C-52.020 of formula Ib-Qa wherein R₁ is CH₂CH₃, R₂ is SO₂CF₃, R₃ is CH₃, X is S, A is N and Q₁ is as defined in table Y.

Table C-53 provides 20 compounds C-53.001 to C-53.020 of formula Ib-Qa wherein R₁ is CH₂CH₃, R₂ is SO₂CF₃, R₃ is CH₃, X is SO, A is N and Q₁ is as defined in table Y.

Table C-54 provides 20 compounds C-54.001 to C-54.020 of formula Ib-Qa wherein R₁ is CH₂CH₃, R₂ is SO₂CF₃, R₃ is CH₃, X is SO₂, A is N and Q₁ is as defined in table Y.

Table C-55 provides 20 compounds C-55.001 to C-55.020 of formula Ia-Qa wherein R₁ is —CH₂cyclopropyl, R₂ is CH₂CF₂CF₃, R₃ is H, X is S, A is N and Q₁ is as defined in table Y.

Table C-56 provides 20 compounds C-56.001 to C-56.020 of formula Ia-Qa wherein R₁ is —CH₂cyclopropyl, R₂ is CH₂CF₂CF₃, R₃ is H, X is SO, A is N and Q₁ is as defined in table Y.

Table C-57 provides 20 compounds C-57.001 to C-57.020 of formula Ia-Qa wherein R₁ is —CH₂cyclopropyl, R₂ is CH₂CF₂CF₃, R₃ is H, X is SO₂, A is N and Q₁ is as defined in table Y.

Table C-58 provides 20 compounds C-58.001 to C-58.020 of formula Ia-Qa wherein R₁ is —CH₂cyclopropyl, R₂ is CH₂CF₂CHF₂, R₃ is H, X is S, A is N and Q₁ is as defined in table Y.

Table C-59 provides 20 compounds C-59.001 to C-59.020 of formula Ia-Qa wherein R₁ is —CH₂cyclopropyl, R₂ is CH₂CF₂CHF₂, R₃ is H, X is SO, A is N and Q₁ is as defined in table Y.

Table C-60 provides 20 compounds C-60.001 to C-60.020 of formula Ia-Qa wherein R₁ is —CH₂cyclopropyl, R₂ is CH₂CF₂CHF₂, R₃ is H, X is SO₂, A is N and Q₁ is as defined in table Y.

Table C-61 provides 20 compounds C-61.001 to C-61.020 of formula Ia-Qa wherein R₁ is —CH₂cyclopropyl, R₂ is CH₂CF₃, R₃ is H, X is S, A is N and Q₁ is as defined in table Y.

Table C-62 provides 20 compounds C-62.001 to C-62.020 of formula Ia-Qa wherein R₁ is —CH₂cyclopropyl, R₂ is CH₂CF₃, R₃ is H, X is SO, A is N and Q₁ is as defined in table Y.

Table C-63 provides 20 compounds C-63.001 to C-63.020 of formula Ia-Qa wherein R₁ is —CH₂cyclopropyl, R₂ is CH₂CF₃, R₃ is H, X is SO₂, A is N and Q₁ is as defined in table Y.

Table C-64 provides 20 compounds C-64.001 to C-64.020 of formula Ia-Qa wherein R₁ is —CH₂cyclopropyl, R₂ is CH₂CF₂CF₃, R₃ is H, X is S, A is CH and Q₁ is as defined in table Y.

Table C-65 provides 20 compounds C-65.001 to C-65.020 of formula Ia-Qa wherein R₁ is —CH₂cyclopropyl, R₂ is CH₂CF₂CF₃, R₃ is H, X is SO, A is CH and Q₁ is as defined in table Y.

Table C-66 provides 20 compounds C-66.001 to C-66.020 of formula Ia-Qa wherein R₁ is —CH₂cyclopropyl, R₂ is CH₂CF₂CF₃, R₃ is H, X is SO₂, A is CH and Q₁ is as defined in table Y.

Table C-67 provides 20 compounds C-67.001 to C-67.020 of formula Ia-Qa wherein R₁ is —CH₂cyclopropyl, R₂ is CH₂CF₂CHF₂, R₃ is H, X is S, A is CH and Q₁ is as defined in table Y.

Table C-68 provides 20 compounds C-68.001 to C-68.020 of formula Ia-Qa wherein R₁ is —CH₂cyclopropyl, R₂ is CH₂CF₂CHF₂, R₃ is H, X is SO, A is CH and Q₁ is as defined in table Y.

Table C-69 provides 20 compounds C-69.001 to C-69.020 of formula Ia-Qa wherein R₁ is —CH₂cyclopropyl, R₂ is CH₂CF₂CHF₂, R₃ is H, X is SO₂, A is CH and Q₁ is as defined in table Y.

Table C-70 provides 20 compounds C-70.001 to C-70.020 of formula Ia-Qa wherein R₁ is —CH₂cyclopropyl, R₂ is CH₂CF₃, R₃ is H, X is S, A is CH and Q₁ is as defined in table Y.

Table C-71 provides 20 compounds C-71.001 to C-71.020 of formula Ia-Qa wherein R₁ is —CH₂cyclopropyl, R₂ is CH₂CF₃, R₃ is H, X is SO, A is CH and Q₁ is as defined in table Y.

Table C-72 provides 20 compounds C-72.001 to C-72.020 of formula Ia-Qa wherein R₁ is —CH₂cyclopropyl, R₂ is CH₂CF₃, R₃ is H, X is SO₂, A is CH and Q₁ is as defined in table Y.

Table C-73 provides 20 compounds C-73.001 to C-73.020 of formula Ia-Qa wherein R₁ is —CH₂cyclopropyl, R₂ is CH₂CF₂CF₃, R₃ is CH₃, X is S, A is N and Q₁ is as defined in table Y.

Table C-74 provides 20 compounds C-74.001 to C-74.020 of formula Ia-Qa wherein R₁ is —CH₂cyclopropyl, R₂ is CH₂CF₂CF₃, R₃ is CH₃, X is SO, A is N and Q₁ is as defined in table Y.

Table C-75 provides 20 compounds C-75.001 to C-75.020 of formula Ia-Qa wherein R₁ is —CH₂cyclopropyl, R₂ is CH₂CF₂CF₃, R₃ is CH₃, X is SO₂, A is N and Q₁ is as defined in table Y.

Table C-76 provides 20 compounds C-76.001 to C-76.020 of formula Ia-Qa wherein R₁ is —CH₂cyclopropyl, R₂ is CH₂CF₂CHF₂, R₃ is CH₃, X is S, A is N and Q₁ is as defined in table Y.

Table C-77 provides 20 compounds C-77.001 to C-77.020 of formula Ia-Qa wherein R₁ is —CH₂cyclopropyl, R₂ is CH₂CF₂CHF₂, R₃ is CH₃, X is SO, A is N and Q₁ is as defined in table Y.

Table C-78 provides 20 compounds C-78.001 to C-78.020 of formula Ia-Qa wherein R₁ is —CH₂cyclopropyl, R₂ is CH₂CF₂CHF₂, R₃ is CH₃, X is SO₂, A is N and Q₁ is as defined in table Y.

Table C-79 provides 20 compounds C-79.001 to C-79.020 of formula Ia-Qa wherein R₁ is —CH₂cyclopropyl, R₂ is CH₂CF₃, R₃ is CH₃, X is S, A is N and Q₁ is as defined in table Y.

Table C-80 provides 20 compounds C-80.001 to C-80.020 of formula Ia-Qa wherein R₁ is —CH₂cyclopropyl, R₂ is CH₂CF₃, R₃ is CH₃, X is SO, A is N and Q₁ is as defined in table Y.

Table C-81 provides 20 compounds C-81.001 to C-81.020 of formula Ia-Qa wherein R₁ is —CH₂cyclopropyl, R₂ is CH₂CF₃, R₃ is CH₃, X is SO₂, A is N and Q₁ is as defined in table Y.

The tables D-1 to D-54 below illustrate further specific compounds of the invention.

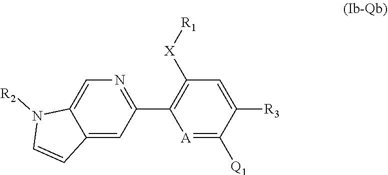

Table D-1 provides 21 compounds D-1.001 to D-1.021 of formula Ib-Qb wherein R₁ is CH₂CH₃, R₂ is CH₂CF₂CF₃, R₃ is H, X is S, A is N and Q₁ is as defined in table Z.

Table D-2 provides 21 compounds D-2.001 to D-2.021 of formula Ib-Qb wherein R₁ is CH₂CH₃, R₂ is CH₂CF₂CF₃, R₃ is H, X is SO, A is N and Q₁ is as defined in table Z.

Table D-3 provides 21 compounds D-3.001 to D-3.021 of formula Ib-Qb wherein R₁ is CH₂CH₃, R₂ is CH₂CF₂CF₃, R₃ is H, X is SO₂, A is N and Q₁ is as defined in table Z.

Table D-4 provides 21 compounds D-4.001 to D-4.021 of formula Ib-Qb wherein R₁ is CH₂CH₃, R₂ is CH₂CF₂CHF₂, R₃ is H, X is S, A is N and Q₁ is as defined in table Z.

Table D-5 provides 21 compounds D-5.001 to D-5.021 of formula Ib-Qb wherein R₁ is CH₂CH₃, R₂ is CH₂CF₂CHF₂, R₃ is H, X is SO, A is N and Q₁ is as defined in table Z.

Table D-6 provides 21 compounds D-6.001 to D-6.021 of formula Ib-Qb wherein R₁ is CH₂CH₃, R₂ is CH₂CF₂CHF₂, R₃ is H, X is SO₂, A is N and Q₁ is as defined in table Z.

Table D-7 provides 21 compounds D-7.001 to D-7.021 of formula Ib-Qb wherein R₁ is CH₂CH₃, R₂ is CH₂CHF₂, R₃ is H, X is S, A is N and Q₁ is as defined in table Z.

Table D-8 provides 21 compounds D-8.001 to D-8.021 of formula Ib-Qb wherein R₁ is CH₂CH₃, R₂ is CH₂CHF₂, R₃ is H, X is SO, A is N and Q₁ is as defined in table Z.

Table D-9 provides 21 compounds D-9.001 to D-9.021 of formula Ib-Qb wherein R₁ is CH₂CH₃, R₂ is CH₂CHF₂, R₃ is H, X is SO₂, A is N and Q₁ is as defined in table Z.

Table D-10 provides 21 compounds D-10.001 to D-10.021 of formula Ib-Qb wherein R₁ is CH₂CH₃, R₂ is CH₂CF₃, R₃ is H, X is S, A is N and Q₁ is as defined in table Z.

Table D-11 provides 21 compounds D-11.001 to D-11.021 of formula Ib-Qb wherein R₁ is CH₂CH₃, R₂ is CH₂CF₃, R₃ is H, X is SO, A is N and Q₁ is as defined in table Z.

Table D-12 provides 21 compounds D-12.001 to D-12.021 of formula Ib-Qb wherein R₁ is CH₂CH₃, R₂ is CH₂CF₃, R₃ is H, X is SO₂, A is N and Q₁ is as defined in table Z.

Table D-13 provides 21 compounds D-13.001 to D-13.021 of formula Ib-Qb wherein R₁ is CH₂CH₃, R₂ is CH₂CF₂CHFCF₃, R₃ is H, X is S, A is N and Q₁ is as defined in table Z.

Table D-14 provides 21 compounds D-14.001 to D-14.021 of formula Ib-Qb wherein R₁ is CH₂CH₃, R₂ is CH₂CF₂CHFCF₃, R₃ is H, X is SO, A is N and Q₁ is as defined in table Z.

Table D-15 provides 21 compounds D-15.001 to D-15.021 of formula Ib-Qb wherein R₁ is CH₂CH₃, R₂ is CH₂CF₂CHFCF₃, R₃ is H, X is SO₂, A is N and Q₁ is as defined in table Z.

Table D-16 provides 21 compounds D-16.001 to D-16.021 of formula Ib-Qb wherein R₁ is CH₂CH₃, R₂ is SO₂CF₃, R₃ is H, X is S, A is N and Q₁ is as defined in table Z.

Table D-17 provides 21 compounds D-17.001 to D-17.021 of formula Ib-Qb wherein R₁ is CH₂CH₃, R₂ is SO₂CF₃, R₃ is H, X is SO, A is N and Q₁ is as defined in table Z.

Table D-18 provides 21 compounds D-18.001 to D-18.021 of formula Ib-Qb wherein R₁ is CH₂CH₃, R₂ is SO₂CF₃, R₃ is H, X is SO₂, A is N and Q₁ is as defined in table Z.

Table D-19 provides 21 compounds D-19.001 to D-19.021 of formula Ib-Qb wherein R₁ is CH₂CH₃, R₂ is CH₂CF₂CF₃, R₃ is H, X is S, A is CH and Q₁ is as defined in table Z.

Table D-20 provides 21 compounds D-20.001 to D-20.021 of formula Ib-Qb wherein R₁ is CH₂CH₃, R₂ is CH₂CF₂CF₃, R₃ is H, X is SO, A is CH and Q₁ is as defined in table Z.

Table D-21 provides 21 compounds D-21.001 to D-21.021 of formula Ib-Qb wherein R₁ is CH₂CH₃, R₂ is CH₂CF₂CF₃, R₃ is H, X is SO₂, A is CH and Q₁ is as defined in table Z.

Table D-22 provides 21 compounds D-22.001 to D-22.021 of formula Ib-Qb wherein R₁ is CH₂CH₃, R₂ is CH₂CF₂CHF₂, R₃ is H, X is S, A is CH and Q₁ is as defined in table Z.

Table D-23 provides 21 compounds D-23.001 to D-23.021 of formula Ib-Qb wherein R₁ is CH₂CH₃, R₂ is CH₂CF₂CHF₂, R₃ is H, X is SO, A is CH and Q₁ is as defined in table Z.

Table D-24 provides 21 compounds D-24.001 to D-24.021 of formula Ib-Qb wherein R₁ is CH₂CH₃, R₂ is CH₂CF₂CHF₂, R₃ is H, X is SO₂, A is CH and Q₁ is as defined in table Z.

Table D-25 provides 21 compounds D-25.001 to D-25.021 of formula Ib-Qb wherein R₁ is CH₂CH₃, R₂ is CH₂CHF₂, R₃ is H, X is S, A is CH and Q₁ is as defined in table Z.

Table D-26 provides 21 compounds D-26.001 to D-26.021 of formula Ib-Qb wherein R₁ is CH₂CH₃, R₂ is CH₂CHF₂, R₃ is H, X is SO, A is CH and Q₁ is as defined in table Z.

Table D-27 provides 21 compounds D-27.001 to D-27.021 of formula Ib-Qb wherein R₁ is CH₂CH₃, R₂ is CH₂CHF₂, R₃ is H, X is SO₂, A is CH and Q₁ is as defined in table Z.

Table D-28 provides 21 compounds D-28.001 to D-28.021 of formula Ib-Qb wherein R₁ is CH₂CH₃, R₂ is CH₂CF₃, R₃ is H, X is S, A is CH and Q₁ is as defined in table Z.

Table D-29 provides 21 compounds D-29.001 to D-29.021 of formula Ib-Qb wherein R₁ is CH₂CH₃, R₂ is CH₂CF₃, R₃ is H, X is SO, A is CH and Q₁ is as defined in table Z.

Table D-30 provides 21 compounds D-30.001 to D-30.021 of formula Ib-Qb wherein R₁ is CH₂CH₃, R₂ is CH₂CF₃, R₃ is H, X is SO₂, A is CH and Q₁ is as defined in table Z.

Table D-31 provides 21 compounds D-31.001 to D-31.021 of formula Ib-Qb wherein R₁ is CH₂CH₃, R₂ is CH₂CF₂CHFCF₃, R₃ is H, X is S, A is CH and Q₁ is as defined in table Z.

Table D-32 provides 21 compounds D-32.001 to D-32.021 of formula Ib-Qb wherein R₁ is CH₂CH₃, R₂ is CH₂CF₂CHFCF₃, R₃ is H, X is SO, A is CH and Q₁ is as defined in table Z.

Table D-33 provides 21 compounds D-33.001 to D-33.021 of formula Ib-Qb wherein R₁ is CH₂CH₃, R₂ is CH₂CF₂CHFCF₃, R₃ is H, X is SO₂, A is CH and Q₁ is as defined in table Z.

Table D-34 provides 21 compounds D-34.001 to D-34.021 of formula Ib-Qb wherein R₁ is CH₂CH₃, R₂ is SO₂CF₃, R₃ is H, X is S, A is CH and Q₁ is as defined in table Z.

Table D-35 provides 21 compounds D-35.001 to D-35.021 of formula Ib-Qb wherein R₁ is CH₂CH₃, R₂ is SO₂CF₃, R₃ is H, X is SO, A is CH and Q₁ is as defined in table Z.

Table D-36 provides 21 compounds D-36.001 to D-36.021 of formula Ib-Qb wherein R₁ is CH₂CH₃, R₂ is SO₂CF₃, R₃ is H, X is SO₂, A is CH and Q₁ is as defined in table Z.

Table D-37 provides 21 compounds D-37.001 to D-37.021 of formula Ia-Qb wherein R₁ is —CH₂cyclopropyl, R₂ is CH₂CF₂CF₃, R₃ is H, X is S, A is N and Q₁ is as defined in table Z.

Table D-38 provides 21 compounds D-38.001 to D-38.021 of formula Ia-Qb wherein R₁ is —CH₂cyclopropyl, R₂ is CH₂CF₂CF₃, R₃ is H, X is SO, A is N and Q₁ is as defined in table Z.

Table D-39 provides 21 compounds D-39.001 to D-39.021 of formula Ia-Qb wherein R₁ is —CH₂cyclopropyl, R₂ is CH₂CF₂CF₃, R₃ is H, X is SO₂, A is N and Q₁ is as defined in table Z.

Table D-40 provides 21 compounds D-40.001 to D-40.021 of formula Ia-Qb wherein R₁ is —CH₂cyclopropyl, R₂ is CH₂CF₂CHF₂, R₃ is H, X is S, A is N and Q₁ is as defined in table Z.

Table D-41 provides 21 compounds D-41.001 to D-41.021 of formula Ia-Qb wherein R₁ is —CH₂cyclopropyl, R₂ is CH₂CF₂CHF₂, R₃ is H, X is SO, A is N and Q₁ is as defined in table Z.

Table D-42 provides 21 compounds D-42.001 to D-42.021 of formula Ia-Qb wherein R₁ is —CH₂cyclopropyl, R₂ is CH₂CF₂CHF₂, R₃ is H, X is SO₂, A is N and Q₁ is as defined in table Z.

Table D-43 provides 21 compounds D-43.001 to D-43.021 of formula Ia-Qb wherein R₁ is —CH₂cyclopropyl, R₂ is CH₂CF₃, R₃ is H, X is S, A is N and Q₁ is as defined in table Z.

Table D-44 provides 21 compounds D-44.001 to D-44.021 of formula Ia-Qb wherein R₁ is —CH₂cyclopropyl, R₂ is CH₂CF₃, R₃ is H, X is SO, A is N and Q₁ is as defined in table Z.

Table D-45 provides 21 compounds D-45.001 to D-45.021 of formula Ia-Qb wherein R₁ is —CH₂cyclopropyl, R₂ is CH₂CF₃, R₃ is H, X is SO₂, A is N and Q₁ is as defined in table Z.

Table D-46 provides 21 compounds D-46.001 to D-46.021 of formula Ia-Qb wherein R₁ is —CH₂cyclopropyl, R₂ is CH₂CF₂CF₃, R₃ is H, X is S, A is CH and Q₁ is as defined in table Z.

Table D-47 provides 21 compounds D-47.001 to D-47.021 of formula Ia-Qb wherein R₁ is —CH₂cyclopropyl, R₂ is CH₂CF₂CF₃, R₃ is H, X is SO, A is CH and Q₁ is as defined in table Z.

Table D-48 provides 21 compounds D-48.001 to D-48.021 of formula Ia-Qb wherein R₁ is —CH₂cyclopropyl, R₂ is CH₂CF₂CF₃, R₃ is H, X is SO₂, A is CH and Q₁ is as defined in table Z.

Table D-49 provides 21 compounds D-49.001 to D-49.021 of formula Ia-Qb wherein R₁ is —CH₂cyclopropyl, R₂ is CH₂CF₂CHF₂, R₃ is H, X is S, A is CH and Q₁ is as defined in table Z.

Table D-50 provides 21 compounds D-50.001 to D-50.021 of formula Ia-Qb wherein R₁ is —CH₂cyclopropyl, R₂ is CH₂CF₂CHF₂, R₃ is H, X is SO, A is CH and Q₁ is as defined in table Z.

Table D-51 provides 21 compounds D-51.001 to D-51.021 of formula Ia-Qb wherein R₁ is —CH₂cyclopropyl, R₂ is CH₂CF₂CHF₂, R₃ is H, X is SO₂, A is CH and Q₁ is as defined in table Z.

Table D-52 provides 21 compounds D-52.001 to D-52.021 of formula Ia-Qb wherein R₁ is —CH₂cyclopropyl, R₂ is CH₂CF₃, R₃ is H, X is S, A is CH and Q₁ is as defined in table Z.

Table D-53 provides 21 compounds D-53.001 to D-53.021 of formula Ia-Qb wherein R₁ is —CH₂cyclopropyl, R₂ is CH₂CF₃, R₃ is H, X is SO, A is CH and Q₁ is as defined in table Z.

Table D-54 provides 21 compounds D-54.001 to D-54.021 of formula Ia-Qb wherein R₁ is —CH₂cyclopropyl, R₂ is CH₂CF₃, R₃ is H, X is SO₂, A is CH and Q₁ is as defined in table Z.

The tables E-1 to E-81 below illustrate specific compounds of the invention.

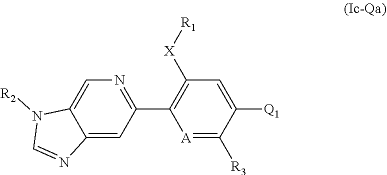

Table E-1 provides 20 compounds E-1.001 to E-1.020 of formula Ic-Qa wherein R₁ is CH₂CH₃, R₂ is CH₂CF₂CF₃, R₃ is H, X is S, A is N and Q₁ is as defined in table Y.

Table E-2 provides 20 compounds E-2.001 to E-2.020 of formula Ic-Qa wherein R₁ is CH₂CH₃, R₂ is CH₂CF₂CF₃, R₃ is H, X is SO, A is N and Q₁ is as defined in table Y.

Table E-3 provides 20 compounds E-3.001 to E-3.020 of formula Ic-Qa wherein R₁ is CH₂CH₃, R₂ is CH₂CF₂CF₃, R₃ is H, X is SO₂, A is N and Q₁ is as defined in table Y.

Table E-4 provides 20 compounds E-4.001 to E-4.020 of formula Ic-Qa wherein R₁ is CH₂CH₃, R₂ is CH₂CF₂CHF₂, R₃ is H, X is S, A is N and Q₁ is as defined in table Y.

Table E-5 provides 20 compounds E-5.001 to E-5.020 of formula Ic-Qa wherein R₁ is CH₂CH₃, R₂ is CH₂CF₂CHF₂, R₃ is H, X is SO, A is N and Q₁ is as defined in table Y.

Table E-6 provides 20 compounds E-6.001 to E-6.020 of formula Ic-Qa wherein R₁ is CH₂CH₃, R₂ is CH₂CF₂CHF₂, R₃ is H, X is SO₂, A is N and Q₁ is as defined in table Y.

Table E-7 provides 20 compounds E-7.001 to E-7.020 of formula Ic-Qa wherein R₁ is CH₂CH₃, R₂ is CH₂CHF₂, R₃ is H, X is S, A is N and Q₁ is as defined in table Y.

Table E-8 provides 20 compounds E-8.001 to E-8.020 of formula Ic-Qa wherein R₁ is CH₂CH₃, R₂ is CH₂CHF₂, R₃ is H, X is SO, A is N and Q₁ is as defined in table Y.

Table E-9 provides 20 compounds E-9.001 to E-9.020 of formula Ic-Qa wherein R₁ is CH₂CH₃, R₂ is CH₂CHF₂, R₃ is H, X is SO₂, A is N and Q₁ is as defined in table Y.

Table E-10 provides 20 compounds E-10.001 to E-10.020 of formula Ic-Qa wherein R₁ is CH₂CH₃, R₂ is CH₂CF₃, R₃ is H, X is S, A is N and Q₁ is as defined in table Y.

Table E-11 provides 20 compounds E-11.001 to E-11.020 of formula Ic-Qa wherein R₁ is CH₂CH₃, R₂ is CH₂CF₃, R₃ is H, X is SO, A is N and Q₁ is as defined in table Y.

Table E-12 provides 20 compounds E-12.001 to E-12.020 of formula Ic-Qa wherein R₁ is CH₂CH₃, R₂ is CH₂CF₃, R₃ is H, X is SO₂, A is N and Q₁ is as defined in table Y.

Table E-13 provides 20 compounds E-13.001 to E-13.020 of formula Ic-Qa wherein R₁ is CH₂CH₃, R₂ is CH₂CF₂CHFCF₃, R₃ is H, X is S, A is N and Q₁ is as defined in table Y.

Table E-14 provides 20 compounds E-14.001 to E-14.020 of formula Ic-Qa wherein R₁ is CH₂CH₃, R₂ is CH₂CF₂CHFCF₃, R₃ is H, X is SO, A is N and Q₁ is as defined in table Y.

Table E-15 provides 20 compounds E-15.001 to E-15.020 of formula Ic-Qa wherein R₁ is CH₂CH₃, R₂ is CH₂CF₂CHFCF₃, R₃ is H, X is SO₂, A is N and Q₁ is as defined in table Y.

Table E-16 provides 20 compounds E-16.001 to E-16.020 of formula Ic-Qa wherein R₁ is CH₂CH₃, R₂ is SO₂CF₃, R₃ is H, X is S, A is N and Q₁ is as defined in table Y.

Table E-17 provides 20 compounds E-17.001 to E-17.020 of formula Ic-Qa wherein R₁ is CH₂CH₃, R₂ is SO₂CF₃, R₃ is H, X is SO, A is N and Q₁ is as defined in table Y.

Table E-18 provides 20 compounds E-18.001 to E-18.020 of formula Ic-Qa wherein R₁ is CH₂CH₃, R₂ is SO₂CF₃, R₃ is H, X is SO₂, A is N and Q₁ is as defined in table Y.

Table E-19 provides 20 compounds E-19.001 to E-19.020 of formula Ic-Qa wherein R₁ is CH₂CH₃, R₂ is CH₂CF₂CF₃, R₃ is H, X is S, A is CH and Q₁ is as defined in table Y.

Table E-20 provides 20 compounds E-20.001 to E-20.020 of formula Ic-Qa wherein R₁ is CH₂CH₃, R₂ is CH₂CF₂CF₃, R₃ is H, X is SO, A is CH and Q₁ is as defined in table Y.

Table E-21 provides 20 compounds E-21.001 to E-21.020 of formula Ic-Qa wherein R₁ is CH₂CH₃, R₂ is CH₂CF₂CF₃, R₃ is H, X is SO₂, A is CH and Q₁ is as defined in table Y.

Table E-22 provides 20 compounds E-22.001 to E-22.020 of formula Ic-Qa wherein R₁ is CH₂CH₃, R₂ is CH₂CF₂CHF₂, R₃ is H, X is S, A is CH and Q₁ is as defined in table Y.

Table E-23 provides 20 compounds E-23.001 to E-23.020 of formula Ic-Qa wherein R₁ is CH₂CH₃, R₂ is CH₂CF₂CHF₂, R₃ is H, X is SO, A is CH and Q₁ is as defined in table Y.

Table E-24 provides 20 compounds E-24.001 to E-24.020 of formula Ic-Qa wherein R₁ is CH₂CH₃, R₂ is CH₂CF₂CHF₂, R₃ is H, X is SO₂, A is CH and Q₁ is as defined in table Y.

Table E-25 provides 20 compounds E-25.001 to E-25.020 of formula Ic-Qa wherein R₁ is CH₂CH₃, R₂ is CH₂CHF₂, R₃ is H, X is S, A is CH and Q₁ is as defined in table Y.

Table E-26 provides 20 compounds E-26.001 to E-26.020 of formula Ic-Qa wherein R₁ is CH₂CH₃, R₂ is CH₂CHF₂, R₃ is H, X is SO, A is CH and Q₁ is as defined in table Y.

Table E-27 provides 20 compounds E-27.001 to E-27.020 of formula Ic-Qa wherein R₁ is CH₂CH₃, R₂ is CH₂CHF₂, R₃ is H, X is SO₂, A is CH and Q₁ is as defined in table Y.

Table E-28 provides 20 compounds E-28.001 to E-28.020 of formula Ic-Qa wherein R₁ is CH₂CH₃, R₂ is CH₂CF₃, R₃ is H, X is S, A is CH and Q₁ is as defined in table Y.

Table E-29 provides 20 compounds E-29.001 to E-29.020 of formula Ic-Qa wherein R₁ is CH₂CH₃, R₂ is CH₂CF₃, R₃ is H, X is SO, A is CH and Q₁ is as defined in table Y.

Table E-30 provides 20 compounds E-30.001 to E-30.020 of formula Ic-Qa wherein R₁ is CH₂CH₃, R₂ is CH₂CF₃, R₃ is H, X is SO₂, A is CH and Q₁ is as defined in table Y.

Table E-31 provides 20 compounds E-31.001 to E-31.020 of formula Ic-Qa wherein R₁ is CH₂CH₃, R₂ is CH₂CF₂CHFCF₃, R₃ is H, X is S, A is CH and Q₁ is as defined in table Y.

Table E-32 provides 20 compounds E-32.001 to E-32.020 of formula Ic-Qa wherein R₁ is CH₂CH₃, R₂ is CH₂CF₂CHFCF₃, R₃ is H, X is SO, A is CH and Q₁ is as defined in table Y.

Table E-33 provides 20 compounds E-33.001 to E-33.020 of formula Ic-Qa wherein R₁ is CH₂CH₃, R₂ is CH₂CF₂CHFCF₃, R₃ is H, X is SO₂, A is CH and Q₁ is as defined in table Y.

Table E-34 provides 20 compounds E-34.001 to E-34.020 of formula Ic-Qa wherein R₁ is CH₂CH₃, R₂ is SO₂CF₃, R₃ is H, X is S, A is CH and Q₁ is as defined in table Y.

Table E-35 provides 20 compounds E-35.001 to E-35.020 of formula Ic-Qa wherein R₁ is CH₂CH₃, R₂ is SO₂CF₃, R₃ is H, X is SO, A is CH and Q₁ is as defined in table Y.

Table E-36 provides 20 compounds E-36.001 to E-36.020 of formula Ic-Qa wherein R₁ is CH₂CH₃, R₂ is SO₂CF₃, R₃ is H, X is SO₂, A is CH and Q₁ is as defined in table Y.

Table E-37 provides 20 compounds E-37.001 to E-37.020 of formula Ic-Qa wherein R₁ is CH₂CH₃, R₂ is CH₂CF₂CF₃, R₃ is CH₃, X is S, A is N and Q₁ is as defined in table Y.

Table E-38 provides 20 compounds E-38.001 to E-38.020 of formula Ic-Qa wherein R₁ is CH₂CH₃, R₂ is CH₂CF₂CF₃, R₃ is CH₃, X is SO, A is N and Q₁ is as defined in table Y.

Table E-39 provides 20 compounds E-39.001 to E-39.020 of formula Ic-Qa wherein R₁ is CH₂CH₃, R₂ is CH₂CF₂CF₃, R₃ is CH₃, X is SO₂, A is N and Q₁ is as defined in table Y.

Table E-40 provides 20 compounds E-40.001 to E-40.020 of formula Ic-Qa wherein R₁ is CH₂CH₃, R₂ is CH₂CF₂CHF₂, R₃ is CH₃, X is S, A is N and Q₁ is as defined in table Y.

Table E-41 provides 20 compounds E-41.001 to E-41.020 of formula Ic-Qa wherein R₁ is CH₂CH₃, R₂ is CH₂CF₂CHF₂, R₃ is CH₃, X is SO, A is N and Q₁ is as defined in table Y.

Table E-42 provides 20 compounds E-42.001 to E-42.020 of formula Ic-Qa wherein R₁ is CH₂CH₃, R₂ is CH₂CF₂CHF₂, R₃ is CH₃, X is SO₂, A is N and Q₁ is as defined in table Y.

Table E-43 provides 20 compounds E-43.001 to E-43.020 of formula Ic-Qa wherein R₁ is CH₂CH₃, R₂ is CH₂CHF₂, R₃ is CH₃, X is S, A is N and Q₁ is as defined in table Y.

Table E-44 provides 20 compounds E-44.001 to E-44.020 of formula Ic-Qa wherein R₁ is CH₂CH₃, R₂ is CH₂CHF₂, R₃ is CH₃, X is SO, A is N and Q₁ is as defined in table Y.

Table E-45 provides 20 compounds E-45.001 to E-45.020 of formula Ic-Qa wherein R₁ is CH₂CH₃, R₂ is CH₂CHF₂, R₃ is CH₃, X is SO₂, A is N and Q₁ is as defined in table Y.

Table E-46 provides 20 compounds E-46.001 to E-46.020 of formula Ic-Qa wherein R₁ is CH₂CH₃, R₂ is CH₂CF₃, R₃ is CH₃, X is S, A is N and Q₁ is as defined in table Y.

Table E-47 provides 20 compounds E-47.001 to E-47.020 of formula Ic-Qa wherein R₁ is CH₂CH₃, R₂ is CH₂CF₃, R₃ is CH₃, X is SO, A is N and Q₁ is as defined in table Y.

Table E-48 provides 20 compounds E-48.001 to E-48.020 of formula Ic-Qa wherein R₁ is CH₂CH₃, R₂ is CH₂CF₃, R₃ is CH₃, X is SO₂, A is N and Q₁ is as defined in table Y.

Table E-49 provides 20 compounds E-49.001 to E-49.020 of formula Ic-Qa wherein R₁ is CH₂CH₃, R₂ is CH₂CF₂CHFCF₃, R₃ is CH₃, X is S, A is N and Q₁ is as defined in table Y.

Table E-50 provides 20 compounds E-50.001 to E-50.020 of formula Ic-Qa wherein R₁ is CH₂CH₃, R₂ is CH₂CF₂CHFCF₃, R₃ is CH₃, X is SO, A is N and Q₁ is as defined in table Y.

Table E-51 provides 20 compounds E-51.001 to E-51.020 of formula Ic-Qa wherein R₁ is CH₂CH₃, R₂ is CH₂CF₂CHFCF₃, R₃ is CH₃, X is SO₂, A is N and Q₁ is as defined in table Y.

Table E-52 provides 20 compounds E-52.001 to E-52.020 of formula Ic-Qa wherein R₁ is CH₂CH₃, R₂ is SO₂CF₃, R₃ is CH₃, X is S, A is N and Q₁ is as defined in table Y.

Table E-53 provides 20 compounds E-53.001 to E-53.020 of formula Ic-Qa wherein R₁ is CH₂CH₃, R₂ is SO₂CF₃, R₃ is CH₃, X is SO, A is N and Q₁ is as defined in table Y.

Table E-54 provides 20 compounds E-54.001 to E-54.020 of formula Ic-Qa wherein R₁ is CH₂CH₃, R₂ is SO₂CF₃, R₃ is CH₃, X is SO₂, A is N and Q₁ is as defined in table Y.

Table E-55 provides 20 compounds E-55.001 to E-55.020 of formula Ia-Qa wherein R₁ is —CH₂cyclopropyl, R₂ is CH₂CF₂CF₃, R₃ is H, X is S, A is N and Q₁ is as defined in table Y.

Table E-56 provides 20 compounds E-56.001 to E-56.020 of formula Ia-Qa wherein R₁ is —CH₂cyclopropyl, R₂ is CH₂CF₂CF₃, R₃ is H, X is SO, A is N and Q₁ is as defined in table Y.

Table E-57 provides 20 compounds E-57.001 to E-57.020 of formula Ia-Qa wherein R₁ is —CH₂cyclopropyl, R₂ is CH₂CF₂CF₃, R₃ is H, X is SO₂, A is N and Q₁ is as defined in table Y.

Table E-58 provides 20 compounds E-58.001 to E-58.020 of formula Ia-Qa wherein R₁ is —CH₂cyclopropyl, R₂ is CH₂CF₂CHF₂, R₃ is H, X is S, A is N and Q₁ is as defined in table Y.

Table E-59 provides 20 compounds E-59.001 to E-59.020 of formula Ia-Qa wherein R₁ is —CH₂cyclopropyl, R₂ is CH₂CF₂CHF₂, R₃ is H, X is SO, A is N and Q₁ is as defined in table Y.

Table E-60 provides 20 compounds E-60.001 to E-60.020 of formula Ia-Qa wherein R₁ is —CH₂cyclopropyl, R₂ is CH₂CF₂CHF₂, R₃ is H, X is SO₂, A is N and Q₁ is as defined in table Y.

Table E-61 provides 20 compounds E-61.001 to E-61.020 of formula Ia-Qa wherein R₁ is —CH₂cyclopropyl, R₂ is CH₂CF₃, R₃ is H, X is S, A is N and Q₁ is as defined in table Y.

Table E-62 provides 20 compounds E-62.001 to E-62.020 of formula Ia-Qa wherein R₁ is —CH₂cyclopropyl, R₂ is CH₂CF₃, R₃ is H, X is SO, A is N and Q₁ is as defined in table Y.

Table E-63 provides 20 compounds E-63.001 to E-63.020 of formula Ia-Qa wherein R₁ is —CH₂cyclopropyl, R₂ is CH₂CF₃, R₃ is H, X is SO₂, A is N and Q₁ is as defined in table Y.

Table E-64 provides 20 compounds E-64.001 to E-64.020 of formula Ia-Qa wherein R₁ is —CH₂cyclopropyl, R₂ is CH₂CF₂CF₃, R₃ is H, X is S, A is CH and Q₁ is as defined in table Y.

Table E-65 provides 20 compounds E-65.001 to E-65.020 of formula Ia-Qa wherein R₁ is —CH₂cyclopropyl, R₂ is CH₂CF₂CF₃, R₃ is H, X is SO, A is CH and Q₁ is as defined in table Y.

Table E-66 provides 20 compounds E-66.001 to E-66.020 of formula Ia-Qa wherein R₁ is —CH₂cyclopropyl, R₂ is CH₂CF₂CF₃, R₃ is H, X is SO₂, A is CH and Q₁ is as defined in table Y.

Table E-67 provides 20 compounds E-67.001 to E-67.020 of formula Ia-Qa wherein R₁ is —CH₂cyclopropyl, R₂ is CH₂CF₂CHF₂, R₃ is H, X is S, A is CH and Q₁ is as defined in table Y.

Table E-68 provides 20 compounds E-68.001 to E-68.020 of formula Ia-Qa wherein R₁ is —CH₂cyclopropyl, R₂ is CH₂CF₂CHF₂, R₃ is H, X is SO, A is CH and Q₁ is as defined in table Y.

Table E-69 provides 20 compounds E-69.001 to E-69.020 of formula Ia-Qa wherein R₁ is —CH₂cyclopropyl, R₂ is CH₂CF₂CHF₂, R₃ is H, X is SO₂, A is CH and Q₁ is as defined in table Y.

Table E-70 provides 20 compounds E-70.001 to E-70.020 of formula Ia-Qa wherein R₁ is —CH₂cyclopropyl, R₂ is CH₂CF₃, R₃ is H, X is S, A is CH and Q₁ is as defined in table Y.

Table E-71 provides 20 compounds E-71.001 to E-71.020 of formula Ia-Qa wherein R₁ is —CH₂cyclopropyl, R₂ is CH₂CF₃, R₃ is H, X is SO, A is CH and Q₁ is as defined in table Y.

Table E-72 provides 20 compounds E-72.001 to E-72.020 of formula Ia-Qa wherein R₁ is —CH₂cyclopropyl, R₂ is CH₂CF₃, R₃ is H, X is SO₂, A is CH and Q₁ is as defined in table Y.

Table E-73 provides 20 compounds E-73.001 to E-73.020 of formula Ia-Qa wherein R₁ is —CH₂cyclopropyl, R₂ is CH₂CF₂CF₃, R₃ is CH₃, X is S, A is N and Q₁ is as defined in table Y.

Table E-74 provides 20 compounds E-74.001 to E-74.020 of formula Ia-Qa wherein R₁ is —CH₂cyclopropyl, R₂ is CH₂CF₂CF₃, R₃ is CH₃, X is SO, A is N and Q₁ is as defined in table Y.

Table E-75 provides 20 compounds E-75.001 to E-75.020 of formula Ia-Qa wherein R₁ is —CH₂cyclopropyl, R₂ is CH₂CF₂CF₃, R₃ is CH₃, X is SO₂, A is N and Q₁ is as defined in table Y.

Table E-76 provides 20 compounds E-76.001 to E-76.020 of formula Ia-Qa wherein R₁ is —CH₂cyclopropyl, R₂ is CH₂CF₂CHF₂, R₃ is CH₃, X is S, A is N and Q₁ is as defined in table Y.

Table E-77 provides 20 compounds E-77.001 to E-77.020 of formula Ia-Qa wherein R₁ is —CH₂cyclopropyl, R₂ is CH₂CF₂CHF₂, R₃ is CH₃, X is SO, A is N and Q₁ is as defined in table Y.

Table E-78 provides 20 compounds E-78.001 to E-78.020 of formula Ia-Qa wherein R₁ is —CH₂cyclopropyl, R₂ is CH₂CF₂CHF₂, R₃ is CH₃, X is SO₂, A is N and Q₁ is as defined in table Y.

Table E-79 provides 20 compounds E-79.001 to E-79.020 of formula Ia-Qa wherein R₁ is —CH₂cyclopropyl, R₂ is CH₂CF₃, R₃ is CH₃, X is S, A is N and Q₁ is as defined in table Y.

Table E-80 provides 20 compounds E-80.001 to E-80.020 of formula Ia-Qa wherein R₁ is —CH₂cyclopropyl, R₂ is CH₂CF₃, R₃ is CH₃, X is SO, A is N and Q₁ is as defined in table Y.

Table E-81 provides 20 compounds E-81.001 to E-81.020 of formula Ia-Qa wherein R₁ is —CH₂cyclopropyl, R₂ is CH₂CF₃, R₃ is CH₃, X is SO₂, A is N and Q₁ is as defined in table Y.

The tables F-1 to F-54 below illustrate further specific compounds of the invention.

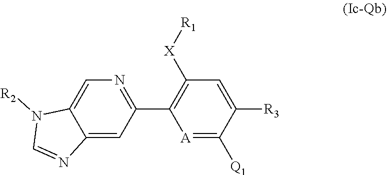

Table F-1 provides 21 compounds F-1.001 to F-1.021 of formula Ic-Qb wherein R₁ is CH₂CH₃, R₂ is CH₂CF₂CF₃, R₃ is H, X is S, A is N and Q₁ is as defined in table Z.

Table F-2 provides 21 compounds F-2.001 to F-2.021 of formula Ic-Qb wherein R₁ is CH₂CH₃, R₂ is CH₂CF₂CF₃, R₃ is H, X is SO, A is N and Q₁ is as defined in table Z.

Table F-3 provides 21 compounds F-3.001 to F-3.021 of formula Ic-Qb wherein R₁ is CH₂CH₃, R₂ is CH₂CF₂CF₃, R₃ is H, X is SO₂, A is N and Q₁ is as defined in table Z.

Table F-4 provides 21 compounds F-4.001 to F-4.021 of formula Ic-Qb wherein R₁ is CH₂CH₃, R₂ is CH₂CF₂CHF₂, R₃ is H, X is S, A is N and Q₁ is as defined in table Z.

Table F-5 provides 21 compounds F-5.001 to F-5.021 of formula Ic-Qb wherein R₁ is CH₂CH₃, R₂ is CH₂CF₂CHF₂, R₃ is H, X is SO, A is N and Q₁ is as defined in table Z.

Table F-6 provides 21 compounds F-6.001 to F-6.021 of formula Ic-Qb wherein R₁ is CH₂CH₃, R₂ is CH₂CF₂CHF₂, R₃ is H, X is SO₂, A is N and Q₁ is as defined in table Z.

Table F-7 provides 21 compounds F-7.001 to F-7.021 of formula Ic-Qb wherein R₁ is CH₂CH₃, R₂ is CH₂CHF₂, R₃ is H, X is S, A is N and Q₁ is as defined in table Z.

Table F-8 provides 21 compounds F-8.001 to F-8.021 of formula Ic-Qb wherein R₁ is CH₂CH₃, R₂ is CH₂CHF₂, R₃ is H, X is SO, A is N and Q₁ is as defined in table Z.

Table F-9 provides 21 compounds F-9.001 to F-9.021 of formula Ic-Qb wherein R₁ is CH₂CH₃, R₂ is CH₂CHF₂, R₃ is H, X is SO₂, A is N and Q₁ is as defined in table Z.

Table F-10 provides 21 compounds F-10.001 to F-10.021 of formula Ic-Qb wherein R₁ is CH₂CH₃, R₂ is CH₂CF₃, R₃ is H, X is S, A is N and Q₁ is as defined in table Z.

Table F-11 provides 21 compounds F-11.001 to F-11.021 of formula Ic-Qb wherein R₁ is CH₂CH₃, R₂ is CH₂CF₃, R₃ is H, X is SO, A is N and Q₁ is as defined in table Z.

Table F-12 provides 21 compounds F-12.001 to F-12.021 of formula Ic-Qb wherein R₁ is CH₂CH₃, R₂ is CH₂CF₃, R₃ is H, X is SO₂, A is N and Q₁ is as defined in table Z.

Table F-13 provides 21 compounds F-13.001 to F-13.021 of formula Ic-Qb wherein R₁ is CH₂CH₃, R₂ is CH₂CF₂CHFCF₃, R₃ is H, X is S, A is N and Q₁ is as defined in table Z.

Table F-14 provides 21 compounds F-14.001 to F-14.021 of formula Ic-Qb wherein R₁ is CH₂CH₃, R₂ is CH₂CF₂CHFCF₃, R₃ is H, X is SO, A is N and Q₁ is as defined in table Z.

Table F-15 provides 21 compounds F-15.001 to F-15.021 of formula Ic-Qb wherein R₁ is CH₂CH₃, R₂ is CH₂CF₂CHFCF₃, R₃ is H, X is SO₂, A is N and Q₁ is as defined in table Z.

Table F-16 provides 21 compounds F-16.001 to F-16.021 of formula Ic-Qb wherein R₁ is CH₂CH₃, R₂ is SO₂CF₃, R₃ is H, X is S, A is N and Q₁ is as defined in table Z.

Table F-17 provides 21 compounds F-17.001 to F-17.021 of formula Ic-Qb wherein R₁ is CH₂CH₃, R₂ is SO₂CF₃, R₃ is H, X is SO, A is N and Q₁ is as defined in table Z.

Table F-18 provides 21 compounds F-18.001 to F-18.021 of formula Ic-Qb wherein R₁ is CH₂CH₃, R₂ is SO₂CF₃, R₃ is H, X is SO₂, A is N and Q₁ is as defined in table Z.

Table F-19 provides 21 compounds F-19.001 to F-19.021 of formula Ic-Qb wherein R₁ is CH₂CH₃, R₂ is CH₂CF₂CF₃, R₃ is H, X is S, A is CH and Q₁ is as defined in table Z.

Table F-20 provides 21 compounds F-20.001 to F-20.021 of formula Ic-Qb wherein R₁ is CH₂CH₃, R₂ is CH₂CF₂CF₃, R₃ is H, X is SO, A is CH and Q₁ is as defined in table Z.

Table F-21 provides 21 compounds F-21.001 to F-21.021 of formula Ic-Qb wherein R₁ is CH₂CH₃, R₂ is CH₂CF₂CF₃, R₃ is H, X is SO₂, A is CH and Q₁ is as defined in table Z.

Table F-22 provides 21 compounds F-22.001 to F-22.021 of formula Ic-Qb wherein R₁ is CH₂CH₃, R₂ is CH₂CF₂CHF₂, R₃ is H, X is S, A is CH and Q₁ is as defined in table Z.

Table F-23 provides 21 compounds F-23.001 to F-23.021 of formula Ic-Qb wherein R₁ is CH₂CH₃, R₂ is CH₂CF₂CHF₂, R₃ is H, X is SO, A is CH and Q₁ is as defined in table Z.

Table F-24 provides 21 compounds F-24.001 to F-24.021 of formula Ic-Qb wherein R₁ is CH₂CH₃, R₂ is CH₂CF₂CHF₂, R₃ is H, X is SO₂, A is CH and Q₁ is as defined in table Z.

Table F-25 provides 21 compounds F-25.001 to F-25.021 of formula Ic-Qb wherein R₁ is CH₂CH₃, R₂ is CH₂CHF₂, R₃ is H, X is S, A is CH and Q₁ is as defined in table Z.

Table F-26 provides 21 compounds F-26.001 to F-26.021 of formula Ic-Qb wherein R₁ is CH₂CH₃, R₂ is CH₂CHF₂, R₃ is H, X is SO, A is CH and Q₁ is as defined in table Z.

Table F-27 provides 21 compounds F-27.001 to F-27.021 of formula Ic-Qb wherein R₁ is CH₂CH₃, R₂ is CH₂CHF₂, R₃ is H, X is SO₂, A is CH and Q₁ is as defined in table Z.

Table F-28 provides 21 compounds F-28.001 to F-28.021 of formula Ic-Qb wherein R₁ is CH₂CH₃, R₂ is CH₂CF₃, R₃ is H, X is S, A is CH and Q₁ is as defined in table Z.

Table F-29 provides 21 compounds F-29.001 to F-29.021 of formula Ic-Qb wherein R₁ is CH₂CH₃, R₂ is CH₂CF₃, R₃ is H, X is SO, A is CH and Q₁ is as defined in table Z.

Table F-30 provides 21 compounds F-30.001 to F-30.021 of formula Ic-Qb wherein R₁ is CH₂CH₃, R₂ is CH₂CF₃, R₃ is H, X is SO₂, A is CH and Q₁ is as defined in table Z.

Table F-31 provides 21 compounds F-31.001 to F-31.021 of formula Ic-Qb wherein R₁ is CH₂CH₃, R₂ is CH₂CF₂CHFCF₃, R₃ is H, X is S, A is CH and Q₁ is as defined in table Z.

Table F-32 provides 21 compounds F-32.001 to F-32.021 of formula Ic-Qb wherein R₁ is CH₂CH₃, R₂ is CH₂CF₂CHFCF₃, R₃ is H, X is SO, A is CH and Q₁ is as defined in table Z.

Table F-33 provides 21 compounds F-33.001 to F-33.021 of formula Ic-Qb wherein R₁ is CH₂CH₃, R₂ is CH₂CF₂CHFCF₃, R₃ is H, X is SO₂, A is CH and Q₁ is as defined in table Z.

Table F-34 provides 21 compounds F-34.001 to F-34.021 of formula Ic-Qb wherein R₁ is CH₂CH₃, R₂ is SO₂CF₃, R₃ is H, X is S, A is CH and Q₁ is as defined in table Z.

Table F-35 provides 21 compounds F-35.001 to F-35.021 of formula Ic-Qb wherein R₁ is CH₂CH₃, R₂ is SO₂CF₃, R₃ is H, X is SO, A is CH and Q₁ is as defined in table Z.

Table F-36 provides 21 compounds F-36.001 to F-36.021 of formula Ic-Qb wherein R₁ is CH₂CH₃, R₂ is SO₂CF₃, R₃ is H, X is SO₂, A is CH and Q₁ is as defined in table Z.

Table F-37 provides 21 compounds F-37.001 to F-37.021 of formula Ia-Qb wherein R₁ is —CH₂cyclopropyl, R₂ is CH₂CF₂CF₃, R₃ is H, X is S, A is N and Q₁ is as defined in table Z.

Table F-38 provides 21 compounds F-38.001 to F-38.021 of formula Ia-Qb wherein R₁ is —CH₂cyclopropyl, R₂ is CH₂CF₂CF₃, R₃ is H, X is SO, A is N and Q₁ is as defined in table Z.

Table F-39 provides 21 compounds F-39.001 to F-39.021 of formula Ia-Qb wherein R₁ is —CH₂cyclopropyl, R₂ is CH₂CF₂CF₃, R₃ is H, X is SO₂, A is N and Q₁ is as defined in table Z.

Table F-40 provides 21 compounds F-40.001 to F-40.021 of formula Ia-Qb wherein R₁ is —CH₂cyclopropyl, R₂ is CH₂CF₂CHF₂, R₃ is H, X is S, A is N and Q₁ is as defined in table Z.

Table F-41 provides 21 compounds F-41.001 to F-41.021 of formula Ia-Qb wherein R₁ is —CH₂cyclopropyl, R₂ is CH₂CF₂CHF₂, R₃ is H, X is SO, A is N and Q₁ is as defined in table Z.

Table F-42 provides 21 compounds F-42.001 to F-42.021 of formula Ia-Qb wherein R₁ is —CH₂cyclopropyl, R₂ is CH₂CF₂CHF₂, R₃ is H, X is SO₂, A is N and Q₁ is as defined in table Z.

Table F-43 provides 21 compounds F-43.001 to F-43.021 of formula Ia-Qb wherein R₁ is —CH₂cyclopropyl, R₂ is CH₂CF₃, R₃ is H, X is S, A is N and Q₁ is as defined in table Z.

Table F-44 provides 21 compounds F-44.001 to F-44.021 of formula Ia-Qb wherein R₁ is —CH₂cyclopropyl, R₂ is CH₂CF₃, R₃ is H, X is SO, A is N and Q₁ is as defined in table Z.

Table F-45 provides 21 compounds F-45.001 to F-45.021 of formula Ia-Qb wherein R₁ is —CH₂cyclopropyl, R₂ is CH₂CF₃, R₃ is H, X is SO₂, A is N and Q₁ is as defined in table Z.

Table F-46 provides 21 compounds F-46.001 to F-46.021 of formula Ia-Qb wherein R₁ is —CH₂cyclopropyl, R₂ is CH₂CF₂CF₃, R₃ is H, X is S, A is CH and Q₁ is as defined in table Z.

Table F-47 provides 21 compounds F-47.001 to F-47.021 of formula Ia-Qb wherein R₁ is —CH₂cyclopropyl, R₂ is CH₂CF₂CF₃, R₃ is H, X is SO, A is CH and Q₁ is as defined in table Z.

Table F-48 provides 21 compounds F-48.001 to F-48.021 of formula Ia-Qb wherein R₁ is —CH₂cyclopropyl, R₂ is CH₂CF₂CF₃, R₃ is H, X is SO₂, A is CH and Q₁ is as defined in table Z.

Table F-49 provides 21 compounds F-49.001 to F-49.021 of formula Ia-Qb wherein R₁ is —CH₂cyclopropyl, R₂ is CH₂CF₂CHF₂, R₃ is H, X is S, A is CH and Q₁ is as defined in table Z.

Table F-50 provides 21 compounds F-50.001 to F-50.021 of formula Ia-Qb wherein R₁ is —CH₂cyclopropyl, R₂ is CH₂CF₂CHF₂, R₃ is H, X is SO, A is CH and Q₁ is as defined in table Z.

Table F-51 provides 21 compounds F-51.001 to F-51.021 of formula Ia-Qb wherein R₁ is —CH₂cyclopropyl, R₂ is CH₂CF₂CHF₂, R₃ is H, X is SO₂, A is CH and Q₁ is as defined in table Z.

Table F-52 provides 21 compounds F-52.001 to F-52.021 of formula Ia-Qb wherein R₁ is —CH₂cyclopropyl, R₂ is CH₂CF₃, R₃ is H, X is S, A is CH and Q₁ is as defined in table Z.

Table F-53 provides 21 compounds F-53.001 to F-53.021 of formula Ia-Qb wherein R₁ is —CH₂cyclopropyl, R₂ is CH₂CF₃, R₃ is H, X is SO, A is CH and Q₁ is as defined in table Z.

Table F-54 provides 21 compounds F-54.001 to F-54.021 of formula Ia-Qb wherein R₁ is —CH₂cyclopropyl, R₂ is CH₂CF₃, R₃ is H, X is SO₂, A is CH and Q₁ is as defined in table Z.

The compounds of formula I according to the invention are preventively and/or curatively valuable active ingredients in the field of pest control, even at low rates of application, which have a very favorable biocidal spectrum and are well tolerated by warm-blooded species, fish and plants. The active ingredients according to the invention act against all or individual developmental stages of normally sensitive, but also resistant, animal pests, such as insects or representatives of the order Acarina. The insecticidal or acaricidal activity of the active ingredients according to the invention can manifest itself directly, i. e. in destruction of the pests, which takes place either immediately or only after some time has elapsed, for example during ecdysis, or indirectly, for example in a reduced oviposition and/or hatching rate, a good activity corresponding to a destruction rate (mortality) of at least 50 to 60%.

Examples of the above mentioned animal pests are:

from the order Acarina, for example,
Acalitus spp, Aculus spp, Acaricalus spp, Aceria spp, Acarus siro, Amblyomma spp., Argas spp., Boophilus spp., Brevipalpus spp., Bryobia spp, Calipitrimerus spp., Chorioptes spp., Dermanyssus gallinae, Dermatophagoides spp, Eotetranychus spp, Eriophyes spp., Hemitarsonemus spp, Hyalomma spp., Ixodes spp., Olygonychus spp, Ornithodoros spp., Polyphagotarsone latus, Panonychus spp., Phyllocoptruta oleivora, Phytonemus spp, Polyphagotarsonemus spp, Psoroptes spp., Rhipicephalus spp., Rhizoglyphus spp., Sarcoptes spp., Steneotarsonemus spp, Tarsonemus spp. and Tetranychus spp.;
from the order Anoplura, for example,

### Haematopinus spp., Linognathus spp., Pediculus spp., Pemphigus spp. and Phylloxera spp.;

from the order Coleoptera, for example,
Agriotes spp., Amphimallon majale, Anomala orientalis, Anthonomus spp., Aphodius spp, Astylus atromaculatus, Ataenius spp, Atomaria linearis, Chaetocnema tibialis, Cerotoma spp, Conoderus spp, Cosmopolites spp., Cotinis nitida, Curculio spp., Cyclocephala spp, Dermestes spp., Diabrotica spp., Diloboderus abderus, Epilachna spp., Eremnus spp., Heteronychus arator, Hypothenemus hampei, Lagria vilosa, Leptinotarsa decemLineata, Lissorhoptrus spp., Liogenys spp, Maecolaspis spp, Maladera castanea, Megascelis spp, Melighetes aeneus, Melolontha spp., Myochrous armatus, Orycaephilus spp., Otiorhynchus spp., Phyllophaga spp, Phlyctinus spp., Popillia spp., Psylliodes spp., Rhyssomatus aubtilis, Rhizopertha spp., Scarabeidae, Sitophilus spp., Sitotroga spp., Somaticus spp, Sphenophorus spp, Sternechus subsignatus, Tenebrio spp., Tribolium spp. and Trogoderma spp.;
from the order Diptera, for example,
Aedes spp., Anopheles spp, Antherigona soccata, Bactrocea oleae, Bibio hortulanus, Bradysia spp, Calliphora erythrocephala, Ceratitis spp., Chrysomyia spp., Culex spp., Cuterebra spp., Dacus spp., Delia spp, Drosophila melanogaster, Fannia spp., Gastrophilus spp., Geomyza tripunctata, Glossina spp., Hypoderma spp., Hyppobosca spp., Liriomyza spp., Lucilia spp., Melanagromyza spp., Musca spp., Oestrus spp., Orseolia spp., Oscinella frit, Pegomyia hyoscyami, Phorbia spp., Rhagoletis spp, Rivelia quadrifasciata, Scatella spp, Sciara spp., Stomoxys spp., Tabanus spp., Tannia spp. and Tipula spp.;
from the order Hemiptera, for example,
Acanthocoris scabrator, Acrosternum spp, Adelphocoris lineolatus, Amblypelta nitida, Bathycoelia thalassina, Blissus spp, Cimex spp., Clavigralla tomentosicollis, Creontiades spp, Distantiella theobroma, Dichelops furcatus, Dysdercus spp., Edessa spp, Euchistus spp., Eurydema pulchrum, Eurygaster spp., Halyomorpha halys, Horcias nobilellus, Leptocorisa spp., Lygus spp, Margarodes spp, Murgantia histrionic, Neomegalotomus spp, Nesidiocoris tenuis, Nezara spp., Nysius simulans, Oebalus insularis, Piesma spp., Piezodorus spp, Rhodnius spp., Sahlbergella singularis, Scaptocoris castanea, Scotinophara spp., Thyanta spp, Triatoma spp., Vatiga illudens; Acyrthosium pisum, Adalges spp, Agalliana ensigera, Agonoscena targionii, Aleurodicus spp, Aleurocanthus spp, Aleurolobus barodensis, Aleurothrixus floccosus, Aleyrodes brassicae, Amarasca biguttula, Amritodus atkinsoni, Aonidiella spp., Aphididae, Aphis spp., Aspidiotus spp., Aulacorthum solani, Bactericera cockerelli, Bemisia spp, Brachycaudus spp, Brevicoryne brassicae, Cacopsylla spp, Cavariella aegopodii Scop., Ceroplaster spp., Chrysomphalus aonidium, Chrysomphalus dictyospermi, Cicadella spp, Cofana spectra, Cryptomyzus spp, Cicadulina spp, Coccus hesperidum, Dalbulus maidis, Dialeurodes spp, Diaphorina citri, Diuraphis noxia, Dysaphis spp, Empoasca spp., Eriosoma larigerum, Erythroneura spp., Gascardia spp., Glycaspis brimblecombei, Hyadaphis pseudobrassicae, Hyalopterus spp, Hyperomyzus pallidus, Idioscopus clypealis, Jacobiasca lybica, Laodelphax spp., Lecanium corni, Lepidosaphes spp., Lopaphis erysimi, Lyogenys maidis, Macrosiphum spp., Mahanarva spp, Metcalfa pruinosa, Metopolophium dirhodum, Myndus crudus, Myzus spp., Neotoxoptera sp, Nephotettix spp., Nilaparvata spp., Nippolachnus piri Mats, Odonaspis ruthae, Oregma lanigera Zehnter, Parabemisia myricae, Paratrioza cockerelli, Parlatoria spp., Pemphigus spp., Peregrinus maidis, Perkinsiella spp, Phorodon humuli, Phylloxera spp, Planococcus spp., Pseudaulacaspis spp., Pseudococcus spp., Pseudatomoscelis seriatus, Psylla spp., Pulvinaria aethiopica, Quadraspidiotus spp., Quesada gigas, Recilia dorsalis, Rhopalosiphum spp., Saissetia spp., Scaphoideus spp., Schizaphis spp., Sitobion spp., Sogatella furcifera, Spissistilus festinus, Tarophagus Proserpina, Toxoptera spp, Trialeurodes spp, Tridiscus sporoboli, Trionymus spp, Trioza erytreae, Unaspis citri, Zygina flammigera, Zyginidia scutellaris;
from the order Hymenoptera, for example,
Acromyrmex, Arge spp, Atta spp., Cephus spp., Diprion spp., Diprionidae, Gilpinia polytoma, Hoplo-campa spp., Lasius spp., Monomorium pharaonis, Neodiprion spp., Pogonomyrmex spp, Slenopsis invicta, Solenopsis spp. and Vespa spp.;
from the order Isoptera, for example,
Coptotermes spp, Corniternes cumulans, Incisitermes spp, Macrotermes spp, Mastotermes spp, Microtermes spp, Reticulitermes spp.; Solenopsis geminate
from the order Lepidoptera, for example,
Acleris spp., Adoxophyes spp., Aegeria spp., Agrotis spp., Alabama argillaceae, Amylois spp., Anticarsia gemmatalis, Archips spp., Argyresthia spp, Argyrotaenia spp., Autographa spp., Bucculatrix thurberiella, Busseola fusca, Cadra cautella, Carposina nipponensis, Chilo spp., Choristoneura spp., Chrysoteuchia topiaria, Clysia ambiguella, Cnaphalocrocis spp., Cnephasia spp., Cochylis spp., Coleophora spp., Colias lesbia, Cosmophila flava, Crambus spp, Crocidolomia binotalis, Cryptophlebia leucotreta, Cydalima perspectalis, Cydia spp., Diaphania perspectalis, Diatraea spp., Diparopsis castanea, Earias spp., Eldana saccharina, Ephestia spp., Epinotia spp, Estigmene acrea, Etiella zinckinella, Eucosma spp., Eupoecilia ambiguella, Euproctis spp., Euxoa spp., Feltia jaculiferia, Grapholita spp., Hedya nubiferana, Heliothis spp., Hellula undalis, Herpetogramma spp, Hyphantria cunea, Keiferia lycopersicella, Lasmopalpus lignosellus, Leucoptera scitella, Lithocollethis spp., Lobesia botrana, Loxostege bifidalis, Lymantria spp., Lyonetia spp., Malacosoma spp., Mamestra brassicae, Manduca sexta, Mythimna spp, Noctua spp, Operophtera spp., Orniodes indica, Ostrinia nubilalis, Pammene spp., Pandemis spp., Panolis flammea, Papaipema nebris, Pectinophora gossypi-ela, Perileucoptera coffeella, Pseudaletia unipuncta, Phthorimaea operculella, Pieris rapae, Pieris spp., Plutella xylostella, Prays spp., Pseudoplusia spp, Rachiplusia nu, Richia albicosta, Scirpophaga spp., Sesamia spp., Sparganothis spp., Spodoptera spp., Sylepta derogate, Synanthedon spp., Thaumetopoea spp., Tortrix spp., Trichoplusia ni, Tuta absoluta, and Yponomeuta spp.;
from the order Mallophaga, for example,

### Damalinea spp. and Trichodectes spp.;

from the order Orthoptera, for example,
Blatta spp., Blattella spp., Gryllotalpa spp., Leucophaea maderae, Locusta spp., Neocurtilla hexadactyla, Periplaneta spp., Scapteriscus spp, and Schistocerca spp.;
from the order Psocoptera, for example,

### Liposcelis spp.;

from the order Siphonaptera, for example,
Ceratophyllus spp., Ctenocephalides spp. and Xenopsylla cheopis;
from the order Thysanoptera, for example,
Calliothrips phaseoli, Frankliniella spp., Heliothrips spp, Hercinothrips spp., Parthenothrips spp, Scirtothrips aurantii, Sericothrips variabilis, Taeniothrips spp., Thrips spp;
from the order Thysanura, for example, Lepisma saccharina.

The active ingredients according to the invention can be used for controlling, i. e. containing or destroying, pests of the abovementioned type which occur in particular on plants, especially on useful plants and ornamentals in agriculture, in horticulture and in forests, or on organs, such as fruits, flowers, foliage, stalks, tubers or roots, of such plants, and in some cases even plant organs which are formed at a later point in time remain protected against these pests.

Suitable target crops are, in particular, cereals, such as wheat, barley, rye, oats, rice, maize or sorghum; beet, such as sugar or fodder beet; fruit, for example pomaceous fruit, stone fruit or soft fruit, such as apples, pears, plums, peaches, almonds, cherries or berries, for example strawberries, raspberries or blackberries; leguminous crops, such as beans, lentils, peas or soya; oil crops, such as oilseed rape, mustard, poppies, olives, sunflowers, coconut, castor, cocoa or ground nuts; cucurbits, such as pumpkins, cucumbers or melons; fibre plants, such as cotton, flax, hemp orjute; citrus fruit, such as oranges, lemons, grapefruit or tangerines; vegetables, such as spinach, lettuce, asparagus, cabbages, carrots, onions, tomatoes, potatoes or bell peppers; Lauraceae, such as avocado, Cinnamonium or camphor; and also tobacco, nuts, coffee, eggplants, sugarcane, tea, pepper, grapevines, hops, the plantain family and latex plants.

The compositions and/or methods of the present invention may be also used on any ornamental and/or vegetable crops, including flowers, shrubs, broad-leaved trees and evergreens.

For example the invention may be used on any of the following ornamental species: Ageratum spp., Alonsoa spp., Anemone spp., Anisodontea capsenisis, Anthemis spp., Antirrhinum spp., Aster spp., Begonia spp. (e.g. B. elatior, B. semperflorens, B. tubereux), Bougainvillea spp., Brachycome spp., Brassica spp. (ornamental), Calceolaria spp., Capsicum annuum, Catharanthus roseus, Canna spp., Centaurea spp., Chrysanthemum spp., Cineraria spp. (C. maritime), Coreopsis spp., Crassula coccinea, Cuphea ignea, Dahlia spp., Delphinium spp., Dicentra spectabilis, Dorotheantus spp., Eustoma grandiflorum, Forsythia spp., Fuchsia spp., Geranium gnaphalium, Gerbera spp., Gomphrena globosa, Heliotropium spp., Helianthus spp., Hibiscus spp., Hortensia spp., Hydrangea spp., Hypoestes phyllostachya, Impatiens spp. (I. walleriana), Iresines spp., Kalanchoe spp., Lantana camara, Lavatera trimestris, Leonotis leonurus, Lilium spp., Mesembryanthemum spp., Mimulus spp., Monarda spp., Nemesia spp., Tagetes spp., Dianthus spp. (carnation), Canna spp., Oxalis spp., Bellis spp., Pelargonium spp. (P. peltatum, P. Zonale), Viola spp. (pansy), Petunia spp., Phlox spp., Plecthranthus spp., Poinsettia spp., Parthenocissus spp. (P. quinquefolia, P. tricuspidata), Primula spp., Ranunculus spp., Rhododendron spp., Rosa spp. (rose), Rudbeckia spp., Saintpaulia spp., Salvia spp., Scaevola aemola, Schizanthus wisetonensis, Sedum spp., Solanum spp., Surfinia spp., Tagetes spp., Nicotinia spp., Verbena spp., Zinnia spp. and other bedding plants.

For example the invention may be used on any of the following vegetable species: Allium spp. (A. sativum, A. cepa, A. oschaninii, A. Porrum, A. ascalonicum, A. fistulosum), Anthriscus cerefolium, Apium graveolus, Asparagus officinalis, Beta vulgarus, Brassica spp. (B. oleracea, B. pekinensis, B. rapa), Capsicum annuum, Cicer arietinum, Cichorium endivia, Cichorum spp. (C. intybus, C. endivia), Citrillus lanatus, Cucumis spp. (C. sativus, C. melo), Cucurbita spp. (C. pepo, C. maxima), Cyanara spp. (C. scolymus, C. cardunculus), Daucus carota, Foeniculum vulgare, Hypericum spp., Lactuca sativa, Lycopersicon spp. (L. esculentum, L. lycopersicum), Mentha spp., Ocimum basilicum, Petroselinum crispum, Phaseolus spp. (P. vulgaris, P. coccineus), Pisum sativum, Raphanus sativus, Rheum rhaponticum, Rosemarinus spp., Salvia spp., Scorzonera hispanica, Solanum melongena, Spinacea oleracea, Valerianella spp. (V. locusta, V. eriocarpa) and Vicia faba.

Preferred ornamental species include African violet, Begonia, Dahlia, Gerbera, Hydrangea, Verbena, Rosa, Kalanchoe, Poinsettia, Aster, Centaurea, Coreopsis, Delphinium, Monarda, Phlox, Rudbeckia, Sedum, Petunia, Viola, Impatiens, Geranium, Chrysanthemum, Ranunculus, Fuchsia, Salvia, Hortensia, rosemary, sage, St. Johnswort, mint, sweet pepper, tomato and cucumber.

The active ingredients according to the invention are especially suitable for controlling Aphis craccivora, Diabrotica balteata, Heliothis virescens, Myzus persicae, Plutella xylostella and Spodoptera littoralis in cotton, vegetable, maize, rice and soya crops. The active ingredients according to the invention are further especially suitable for controlling Mamestra (preferably in vegetables), Cydia pomonella (preferably in apples), Empoasca (preferably in vegetables, vineyards), Leptinotarsa (preferably in potatos) and Chilo supressalis (preferably in rice).

The active ingredients according to the invention are especially suitable for controlling Aphis craccivora, Diabrotica balteata, Heliothis virescens, Myzus persicae, Plutella xylostella and Spodoptera littoralis in cotton, vegetable, maize, rice and soya crops. The active ingredients according to the invention are further especially suitable for controlling Mamestra (preferably in vegetables), Cydia pomonella (preferably in apples), Empoasca (preferably in vegetables, vineyards), Leptinotarsa (preferably in potatos) and Chilo supressalis (preferably in rice).

In a further aspect, the invention may also relate to a method of controlling damage to plant and parts thereof by plant parasitic nematodes (Endoparasitic-, Semiendoparasitic- and Ectoparasitic nematodes), especially plant parasitic nematodes such as root knot nematodes, Meloidogyne hapla, Meloidogyne incognita, Meloidogyne javanica, Meloidogyne arenaria and other Meloidogyne species; cyst-forming nematodes, Globodera rostochiensis and other Globodera species; Heterodera avenae, Heterodera glycines, Heterodera schachtii, Heterodera trifolii, and other Heterodera species; Seed gall nematodes, Anguina species; Stem and foliar nematodes, Aphelenchoides species; Sting nematodes, Belonolaimus longicaudatus and other Belonolaimus species; Pine nematodes, Bursaphelenchus xylophilus and other Bursaphelenchus species; Ring nematodes, Criconema species, Criconemella species, Criconemoides species, Mesocriconema species; Stem and bulb nematodes, Ditylenchus destructor, Ditylenchus dipsaci and other Ditylenchus species; Awl nematodes, Dolichodorus species; Spiral nematodes, Heliocotylenchus multicinctus and other Helicotylenchus species; Sheath and sheathoid nematodes, Hemicycliophora species and Hemicriconemoides species; Hirshmanniella species; Lance nematodes, Hoploaimus species; false rootknot nematodes, Nacobbus species; Needle nematodes, Longidorus elongatus and other Longidorus species; Pin nematodes, Pratylenchus species; Lesion nematodes, Pratylenchus neglectus, Pratylenchus penetrans, Pratylenchus curvitatus, Pratylenchus goodeyi and other Pratylenchus species; Burrowing nematodes, Radopholus similis and other Radopholus species; Reniform nematodes, Rotylenchus robustus, Rotylenchus reniformis and other Rotylenchus species; Scutellonema species; Stubby root nematodes, Trichodorus primitivus and other Trichodorus species, Paratrichodorus species; Stunt nematodes, Tylenchorhynchus claytoni, Tylenchorhynchus dubius and other Tylenchorhynchus species; Citrus nematodes, Tylenchulus species; Dagger nematodes, Xiphinema species; and other plant parasitic nematode species, such as Subanguina spp., Hypsoperine spp., Macroposthonia spp., Melinius spp., Punctodera spp., and Quinisulcius spp.

The compounds of the invention may also have activity against the molluscs. Examples of which include, for example, Ampullariidae; Arion (A. ater, A. circumscriptus, A. hortensis, A. rufus); Bradybaenidae (Bradybaena fruticum); Cepaea (C. hortensis, C. nemoralis); ochlodina; Deroceras (D. agrestis, D. empiricorum, D. laeve, D. reticulatum); Discus (D. rotundatus); Euomphalia; Galba (G. trunculata); Helicelia (H. itala, H. obvia); Helicidae Helicigona arbustorum); Helicodiscus; Helix (H. aperta); Limax (L. cinereoniger, L. flavus, L. marginatus, L. maximus, L. tenellus); Lymnaea; Milax (M. gagates, M. marginatus, M. sowerbyi); Opeas; Pomacea (P. canaticulata); Vallonia and Zanitoides.

The term “crops” is to be understood as including also crop plants which have been so transformed by the use of recombinant DNA techniques that they are capable of synthesising one or more selectively acting toxins, such as are known, for example, from toxin-producing bacteria, especially those of the genus Bacillus.

Toxins that can be expressed by such transgenic plants include, for example, insecticidal proteins, for example insecticidal proteins from Bacillus cereus or Bacillus popilliae; or insecticidal proteins from Bacillus thuringiensis, such as 6-endotoxins, e.g. Cry1Ab, Cry1Ac, Cry1F, Cry1Fa2, Cry2Ab, Cry3A, Cry3Bb1 or Cry9C, or vegetative insecticidal proteins (Vip), e.g. Vip1, Vip2, Vip3 or Vip3A; or insecticidal proteins of bacteria colonising nematodes, for example Photorhabdus spp. or Xenorhabdus spp., such as Photorhabdus luminescens, Xenorhabdus nematophilus; toxins produced by animals, such as scorpion toxins, arachnid toxins, wasp toxins and other insect-specific neurotoxins; toxins produced by fungi, such as Streptomycetes toxins, plant lectins, such as pea lectins, barley lectins or snowdrop lectins; agglutinins; proteinase inhibitors, such as trypsin inhibitors, serine protease inhibitors, patatin, cystatin, papain inhibitors; ribosome-inactivating proteins (RIP), such as ricin, maize-RIP, abrin, luffin, saporin or bryodin; steroid metabolism enzymes, such as 3-hydroxysteroidoxidase, ecdysteroid-UDP-glycosyl-transferase, cholesterol oxidases, ecdysone inhibitors, HMG-COA-reductase, ion channel blockers, such as blockers of sodium or calcium channels, juvenile hormone esterase, diuretic hormone receptors, stilbene synthase, bibenzyl synthase, chitinases and glucanases.

In the context of the present invention there are to be understood by 6-endotoxins, for example Cry1Ab, Cry1Ac, Cry1F, Cry1Fa2, Cry2Ab, Cry3A, Cry3Bb1 or Cry9C, or vegetative insecticidal proteins (Vip), for example Vip1, Vip2, Vip3 or Vip3A, expressly also hybrid toxins, truncated toxins and modified toxins. Hybrid toxins are produced recombinantly by a new combination of different domains of those proteins (see, for example, WO 02/15701). Truncated toxins, for example a truncated Cry1Ab, are known. In the case of modified toxins, one or more amino acids of the naturally occurring toxin are replaced. In such amino acid replacements, preferably non-naturally present protease recognition sequences are inserted into the toxin, such as, for example, in the case of Cry3A055, a cathepsin-G-recognition sequence is inserted into a Cry3A toxin (see WO 03/018810). Examples of such toxins or transgenic plants capable of synthesising such toxins are disclosed, for example, in EP-A-0 374 753, WO 93/07278, WO 95/34656, EP-A-0 427 529, EP-A-451 878 and WO 03/052073.

The processes for the preparation of such transgenic plants are generally known to the person skilled in the art and are described, for example, in the publications mentioned above. Cryl-type deoxyribonucleic acids and their preparation are known, for example, from WO 95/34656, EP-A-0 367 474, EP-A-0 401 979 and WO 90/13651.

The toxin contained in the transgenic plants imparts to the plants tolerance to harmful insects. Such insects can occur in any taxonomic group of insects, but are especially commonly found in the beetles (Coleoptera), two-winged insects (Diptera) and moths (Lepidoptera).

Transgenic plants containing one or more genes that code for an insecticidal resistance and express one or more toxins are known and some of them are commercially available. Examples of such plants are: YieldGard® (maize variety that expresses a Cry1Ab toxin); YieldGard Rootworm® (maize variety that expresses a Cry3Bb1 toxin); YieldGard Plus® (maize variety that expresses a Cry1Ab and a Cry3Bb1 toxin); Starlink® (maize variety that expresses a Cry9C toxin); Herculex I® (maize variety that expresses a Cry1Fa2 toxin and the enzyme phosphinothricine N-acetyltransferase (PAT) to achieve tolerance to the herbicide glufosinate ammonium); NuCOTN 33B® (cotton variety that expresses a Cry1Ac toxin); Bollgard I® (cotton variety that expresses a Cry1Ac toxin); Bollgard II® (cotton variety that expresses a Cry1Ac and a Cry2Ab toxin); VipCot® (cotton variety that expresses a Vip3A and a Cry1Ab toxin); NewLeaf® (potato variety that expresses a Cry3A toxin); NatureGard®, Agrisure® GT Advantage (GA21 glyphosate-tolerant trait), Agrisure® CB Advantage (Bt11 corn borer (CB) trait) and Protecta®.

Further examples of such transgenic crops are:

1. Bt11 Maize from Syngenta Seeds SAS, Chemin de l'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a truncated Cry1Ab toxin. Bt11 maize also transgenically expresses the enzyme PAT to achieve tolerance to the herbicide glufosinate ammonium.
2. Bt176 Maize from Syngenta Seeds SAS, Chemin de l'Hobit 27, F-31 790 St. Sauveur, France, registration number C/FR/96/05/10. Genetically modified Zea mays which has been rendered resistant to attack by the European corn borer (Ostrinia nubilalis and Sesamia nonagrioides) by transgenic expression of a Cry1Ab toxin. Bt176 maize also transgenically expresses the enzyme PAT to achieve tolerance to the herbicide glufosinate ammonium.
3. MIR604 Maize from Syngenta Seeds SAS, Chemin de l'Hobit 27, F-31 790 St. Sauveur, France, registration number C/FR/96/05/10. Maize which has been rendered insect-resistant by transgenic expression of a modified Cry3A toxin. This toxin is Cry3A055 modified by insertion of a cathepsin-G-protease recognition sequence. The preparation of such transgenic maize plants is described in WO 03/018810.
4. MON 863 Maize from Monsanto Europe S.A. 270-272 Avenue de Tervuren, B-1150 Brussels, Belgium, registration number C/DE/02/9. MON 863 expresses a Cry3Bb1 toxin and has resistance to certain Coleoptera insects.
5. IPC 531 Cotton from Monsanto Europe S.A. 270-272 Avenue de Tervuren, B-1150 Brussels, Belgium, registration number C/ES/96/02.
6. 1507 Maize from Pioneer Overseas Corporation, Avenue Tedesco, 7 B-1160 Brussels, Belgium, registration number C/NL/00/10. Genetically modified maize for the expression of the protein Cry1F for achieving resistance to certain Lepidoptera insects and of the PAT protein for achieving tolerance to the herbicide glufosinate ammonium.
7. NK603×MON 810 Maize from Monsanto Europe S.A. 270-272 Avenue de Tervuren, B-1150 Brussels, Belgium, registration number C/GB/02/M3/03. Consists of conventionally bred hybrid maize varieties by crossing the genetically modified varieties NK603 and MON 810. NK603×MON 810 Maize transgenically expresses the protein CP4 EPSPS, obtained from Agrobacterium sp. strain CP4, which imparts tolerance to the herbicide Roundup® (contains glyphosate), and also a Cry1Ab toxin obtained from Bacillus thuringiensis subsp. kurstaki which brings about tolerance to certain Lepidoptera, include the European corn borer.

Transgenic crops of insect-resistant plants are also described in BATS (Zentrum für Biosicherheit und Nachhaltigkeit, Zentrum BATS, Clarastrasse 13, 4058 Basel, Switzerland) Report 2003, (http://bats.ch).

The term “crops” is to be understood as including also crop plants which have been so transformed by the use of recombinant DNA techniques that they are capable of synthesising antipathogenic substances having a selective action, such as, for example, the so-called “pathogenesis-related proteins” (PRPs, see e.g. EP-A-0 392 225). Examples of such antipathogenic substances and transgenic plants capable of synthesising such antipathogenic substances are known, for example, from EP-A-0 392 225, WO 95/33818 and EP-A-0 353 191. The methods of producing such transgenic plants are generally known to the person skilled in the art and are described, for example, in the publications mentioned above.

Crops may also be modified for enhanced resistance to fungal (for example Fusarium, Anthracnose, or Phytophthora), bacterial (for example Pseudomonas) or viral (for example potato leafroll virus, tomato spotted wilt virus, cucumber mosaic virus) pathogens.

Crops also include those that have enhanced resistance to nematodes, such as the soybean cyst nematode.

Crops that are tolerance to abiotic stress include those that have enhanced tolerance to drought, high salt, high temperature, chill, frost, or light radiation, for example through expression of NF-YB or other proteins known in the art.

Antipathogenic substances which can be expressed by such transgenic plants include, for example, ion channel blockers, such as blockers for sodium and calcium channels, for example the viral KP1, KP4 or KP6 toxins; stilbene synthases; bibenzyl synthases; chitinases; glucanases; the so-called “pathogenesis-related proteins” (PRPs; see e.g. EP-A-0 392 225); antipathogenic substances produced by microorganisms, for example peptide antibiotics or heterocyclic antibiotics (see e.g. WO 95/33818) or protein or polypeptide factors involved in plant pathogen defence (so-called “plant disease resistance genes”, as described in WO 03/000906).

Further areas of use of the compositions according to the invention are the protection of stored goods and store rooms and the protection of raw materials, such as wood, textiles, floor coverings or buildings, and also in the hygiene sector, especially the protection of humans, domestic animals and productive livestock against pests of the mentioned type.

The present invention also provides a method for controlling pests (such as mosquitoes and other disease vectors; see also http://www.who.int/malaria/vector_control/irs/en/). In one embodiment, the method for controlling pests comprises applying the compositions of the invention to the target pests, to their locus or to a surface or substrate by brushing, rolling, spraying, spreading or dipping. By way of example, an IRS (indoor residual spraying) application of a surface such as a wall, ceiling or floor surface is contemplated by the method of the invention. In another embodiment, it is contemplated to apply such compositions to a substrate such as non-woven or a fabric material in the form of (or which can be used in the manufacture of) netting, clothing, bedding, curtains and tents.

In one embodiment, the method for controlling such pests comprises applying a pesticidally effective amount of the compositions of the invention to the target pests, to their locus, or to a surface or substrate so as to provide effective residual pesticidal activity on the surface or substrate. Such application may be made by brushing, rolling, spraying, spreading or dipping the pesticidal composition of the invention. By way of example, an IRS application of a surface such as a wall, ceiling or floor surface is contemplated by the method of the invention so as to provide effective residual pesticidal activity on the surface. In another embodiment, it is contemplated to apply such compositions for residual control of pests on a substrate such as a fabric material in the form of (or which can be used in the manufacture of) netting, clothing, bedding, curtains and tents.

Substrates including non-woven, fabrics or netting to be treated may be made of natural fibres such as cotton, raffia, jute, flax, sisal, hessian, or wool, or synthetic fibres such as polyamide, polyester, polypropylene, polyacrylonitrile or the like. The polyesters are particularly suitable. The methods of textile treatment are known, e.g. WO 2008/151984, WO 2003/034823, U.S. Pat. No. 5,631,072, WO 2005/64072, WO2006/128870, EP 1724392, WO 2005113886 or WO 2007/090739.

Further areas of use of the compositions according to the invention are the field of tree injection/trunk treatment for all ornamental trees as well all sort of fruit and nut trees.

In the field of tree injection/trunk treatment, the compounds according to the present invention are especially suitable against wood-boring insects from the order Lepidoptera as mentioned above and from the order Coleoptera, especially against woodborers listed in the following tables A and B:

TABLE A
Examples of exotic woodborers of economic importance.

| Family | Species | Host or Crop Infested |
|---|---|---|
| Buprestidae | Agrilus planipennis | Ash |
| Cerambycidae | Anoplura glabripennis | Hardwoods |
| Scolytidae | Xylosandrus crassiusculus | Hardwoods |
| | X. mutilatus | Hardwoods |
| | Tomicus piniperda | Conifers |

TABLE B
Examples of native woodborers of economic importance.

| Family | Species | Host or Crop Infested |
|---|---|---|
| Buprestidae | Agrilus anxius | Birch |
| | Agrilus politus | Willow, Maple |
| | Agrilus sayi | Bayberry, Sweetfern |
| | Agrilus vittaticolllis | Apple, Pear, Cranberry, |
| | | Serviceberry, Hawthorn |
| | Chrysobothris | Apple, Apricot, Beech, Boxelder, |
| | femorata | Cherry, Chestnut, Currant, Elm, |
| | | Hawthorn, Hackberry, Hickory, |
| | | Horsechestnut, Linden, Maple, |
| | | Mountain-ash, Oak, Pecan, Pear, |
| | | Peach, Persimmon, Plum, Poplar, |
| | | Quince, Redbud, Serviceberry, |
| | | Sycamore, Walnut, Willow |
| | Texania campestris | Basswood, Beech, Maple, Oak, |
| | | Sycamore, Willow, |
| | | Yellow-poplar |
| Cerambycidae | Goes pulverulentus | Beech, Elm, Nuttall, Willow, |
| | | Black oak, Cherrybark oak, |
| | | Water oak, Sycamore |
| | Goes tigrinus | Oak |
| | Neoclytus | Ash, Hickory, Oak, Walnut, |
| | acuminatus | Birch, Beech, Maple, Eastern |
| | | hophornbeam, Dogwood, |
| | | Persimmon, Redbud, Holly, |
| | | Hackberry, Black locust, |
| | | Honeylocust, Yellow-poplar, |
| | | Chestnut, Osage-orange, |
| | | Sassafras, Lilac, Mountain- |
| | | mahogany, Pear, Cherry, Plum, |
| | | Peach, Apple, Elm, Basswood, |
| | | Sweetgum |
| | Neoptychodes | Fig, Alder, Mulberry, Willow, |
| | trilineatus | Netleaf hackberry |
| | Oberea ocellata | Sumac, Apple, Peach, Plum, |
| | | Pear, Currant, Blackberry |
| | Oberea tripunctata | Dogwood, Viburnum, Elm, |
| | | Sourwood, Blueberry, |
| | | Rhododendron, Azalea, Laurel, |
| | | Poplar, Willow, Mulberry |
| | Oncideres cingulata | Hickory, Pecan, Persimmon, |
| | | Elm, Sourwood, Basswood, |
| | | Honeylocust, Dogwood, |
| | | Eucalyptus, Oak, Hackberry, |
| | | Maple, Fruit trees |
| | Saperda calcarata | Poplar |
| | Strophiona nitens | Chestnut, Oak, Hickory, Walnut, |
| | | Beech, Maple |
| Scolytidae | Corthylus | Maple, Oak, Yellow-poplar, |
| | columbianus | Beech, Boxelder, Sycamore, |
| | | Birch, Basswood, Chestnut, Elm |
| | Dendroctonus | Pine |
| | frontalis | |
| | Dryocoetes betulae | Birch, Sweetgum, Wild cherry, |
| | | Beech, Pear |
| | Monarthrum | Oak, Maple, Birch, Chestnut, |
| | fasciatum | Sweetgum, Blackgum, Poplar, |
| | | Hickory, Mimosa, Apple, |
| | | Peach, Pine |
| | Phloeotribus | Peach, Cherry, Plum, Black |
| | liminaris | cherry, Elm, Mulberry, |
| | | Mountain-ash |
| | Pseudopityophthorus | Oak, American beech, Black |
| | pruinosus | cherry, Chickasaw plum, |
| | | Chestnut, Maple, Hickory, |
| | | Hornbeam, Hophornbeam |
| Sesiidae | Paranthrene | Oak, American chestnut |
| | simulans | |
| | Sannina | Persimmon |
| | uroceriformis | |
| | Synanthedon exitiosa | Peach, Plum, Nectarine, Cherry, |
| | | Apricot, Almond, Black cherry |
| | Synanthedon pictipes | Peach, Plum, Cherry, Beach, |
| | | Black Cherry |
| | Synanthedon | Tupelo |
| | rubrofascia | |
| | Synanthedon scitula | Dogwood, Pecan, Hickory, Oak, |
| | | Chestnut, Beech, Birch, Black |
| | | cherry, Elm, Mountain-ash, |
| | | Viburnum, Willow, Apple, |
| | | Loquat, Ninebark, Bayberry |
| | Vitacea polistiformis | Grape |

The present invention may be also used to control any insect pests that may be present in turfgrass, including for example beetles, caterpillars, fire ants, ground pearls, millipedes, sow bugs, mites, mole crickets, scales, mealybugs ticks, spittlebugs, southern chinch bugs and white grubs. The present invention may be used to control insect pests at various stages of their life cycle, including eggs, larvae, nymphs and adults.

In particular, the present invention may be used to control insect pests that feed on the roots of turfgrass including white grubs (such as Cyclocephala spp. (e.g. masked chafer, C. lurida), Rhizotrogus spp. (e.g. European chafer, R. majalis), Cotinus spp. (e.g. Green June beetle, C. nitida), Popillia spp. (e.g. Japanese beetle, P. japonica), Phyllophaga spp. (e.g. May/June beetle), Ataenius spp. (e.g. Black turfgrass ataenius, A. spretulus), Maladera spp. (e.g. Asiatic garden beetle, M. castanea) and Tomarus spp.), ground pearls (Margarodes spp.), mole crickets (tawny, southern, and short-winged; Scapteriscus spp., Gryllotalpa africana) and leatherjackets (European crane fly, Tipula spp.).

The present invention may also be used to control insect pests of turfgrass that are thatch dwelling, including armyworms (such as fall armyworm Spodoptera frugiperda, and common armyworm Pseudaletia unipuncta), cutworms, billbugs (Sphenophorus spp., such as S. venatus verstitus and S. parvulus), and sod webworms (such as Crambus spp. and the tropical sod webworm, Herpetogramma phaeopteralis).

The present invention may also be used to control insect pests of turfgrass that live above the ground and feed on the turfgrass leaves, including chinch bugs (such as southern chinch bugs, Blissus insularis), Bermudagrass mite (Eriophyes cynodoniensis), rhodesgrass mealybug (Antonina graminis), two-lined spittlebug (Propsapia bicincta), leafhoppers, cutworms (Noctuidae family), and greenbugs. The present invention may also be used to control other pests of turfgrass such as red imported fire ants (Solenopsis invicta) that create ant mounds in turf.

In the hygiene sector, the compositions according to the invention are active against ectoparasites such as hard ticks, soft ticks, mange mites, harvest mites, flies (biting and licking), parasitic fly larvae, lice, hair lice, bird lice and fleas.

Examples of such parasites are:

Of the order Anoplurida: Haematopinus spp., Linognathus spp., Pediculus spp. and Phtirus spp., Solenopotes spp.
Of the order Mallophagida: Trimenopon spp., Menopon spp., Trinoton spp., Bovicola spp., Werneckiella spp., Lepikentron spp., Damalina spp., Trichodectes spp. and Felicola spp.
Of the order Diptera and the suborders Nematocerina and Brachycerina, for example Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp. and Melophagus spp.
Of the order Siphonapterida, for example Pulex spp., Ctenocephalides spp., Xenopsylla spp., Ceratophyllus spp.
Of the order Heteropterida, for example Cimex spp., Triatoma spp., Rhodnius spp., Panstrongylus spp.
Of the order Blattarida, for example Blatta orientalis, Periplaneta americana, Blattelagermanica and Supella spp.
Of the subclass Acaria (Acarida) and the orders Meta- and Meso-stigmata, for example Argas spp., Ornithodorus spp., Otobius spp., Ixodes spp., Amblyomma spp., Boophilus spp., Dermacentor spp., Haemophysalis spp., Hyalomma spp., Rhipicephalus spp., Dermanyssus spp., Raillietia spp., Pneumonyssus spp., Sternostoma spp. and Varroa spp.
Of the orders Actinedida (Prostigmata) and Acaridida (Astigmata), for example Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp. and Laminosioptes spp.

The compositions according to the invention are also suitable for protecting against insect infestation in the case of materials such as wood, textiles, plastics, adhesives, glues, paints, paper and card, leather, floor coverings and buildings.

The compositions according to the invention can be used, for example, against the following pests: beetles such as Hylotrupes bajulus, Chlorophorus pilosis, Anobium punctatum, Xestobium rufovillosum, Ptilinuspecticornis, Dendrobium pertinex, Ernobius mollis, Priobium carpini, Lyctus brunneus, Lyctus africanus, Lyctus planicollis, Lyctus linearis, Lyctus pubescens, Trogoxylon aequale, Minthesrugicollis, Xyleborus spec., Tryptodendron spec., Apate monachus, Bostrychus capucins, Heterobostrychus brunneus, Sinoxylon spec. and Dinoderus minutus, and also hymenopterans such as Sirexjuvencus, Urocerus gigas, Urocerus gigas taignus and Urocerus augur, and termites such as Kalotermes flavicollis, Cryptotermes brevis, Heterotermes indicola, Reticulitermes flavipes, Reticulitermes santonensis, Reticulitermes lucifugus, Mastotermes darwiniensis, Zootermopsis nevadensis and Coptotermes formosanus, and bristletails such as Lepisma saccharina.

The compounds according to the invention can be used as pesticidal agents in unmodified form, but they are generally formulated into compositions in various ways using formulation adjuvants, such as carriers, solvents and surface-active substances. The formulations can be in various physical forms, e.g. in the form of dusting powders, gels, wettable powders, water-dispersible granules, water-dispersible tablets, effervescent pellets, emulsifiable concentrates, microemulsifiable concentrates, oil-in-water emulsions, oil-flowables, aqueous dispersions, oily dispersions, suspo-emulsions, capsule suspensions, emulsifiable granules, soluble liquids, water-soluble concentrates (with water or a water-miscible organic solvent as carrier), impregnated polymer films or in other forms known e.g. from the Manual on Development and Use of FAO and WHO Specifications for Pesticides, United Nations, First Edition, Second Revision (2010). Such formulations can either be used directly or diluted prior to use. The dilutions can be made, for example, with water, liquid fertilisers, micronutrients, biological organisms, oil or solvents.

The formulations can be prepared e.g. by mixing the active ingredient with the formulation adjuvants in order to obtain compositions in the form of finely divided solids, granules, solutions, dispersions or emulsions. The active ingredients can also be formulated with other adjuvants, such as finely divided solids, mineral oils, oils of vegetable or animal origin, modified oils of vegetable or animal origin, organic solvents, water, surface-active substances or combinations thereof.

The active ingredients can also be contained in very fine microcapsules. Microcapsules contain the active ingredients in a porous carrier. This enables the active ingredients to be released into the environment in controlled amounts (e.g. slow-release). Microcapsules usually have a diameter of from 0.1 to 500 microns. They contain active ingredients in an amount of about from 25 to 95% by weight of the capsule weight. The active ingredients can be in the form of a monolithic solid, in the form of fine particles in solid or liquid dispersion or in the form of a suitable solution. The encapsulating membranes can comprise, for example, natural or synthetic rubbers, cellulose, styrene/butadiene copolymers, polyacrylonitrile, polyacrylate, polyesters, polyamides, polyureas, polyurethane or chemically modified polymers and starch xanthates or other polymers that are known to the person skilled in the art. Alternatively, very fine microcapsules can be formed in which the active ingredient is contained in the form of finely divided particles in a solid matrix of base substance, but the microcapsules are not themselves encapsulated.

The formulation adjuvants that are suitable for the preparation of the compositions according to the invention are known per se. As liquid carriers there may be used: water, toluene, xylene, petroleum ether, vegetable oils, acetone, methyl ethyl ketone, cyclohexanone, acid anhydrides, acetonitrile, acetophenone, amyl acetate, 2-butanone, butylene carbonate, chlorobenzene, cyclohexane, cyclohexanol, alkyl esters of acetic acid, diacetone alcohol, 1,2-dichloropropane, diethanolamine, p-diethylbenzene, diethylene glycol, diethylene glycol abietate, diethylene glycol butyl ether, diethylene glycol ethyl ether, diethylene glycol methyl ether, N,N-dimethylformamide, dimethyl sulfoxide, 1,4-dioxane, dipropylene glycol, dipropylene glycol methyl ether, dipropylene glycol dibenzoate, diproxitol, alkylpyrrolidone, ethyl acetate, 2-ethylhexanol, ethylene carbonate, 1,1,1-trichloroethane, 2-heptanone, alpha-pinene, d-limonene, ethyl lactate, ethylene glycol, ethylene glycol butyl ether, ethylene glycol methyl ether, gamma-butyrolactone, glycerol, glycerol acetate, glycerol diacetate, glycerol triacetate, hexadecane, hexylene glycol, isoamyl acetate, isobornyl acetate, isooctane, isophorone, isopropylbenzene, isopropyl myristate, lactic acid, laurylamine, mesityl oxide, methoxy-propanol, methyl isoamyl ketone, methyl isobutyl ketone, methyl laurate, methyl octanoate, methyl oleate, methylene chloride, m-xylene, n-hexane, n-octylamine, octadecanoic acid, octylamine acetate, oleic acid, oleylamine, o-xylene, phenol, polyethylene glycol, propionic acid, propyl lactate, propylene carbonate, propylene glycol, propylene glycol methyl ether, p-xylene, toluene, triethyl phosphate, triethylene glycol, xylenesulfonic acid, paraffin, mineral oil, trichloroethylene, perchloroethylene, ethyl acetate, amyl acetate, butyl acetate, propylene glycol methyl ether, diethylene glycol methyl ether, methanol, ethanol, isopropanol, and alcohols of higher molecular weight, such as amyl alcohol, tetrahydrofurfuryl alcohol, hexanol, octanol, ethylene glycol, propylene glycol, glycerol, N-methyl-2-pyrrolidone and the like.

Suitable solid carriers are, for example, talc, titanium dioxide, pyrophyllite clay, silica, attapulgite clay, kieselguhr, limestone, calcium carbonate, bentonite, calcium montmorillonite, cottonseed husks, wheat flour, soybean flour, pumice, wood flour, ground walnut shells, lignin and similar substances.

A large number of surface-active substances can advantageously be used in both solid and liquid formulations, especially in those formulations which can be diluted with a carrier prior to use. Surface-active substances may be anionic, cationic, non-ionic or polymeric and they can be used as emulsifiers, wetting agents or suspending agents or for other purposes. Typical surface-active substances include, for example, salts of alkyl sulfates, such as diethanolammonium lauryl sulfate; salts of alkylarylsulfonates, such as calcium dodecylbenzenesulfonate; alkylphenol/alkylene oxide addition products, such as nonylphenol ethoxylate; alcohol/alkylene oxide addition products, such as tridecylalcohol ethoxylate; soaps, such as sodium stearate; salts of alkylnaphthalenesulfonates, such as sodium dibutylnaphthalenesulfonate; dialkyl esters of sulfosuccinate salts, such as sodium di(2-ethylhexyl)sulfosuccinate; sorbitol esters, such as sorbitol oleate; quaternary amines, such as lauryltrimethylammonium chloride, polyethylene glycol esters of fatty acids, such as polyethylene glycol stearate; block copolymers of ethylene oxide and propylene oxide; and salts of mono- and di-alkylphosphate esters; and also further substances described e.g. in McCutcheon's Detergents and Emulsifiers Annual, MC Publishing Corp., Ridgewood N.J. (1981).

Further adjuvants that can be used in pesticidal formulations include crystallisation inhibitors, viscosity modifiers, suspending agents, dyes, anti-oxidants, foaming agents, light absorbers, mixing auxiliaries, antifoams, complexing agents, neutralising or pH-modifying substances and buffers, corrosion inhibitors, fragrances, wetting agents, take-up enhancers, micronutrients, plasticisers, glidants, lubricants, dispersants, thickeners, antifreezes, microbicides, and liquid and solid fertilisers.

The compositions according to the invention can include an additive comprising an oil of vegetable or animal origin, a mineral oil, alkyl esters of such oils or mixtures of such oils and oil derivatives. The amount of oil additive in the composition according to the invention is generally from 0.01 to 10%, based on the mixture to be applied. For example, the oil additive can be added to a spray tank in the desired concentration after a spray mixture has been prepared. Preferred oil additives comprise mineral oils or an oil of vegetable origin, for example rapeseed oil, olive oil or sunflower oil, emulsified vegetable oil, alkyl esters of oils of vegetable origin, for example the methyl derivatives, or an oil of animal origin, such as fish oil or beef tallow. Preferred oil additives comprise alkyl esters of C₈-C₂₂ fatty acids, especially the methyl derivatives of C₁₂-C₁₈ fatty acids, for example the methyl esters of lauric acid, palmitic acid and oleic acid (methyl laurate, methyl palmitate and methyl oleate, respectively). Many oil derivatives are known from the Compendium of Herbicide Adjuvants, 10^{th} Edition, Southern Illinois University, 2010.

The inventive compositions generally comprise from 0.1 to 99% by weight, especially from 0.1 to 95% by weight, of compounds of the present invention and from 1 to 99.9% by weight of a formulation adjuvant which preferably includes from 0 to 25% by weight of a surface-active substance. Whereas commercial products may preferably be formulated as concentrates, the end user will normally employ dilute formulations.

The rates of application vary within wide limits and depend on the nature of the soil, the method of application, the crop plant, the pest to be controlled, the prevailing climatic conditions, and other factors governed by the method of application, the time of application and the target crop. As a general guideline compounds may be applied at a rate of from 1 to 2000 I/ha, especially from 10 to 1000 l/ha.

Preferred formulations can have the following compositions (weight %):

### Emulsifiable Concentrates:

active ingredient: 1 to 95%, preferably 60 to 90%
surface-active agent: 1 to 30%, preferably 5 to 20%
liquid carrier: 1 to 80%, preferably 1 to 35%

### Dusts:

active ingredient: 0.1 to 10%, preferably 0.1 to 5%
solid carrier: 99.9 to 90%, preferably 99.9 to 99%

### Suspension Concentrates:

active ingredient: 5 to 75%, preferably 10 to 50%
water: 94 to 24%, preferably 88 to 30%
surface-active agent: 1 to 40%, preferably 2 to 30%

### Wettable Powders:

active ingredient: 0.5 to 90%, preferably 1 to 80%
surface-active agent: 0.5 to 20%, preferably 1 to 15%
solid carrier: 5 to 95%, preferably 15 to 90%

### Granules:

active ingredient: 0.1 to 30%, preferably 0.1 to 15%
solid carrier: 99.5 to 70%, preferably 97 to 85% The following Examples further illustrate, but do not limit, the invention.

| Wettable powders | a) | b) | c) |
|---|---|---|---|
| active ingredients | 25% | 50% | 75% |
| sodium lignosulfonate | 5% | 5% | — |
| sodium lauryl sulfate | 3% | — | 5% |
| sodium diisobutylnaphthalenesulfonate | — | 6% | 10% |
| phenol polyethylene glycol ether | — | 2% | — |
| (7-8 mol of ethylene oxide) | | | |
| highly dispersed silicic acid | 5% | 10% | 10% |
| Kaolin | 62% | 27% | — |

The combination is thoroughly mixed with the adjuvants and the mixture is thoroughly ground in a suitable mill, affording wettable powders that can be diluted with water to give suspensions of the desired concentration.

| Powders for dry seed treatment | a) | b) | c) |
|---|---|---|---|
| active ingredients | 25% | 50% | 75% |
| light mineral oil | 5% | 5% | 5% |
| highly dispersed silicic acid | 5% | 5% | — |
| Kaolin | 65% | 40% | — |
| Talcum | — | | 20% |

The combination is thoroughly mixed with the adjuvants and the mixture is thoroughly ground in a suitable mill, affording powders that can be used directly for seed treatment.

| Emulsifiable concentrate | |
|---|---|
| active ingredients | 10% |
| octylphenol polyethylene glycol ether | 3% |
| (4-5 mol of ethylene oxide) | |
| calcium dodecylbenzenesulfonate | 3% |
| castor oil polyglycol ether (35 mol | 4% |
| of ethylene oxide) | |
| Cyclohexanone | 30% |
| xylene mixture | 50% |

Emulsions of any required dilution, which can be used in plant protection, can be obtained from this concentrate by dilution with water.

| Dusts | a) | b) | c) |
|---|---|---|---|
| Active ingredients | 5% | 6% | 4% |
| Talcum | 95% | — | — |
| Kaolin | — | 94% | — |
| mineral filler | — | — | 96% |

Ready-for-use dusts are obtained by mixing the combination with the carrier and grinding the mixture in a suitable mill. Such powders can also be used for dry dressings for seed.

| Extruder granules | |
|---|---|
| Active ingredients | 15% |
| sodium lignosulfonate | 2% |
| carboxymethylcellulose | 1% |
| Kaolin | 82% |

The combination is mixed and ground with the adjuvants, and the mixture is moistened with water. The mixture is extruded and then dried in a stream of air.

| Coated granules | |
|---|---|
| Active ingredients | 8% |
| polyethylene glycol (mol. wt. 200) | 3% |
| Kaolin | 89% |

The finely ground combination is uniformly applied, in a mixer, to the kaolin moistened with polyethylene glycol. Non-dusty coated granules are obtained in this manner.

### Suspension Concentrate

| | |
|---|---|
| active ingredients | 40% |
| propylene glycol | 10% |
| nonylphenol polyethylene glycol ether | 6% |
| (15 mol of ethylene oxide) | |
| Sodium lignosulfonate | 10% |
| carboxymethylcellulose | 1% |
| silicone oil (in the form of a 75% | 1% |
| emulsion in water) | |
| Water | 32% |

The finely ground combination is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

### Flowable Concentrate for Seed Treatment

| | |
|---|---|
| active ingredients | 40% |
| propylene glycol | 5% |
| copolymer butanol PO/EO | 2% |
| Tristyrenephenole with 10-20 moles EO | 2% |
| 1,2-benzisothiazolin-3-one (in the form | 0.5% |
| of a 20% solution in water) | |
| monoazo-pigment calcium salt | 5% |
| Silicone oil (in the form of a 75% | 0.2% |
| emulsion in water) | |
| Water | 45.3% |

The finely ground combination is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

### Slow Release Capsule Suspension

28 parts of the combination are mixed with 2 parts of an aromatic solvent and 7 parts of toluene diisocyanate/polymethylene-polyphenylisocyanate-mixture (8:1). This mixture is emulsified in a mixture of 1.2 parts of polyvinylalcohol, 0.05 parts of a defoamer and 51.6 parts of water until the desired particle size is achieved. To this emulsion a mixture of 2.8 parts 1,6-diaminohexane in 5.3 parts of water is added. The mixture is agitated until the polymerization reaction is completed. The obtained capsule suspension is stabilized by adding 0.25 parts of a thickener and 3 parts of a dispersing agent. The capsule suspension formulation contains 28% of the active ingredients. The medium capsule diameter is 8-15 microns. The resulting formulation is applied to seeds as an aqueous suspension in an apparatus suitable for that purpose.

Formulation types include an emulsion concentrate (EC), a suspension concentrate (SC), a suspo-emulsion (SE), a capsule suspension (CS), a water dispersible granule (WG), an emulsifiable granule (EG), an emulsion, water in oil (EO), an emulsion, oil in water (EW), a micro-emulsion (ME), an oil dispersion (OD), an oil miscible flowable (OF), an oil miscible liquid (OL), a soluble concentrate (SL), an ultra-low volume suspension (SU), an ultra-low volume liquid (UL), a technical concentrate (TK), a dispersible concentrate (DC), a wettable powder (WP), a soluble granule (SG) or any technically feasible formulation in combination with agriculturally acceptable adjuvants.

### PREPARATORY EXAMPLES

“Mp” means melting point in ° C. Free radicals represent methyl groups. ¹H NMR measurements were recorded on a Brucker 400 MHz spectrometer, chemical shifts are given in ppm relevant to a TMS standard. Spectra measured in deuterated solvents as indicated. Either one of the LCMS methods below was used to characterize the compounds. The characteristic LCMS values obtained for each compound were the retention time (“Rt”, recorded in minutes) and the measured molecular ion (M+H)⁺ or (M−H)⁻.

### LCMS Methods:

### Method 1:

Spectra were recorded on a Mass Spectrometer from Waters (ZQ Single quadrupole mass spectrometer) equipped with an electrospray source (Polarity: positive or negative ions, Capillary: 3.00 kV, Cone range: 30-60 V, Extractor: 2.00 V, Source Temperature: 150° C., Desolvation Temperature: 350° C., Cone Gas Flow: 0 L/Hr, Desolvation Gas Flow: 650 L/Hr, Mass range: 100 to 900 Da) and an Acquity UPLC from Waters: Binary pump, heated column compartment and diode-array detector. Solvent degasser, binary pump, heated column compartment and diode-array detector. Column: Waters UPLC HSS T3, 1.8 μm, 30×2.1 mm, Temp: 60° C., DAD Wavelength range (nm): 210 to 500, Solvent Gradient: A=water+5% MeOH+0.05% HCOOH, B=Acetonitrile+0.05% HCOOH: gradient: 0 min 0% B, 100% A; 1.2-1.5 min 100% B; Flow (ml/min) 0.85.

### Method 2:

Spectra were recorded on a Mass Spectrometer from Waters (SQD or ZQ Single quadrupole mass spectrometer) equipped with an electrospray source (Polarity: positive or negative ions, Capillary: 3.00 kV, Cone range: 30-60 V, Extractor: 2.00 V, Source Temperature: 150° C., Desolvation Temperature: 350° C., Cone Gas Flow: 0 L/Hr, Desolvation Gas Flow: 650 L/Hr, Mass range: 100 to 900 Da) and an Acquity UPLC from Waters: Binary pump, heated column compartment and diode-array detector. Solvent degasser, binary pump, heated column compartment and diode-array detector. Column: Waters UPLC HSS T3, 1.8 μm, 30×2.1 mm, Temp: 60° C., DAD Wavelength range (nm): 210 to 500, Solvent Gradient: A=water+5% MeOH+0.05% HCOOH, B=Acetonitrile+0.05% HCOOH; gradient: 0 min 0% B, 100% A; 2.7-3.0 min 100% B; Flow (ml/min) 0.85.

### Method 3:

Spectra were recorded on a Mass Spectrometer from Agilent Technologies (6410 Triple Quadrupole mass spectrometer) equipped with an equipped with an electrospray source (Polarity: positive or negative ions, MS2 Scan, Capillary: 4.00 kV, Fragmentor: 100 V, Desolvatation Temperature: 350° C., Gas Flow: 11 L/min, Nebulizer Gas: 45 psi, Mass range: 110 to 1000 Da) and a 1200 Series HPLC from Agilent: quaternary pump, heated column compartment and diode-array detector. Column: KINETEX EVO C18, 2.6 μm, 50×4.6 mm, Temp: 40° C., DAD Wavelength range (nm): 210 to 400, Solvent Gradient: A=water+5% Acetonitrile+0.1% HCOOH, B=Acetonitrile+0.1% HCOOH: gradient: 0 min 0% B, 100% A; 0.9-1.8 min 100% B; Flow (mL/min) 1.8.

### Method 4:

Spectra were recorded on a Mass Spectrometer from Waters (SQD Single quadrupole mass spectrometer) equipped with an electrospray source (Polarity: positive or negative ions, Full Scan, Capillary: 3.00 kV, Cone range: 41 V, Source Temperature: 150° C., Desolvation Temperature: 500° C., Cone Gas Flow: 50 L/Hr, Desolvation Gas Flow: 1000 L/Hr, Mass range: 110 to 800 Da) and a H-Class UPLC from Waters: Binary pump, heated column compartment and diode-array detector. Column: Waters UPLC HSS T3 C18, 1.8 μm, 30×2.1 mm, Temp: 40° C., DAD Wavelength range (nm): 210 to 400, Solvent Gradient: A=water+5% Acetonitrile+0.1% HCOOH, B=Acetonitrile+0.1% HCOOH: gradient: 0 min 10% B; 0-0.2 min 10-50% B; 0.2-0.7 min 50-100% B; Flow (mL/min) 0.8.

### Method 5:

Spectra were recorded on a Mass Spectrometer from Waters (SQD Two mass spectrometer) equipped with an electrospray source (Polarity: positive or negative ions, Capillary: 3.00 kV, Cone range: 41 V, Source Temperature: 150° C., Desolvation Temperature: 500° C., Cone Gas Flow: 50 L/Hr, Desolvation Gas Flow: 1000 L/Hr, Mass range: 110 to 800 Da) and an H-class UPLC from Waters: Quaternary pump, heated column compartment and diode-array detector. Column: Acquity UPLC HSS T3, 1.8 μm, 30×2.1 mm, Temp: 40° C., DAD Wavelength range (nm): 200 to 400, Solvent Gradient: A=water+0.1% HCOOH:Acetonitrile:95:5 v/v, B=Acetonitrile+0.05% HCOOH: gradient: 0-1.0 min 10% B, 90% A; 1.0-5.3 min 10-100% B; 5.3-6.0 min 100-10% B; Flow (ml/min) 0.6.

### Example H1: Preparation of N-[5-ethylsulfonyl-6-[1-(2,2,3,3,3-pentafluoropropyl)pyrazolo[3,4-c]pyridin-5-yl]-3-pyridyl]-N-methyl-acetamide (Compound P2)

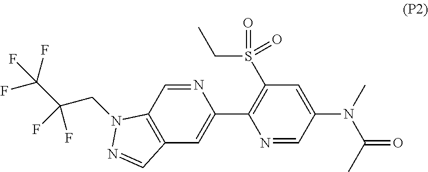 [SMILES: CCS(=O)(=O)c1cc(N(C)C(C)=O)cnc1-c1cc2cnn(CC(F)(F)C(F)(F)F)c2cn1]

### Step 1: Preparation of tert-butyl N-(5-fluoro-3-pyridyl)-N-methyl-carbamate (Intermediate I5)

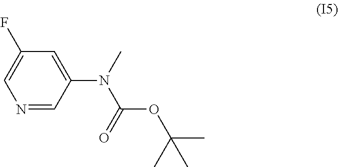 [SMILES: CN(C(=O)OC(C)(C)C)c1cncc(F)c1]

Methyl iodide (0.877 mL, 14.2 mmol, 2.0 eq.) was added dropwise at room temperature to a solution of tert-butyl N-[5-(trifluoromethyl)-3-pyridyl]carbamate (CAS 342603-20-7) (1.50 g, 7.10 mmol) and cesium carbonate (4.70 g, 14.2 mmol, 2.0 eq.) in N,N-dimethylformamide (30 mL) in a sealed tube under argon. The yellow cloudy suspension was heated at 80° C. and stirred for 4 hours. After cooling to room temperature, the reaction mixture was poured over water and the aqueous phase was extracted twice with ethyl acetate. The combined organic phases were washed with brine, dried over magnesium sulfate, filtered and concentrated. The crude material was purified by flash chromatography over silica gel (ethyl acetate in cyclohexane) to give the desired product as a brown oil (537 mg). LCMS (method 1): 227 (M+H)⁺, Rt 0.90 min.

### Step 2: Preparation of tert-butyl N-(5-fluoro-1-oxido-pyridin-1-ium-3-yl)-N-methyl-carbamate (Intermediate I6)

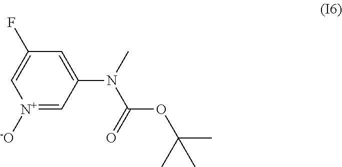

A solution of 3-chloroperbenzoic acid (1.12 g, 4.86 mmol, 2.05 eq.) in dichloromethane (2.0 mL) was added to a solution of tert-butyl N-(5-fluoro-3-pyridyl)-N-methyl-carbamate (intermediate 15 prepared as described above) (537 mg, 2.37 mmol) in dichloromethane (7.0 mL) at 0° C. After stirring for 2 hours at room temperature, the reaction mixture was diluted with tert-butyl methyl ether (25 mL) and washed with a 10% sodium hydrogenosulfite aqueous solution to remove any remaining peroxide (controlled by a starch test). The organic layer was further washed with a saturated sodium hydrogenocarbonate aqueous solution, water and brine, dried over sodium sulfate, filtered and concentrated. Purification of the crude product by flash chromatography over silica gel (methanol in dichloromethane) afforded the desired compound. LCMS (method 1): 243 (M+H)⁺, Rt 0.71 min.

### Step 3: Preparation of tert-butyl N-[5-fluoro-1-oxido-6-[1-(2,2,3,3,3-pentafluoropropyl)pyrazolo[3,4-c]pyridin-5-yl]pyridin-1-ium-3-yl]-N-methyl-carbamate (Intermediate I7)

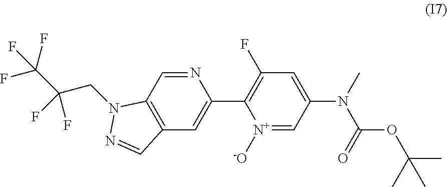

Obtained according to the procedure described in step 2 of example H2 by reaction of intermediate 11 (prepared as described in step 1 of example H2 below) and intermediate 16 (prepared as described above). LCMS (method 1): 492 (M+H)⁺, Rt 0.98 min.

### Step 4: Preparation of tert-butyl N-[5-ethylsulfanyl-1-oxido-6-[1-(2,2,3,3,3-pentafluoropropyl) pyrazolo[3,4-c]pyridin-5-yl]pyridin-1-ium-3-yl]-N-methyl-carbamate (Intermediate I8)

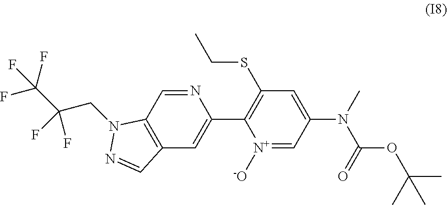

Obtained according to the procedure described in step 3 of example H2 by reaction of intermediate 17 (prepared as described above). LCMS (method 1): 534 (M+H)⁺, Rt 1.01 min.

### Step 5: Preparation of tert-butyl N-[5-ethylsulfanyl-6-[1-(2,2,3,3,3-pentafluoropropyl)pyrazolo[3,4-c]pyridin-5-yl]-3-pyridyl]-N-methyl-carbamate (Intermediate I9)

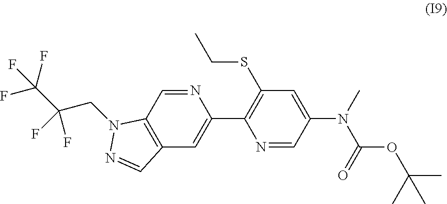 [SMILES: CCSc1cc(N(C)C(=O)OC(C)(C)C)cnc1-c1cc2cnn(CC(F)(F)C(F)(F)F)c2cn1]

Obtained according to the procedure described in step 4 of example H2 by reaction of intermediate 18 (prepared as described above). LCMS (method 1): 518 (M+H)⁺, Rt 1.16 min.

### Step 6: Preparation of tert-butyl N-[5-ethylsulfonyl-6-[1-(2,2,3,3,3-pentafluoropropyl)pyrazolo[3,4-c]pyridin-5-yl]-3-pyridyl]-N-methyl-carbamate (Intermediate I10)

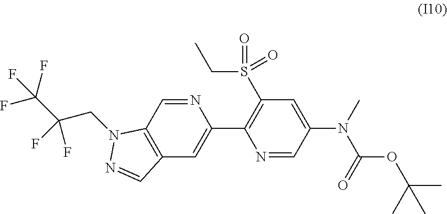 [SMILES: CCS(=O)(=O)c1cc(N(C)C(=O)OC(C)(C)C)cnc1-c1cc2cnn(CC(F)(F)C(F)(F)F)c2cn1]

Obtained according to the procedure described in example H3 by reaction of intermediate 19 (prepared as described above). LCMS (method 1): 550 (M+H)⁺, Rt 1.09 min.

### Step 7: Preparation of 5-ethylsulfonyl-N-methyl-6-[1-(2,2,3,3,3-pentafluoropropyl)pyrazolo[3,4-c]pyridin-5-yl]pyridin-3-amine (Compound P1)

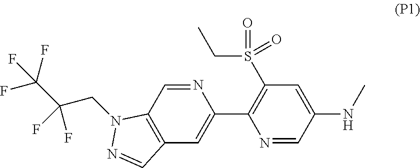 [SMILES: CCS(=O)(=O)c1cc(NC)cnc1-c1cc2cnn(CC(F)(F)C(F)(F)F)c2cn1]

2,2,2-trifluoroacetic acid (1.0 mL) was added dropwise to a solution of tert-butyl N-[5-ethylsulfonyl-6-[1-(2,2,3,3,3-pentafluoropropyl)pyrazolo[3,4-c]pyridin-5-yl]-3-pyridyl]-N-methyl-carbamate (intermediate 110 prepared as described above) (108 mg, 0.200 mmol) in dichloromethane (2.00 mL) at 0° C. After stirring overnight at room temperature, the reaction mixture was concentrated. The crude material was dissolved in ethyl acetate, and the organic phase was washed three times with water, dried over magnesium sulfate, filtered and concentrated. Purification of the crude material by flash chromatography over silica gel (ethyl acetate in cyclohexane) afforded the desired compound.

LCMS (method 1): 450 (M+H)⁺, Rt 0.89 min.

### Step 8: Preparation of N-[5-ethylsulfonyl-6-[1-(2,2,3,3,3-pentafluoropropyl)pyrazolo[3,4-c]pyridin-5-yl]-3-pyridyl]-N-methyl-acetamide (Compound P2)

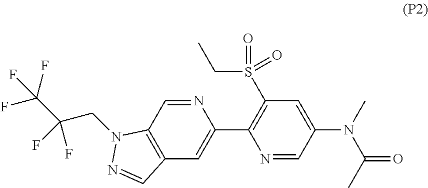 [SMILES: CCS(=O)(=O)c1cc(N(C)C(C)=O)cnc1-c1cc2cnn(CC(F)(F)C(F)(F)F)c2cn1]

Acetyl chloride (0.160 mmol, 1.0 eq.) was added to a 0° C. cooled solution of 5-ethylsulfonyl-N-methyl-6-[1-(2,2,3,3,3-pentafluoropropyl)pyrazolo[3,4-c]pyridin-5-yl]pyridin-3-amine (compound P1 prepared as described above) (72 mg, 0.160 mmol) in dichloromethane (2.0 mL) with triethylamine (0.176 mmol, 1.10 eq.). After stirring for 30 min, the reaction mixture was concentrated. The crude material was dissolved in ethyl acetate, and the organic phase was washed three times with water, dried over magnesium sulfate, filtered and concentrated. Purification of the crude material by flash chromatography over silica gel (ethyl acetate in cyclohexane) afforded the desired compound (73 mg) as a solid, mp 189-190° C. LCMS (method 1): 492 (M+H)⁺, Rt 0.88 min.

### Example H2: Preparation of 5-(3-ethylsulfanyl-2-pyridyl)-1-(2,2,3,3,3-pentafluoropropyl)pyrazolo[3,4-c]pyridine (Compound P3)

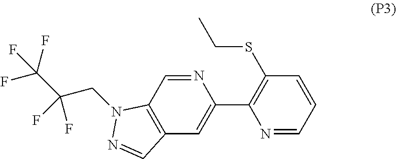 [SMILES: CCSc1cccnc1-c1cc2cnn(CC(F)(F)C(F)(F)F)c2cn1]

### Step 1: Preparation of 5-bromo-1-(2,2,3,3,3-pentafluoropropyl)pyrazolo[3,4-c]pyridine (Intermediate I1)

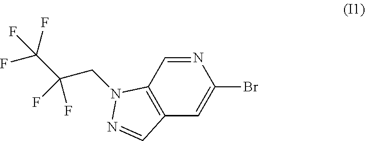 [SMILES: FC(F)(F)C(F)(F)Cn1ncc2cc(Br)ncc21]

Potassium carbonate (237 mg, 1.72 mmol, 2.0 eq.) and 2,2,3,3,3-pentafluoropropyl trifluoromethane-sulfonate (CAS 6401-00-9) (315 mg, 1.12 mmol, 1.30 eq.) were added to a solution of 5-bromo-1H-pyrazolo[3,4-c]pyridine (CAS 929617-35-6) (170 mg, 0.86 mmol) in N,N-dimethylformamide (4.30 mL). After stirring for 2 hours at room temperature, the reaction mixture was poured over water. The aqueous phase was thoroughly extracted with ethyl acetate, the combined organic phases were dried over sodium sulfate, filtered and concentrated. Purification of the crude material by flash chromatography over silica gel (ethyl acetate in cyclohexane) afforded the desired product as a white solid (150 mg). LCMS (method 1): 330/332 (M+H)⁺, Rt 0.97 min.

Similarly, 5-bromo-1-(2,2,3,3,3-pentafluoropropyl)pyrrolo[2,3-c]pyridine (intermediate 13) can be prepared from 5-bromo-1H-pyrrolo[2,3-c]pyridine (CAS 1215387-58-8) and 2,2,3,3,3-pentafluoropropyl trifluoromethane-sulfonate (CAS 6401-00-9). LCMS (method 1): 329/331 (M+H)⁺, Rt 1.02 min.

Similarly, 6-bromo-3-(2,2,3,3,3-pentafluoropropyl)imidazo[4,5-c]pyridine (intermediate 14) can be prepared from 6-bromo-3H-imidazo[4,5-c]pyridine (CAS 1312440-90-6) and 2,2,3,3,3-pentafluoro-propyl trifluoromethane-sulfonate (CAS 6401-00-9). LCMS (method 1): 330/332 (M+H)⁺, Rt 0.87 min.

### Step 2: Preparation of 5-(3-fluoro-1-oxido-pyridin-1-ium-2-yl)-1-(2,2,3,3,3-pentafluoropropyl) pyrazolo[3,4-c]pyridine (Intermediate I2)

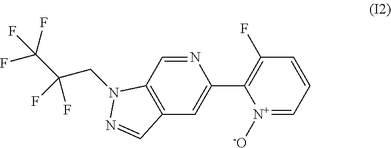

A 1.0 M solution of tetramethylpiperidinyl zinc chloride lithium chloride complex in tetrahydrofuran (3.65 mL, 3.65 mmol, 1.50 eq.) was added dropwise at room temperature to a solution of 3-fluoropyridine N-oxide (CAS 695-37-4) (0.275 g, 2.43 mmol, 1.0 eq.) and 5-bromo-1-(2,2,3,3,3-pentafluoropropyl)pyrazolo[3,4-c]pyridine (intermediate 11 prepared as described above) (0.802 g, 2.43 mmol, 1.0 eq.) in tetrahydrofuran (2.75 mL) under argon. After complete addition, the mixture was degassed for 5 min before adding [1,1′-bis(diphenylphosphino)ferrocene] dichloropalladium (complex with dichloromethane) and heating at 60° C. for 4 hours. After cooling to room temperature, the reaction mixture was poured over a saturated aqueous sodium hydrogenocarbonate solution, and the aqueous phase was extracted twice with ethyl acetate. The combined organic phases were washed with a saturated sodium hydrogenocarbonate solution, dried over sodium sulfate, filtered, and concentrated. The crude material was purified by flash chromatography over silica gel (methanol in dichloromethane) to afford the desired product as a brown solid. LCMS (method 1): 363 (M+H)⁺, Rt 0.76 min.

Similarly, 6-(3-fluoro-1-oxido-pyridin-1-ium-2-yl)-3-(2,2,3,3,3-pentafluoropropyl)imidazo[4,5-c]pyridine (intermediate 128) can be prepared from 6-bromo-3-(2,2,3,3,3-pentafluoropropyl)imidazo[4,5-c]pyridine (intermediate 14 prepared as described above). LCMS (method 1): 363 (M+H)⁺, Rt 0.69 min.

### Step 3: Preparation of 5-(3-ethylsulfanyl-1-oxido-pyridin-1-ium-2-yl)-1-(2,2,3,3,3-pentafluoropropyl) pyrazolo[3,4-c]pyridine (Pyridine N-Oxide PN1)

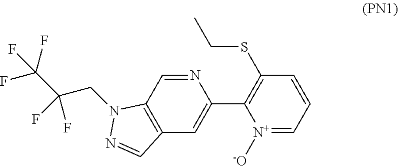

Sodium ethanethiolate (217 mg, 2.58 mmol, 3.0 eq.) was added in one portion to a solution of 5-(3-fluoro-1-methyl-1-oxido-2-pyridyl)-1-(2,2,3,3,3-pentafluoropropyl)pyrazolo[3,4-c]pyridine (intermediate 12 prepared as described above) (325 mg, 0.86 mmol) in N,N-dimethylformamide (1.0 mL) cooled at 0° C. After stirring for 2 hours at room temperature, the reaction mixture was poured over water, and the aqueous phase was extracted three times with ethyl acetate. The combined organic phases were washed with water, brine, dried over sodium sulfate, filtered and concentrated. The crude material was used directly without any purification. LCMS (method 1): 405 (M+H)⁺, Rt 0.83 min.

Similarly, 6-(3-ethylsulfanyl-1-oxido-pyridin-1-ium-2-yl)-3-(2,2,3,3,3-pentafluoropropyl)imidazo[4,5-c]pyridine (pyridine N-oxide PN3) can be prepared from 6-(3-fluoro-1-oxido-pyridin-1-ium-2-yl)-3-(2,2,3,3,3-pentafluoropropyl)imidazo[4,5-c]pyridine (intermediate 128 prepared as described above). LCMS (method 1): 405 (M+H)⁺, Rt 0.77 min.

### Step 4: Preparation of 5-(3-ethylsulfanyl-2-pyridyl)-1-(2,2,3,3,3-pentafluoropropyl)pyrazolo[3,4-c]pyridine (Compound P3)

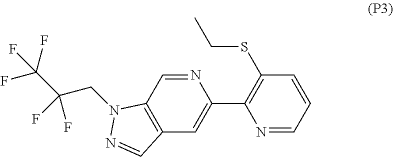 [SMILES: CCSc1cccnc1-c1cc2cnn(CC(F)(F)C(F)(F)F)c2cn1]

Zinc (91 mg, 1.39 mmol, 1.5 eq.) was added at 10° C. to a suspension of 5-(3-ethylsulfanyl-1-oxido-pyridin-1-ium-2-yl)-1-(2,2,3,3,3-pentafluoropropyl)pyrazolo[3,4-c]pyridine (pyridine N-oxide PN1 prepared as described above) (374 mg, 0.93 mmol) in tetrahydrofuran (10 mL) and a saturated ammonium chloride aqueous solution (5 mL). After stirring for 2 hours at room temperature, the reaction mixture was diluted with ethyl acetate and filtered over Celite. The organic phase was washed with water, brined, dried over sodium sulfate, filtered and concentrated. The crude yellow oil was purified by flash chromatography over silica gel (methanol in dichloromethane) to afford the desired product as a yellow solid (225 mg). LCMS (method 1): 389 (M+H)⁺, Rt 0.98 min.

### Example H3: Preparation of 5-(3-ethylsulfonyl-2-pyridyl)-1-(2,2,3,3,3-pentafluoropropyl)pyrazolo[3,4-c]pyridine (Compound P4)

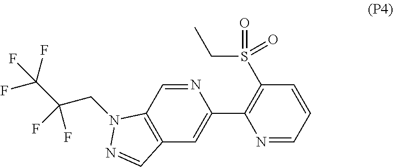 [SMILES: CCS(=O)(=O)c1cccnc1-c1cc2cnn(CC(F)(F)C(F)(F)F)c2cn1]

A solution of 3-chloroperbenzoic acid (182 mg, 0.79 mmol, 2.05 eq.) in dichloromethane (2.0 mL) was added to a solution of 5-(3-ethylsulfanyl-2-pyridyl)-1-(2,2,3,3,3-pentafluoropropyl)pyrazolo[3,4-c]pyridine (compound P3 prepared as described above) (150 mg, 0.39 mmol) in dichloromethane (3.0 mL) at 0° C. After stirring for 30 min at room temperature, the reaction mixture was diluted with tert-butyl methyl ether (25 mL) and washed with a 10% sodium hydrogenosulfite aqueous solution to remove any remaining peroxide (controlled by a starch test). The organic layer was further washed with a saturated sodium hydrogenocarbonate aqueous solution, water and brine, dried over sodium sulfate, filtered and concentrated. Purification of the crude product by flash chromatography over silica gel (methanol in dichloromethane) afforded the desired compound (91 mg).

LCMS (method 1): 421 (M+H)⁺, Rt 0.91 min.

### Example H4: Preparation of 2-[5-ethylsulfonyl-6-[1-(2,2,3,3,3-pentafluoropropyl)pyrrolo[2,3-c]pyridin-5-yl]-3-pyridyl]-2-methyl-propanenitrile (Compound P19)

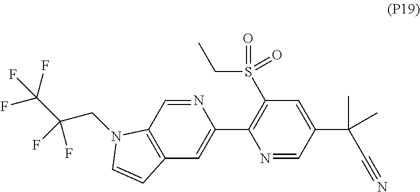 [SMILES: CCS(=O)(=O)c1cc(C(C)(C)C#N)cnc1-c1cc2ccn(CC(F)(F)C(F)(F)F)c2cn1]

### Step 1: Preparation of 2-(5-fluoro-3-pyridyl)-2-methyl-propanenitrile (Intermediate I34)

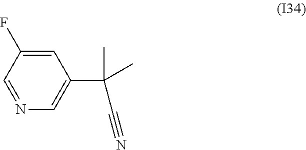

To a solution of 2-(5-fluoro-3-pyridyl)acetonitrile (CAS 39891-06-0) (2.77 g, 20.3 mmol) in tetrahydrofuran (102 mL) was added sodium hydride (2.44 g, 61.0 mmol) portionwise at 0° C. The reaction mixture was stirred at 0-5° C. for 30 minutes, then iodomethane (3.88 mL, 61.0 mmol) was added dropwise and the mixture was stirred at room temperature for 2 hour. After completion, the reaction mixture was extracted with ethyl acetate and brine, the combined organic layers were dried over magnesium sulfate, filtered and evaporated. Purification by flash chromatography over silica gel (ethyl acetate in cyclohexane) afforded the desired product as yellow solid (2.80 g). LCMS (method 1): 165 (M+H)⁺, Rt 0.72 min. ¹H NMR (400 MHz, CDCl₃) δ ppm 1.80 (s, 6H) 7.54 (m, 1H) 8.49 (s, 1H) 8.61 (s, 1H).

### Step 2: Preparation of 2-(5-fluoro-1-oxido-pyridin-1-ium-3-yl)-2-methyl-propanenitrile (Intermediate I25)

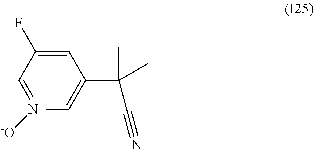

To a solution of 2-(5-fluoro-3-pyridyl)-2-methyl-propanenitrile (intermediate 134 prepared as described above) (2.75 g, 16.7 mmol) in acetonitrile (83.7 mL) was added hydrogen peroxide urea complex (4.06 g, 41.9 mmol) at 0° C., followed by dropwise addition of trifluoroacetic anhydride (5.88 mL, 41.9 mmol). The reaction mixture was stirred at 0° C. for 15 minutes and then at room temperature for 1.5 hours. After completion, it was dissolved with ethyl acetate and washed with an aqueous sodium bisulfite solution (˜20%). The aqueous layer was basified at 0-5° C. with sodium hydroxide (4 M) and extracted again with ethyl acetate. The combined organic layers were washed with sat. aq. sodium bicarbonate, dried over sodium sulfate, filtered and concentrated in vacuo. The residue was suspended in diisopropyl ether (˜5-7 mL) and stirred for 30 minutes. The white suspension was filtered and the solid was washed with fresh diisopropyl ether (2×2 mL) and pentane (2×5 mL). The residue was dried under vacuum to afford the desired product as white solid (2.73 g). LCMS (method 1): 181 (M+H)⁺, Rt 0.34 min. ¹H NMR (400 MHz, CDCl₃) δ ppm 1.78 (s, 6H) 7.21 (m, 1H) 8.12 (m, 1H) 8.21 (s, 1H).

### Step 3: Preparation of 2-[5-fluoro-1-oxido-6-[1-(2,2,3,3,3-pentafluoropropyl)pyrrolo[2,3-c]pyridin-5-yl]pyridin-1-ium-3-yl]-2-methyl-propanenitrile (Intermediate I18)

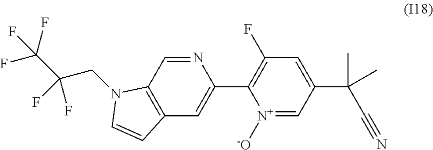

To a solution of 5-bromo-1-(2,2,3,3,3-pentafluoropropyl)pyrrolo[2,3-c]pyridine (intermediate 13 prepared as described above) (350 mg, 1.10 mmol) in tetrahydrofuran (3.50 mL) was added 2-(5-fluoro-1-oxido-pyridin-1-ium-3-yl)-2-methyl-propanenitrile (intermediate 125 prepared as described above) (230 mg, 1.30 mmol), followed by 2,2,6,6-tetramethylpiperidinylzinc chloride lithium chloride complex (1.0M in THF, 1.60 mL, 1.60 mmol). The reaction mixture was stirred at room temperature and degassed with argon for 5-10 minutes. Then [1,1′-bis(diphenylphosphino)ferrocene] palladium(II) dichloride dichloromethane adduct (44.0 mg, 0.0530 mmol) was added and the reaction mixture was stirred at 60° C. for 4.5 hours. Another portion of 2,2,6,6-tetramethylpiperidinylzinc chloride lithium chloride complex (1.0M in THF, 0.32 mL, 0.32 mmol) was added, the reaction was degassed again with argon for 5 min and the same amount of [1,1′-bis(diphenylphosphino)ferrocene] palladium(II) dichloride dichloromethane adduct (44.0 mg, 0.0530 mmol) was added. The reaction mixture was stirred at 60° C. overnight. After cooling to room temperature, the reaction mixture was extracted with ethyl acetate and brine. The combined organic layers were dried over magnesium sulfate, filtered and concentrated in vacuo. Purification by reversed phase chromatography (C-18 column, acetonitrile in water) afforded the desired product as beige solid (216 mg). LCMS (method 1): 429 (M+H)⁺, Rt 0.86 min. ¹H NMR (400 MHz, CDCl₃) δ ppm 1.80 (s, 6H) 4.80-4.90 (t, 2H) 6.73 (d, 1H) 7.34 (m, 2H) 8.01 (s, 1H) 8.40 (s, 1H) 8.95 (s, 1H).

### Step 4: Preparation of 2-[5-ethylsulfanyl-1-oxido-6-[1-(2,2,3,3,3-pentafluoropropyl)pyrrolo[2,3-c]pyridin-5-yl]pyridin-1-ium-3-yl]-2-methyl-propanenitrile (Pyridine N-Oxide PN6)

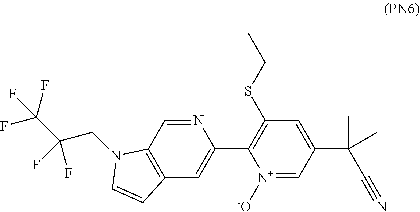

To a degassed solution of 2-[5-fluoro-1-oxido-6-[1-(2,2,3,3,3-pentafluoropropyl)pyrrolo[2,3-c]pyridin-5-yl]pyridin-1-ium-3-yl]-2-methyl-propanenitrile (intermediate 118 prepared as described above) (150 mg, 0.350 mmol) in N,N-dimethylformamide (3.50 mL) was added sodium ethanethiolate (77.5 mg, 0.876 mmol). The reaction mixture was stirred at room temperature for 30 minutes, then it was extracted with ethyl acetate and brine. The combined organic layers were dried over magnesium sulfate, filtered and concentrated in vacuo. Purification by reversed phase chromatography (C-18 column, acetonitrile in water) afforded the desired product as beige solid (105 mg). LCMS (method 1): 472 (M+H)⁺, Rt 0.91 min. ¹H NMR (400 MHz, CDCl₃) δ ppm 1.30 (t, 3H) 1.80 (s, 6H) 2.90-2.99 (q, 2H) 4.78-4.88 (t, 2H) 6.73 (d, 1H) 7.32 (m, 1H) 7.39 (d, 1H) 7.90 (s, 1H) 8.21 (s, 1H) 8.95 (s, 1H).

### Step 5: Preparation of 2-[5-ethylsulfanyl-6-[1-(2,2,3,3,3-pentafluoropropyl)pyrrolo[2,3-c]pyridin-5-yl]-3-pyridyl]-2-methyl-propanenitrile (Compound P29)

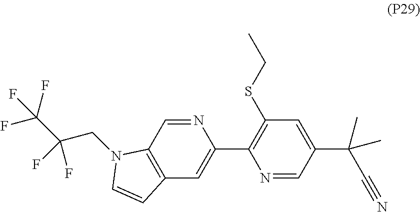 [SMILES: CCSc1cc(C(C)(C)C#N)cnc1-c1cc2ccn(CC(F)(F)C(F)(F)F)c2cn1]

To a solution of 2-[5-ethylsulfanyl-1-oxido-6-[1-(2,2,3,3,3-pentafluoropropyl)pyrrolo[2,3-c]pyridin-5-yl]pyridin-1-ium-3-yl]-2-methyl-propanenitrile (pyridine N-oxide PN6 prepared as described above) (76.0 mg, 0.160 mmol) in methanol (1.60 mL) was added zinc (activated) (21.0 mg, 0.320 mmol) followed by ammonium formate (31.0 mg, 0.480 mmol) and silica gel (380 mg, 6.30 mmol). The reaction mixture was stirred at room temperature for two nights, the same amounts of zinc and ammonium formate were added twice during this time. The suspension was filtered through a sintered glass filter plate and evaporated. Purification by reversed phase chromatography (C-18 Column, acetonitrile in water) afforded the desired product as a solid (49.0 mg). LCMS (method 1): 455 (M+H)⁺, Rt 0.94 min. ¹H NMR (400 MHz, CDCl₃) δ ppm 1.31 (t, 3H) 1.80 (s, 6H) 2.98-3.01 (q, 2H) 4.78-4.88 (t, 2H) 6.73 (d, 1H) 7.32 (m, 1H) 7.81 (d, 1H) 8.27 (s, 1H) 8.55 (s, 1H) 8.92 (s, 1H).

### Step 6: Preparation of 2-[5-ethylsulfonyl-6-[1-(2,2,3,3,3-pentafluoropropyl)pyrrolo[2,3-c]pyridin-5-yl]-3-pyridyl]-2-methyl-propanenitrile (Compound P19)

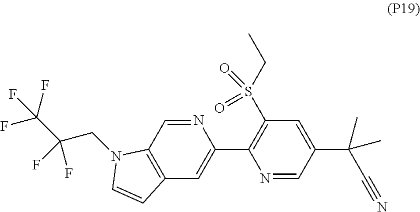 [SMILES: CCS(=O)(=O)c1cc(C(C)(C)C#N)cnc1-c1cc2ccn(CC(F)(F)C(F)(F)F)c2cn1]

To a stirred solution of 2-[5-ethylsulfanyl-6-[1-(2,2,3,3,3-pentafluoropropyl)pyrrolo[2,3-c]pyridin-5-yl]-3-pyridyl]-2-methyl-propanenitrile (compound P29 prepared as described above) (71.0 mg, 0.160 mmol) in dichloromethane (1.60 mL) was added 3-chloroperbenzoic acid (79.0 mg, 0.340 mmol) at 0° C. The reaction mixture was stirred for 4 hours, then another portion of 3-chloroperbenzoic acid (18.00 mg, 0.078 mmol) was added and stirring continued for 45 minutes. After completion, the reaction mixture was extracted with ethyl acetate and brine, the combined organic layers were dried over magnesium sulfate, filtered and evaporated. Purification by reversed phase chromatography (C-18 Column, acetonitrile in water) afforded the desired product as a beige solid (45.0 mg). LCMS (method 1): 487 (M+H)⁺, Rt 1.00 min. ¹H NMR (400 MHz, CDCl₃) δ ppm 1.40 (t, 3H) 1.88 (s, 6H) 4.00 (q, 2H) 4.78-4.88 (t, 2H) 6.73 (d, 1H) 7.32 (m, 1H) 8.17 (s, 1H) 8.55 (d, 1H) 8.80 (s, 1H) 9.08 (s, 1H).

### Example H5: Preparation of 5-[3-ethylsulfonyl-5-(2-pyridyloxy)-2-pyridyl]-1-(2,2,3,3,3-pentafluoro propyl)pyrrolo[2,3-c]pyridine (Compound P17)

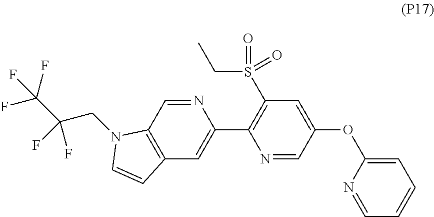 [SMILES: CCS(=O)(=O)c1cc(Oc2ccccn2)cnc1-c1cc2ccn(CC(F)(F)C(F)(F)F)c2cn1]

### Step 1: Preparation of 3-fluoro-5-(2-pyridyloxy)pyridine (Intermediate I36)

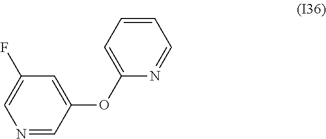 [SMILES: Fc1cncc(Oc2ccccn2)c1]

To a solution of 5-fluoropyridin-3-ol (CAS 209328-55-2) (1.00 g, 8.40 mmol) in N,N-dimethylformamide (10.0 mL) were added potassium carbonate (2.40 g, 17.0 mmol), copper(I) iodide (160 mg, 0.840 mmol) and 2-iodopyridine (2.70 g, 13.0 mmol). The reaction mixture was stirred at 110° C. for 7 hours. After completion, the reaction mixture was extracted with ethyl acetate and brine. Combined organic layers were dried over magnesium sulfate, filtered and concentrated in vacuo. Purification by flash chromatography over silica gel (ethyl acetate in cyclohexane) afforded the desired product as bright yellow solid (1.28 g). LCMS (method 1): 191 (M+H)⁺, Rt 0.78 min. ¹H NMR (400 MHz, CDCl₃) δ ppm 7.02 (d, 1H) 7.11 (m, 1H) 7.35 (m, 1H) 7.79 (m, 1H) 8.20 (m, 1H) 8.36 (m, 2H).

### Step 2: Preparation of 3-fluoro-1-oxido-5-(2-pyridyloxy)pyridin-1-ium (Intermediate 27)

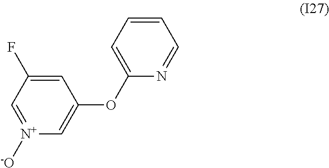

To a solution of 3-fluoro-5-(2-pyridyloxy)pyridine (intermediate 136 prepared as described above) (1.20 g, 6.30 mmol) in acetonitrile (32.0 mL) at 0° C. was added hydrogen peroxide urea complex (1.20 g, 13.0 mmol), followed by a dropwise addition of trifluoroacetic anhydride (1.80 mL, 13.0 mmol). The reaction mixture was stirred at 0-5° C. for 1 hour. After completion, the reaction mixture was carefully quenched with sodium bisulfite solution (˜40 mass %) and stirred for 15 minutes. A peroxide test was done before the mixture was basified at 0° C. with sodium hydroxide (4 N) to pH 10. Then it was diluted with ethyl acetate and washed with brine. The combined aqueous layer was extracted again with ethyl acetate and the combined organic layers were dried with sodium sulfate, filtered and concentrated in vacuo. Purification by flash chromatography over silica gel (methanol in dichloromethane) afforded the desired product as white solid (537 mg). LCMS (method 1): 207 (M+H)⁺, Rt 0.53 min. ¹H NMR (400 MHz, CDCl₃) δ ppm 7.02 (m, 2H) 7.16 (m, 1H) 7.80 (m, 1H) 8.01 (m, 1H) 8.10 (m, 1H) 8.22 (m, 1H).

### Step 3: Preparation of 5-[3-fluoro-1-oxido-5-(2-pyridyloxy)pyridin-1-ium-2-yl]-1-(2,2,3,3,3-pentafluoropropyl)pyrrolo[2,3-c]pyridine (Intermediate I32)

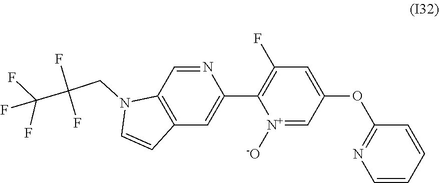

To a solution of 5-bromo-1-(2,2,3,3,3-pentafluoropropyl)pyrrolo[2,3-c]pyridine (intermediate 13 prepared as described above) (300 mg, 0.912 mmol) in tetrahydrofuran (3.00 mL) was added 3-fluoro-1-oxido-5-(2-pyridyloxy)pyridin-1-ium (intermediate 27 prepared as described above) (263 mg, 1.28 mmol), followed by slow addition of 2,2,6,6-tetramethylpiperidinylzinc chloride lithium chloride complex (1.0 M in THF, 2.30 mL, 2.28 mmol). The reaction mixture was stirred at room temperature and degassed with argon for 5-10 minutes. Then [1,1′-bis(diphenylphosphino)ferrocene] palladium(II) dichloride dichloromethane adduct (37.6 mg, 0.0456 mmol) was added and the reaction mixture was stirred at 75° C. for 2.5 hours. After cooling to room temperature, the reaction mixture was extracted with ethyl acetate and brine. The combined organic layers were dried over magnesium sulfate, filtered and concentrated in vacuo. Purification by reversed phase chromatography (C-18 column, acetonitrile in water) afforded the desired product as beige-brown solid (230 mg). LCMS (method 1): 455 (M+H)⁺, Rt 0.85 min. ¹H NMR (400 MHz, CDCl₃) δ ppm 4.78-4.90 (t, 2H) 6.73 (d, 1H) 7.08 (d, 1H) 7.18 (m, 2H) 7.32 (d, 1H) 7.80-7.85 (m, 1H) 8.01 (s, 1H) 8.28 (m, 2H) 8.94 (s, 1H).

### Step 4: Preparation of 5-[3-ethylsulfanyl-1-oxido-5-(2-pyridyloxy)pyridin-1-ium-2-yl]-1-(2,2,3,3,3-pentafluoropropyl)pyrrolo[2,3-c]pyridine (Pyridine N-Oxide PN10)

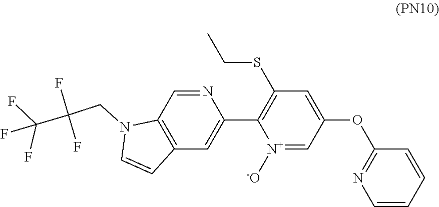

To a degassed solution of 5-[3-fluoro-1-oxido-5-(2-pyridyloxy)pyridin-1-ium-2-yl]-1-(2,2,3,3,3-pentafluoropropyl)pyrrolo[2,3-c]pyridine (intermediate 132 prepared as described above) (130 mg, 0.286 mmol) in N,N-dimethylformamide (2.86 mL) was added sodium ethanethiolate (27.9 mg, 0.315 mmol) at 0° C. The reaction mixture was stirred at 0-5° C. for 5.5 hours, then sodium ethanethiolate (12.7 mg, 0.143 mmol. 0.500 eq.) was again added and it was stirred two days at room temperature. Another portion of sodium ethanethiolate (12.7 mg, 0.143 mmol) was added and stirring continued for 2.5 hours. The reaction mixture was extracted with ethyl acetate and brine, the combined organic layers were dried over magnesium sulfate, filtered and concentrated in vacuo. Purification by reversed phase chromatography (C-18 column, acetonitrile in water) afforded the desired product as yellow foam (96 mg). LCMS (method 1): 498 (M+H)⁺, Rt 0.90 min. ¹H NMR (400 MHz, CDCl₃) δ ppm 1.27 (t, 3H) 2.82 (q, 2H) 4.78-4.88 (t, 2H) 6.72 (d, 1H) 7.08 (d, 1H) 7.13 (m, 2H) 7.32 (d, 1H) 7.78-7.83 (m, 1H) 7.91 (s, 1H) 8.20 (d, 1H) 8.23 (m, 1H) 8.92 (s, 1H).

### Step 5: Preparation of 5-[3-ethylsulfanyl-5-(2-pyridyloxy)-2-pyridyl]-1-(2,2,3,3,3-pentafluoropropyl) pyrrolo[2,3-c]pyridine (Compound P54)

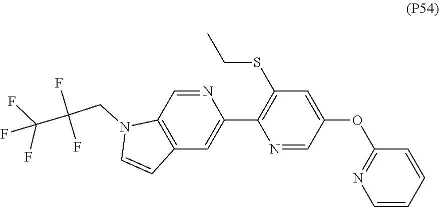 [SMILES: CCSc1cc(Oc2ccccn2)cnc1-c1cc2ccn(CC(F)(F)C(F)(F)F)c2cn1]

To a solution of 5-[3-ethylsulfanyl-1-oxido-5-(2-pyridyloxy)pyridin-1-ium-2-yl]-1-(2,2,3,3,3-pentafluoropropyl)pyrrolo[2,3-c]pyridine (pyridine N-oxide PN10 prepared as described above) (90.0 mg, 0.180 mmol) in methanol (1.80 mL) were added zinc (activated) (24.0 mg, 0.360 mmol), ammonium formate (35.0 mg, 0.540 mmol) and silica gel (450 mg, 7.40 mmol). The reaction mixture was stirred at room temperature over two days, then another portion of ammonium formate (35.0 mg, 0.540 mmol) and zinc (activated) (24.0 mg, 0.360 mmol) were added, and stirring continued for 4.5 hours. Addition of ammonium formate (35.0 mg, 0.540 mmol) and zinc (activated) (24.0 mg, 0.360 mmol) were repeated, and stirring continued overnight at room temperature. The reaction mixture was filtered through a pad of celite and washed with methanol. The filtrate was evaporated, and the residue was extracted with ethyl acetate and brine. The combined organic layers were dried over magnesium sulfate, filtered and concentrated in vacuo. Purification by flash chromatography over silica gel (ethyl acetate in cyclohexane) afforded the desired product as white solid (65.0 mg).

LCMS (method 1): 481 (M+H)⁺, Rt 0.91 min. ¹H NMR (400 MHz, CDCl₃) δ ppm 1.31 (t, 3H) 2.89 (q, 2H) 4.78-4.88 (t, 2H) 6.71 (d, 1H) 7.08 (d, 1H) 7.09 (m, 1H) 7.38 (s, 1H) 7.52 (d, 1H) 7.73-7.80 (m, 1H) 8.21 (d, 2H) 8.33 (d, 1H) 8.89 (s, 1H).

### Step 6: Preparation of 5-[3-ethylsulfonyl-5-(2-pyridyloxy)-2-pyridyl]-1-(2,2,3,3,3-pentafluoropropyl) pyrrolo[2,3-c]pyridine (Compound P17)

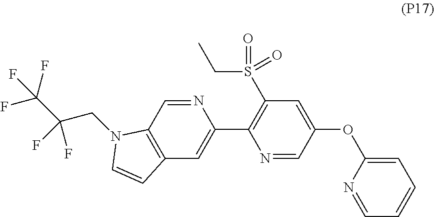 [SMILES: CCS(=O)(=O)c1cc(Oc2ccccn2)cnc1-c1cc2ccn(CC(F)(F)C(F)(F)F)c2cn1]

To a solution of 5-[3-ethylsulfanyl-5-(2-pyridyloxy)-2-pyridyl]-1-(2,2,3,3,3-pentafluoropropyl) pyrrolo[2,3-c]pyridine (compound P54 prepared as described above) (65.0 mg, 0.140 mmol) in dichloromethane (1.40 mL) was added 3-chloroperbenzoic acid (68.0 mg, 0.300 mmol). The reaction mixture was stirred at room temperature for 1 hour, then it was poured onto sat. aq. sodium bicarbonate (60.0 mL) and stirred for 30 minutes. The mixture was extracted with ethyl acetate and sat. aq. sodium bicarbonate and the combined organic layers were dried over magnesium sulfate, filtered and concentrated in vacuo. Purification by reversed phase chromatography (C-18 column, acetonitrile in water) afforded the desired product as beige solid (45 mg). LCMS (method 1): 513 (M+H)⁺, Rt 1.01 min. ¹H NMR (400 MHz, CDCl₃) δ ppm 1.33 (t, 3H) 3.55-3.62 (m, 1H) 3.82-3.92 (m, 1H) 4.61-4.71 (t, 2H) 6.71 (d, 1H) 7.10-7.17 (m, 2H) 7.29 (s, 1H) 7.69 (s, 1H) 7.80-7.86 (m, 1H) 8.20 (m, 1H) 8.28 (d, 1H) 8.55 (s, 1H) 8.82 (d, 1H).

### Example H6: Preparation of 1-[5-ethylsulfonyl-6-[1-(2,2,3,3,3-pentafluoropropyl)pyrazolo[3,4-c]pyridin-5-yl]-3-pyridyl]cyclopropanecarbonitrile (Compound P11)

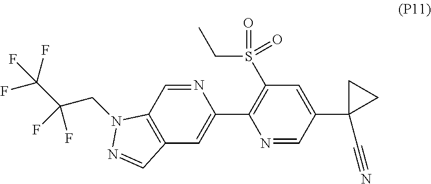 [SMILES: CCS(=O)(=O)c1cc(C2(C#N)CC2)cnc1-c1cc2cnn(CC(F)(F)C(F)(F)F)c2cn1]

### Step 1: Preparation of 1-(5-fluoro-3-pyridyl)cyclopropanecarbonitrile (Intermediate I35)

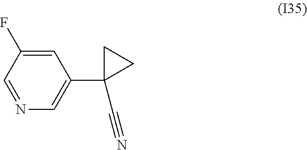 [SMILES: N#CC1(c2cncc(F)c2)CC1]

To a solution of 2-(5-fluoro-3-pyridyl) acetonitrile (CAS 39891-06-0) (4.00 g, 29.38 mmol) in dry acetonitrile (40.0 mL) was added cesium carbonate (28.7 g, 88.1 mmol) and 1,2-dibromoethane (5.06 mL, 58.8 mmol). The reaction mixture was stirred at 80° C. overnight, then cooled to room temperature before being concentrated in vacuo. The residue was diluted with water and extracted with ethyl acetate. The organic layer was washed with water and sat. aq. sodium bicarbonate, dried over magnesium sulfate, filtered and concentrated. Purification by flash chromatography over silica gel (ethyl acetate in cyclohexane) afforded the desired compound as light yellow solid. LCMS (method 1): 163 (M+H)⁺, Rt 0.67 min. ¹H NMR (400 MHz, CDCl₃) δ ppm 1.45-1.55 (m, 2H) 1.83-1.92 (m, 2H) 7.32-7.45 (m, 1H) 8.38-8.55 (m, 2H).

### Step 2: Preparation of 1-(5-fluoro-1-oxido-pyridin-1-ium-3-yl)cyclopropanecarbonitrile (Intermediate I26)

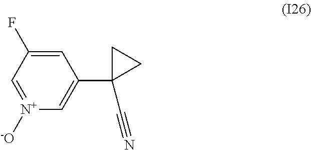

To a solution of 1-(5-fluoro-3-pyridyl)cyclopropanecarbonitrile (intermediate 135 prepared as described above) (150 mg, 0.925 mmol) in tetrahydrofuran (1.85 mL) was added hydrogen peroxide urea complex (182.7 mg, 1.94 mmol) at 0° C., followed by a dropwise addition of 2,2,2-trifluoroacetic-anhydride (0.261 mL, 1.85 mmol). The reaction mixture was stirred at room temperature for 16 hours and was quenched with an aqueous solution of sodium sulfite. It was stirred for 30 minutes, then aq. sat. sodium bicarbonate was added, and it was extracted with ethyl acetate twice. The combined organic layers were washed with aq. sat. sodium bicarbonate, dried over magnesium sulfate, filtered and concentrated. Purification by flash chromatography over silica gel (methanol in ethyl acetate) afforded the desired product as a white solid. LCMS (method 1): 179 (M+H)⁺, Rt 0.29 min. ¹H NMR (400 MHz, CDCl₃) δ ppm 1.46-1.55 (m, 2H) 1.86-1.98 (m, 2H) 7.01-7.09 (m, 1H) 7.95-8.02 (m, 1H) 8.05-8.13 (m, 1H)

### Step 3: Preparation of 1-[5-fluoro-1-oxido-6-[1-(2,2,3,3,3-pentafluoropropyl)pyrazolo[3,4-c]pyridin-5-yl]pyridin-1-ium-3-yl]cyclopropanecarbonitrile (Intermediate I15)

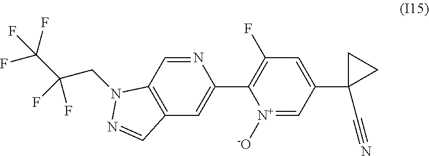

To a degassed solution of 1-(5-fluoro-1-oxido-pyridin-1-ium-3-yl)cyclopropanecarbonitrile (intermediate 126 prepared as described above) (1.60 g, 9.10 mmol) in tetrahydrofuran (20.0 mL) at 10° C. was added 2,2,6,6-tetramethylpiperidinylzinc chloride LiCl-complex (1.0 mol/L in THF) (9.10 mL, 9.10 mmol) dropwise. The reaction mixture was stirred for 15 minutes, then a degassed solution of 5-bromo-1-(2,2,3,3,3-pentafluoropropyl)pyrazolo[3,4-c]pyridine (intermediate 11 prepared as described above) (2.00 g, 6.1 mmol) in tetrahydrofuran was added, followed by [1,1′-bis(diphenylphosphino)ferrocene]dichloropalladium(II) (290 mg, 0.390 mmol). The reaction mixture was heated at 60° C. for 17 hours, cooled to room temperature and quenched with aq. sat. sodium bicarbonate, before being extracted with ethyl acetate. The combined organic layers were washed with brine, dried over sodium sulfate, filtered and concentrated in vacuo. Purification by flash chromatography over silica gel (methanol in ethyl acetate) afforded the desired product (735 mg). LCMS (method 5): 428 (M+H)⁺, Rt 0.92 min.

¹H NMR (400 MHz, CDCl₃) δ ppm 1.53-1.58 (m, 2H) 1.91-1.96 (m, 2H) 5.15 (t, 2H) 7.18 (dd, 1H) 8.15-8.28 (m, 3H) 9.14 (s, 1H).

### Step 4: Preparation of 1-[5-fluoro-6-[1-(2,2,3,3,3-pentafluoropropyl)pyrazolo[3,4-c]pyridin-5-yl]-3-pyridyl]cyclopropanecarbonitrile (Intermediate I16)

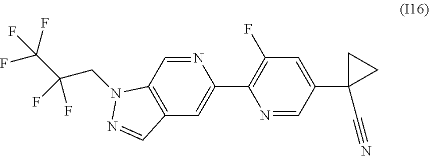 [SMILES: N#CC1(c2cnc(-c3cc4cnn(CC(F)(F)C(F)(F)F)c4cn3)c(F)c2)CC1]

To a suspension of 1-[5-fluoro-1-oxido-6-[1-(2,2,3,3,3-pentafluoropropyl)pyrazolo[3,4-c]pyridin-5-yl]pyridin-1-ium-3-yl]cyclopropanecarbonitrile (intermediate 115 prepared as described above) (820 mg, 1.92 mmol) in tetrahydrofuran (20.0 mL) and sat. ammonium chloride in water (5.00 mL) at 10° C. was added zinc (377 mg, 5.76 mmol). The reaction mixture was stirred overnight at 50° C., cooled to room temperature and dissolved with ethyl acetate. The suspension was filtered to remove the zinc, and the filtrate was washed with water and brine, dried over sodium sulfate, filtered and concentrated in vacuo. Purification by flash chromatography over silica gel (ethyl acetate in cyclohexane) afforded the desired product. LCMS (method 5): 412 (M+H)⁺, Rt 1.07 min. ¹H NMR (400 MHz, CDCl₃) δ ppm 1.55-1.59 (m, 2H) 1.86-1.96 (m, 2H) 5.16 (t, 2H) 7.53 (dd, 1H) 8.26 (s, 1H) 8.40 (s, 1H) 8.53 (s, 1H) 9.16 (s, 1H).

### Step 5: Preparation of 1-[5-ethylsulfanyl-6-[1-(2,2,3,3,3-pentafluoropropyl)pyrazolo[3,4-c]pyridin-5-yl]-3-pyridyl]cyclopropanecarbonitrile (Compound P10)

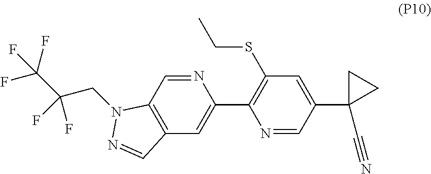 [SMILES: CCSc1cc(C2(C#N)CC2)cnc1-c1cc2cnn(CC(F)(F)C(F)(F)F)c2cn1]

To a solution of 1-[5-fluoro-6-[1-(2,2,3,3,3-pentafluoropropyl)pyrazolo[3,4-c]pyridin-5-yl]-3-pyridyl]cyclopropanecarbonitrile (intermediate 116 prepared as described above) (400 mg, 0.973 mmol) in dry dimethyl formamide (6.0 mL) at 0° C. under nitrogen was added sodium ethanethiolate (90% mass %, 200 mg, 2.14 mmol). The reaction mixture was stirred at room temperature for 3 hours. After completion it was diluted with water and extracted with ethyl acetate. Combined organic layers were washed with water and brine, dried over sodium sulfate, filtered and concentrated in vacuo. Purification by flash chromatography over silica gel (ethyl acetate in cyclohexane) afforded the desired product (190 mg). LCMS (method 5): 454 (M+H)⁺, Rt 1.14 min. ¹H NMR (400 MHz, CDCl₃) δ ppm 1.35 (t, 3H) 1.53-1.56 (m, 2H) 1.84-1.89 (m, 2H) 2.97 (q, 2H) 5.14 (t, 2H) 7.72 (d, 1H) 8.23-8.31 (m, 2H) 8.37 (d, 1H) 9.09 (s, 1H).

### Step 6: Preparation of 1-[5-ethylsulfonyl-6-[1-(2,2,3,3,3-pentafluoropropyl)pyrazolo[3,4-c]pyridin-5-yl]-3-pyridyl]cyclopropanecarbonitrile (Compound P11)

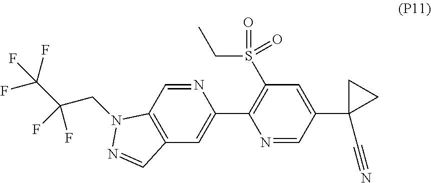 [SMILES: CCS(=O)(=O)c1cc(C2(C#N)CC2)cnc1-c1cc2cnn(CC(F)(F)C(F)(F)F)c2cn1]

To a solution of 1-[5-ethylsulfanyl-6-[1-(2,2,3,3,3-pentafluoropropyl)pyrazolo[3,4-c]pyridin-5-yl]-3-pyridyl]cyclopropanecarbonitrile (compound P10 prepared as described above) (175 mg, 0.386 mmol) in dichloromethane (10.0 mL) at 0° C. was added 3-chloroperbenzoic acid (199.8 mg, 0.811 mmol) portionwise. The reaction mixture was stirred for 2 hours warming from 0° C. to room temperature. After completion of reaction, dichloromethane and sodium hydroxide (2 N) were added, the aqueous layer was separated and extracted with dichloromethane. The combined organic layers were dried over sodium sulfate, filtered and concentrated in vacuo. Purification by flash chromatography over silica gel (ethyl acetate in cyclohexane) afforded the desired product (148 mg). LCMS (method 5): 486 (M+H)⁺, Rt 1.05 min. ¹H NMR (400 MHz, CDCl₃) δ ppm 1.43 (t, 3H) 1.59-1.64 (m, 2H) 1.93-1.98 (m, 2H) 4.00 (q, 2H) 5.15 (t, 2H) 8.25-8.31 (m, 3H) 8.95-9.01 (m, 2H).

### Example H7: Preparation of 5-(3-ethylsulfonyl-6-pyrimidin-2-yl-2-pyridyl)-1-(2,2,3,3,3-pentafluoro propyl)pyrazolo[3,4-c]pyridine (Compound P25)

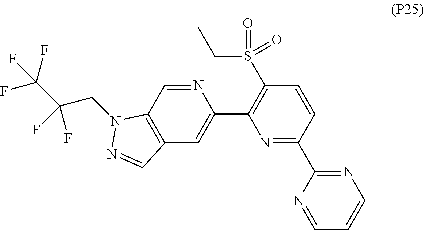 [SMILES: CCS(=O)(=O)c1ccc(-c2ncccn2)nc1-c1cc2cnn(CC(F)(F)C(F)(F)F)c2cn1]

### Step 1: Preparation of 5-(6-chloro-3-ethylsulfanyl-2-pyridyl)-1-(2,2,3,3,3-pentafluoropropyl) pyrazolo[3,4-c]pyridine (Compound P53)

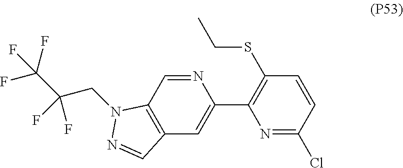 [SMILES: CCSc1ccc(Cl)nc1-c1cc2cnn(CC(F)(F)C(F)(F)F)c2cn1]

A solution of 5-(3-ethylsulfanyl-1-oxido-pyridin-1-ium-2-yl)-1-(2,2,3,3,3-pentafluoropropyl) pyrazolo[3,4-c]pyridine (pyridine N-oxide PN1 prepared as described above) (3.50 g, 8.70 mmol) in phosphoryl chloride (35.0 mL, 370 mmol) was stirred at room temperature for 2 hours. The reaction mixture was poured onto ice cold water and neutralized with sat. aq. sodium bicarbonate. Then it was extracted with ethyl acetate (3×50 mL), the combined organic layers were dried over magnesium sulfate, filtered and evaporated. Purification by flash chromatography over silica gel (ethyl acetate in cyclohexane) afforded the desired product (2.00 g). LCMS (method 5): 424/426 (M+H)⁺, Rt 1.26 min.

Similarly, 6-(6-chloro-3-ethylsulfanyl-2-pyridyl)-3-(2,2,3,3,3-pentafluoropropyl)imidazo[4,5-c]pyridine (compound P43) can be prepared from 6-(3-ethylsulfanyl-1-oxido-pyridin-1-ium-2-yl)-3-(2,2,3,3,3-pentafluoropropyl)imidazo[4,5-c]pyridine (pyridine N-oxide PN3 prepared as described above). LCMS (method 1): 423/425 (M+H)⁺, Rt 1.03 min.

### Step 2: Preparation of 5-(6-chloro-3-ethylsulfonyl-2-pyridyl)-1-(2,2,3,3,3-pentafluoropropyl) pyrazolo[3,4-c]pyridine (Compound P49)

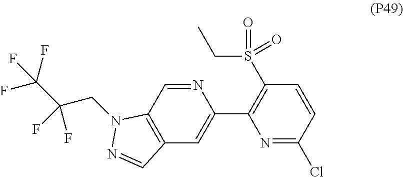 [SMILES: CCS(=O)(=O)c1ccc(Cl)nc1-c1cc2cnn(CC(F)(F)C(F)(F)F)c2cn1]

To a solution of 5-(6-chloro-3-ethylsulfanyl-2-pyridyl)-1-(2,2,3,3,3-pentafluoropropyl) pyrazolo[3,4-c]pyridine (compound P53 prepared as described above) (1.90 g, 4.50 mmol) in dichloromethane (19.0 mL) was added 3-chloroperbenzoic acid (2.30 g, 9.40 mmol) at 0° C. portionwise. The reaction mixture was stirred at room temperature for 2-3 hours, then it was poured onto sat. aq. sodium bicarbonate and extracted with dichloromethane. The combined organic layers were dried over sodium sulfate, filtered and concentrated in vacuo. Purification by flash chromatography over silica gel (ethyl acetate in cyclohexane) afforded the desired product (780 mg). LCMS (method 5): 455/457 (M+H)⁺, Rt 1.16 min. ¹H NMR (400 MHz, CDCl₃) δ ppm 1.42 (t, 3H) 4.00 (q, 2H) 5.15 (t, 2H) 7.56 (d, 1H) 8.27 (s, 1H) 8.34 (d, 1H) 8.46 (d, 1H) 8.99 (s, 1H).

Similarly, 6-(6-chloro-3-ethylsulfonyl-2-pyridyl)-3-(2,2,3,3,3-pentafluoropropyl)imidazo[4,5-c]pyridine (compound P41) can be prepared from 6-(6-chloro-3-ethylsulfanyl-2-pyridyl)-3-(2,2,3,3,3-pentafluoropropyl)imidazo[4,5-c]pyridine (compound P43 prepared as described above). LCMS (method 1): 455/457 (M+H)+, Rt 0.96 min.

### Step 3: Preparation of 5-(3-ethylsulfonyl-6-pyrimidin-2-yl-2-pyridyl)-1-(2,2,3,3,3-pentafluoropropyl) pyrazolo[3,4-c]pyridine (Compound P25)

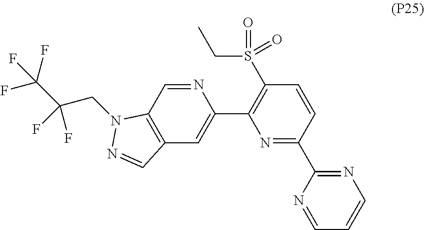 [SMILES: CCS(=O)(=O)c1ccc(-c2ncccn2)nc1-c1cc2cnn(CC(F)(F)C(F)(F)F)c2cn1]

In a microwave vial, a solution of 5-(6-chloro-3-ethylsulfonyl-2-pyridyl)-1-(2,2,3,3,3-pentafluoropropyl) pyrazolo[3,4-c]pyridine (compound P49 prepared as described above) (230 mg, 0.506 mmol) in 1,4-dioxane (4.60 mL) was degassed with nitrogen for 10 minutes. Then tributyl(pyrimidin-2-yl)stannane (0.176 mL, 0.556 mmol) and tetrakis(triphenylphosphine)palladium(0) (117 mg, 0.101 mmol) were added, and the reaction mixture was stirred in the microwave at 150° C. for 2 hours. After cooling to room temperature, the reaction mixture was evaporated and the residue was purified by flash chromatography over silica gel (ethyl acetate in cyclohexane) to afford the desired product (130 mg).

LCMS (method 5): 499 (M+H)⁺, Rt 1.02 min. ¹H NMR (400 MHz, CDCl₃) δ ppm 1.44 (t, 3H) 3.97 (q, 2H) 5.16 (t, 2H) 7.42 (t, 1H) 8.27 (s, 1H) 8.43 (d, 1H) 8.68 (d, 1H) 8.77 (d, 1H) 9.00 (d, 3H).

### Example H8: Preparation of 6-(5-cyclopropyl-3-ethylsulfanyl-1-oxido-pyridin-1-ium-2-yl)-3-(2,2,3,3,3-pentafluoropropyl)imidazo[4,5-c]pyridine (Compound PN4)

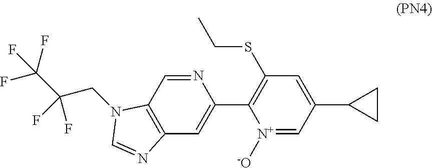

### Step 1: Preparation of 3-cyclopropyl-5-fluoro-1-oxido-pyridin-1-ium (Intermediate I24)

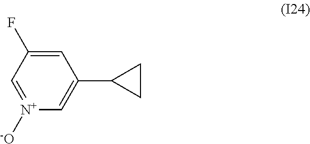

To a solution of 3-bromo-5-fluoropyridine 1-oxide (CAS 1221793-60-7) (200 mg, 0.990 mmol) in toluene (3.00 mL) and water (0.089 mL) were added cyclopropylboronic acid (260 mg, 3.00 mmol) and tripotassium phosphate (1.30 g, 5.90 mmol). The reaction mixture was degassed with argon for 5 minutes, then tetrakis(triphenylphosphine)palladium(0) (110 mg, 0.099 mmol) was added, and it was stirred in the microwave for 40 minutes at 120° C. After cooling to room temperature, the reaction mixture was diluted with water and extracted with ethyl acetate. The combined organic layers were washed with brine, dried over magnesium sulfate, filtered and concentrated in vacuo. Purification by flash chromatography over silica gel (ethyl acetate in cyclohexane) afforded the desired product as beige solid (87.3 mg). LCMS (method 1): 154 (M+H)⁺, Rt 0.43 min. 1H NMR (400 MHz, CDCl₃) δ ppm 0.82 (s, 2H) 1.10-1.19 (m, 2H) 1.79-1.93 (m, 1H) 6.69-6.78 (m, 1H) 7.85-7.91 (m, 1H) 7.94-8.02 (m, 1H).

### Step 2: Preparation of 6-(5-cyclopropyl-3-fluoro-1-oxido-pyridin-1-ium-2-yl)-3-(2,2,3,3,3-pentafluoro propyl)imidazo[4.5-c]pyridine (Intermediate I29)

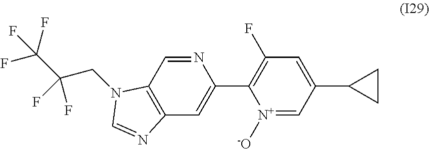

To a solution of 6-bromo-3-(2,2,3,3,3-pentafluoropropyl)imidazo[4,5-c]pyridine (intermediate 14 prepared as described above) (1.80 g, 5.45 mmol) in tetrahydrofuran (18.0 mL) was added 3-cyclopropyl-5-fluoro-1-oxido-pyridin-1-ium (intermediate 124) (919 mg, 6.00 mmol), followed by dropwise addition of 2,2,6,6-tetramethylpiperidinylzinc chloride lithium chloride complex (1.0M in THF, 24.5 mL, 24.5 mmol). The reaction mixture was stirred at room temperature and degassed with argon for 5-10 minutes. Then [1,1′-bis(diphenylphosphino)ferrocene] palladium(II) dichloride dichloro-methane adduct (156 mg, 0.191 mmol) was added, and the reaction mixture was stirred at 60° C. overnight. Another portion of 2,2,6,6-tetramethylpiperidinylzinc chloride lithium chloride complex (1.0M in THF, 5.00 mL) and [1,1′-bis(diphenylphosphino)ferrocene] palladium(II) dichloride dichloromethane adduct (15.0 mg) were added. The reaction mixture stirred again at 65° C. overnight. After cooling to room temperature, the reaction mixture was dissolved with ethyl acetate and aq. sat. sodium bicarbonate was added slowly. The mixture was filtered through hyflo and the filtrate was extracted with ethyl acetate and sat. aq. sodium bicarbonate. The combined organic layers were dried over magnesium sulfate, filtered and concentrated in vacuo. Purification by flash chromatography over silica gel (methanol in ethylacetate) afforded the desired product as light brown solid (1.35 g). LCMS (method 1): 403 (M+H)⁺, Rt 0.80 min. 1H NMR (400 MHz, MeOD) δ ppm 0.90-1.01 (m, 2H) 1.15-1.30 (m, 2H) 2.05-2.17 (m, 1H) 5.40-5.60 (m, 2H) 7.24-7.38 (m, 1H) 8.08-8.18 (m, 1H) 8.21-8.31 (m, 1H) 8.57-8.69 (m, 1H) 9.12-9.24 (m, 1H).

### Step 3: Preparation of 6-(5-cyclopropyl-3-ethylsulfanyl-1-oxido-pyridin-1-ium-2-yl)-3-(2,2,3,3,3-pentafluoropropyl)imidazo[4,5-c]pyridine (Pyridine N-Oxide PN4)

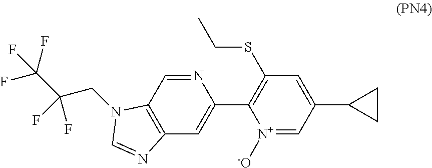

To a solution of 6-(5-cyclopropyl-3-fluoro-1-oxido-pyridin-1-ium-2-yl)-3-(2,2,3,3,3-pentafluoro propyl)imidazo[4,5-c]pyridine (intermediate 129) (1.34 g, 3.33 mmol) in N,N-dimethylformamide (33.3 mL) was added sodium ethanethiolate (1.09 g, 11.7 mmol) at 10° C. portionwise. The reaction mixture was stirred at room temperature for 4 hours, then additional sodium ethanethiolate (250 mg) was added, and stirring continued for 6 hours. The reaction mixture was poured onto sat. aq. sodium carbonate, the aqueous layer was extracted with ethyl acetate and the combined organic layers were washed with water and sat. aq. sodium carbonate before being dried over magnesium sulfate, filtered and concentrated in vacuo. Purification by flash chromatography over silica gel (methanol in ethyl acetate) afforded the desired product as brown amorph solid (205.6 mg). LCMS (method 1): 445 (M+H)⁺, Rt 0.86 min. 1H NMR (400 MHz, MeOD) δ ppm 0.87-1.01 (m, 2H) 1.10-1.23 (m, 2H) 1.24-1.32 (m, 3H) 2.04-2.16 (m, 1H) 2.89-3.06 (m, 2H) 5.40-5.59 (m, 2H) 7.32-7.39 (m, 1H) 7.92 (d, 1H) 8.05 (d, 1H) 8.57-8.67 (m, 1H) 9.11-9.20 (m, 1H).

### Example H9: Preparation of 6-[3-ethylsulfonyl-6-(1,2,4-triazol-1-yl)-2-pyridyl]-3-(2,2,3,3,3-pentafluoropropyl)imidazo[4,5-c]pyridine (Compound P32)

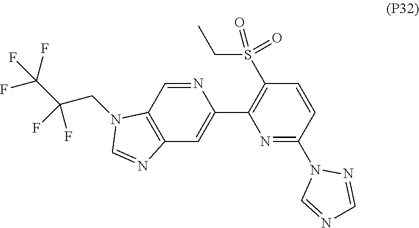 [SMILES: CCS(=O)(=O)c1ccc(-n2cncn2)nc1-c1cc2ncn(CC(F)(F)C(F)(F)F)c2cn1]

To a solution of 6-(6-chloro-3-ethylsulfonyl-2-pyridyl)-3-(2,2,3,3,3-pentafluoropropyl)imidazo[4,5-c]pyridine (compound P41 prepared as described above) (255 mg, 0.561 mmol) in pyridine (4.49 mL) was added 1H-1,2,4-triazole (58.1 mg, 0.841 mmol). The reaction mixture was stirred at 120° C. overnight. After cooling to room temperature, it was quenched with ice-water (35 mL) and extracted with ethyl acetate. The combined organic layers were washed with water and brine, dried over sodium sulfate, filtered and concentrated in vacuo. Purification by flash chromatography over silica gel (ethyl acetate in cyclohexane) afforded the desired product as a solid (75.8 mg). LCMS (method 1): 488 (M+H)⁺, Rt 0.89 min. 1H NMR (400 MHz, N,N-DMF-dr) δ ppm 1.26-1.38 (m, 3H) 4.14 (q, 2H) 5.56-5.78 (m, 2H) 8.10-8.19 (m, 1H) 8.41 (d, 1H) 8.47 (d, 1H) 8.69-8.78 (m, 2H) 9.16 (s, 1H) 9.62 (s, 1H).

TABLE P
Examples of compounds of formula (I)

| | | | LCMS | | | |
|---|---|---|---|---|---|---|
| | | | | [M + H]⁺ | | Mp |
| No. | IUPAC name | Structures | Rₜ (min) | (measured) | Method | (° C.) |
| P1 | 5-ethylsulfonyl-N- methyl-6- [1-(2,2,3,3,3- pentafluoropropyl) pyrazolo[3,4-c] pyridin-5-yl]pyridin- 3-amine | 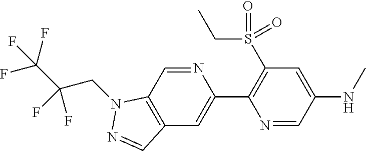 [SMILES: CCS(=O)(=O)c1cc(NC)cnc1-c1cc2cnn(CC(F)(F)C(F)(F)F)c2cn1] | 0.89 | 450 | 1 | |
| P2 | N-[5-ethylsulfonyl-6-[1- (2,2,3,3,3- pentafluoropropyl) pyrazolo[3,4-c] pyridin-5-yl]-3-pyridyl]- N-methyl-acetamide | 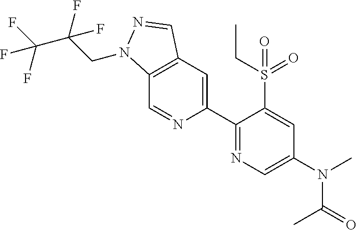 [SMILES: CCS(=O)(=O)c1cc(N(C)C(C)=O)cnc1-c1cc2cnn(CC(F)(F)C(F)(F)F)c2cn1] | 0.88 | 492 | 1 | 189- 190 |
| P3 | 5-(3-ethylsulfanyl-2- pyridyl)-1-(2,2,3,3,3- pentafluoropropyl] pyrazolo[3,4-c]pyridine | 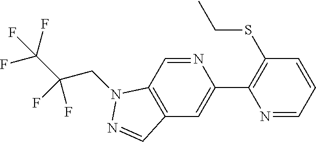 [SMILES: CCSc1cccnc1-c1cc2cnn(CC(F)(F)C(F)(F)F)c2cn1] | 0.98 | 389 | 1 | |
| P4 | 5-(3-ethylsulfonyl-2- pyridyl)-1-(2,2,3,3,3- pentafluoropropyl) pyrazolo[3,4-c]pyridine | 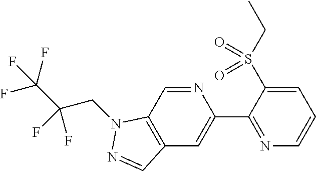 [SMILES: CCS(=O)(=O)c1cccnc1-c1cc2cnn(CC(F)(F)C(F)(F)F)c2cn1] | 0.91 | 421 | 1 | 143- 144 |
| P5 | 2-[5-ethylsulfonyl-6-[1- (2,2,3,3,3- pentafluoropropyl) pyrazolo[3,4-c]pyridin- 5-yl]-3- pyridyl]-2-methyl- propanenitrile | 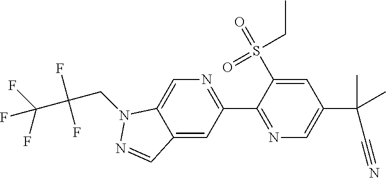 [SMILES: CCS(=O)(=O)c1cc(C(C)(C)C#N)cnc1-c1cc2cnn(CC(F)(F)C(F)(F)F)c2cn1] | 1.06 | 488 | 5 | 170- 172 |
| P6 | 2-[5-ethylsulfanyl-6-[1- (2,2,3,3,3- pentafluoropropyl) pyrazolo[3,4-c]pyridin- 5-yl]-3-pyridyl]-2- methyl-propanenitrile | 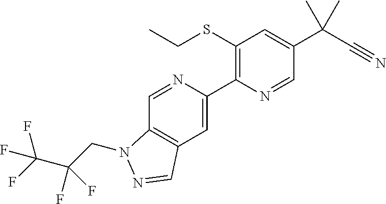 [SMILES: CCSc1cc(C(C)(C)C#N)cnc1-c1cc2cnn(CC(F)(F)C(F)(F)F)c2cn1] | 1.13 | 456 | 5 | |
| P7 | 5-(3-ethylsulfinyl-2- pyridyl)-1-(2,2,3,3,3- pentafluoropropyl) pyrrolo[2,3-c]pyridine | 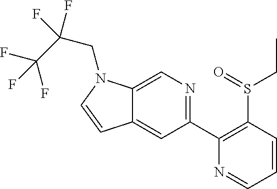 [SMILES: CCS(=O)c1cccnc1-c1cc2ccn(CC(F)(F)C(F)(F)F)c2cn1] | 0.98 | 404 | 1 | |
| P8 | 5-(3-ethylsulfonyl-2- pyridyl)-1-(2,2,3,3,3- pentafluoropropyl) pyrrolo[2,3-c]pyridine | 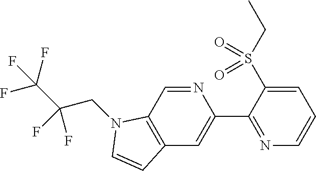 [SMILES: CCS(=O)(=O)c1cccnc1-c1cc2ccn(CC(F)(F)C(F)(F)F)c2cn1] | 0.89 | 420 | 1 | |
| P9 | 6-(5-cyclopropyl-3- ethylsulfanyl-2-pyridyl)- 3-(2,2,3,3,3- pentafluoropropyl) imidazo[4,5-c]pyridine | 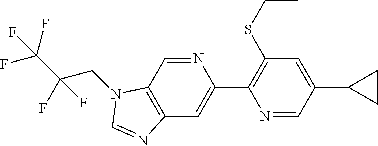 [SMILES: CCSc1cc(C2CC2)cnc1-c1cc2ncn(CC(F)(F)C(F)(F)F)c2cn1] | 0.97 | 429 | 1 | |
| P10 | 1-[5-ethylsulfanyl-6-[1- (2,2,3,3,3- pentafluoropropyl) pyrazolo[3,4-c]pyridin- 5-yl]-3-pyridyl] cyclopropane- carbonitrile | 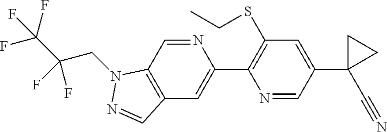 [SMILES: CCSc1cc(C2(C#N)CC2)cnc1-c1cc2cnn(CC(F)(F)C(F)(F)F)c2cn1] | 114 | 454 | 5 | 163- 165 |
| P11 | 1-[5-ethylsulfonyl-6-[1- (2,2,3,3,3- pentafluoropropyl) pyrazolo[3,4-c]pyridin- 5-yl]-3-pyridyl] cyclopropane- carbonitrile | 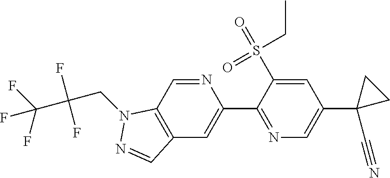 [SMILES: CCS(=O)(=O)c1cc(C2(C#N)CC2)cnc1-c1cc2cnn(CC(F)(F)C(F)(F)F)c2cn1] | 1.05 | 486 | 5 | 182- 184 |
| P12 | N-[5-ethylsulfonyl-6-[1- (2,2,3,3,3- pentafluoropropyl) pyrazolo[3,4-c]pyridin- 5-yl]-2-pyridyl]-N- methyl-acetamide | 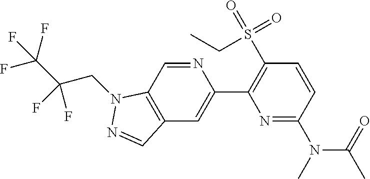 [SMILES: CCS(=O)(=O)c1ccc(N(C)C(C)=O)nc1-c1cc2cnn(CC(F)(F)C(F)(F)F)c2cn1] | 1.32 | 492 | 5 | 208- 210 |
| P13 | 1-[5-ethylsulfonyl-6-[1- (2,2,3,3,3- pentafluoropropyl) pyrrolo[2,3-c]pyridin- 5-yl]-2-pyridyl]- 1,3-dimethyl-urea | 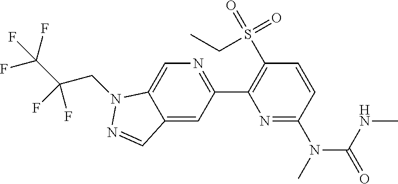 [SMILES: CCS(=O)(=O)c1ccc(N(C)C(=O)NC)nc1-c1cc2cnn(CC(F)(F)C(F)(F)F)c2cn1] | 0.93 | 506 | 1 | 214- 217 |
| P14 | 5-ethylsulfonyl-N- methyl-6- [1-(2,2,3,3,3- pentafluoropropyl) pyrazolo[3,4-c]pyridin- 5-yl]pyridin-2-amine | 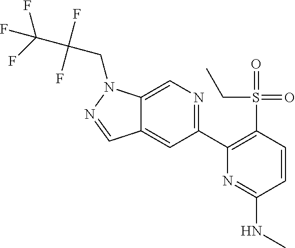 [SMILES: CCS(=O)(=O)c1ccc(NC)nc1-c1cc2cnn(CC(F)(F)C(F)(F)F)c2cn1] | 1.04 | 450 | 5 | 180- 182 |
| P15 | N-[5-ethylsulfonyl-6- [1-(2,2,3,3,3- pentafluoropropyl) pyrazolo[3,4-c]pyridin- 5-yl]-2-pyridyl]-N- methyl-cyclopropane- carboxamide | 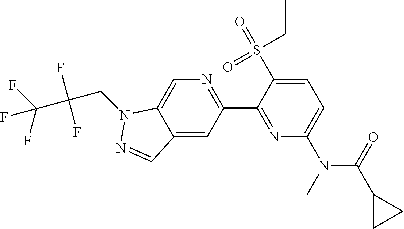 [SMILES: CCS(=O)(=O)c1ccc(N(C)C(=O)C2CC2)nc1-c1cc2cnn(CC(F)(F)C(F)(F)F)c2cn1] | 1.35 | 518 | 5 | 196- 198 |
| P16 | N-[5-ethylsulfonyl-6- [1-(2,2,3,3,3- pentafluoropropyl) pyrrolo[2,3-c]pyridin- 5-yl]-3-pyridyl]- N-methyl-acetamide | 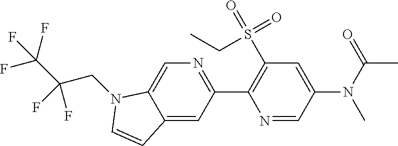 [SMILES: CCS(=O)(=O)c1cc(N(C)C(C)=O)cnc1-c1cc2ccn(CC(F)(F)C(F)(F)F)c2cn1] | 0.87 | 491 | 1 | 224- 227 |
| P17 | 5-[3-[3-5-(2- pyridyloxy)-2-pyridyl]- 1-(2,2,3,3,3- pentafluoropropyl) pyrrolo[2,3-c]pyridine | 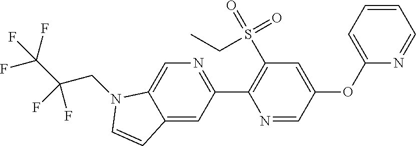 [SMILES: CCS(=O)(=O)c1cc(Oc2ccccn2)cnc1-c1cc2ccn(CC(F)(F)C(F)(F)F)c2cn1] | 1.01 | 513 | 1 | 193- 196 |
| P18 | 5-ethylsulfonyl-N- methyl-6-[1-(2,2,3,3,3- pentafluoropropyl) pyrrolo[2,3-c]pyridin- 5-yl]pyridin-3-amine | 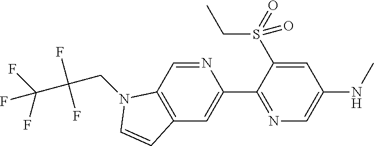 [SMILES: CCS(=O)(=O)c1cc(NC)cnc1-c1cc2ccn(CC(F)(F)C(F)(F)F)c2cn1] | 0.76 | 449 | 1 | |
| P19 | 2-[5-ethylsulfonyl-6- [1-(2,2,3,3,3- pentafluoropropyl) pyrrolo[2,3-c]pyridin- 5-yl]-3-pyridyl]- 2-methyl-propanenitrile | 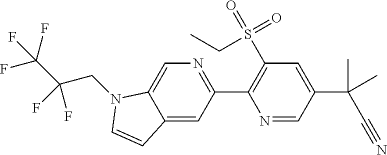 [SMILES: CCS(=O)(=O)c1cc(C(C)(C)C#N)cnc1-c1cc2ccn(CC(F)(F)C(F)(F)F)c2cn1] | 1.00 | 487 | 1 | 193- 197 |
| P20 | 5-[3-ethylsulfanyl-5-(2- pyridyloxy)-2-pyridyl]- 1-(2,2,3,3,3- pentafluoropropyl) pyrazolo [3,4-c]pyridine | 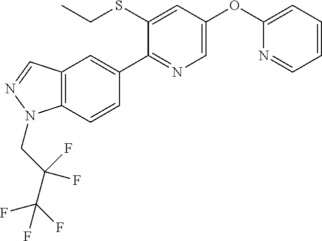 [SMILES: CCSc1cc(Oc2ccccn2)cnc1-c1ccc2c(cnn2CC(F)(F)C(F)(F)F)c1] | 1.30 | 482 | 5 | 154- 156 |
| P21 | 5-[3-ethylsulfonyl-5-(2- pyridyloxy)-2-pyridyl]- 1-pentafluoropropyl) pyrazolo[3,4-c]pyridine | 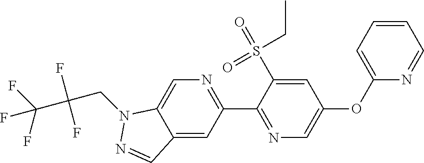 [SMILES: CCS(=O)(=O)c1cc(Oc2ccccn2)cnc1-c1cc2cnn(CC(F)(F)C(F)(F)F)c2cn1] | 1.17 | 514 | 5 | 166- 168 |
| P22 | 5-[3-ethylsulfonyl-6- (1,2,4-triazol-1-yl)-2- pyridyl]-1-(2,2,3,3,3- pentafluoropropyl) pyrazolo[3,4-c] pyridine | 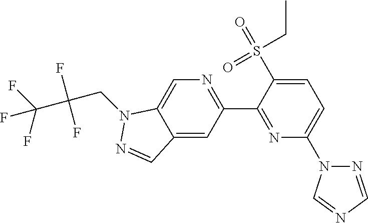 [SMILES: CCS(=O)(=O)c1ccc(-n2cncn2)nc1-c1cc2cnn(CC(F)(F)C(F)(F)F)c2cn1] | 1.43 | 488 | 3 | 240- 242 |
| P23 | 1-[5-ethylsulfonyl-6- [1-(2,2,3,3,3- pentafluoropropyl) pyrrolo[2,3-c]pyridin- 5-yl]-3-pyridyl] cyclopropane- carbonitrile | 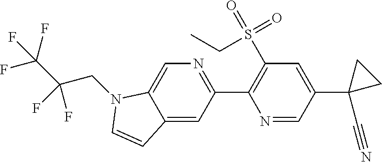 [SMILES: CCS(=O)(=O)c1cc(C2(C#N)CC2)cnc1-c1cc2ccn(CC(F)(F)C(F)(F)F)c2cn1] | 0.96 | 485 | 1 | 181- 184 |
| P24 | 5-ethylsulfonyl-N- methyl-6- [1-(2,2,3,3,3- pentafluoropropyl) pyrrolo[2,3-c]pyridin- 5-yl]pyridin-2- amine | 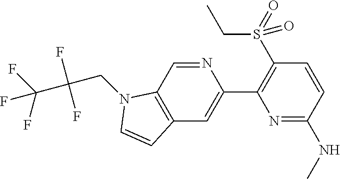 [SMILES: CCS(=O)(=O)c1ccc(NC)nc1-c1cc2ccn(CC(F)(F)C(F)(F)F)c2cn1] | 0.80 | 449 | 1 | |
| P25 | 5-(3-ethylsulfonyl-6- pyrimidin-2-yl-2- pyridyl)-1-(2,2,3,3,3- pentafluoropropyl) pyrazolo[3,4-c]pyridine | 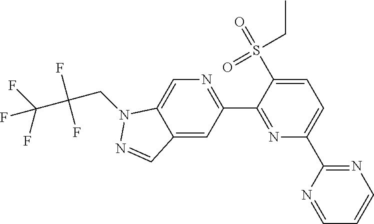 [SMILES: CCS(=O)(=O)c1ccc(-c2ncccn2)nc1-c1cc2cnn(CC(F)(F)C(F)(F)F)c2cn1] | 1.02 | 499 | 5 | 176- 178 |
| P26 | 5-(6-cyclopropyl-3- ethylsulfonyl-2- pyridyl)-1-(2,2,3,3,3,- pentafluoropropyl) pyrazolo[3,4-c]pyridine | 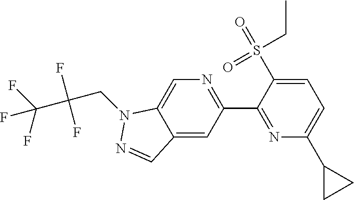 [SMILES: CCS(=O)(=O)c1ccc(C2CC2)nc1-c1cc2cnn(CC(F)(F)C(F)(F)F)c2cn1] | 1.14 | 461 | 5 | 196- 197 |
| P27 | 3-[5-ethylsulfonyl-6- [1-(2,2,3,3,3- pentafluoropropyl) pyrazolo[3,4-c] pyridin-5-yl]-2- pyridyl]oxazolidin- 2-one | 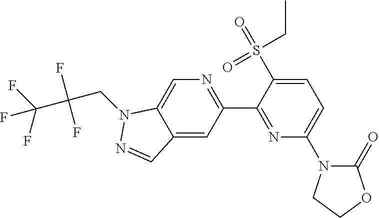 [SMILES: CCS(=O)(=O)c1ccc(N2CCOC2=O)nc1-c1cc2cnn(CC(F)(F)C(F)(F)F)c2cn1] | 1.07 | 506 | 5 | 242- 244 |
| P28 | 5-(5-cyclopropyl-3- ethylsulfonyl-2- pyridyl)-1-(2,2,3,3,3- pentafluoropropyl) pyrazolo[3,4-c] pyridine | 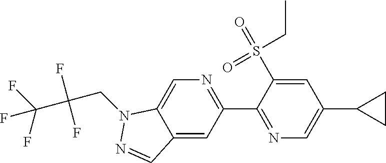 [SMILES: CCS(=O)(=O)c1cc(C2CC2)cnc1-c1cc2cnn(CC(F)(F)C(F)(F)F)c2cn1] | 1.07 | 461 | 5 | 218- 221 |
| P29 | 2-[5-ethylsulfanyl-6- [1-(2,2,3,3,3- pentafluoropropyl) pyrrolo[2,3-c]pyridin- 5-yl]-3-pyridyl]- 2-methyl-propanenitrile | 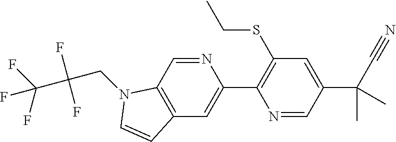 [SMILES: CCSc1cc(C(C)(C)C#N)cnc1-c1cc2ccn(CC(F)(F)C(F)(F)F)c2cn1] | 0.94 | 455 | 1 | 132- 135 |
| P30 | 5-[3-ethylsulfanyl-5- (trifluoromethyl)-2- pyridyl]-1-(2,2,3,3,3- pentafluoropropyl) pyrazolo[3,4-c] pyridine | 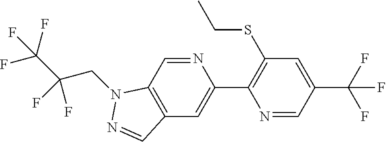 [SMILES: CCSc1cc(C(F)(F)F)cnc1-c1cc2cnn(CC(F)(F)C(F)(F)F)c2cn1] | 1.23 | 457 | 5 | 126- 128 |
| P31 | 5-[3-ethylsulfonyl-5- (trifluoromethyl)-2- pyridyl]-1-(2,2,3,3,3- pentafluoropropyl) pyrazolo[3,4-c] pyridine | 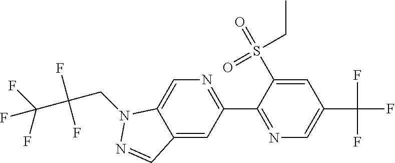 [SMILES: CCS(=O)(=O)c1cc(C(F)(F)F)cnc1-c1cc2cnn(CC(F)(F)C(F)(F)F)c2cn1] | 1.15 | 489 | 5 | 182- 184 |
| P32 | 6-[3-ethylsulfonyl-6- (1,2,4-triazol-1-yl)- 2-pyridyl]-3- (2,2,3,3,3- pentafluoropropyl) imidazo[4,5-c] pyridine | 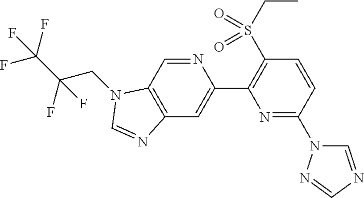 [SMILES: CCS(=O)(=O)c1ccc(-n2cncn2)nc1-c1cc2ncn(CC(F)(F)C(F)(F)F)c2cn1] | 0.89 | 488 | 1 | 222- 227 |
| P33 | 1-[5-ethylsulfonyl-6- [1-(2,2,3,3,3- pentafluoropropyl) pyrazolo[3,4-c] pyridin-5-yl]-2- pyridyl]-1,3- dimethyl-urea | 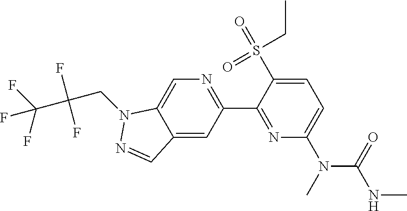 [SMILES: CCS(=O)(=O)c1ccc(N(C)C(=O)NC)nc1-c1cc2cnn(CC(F)(F)C(F)(F)F)c2cn1] | 0.98 | 507 | 5 | 200- 202 |
| P34 | 1-[5-ethylsulfonyl-6- [3-(2,2,3,3,3- pentafluoropropyl) imidazo [4,5-c]pyridin-6-yl]-3- pyridyl]cyclopropane- carbonitrile | 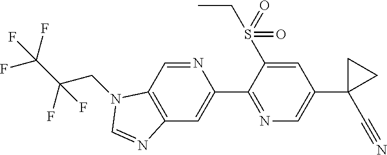 [SMILES: CCS(=O)(=O)c1cc(C2(C#N)CC2)cnc1-c1cc2ncn(CC(F)(F)C(F)(F)F)c2cn1] | 0.89 | 486 | 1 | 270- 275 |
| P35 | 5-[3-ethylsulfonyl-6- (1,2,4-triazol-1-yl)- 2-pyridyl]-1- (2,2,3,3,3- pentafluoropropyl) pyrrolo[2,3-c]pyridine | 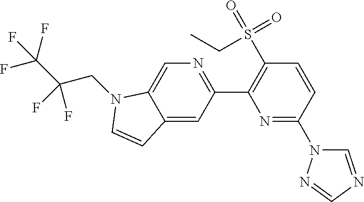 [SMILES: CCS(=O)(=O)c1ccc(-n2cncn2)nc1-c1cc2ccn(CC(F)(F)C(F)(F)F)c2cn1] | 0.97 | 487 | 1 | |
| P36 | 5-(6-chloro-3- ethylsulfonyl-2-pyridyl)- 1-(2,2,3,3,3- pentafluoropropyl) pyrrolo[2,3-c]pyridine | 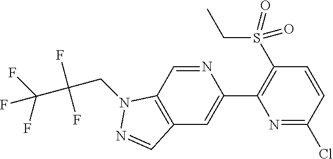 [SMILES: CCS(=O)(=O)c1ccc(Cl)nc1-c1cc2cnn(CC(F)(F)C(F)(F)F)c2cn1] | 1.07 | 454/456 | 1 | 194- 196 |
| P37 | 1-[5-ethylsulfanyl-6- [1-(2,2,3,3,3- pentafluoropropyl) pyrrolo[2,3-c]pyridin- 5-yl]-3-pyridyl] cyclopropane- carbonitrile | 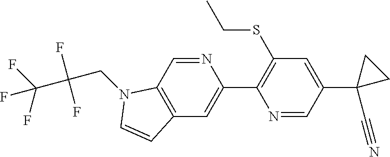 [SMILES: CCSc1cc(C2(C#N)CC2)cnc1-c1cc2ccn(CC(F)(F)C(F)(F)F)c2cn1] | 0.90 | 453 | 1 | 155- 158 |
| P38 | 1-[5-ethylsulfanyl-6- [3-(2,2,3,3,3- pentafluoropropyl) imidazo[4,5-c]pyridin- 6-yl]-3-pyridyl] cyclopropane- carbonitrile | 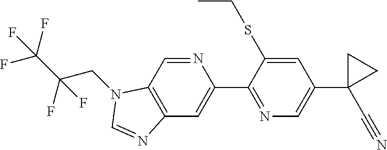 [SMILES: CCSc1cc(C2(C#N)CC2)cnc1-c1cc2ncn(CC(F)(F)C(F)(F)F)c2cn1] | 0.95 | 454 | 1 | |
| P39 | 5-[5-(3-chloropyrazol- 1-yl)-3-ethylsulfanyl- 2-pyridyl]-1- (2,2,3,3,3- pentafluoropropyl) pyrazolo[3,4-c]pyridine | 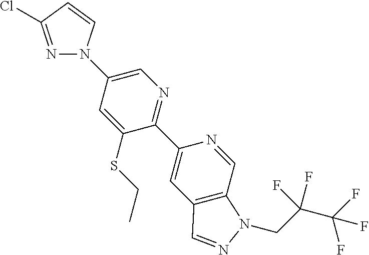 [SMILES: CCSc1cc(-n2ccc(Cl)n2)cnc1-c1cc2cnn(CC(F)(F)C(F)(F)F)c2cn1] | 1.22 | 489/491 | 3 | 185- 187 |
| P40 | 2-[5-ethylsulfonyl-6- [3-(2,2,3,3,3- pentafluoropropyl) imidazo[4,5-c] pyridin-6-yl]-3- pyridyl]-2-methyl- propanenitrile | 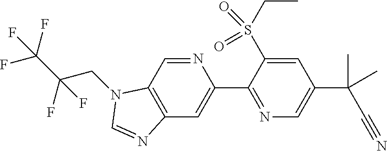 [SMILES: CCS(=O)(=O)c1cc(C(C)(C)C#N)cnc1-c1cc2ncn(CC(F)(F)C(F)(F)F)c2cn1] | 0.93 | 488 | 1 | 212- 226 |
| P41 | 6-(6-chloro-3- ethylsulfonyl-2- pyridyl)-3-(2,2,3,3,3- pentafluoropropyl) imidazo[4,5-c]pyridine | 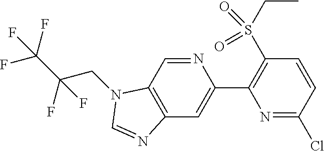 [SMILES: CCS(=O)(=O)c1ccc(Cl)nc1-c1cc2ncn(CC(F)(F)C(F)(F)F)c2cn1] | 0.96 | 455/457 | 1 | |
| P42 | 6-(3-ethylsulfonyl-2- pyridyl)-3-(2,2,3,3,3- pentafluoropropyl) imidazo[4,5-c]pyridine | 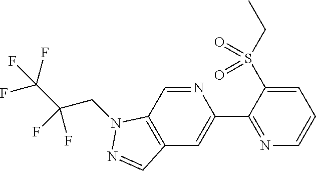 [SMILES: CCS(=O)(=O)c1cccnc1-c1cc2cnn(CC(F)(F)C(F)(F)F)c2cn1] | 0.83 | 421 | 1 | |
| P43 | 6-(6-chloro-3- ethylsulfanyl-2- pyridyl)-3-(2,2,3,3,3- pentafluoropropyl) imidazo[4,5-c] pyridine | 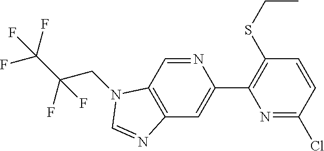 [SMILES: CCSc1ccc(Cl)nc1-c1cc2ncn(CC(F)(F)C(F)(F)F)c2cn1] | 1.03 | 423/425 | 1 | |
| P44 | 6-(3-ethylsulfanyl-2- pyridyl)-3-(2,2,3,3,3- pentafluoropropyl) imidazo[4,5-c]pyridine | 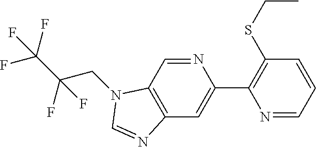 [SMILES: CCSc1cccnc1-c1cc2ncn(CC(F)(F)C(F)(F)F)c2cn1] | 0.87 | 389 | 1 | |
| P45 | 5-[3-ethylsulfonyl-5- [3-(trifluoromethyl) pyrazol-1-yl]-2-pyridyl]- 1-(2,2,3,3,3- pentafluoropropyl) pyrazolo[3,4-c]pyridine | 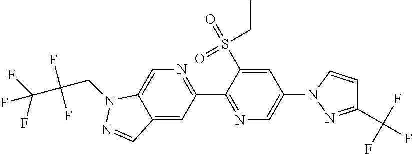 [SMILES: CCS(=O)(=O)c1cc(-n2ccc(C(F)(F)F)n2)cnc1-c1cc2cnn(CC(F)(F)C(F)(F)F)c2cn1] | 1.15 | 555 | 5 | 219- 221 |
| P46 | 5-[5-(3-chloropyrazol- 1-yl)-3-ethylsulfonyl- 2-pyridyl]-1-(2,2,3,3,3- pentafluoropropyl) pyrazolo[3,4-c]pyridine | 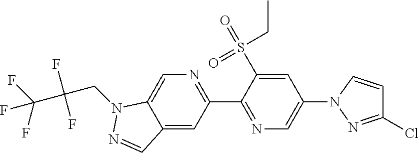 [SMILES: CCS(=O)(=O)c1cc(-n2ccc(Cl)n2)cnc1-c1cc2cnn(CC(F)(F)C(F)(F)F)c2cn1] | 1.15 | 521/523 | 5 | 220- 222 |
| P47 | 2-[5-ethylsulfanyl-6- [3-(2,2,3,3,3- pentafluoropropyl) imidazo[4,5-c]pyridin- 6-yl]-3-pyridyl]-2- methyl-propanenitrile | 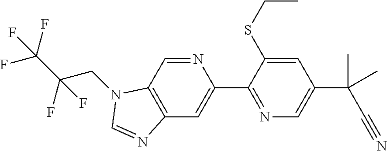 [SMILES: CCSc1cc(C(C)(C)C#N)cnc1-c1cc2ncn(CC(F)(F)C(F)(F)F)c2cn1] | 0.98 | 456 | 1 | |
| P48 | 5-(6-chloro-3- ethylsulfanyl-2-pyridyl)- 1-(2,2,3,3,3- pentafluoropropyl) pyrrolo[2,3-c]pyridine | 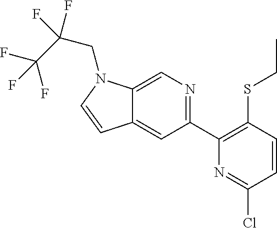 [SMILES: CCSc1ccc(Cl)nc1-c1cc2ccn(CC(F)(F)C(F)(F)F)c2cn1] | 1.03 | 422/424 | 1 | 185- 188 |
| P49 | 5-(6-chloro-3- ethylsulfonyl-2-pyridyl)- 1-(2,2,3,3,3- pentafluoropropyl) pyrazolo[3,4-c]pyridine | 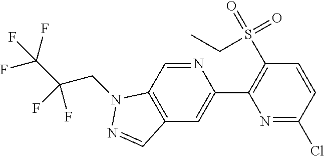 [SMILES: CCS(=O)(=O)c1ccc(Cl)nc1-c1cc2cnn(CC(F)(F)C(F)(F)F)c2cn1] | 1.16 | 455/457 | 5 | 154- 156 |
| P50 | 5-[3-ethylsulfanyl-5- [3-(trifluoromethyl) pyrazol-1-yl]-2- pyridyl]-1-(2,2,3,3,3- pentafluoropropyl) pyrazolo[3,4-c]pyridine | 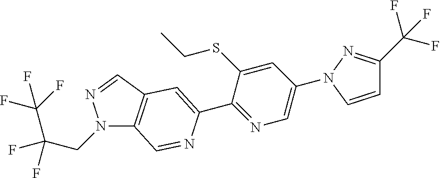 [SMILES: CCSc1cc(-n2ccc(C(F)(F)F)n2)cnc1-c1cc2cnn(CC(F)(F)C(F)(F)F)c2cn1] | 1.24 | 523 | 5 | 178- 180 |
| P51 | 5-(5-cyclopropyl-3- ethylsulfonyl-2-pyridyl)- 1-(2,2,3,3,3- pentafluoropropyl) pyrrolo[2,3-c]pyridine | 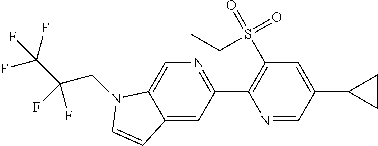 [SMILES: CCS(=O)(=O)c1cc(C2CC2)cnc1-c1cc2ccn(CC(F)(F)C(F)(F)F)c2cn1] | 0.97 | 460 | 1 | 223- 226 |
| P52 | 6-(5-cyclopropyl-3- ethylsulfonyl-2-pyridyl)- 3-(2,2,3,3,3- pentafluoropropyl) imidazo[4,5-c]pyridine | 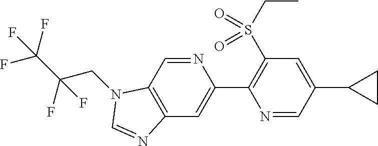 [SMILES: CCS(=O)(=O)c1cc(C2CC2)cnc1-c1cc2ncn(CC(F)(F)C(F)(F)F)c2cn1] | 0.95 | 461 | 1 | 228- 230 |
| P53 | 5-(6-chloro-3- ethylsulfanyl-2-pyridyl)- 1-(2,2,3,3,3- pentafluoropropyl) pyrazolo[3,4-c]pyridine | 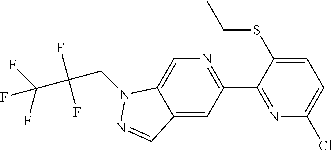 [SMILES: CCSc1ccc(Cl)nc1-c1cc2cnn(CC(F)(F)C(F)(F)F)c2cn1] | 1.26 | 424/426 | 5 | |
| P54 | 5-[3-ethylsulfanyl-5-(2- pyridyloxy)-2-pyridyl]- 1-(2,2,3,3,3- pentafluoropropyl) pyrrolo[2,3-c]pyridine | 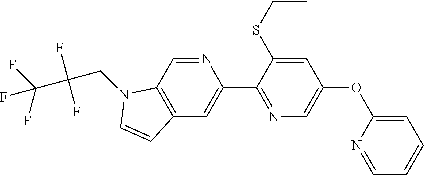 [SMILES: CCSc1cc(Oc2ccccn2)cnc1-c1cc2ccn(CC(F)(F)C(F)(F)F)c2cn1] | 0.91 | 481 | 1 | |

TABLE PN
Examples of pyridine N-oxides of compounds of formula (I)

| | | | LCMS | | | |
|---|---|---|---|---|---|---|
| | | | | [M + H]⁺ | | Mp |
| No. | IUPAC name | Structures | Rₜ (min) | (measured) | Method | (° C.) |
| PN1 | 5-(3-ethylsulfanyl-1- oxido-pyridin-1-ium- 2-yl)-1-(2,2,3,3,3- pentafluoropropyl) pyrazolo[3,4-c] pyridine | 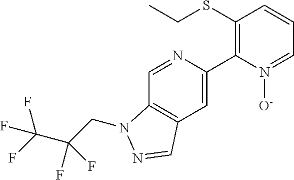 | 0.83 | 405 | 1 | |
| PN2 | 5-(3-ethylsulfanyl-1- oxido-pyridin-1-ium- 2-yl)-1-(2,2,3,3,3- pentafluoropropyl) pyrrolo[2,3-c]pyridine | 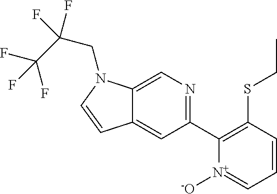 | 0.80 | 404 | 1 | 181- 185 |
| PN3 | 6-(3-ethylsulfanyl-1- oxido-pyridin-1-ium- 2-yl)-3- pentafluoropropyl) imidazo[4,5-c]pyridine | 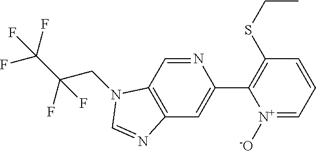 | 0.77 | 405 | 1 | |
| PN4 | 6-(5-cyclopropyl-3- ethylsulfanyl-1-oxido- pyridin-1-ium-2-yl)-3- pentafluoropropyl) imidazo[4,5-c]pyridine | 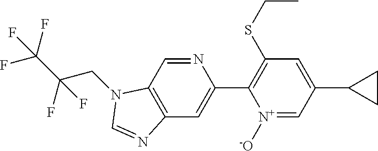 | 0.86 | 445 | 1 | |
| PN5 | 5-(5-cyclopropyl-3- ethylsulfanyl-1-oxido- pyridin-1-ium-2-yl)-1- pentafluoropropyl) pyrrolo[2,3-c]pyridine | 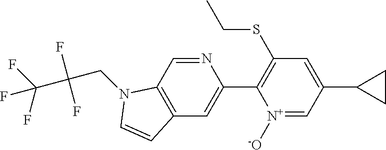 | 0.88 | 444 | 1 | |
| PN6 | 2-[5-ethylsulfanyl-1- oxido-6-[1-(2,2,3,3,3- pentafluoropropyl) pyrrolo[2,3-c]pyridin- 5-yl]pyridin-1- ium-3-yl]-2-methyl- propanenitrile | 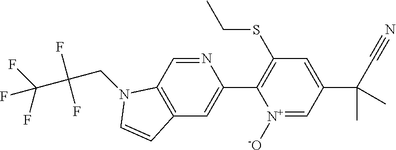 | 0.91 | 471 | 1 | 189- 194 |
| PN7 | 2-[5-ethylsulfanyl-1- oxido-6-[3-(2,2,3,3,3- pentafluoropropyl) imidazo[4,5-c] pyridin-6-yl]pyridin- 1-ium-3-yl]-2-methyl- propanenitrile | 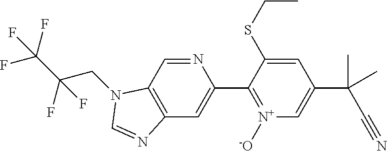 | 0.86 | 472 | 1 | |
| PN8 | 1-[5-ethylsulfanyl-1- oxido-6-[3-(2,2,3,3,3- pentafluoropropyl) imidazo[4,5-c]pyridin- 6-yl]pyridin -1-ium-3- yl]cyclopropane- carbonitrile | 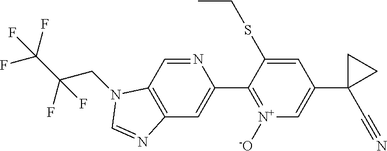 | 0.83 | 470 | 1 | |
| PN9 | 1-[5-ethylsulfanyl-1- oxido-6-[1-(2,2,3,3,3- pentafluoropropyl) pyrrolo[2,3-c]pyridin- 5-yl]pyridin-1-ium-3- yl]cyclopropane- carbonitrile | 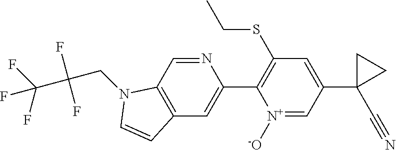 | 0.88 | 469 | 1 | |
| PN10 | 5-[3-ethylsulfanyl- 1-oxido-5-(2- pyridyloxy)pyridin- 1-ium-2-yl]-1- (2,2,3,3,3- pentafluoropropyl) pyrrolo[2,3-c]pyridine | 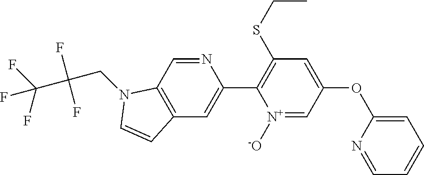 | 0.90 | 497 | 1 | |
| PN11 | 5-[3-ethylsulfanyl-1- oxido-5-(2-pyridyloxy) pyridin-1-ium-2yl]- 1-(2,2,3,3,3- pentafluoropropyl) pyrazolo[3,4-c]pyridine | 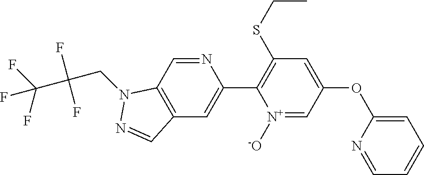 | 1.02 | 498 | 5 | |

TABLE I
Examples of intermediates for the preparation of compounds of formula (I)

| | | | LCMS | | | |
|---|---|---|---|---|---|---|
| | | | | [M + H]⁺ | | Mp |
| No. | IUPAC name | Structures | Rₜ (min) | (measured) | Method | (° C.) |
| I1 | 5-bromo-1-(2,2,3,3,3- pentafluoropropyl) pyrazolo[3,4-c]pyridine | 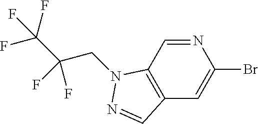 [SMILES: FC(F)(F)C(F)(F)Cn1ncc2cc(Br)ncc21] | 0.97 | 330/332 | 1 | |
| I2 | 5-(3-fluoro-1-oxido- pyridin-1-ium-2-yl)- 1-(2,2,3,3,3- pentafluoropropyl) pyrazolo[3,4-c] pyridine | 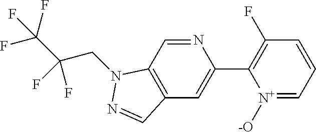 | 0.76 | 363 | 1 | |
| I3 | 5-bromo-1-(2,2,3,3,3- pentafluoropropyl) pyrrolo[2,3-c]pyridine | 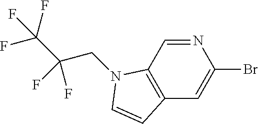 [SMILES: FC(F)(F)C(F)(F)Cn1ccc2cc(Br)ncc21] | 1.02 | 329/331 | 1 | 98- 102 |
| I4 | 6-bromo-3-(2,2,3,3,3- pentafluoropropyl) imidazo[4,5-c]pyridine | 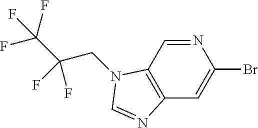 [SMILES: FC(F)(F)C(F)(F)Cn1cnc2cc(Br)ncc21] | 0.87 | 330/332 | 1 | 95- 98 |
| I5 | tert-butyl N-(5-fluoro- 3-pyridyl)-N-methyl- carbamate | 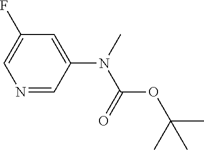 [SMILES: CN(C(=O)OC(C)(C)C)c1cncc(F)c1] | 0.90 | 227 | 1 | |
| I6 | tert-butyl N-(5-fluoro- 1-oxido-pyridin-1-ium- 3-yl)-N-methyl- carbamate | 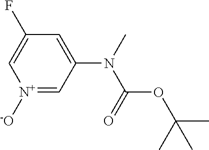 | 0.71 | 243 | 1 | |
| I7 | tert-butyl N-[5-fluoro- 1-oxido-6-[1-(2,2,3,3,3- pentafluoropropyl) pyrazolo[3,4-c]pyridin- 5-yl]pyridin-1- ium-3-yl]-N-methyl- carbamate | 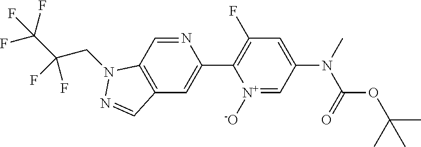 | 0.98 | 492 | 1 | |
| I8 | tert-butyl N-[5- ethylsulfanyl-1-oxido- 6-[1-(2,2,3,3,3- pentafluoropropyl) pyrazolo[3,4-c] pyridin-5-yl]pyridin- 1-ium-3-yl]-N- methyl-carbamate | 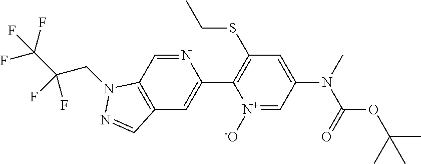 | | 1.01 | 534 | 1 |
| I9 | tert-butyl N-[5- ethylsulfanyl- 6-[1-(2,2,3,3,3- pentafluoropropyl) pyrazolo[3,4-c] pyridin-5-yl]-3- pyridyl]-N-methyl- carbamate | 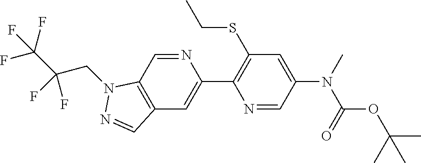 [SMILES: CCSc1cc(N(C)C(=O)OC(C)(C)C)cnc1-c1cc2cnn(CC(F)(F)C(F)(F)F)c2cn1] | 1.16 | 518 | 1 | |
| I10 | tert-butyl N-[5- ethylsulfonyl-6-[1- (2,2,3,3,3- pentafluoropropyl) pyrazolo[3,4-c] pyridin-5-yl]-3- pyridyl]-N-methyl- carbamate | 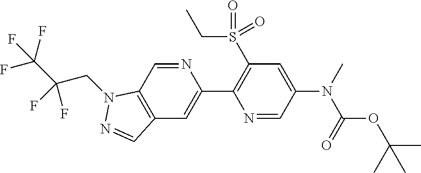 [SMILES: CCS(=O)(=O)c1cc(N(C)C(=O)OC(C)(C)C)cnc1-c1cc2cnn(CC(F)(F)C(F)(F)F)c2cn1] | 1.09 | 550 | 1 | |
| I11 | 5-(3-fluoro-1-oxido- pyridin-1-ium-2-yl)- 1-(2,2,3,3,3- pentafluoropropyl) pyrrolo[2,3-c]pyridine | 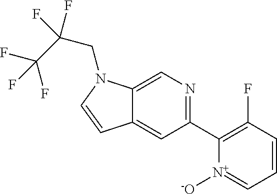 | 0.74 | 362 | 1 | |
| I12 | 2-[5-fluoro-1-oxido- 6-[1-(2,2,3,3,3 - pentafluoropropyl) pyrazolo[3,4-c] pyridin-5-yl]pyridin- 1-ium-3-yl]-2-methyl- propanenitrile | 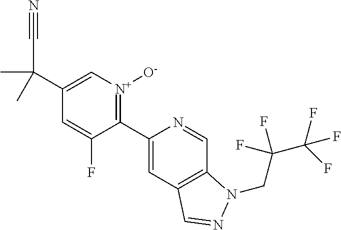 [SMILES: CC(C)(C#N)c1cc(F)c(-c2cc3cnn(CC(F)(F)C(F)(F)F)c3cn2)[n+]([O-])c1] | 0.92 | 430 | 5 | 180- 182 |
| I13 | 2-[5-fluoro-6-[1- (2,2,3,3,3- pentafluoropropyl) pyrazolo[3,4-c] pyridin-5-yl]-3- pyridyl]-2-methyl- propanenitrile | 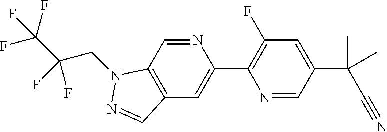 [SMILES: CC(C)(C#N)c1cnc(-c2cc3cnn(CC(F)(F)C(F)(F)F)c3cn2)c(F)c1] | 1.05 | 414 | 5 | 174- 176 |
| I14 | 5-(5-chloro-3- fluoro-1-oxido- pyridin-1-ium-2- yl)-1-(2,2,3,3,3- pentafluoropropyl) pyrrolo[2, 3-c]pyridine | 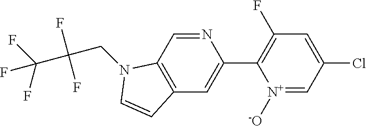 | 0.85 | 396/398 | 1 | |
| I15 | 1-[5-fluoro-1-oxido- 6-[1-(2,2,3,3,3- pentafluoropropyl) pyrazolo[3,4-c] pyridin-5-yl] pyridin-1- ium-3-yl] cyclopropanecarbonitrile | 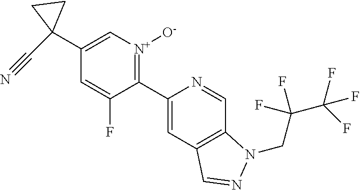 [SMILES: N#CC1(c2cc(F)c(-c3cc4cnn(CC(F)(F)C(F)(F)F)c4cn3)[n+]([O-])c2)CC1] | 0.92 | 428 | 5 | 180- 182 |
| I16 | 1-[5-fluoro-6-[1- (2,2,3,3,3- pentafluoropropyl) pyrazolo[3,4-c] pyridin-5-yl]-3- pyridyl]cyclopropane- carbonitrue | 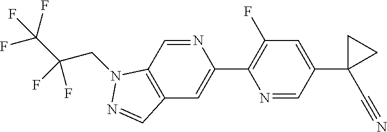 [SMILES: N#CC1(c2cnc(-c3cc4cnn(CC(F)(F)C(F)(F)F)c4cn3)c(F)c2)CC1] | 1.07 | 412 | 5 | 178- 179 |
| I17 | 5-(5-cyclopropyl-3- fluoro-1-oxido- pyridin-1-ium-2-yl)- 1-(2,2,3,3,3- pentafluoropropyl) pyrrolo[2,3-c]pyridine | 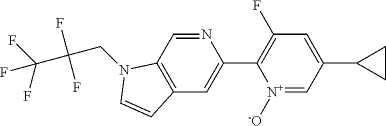 | 0.84 | 402 | 1 | 202- 205 |
| I18 | 2-[5-fluoro-1-oxido- 6-[1-(2,2,3,3,3- pentafluoropropyl) pyrrolo[2,3-c]pyridin- 5-yl]pyridin-1- ium-3-yl]-2-methyl- propanenitrile | 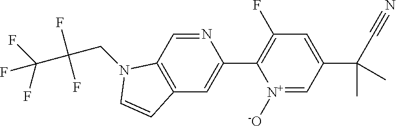 | 0.86 | 429 | 1 | 210 |
| I19 | 1-[5-fluoro-1-oxido- 6-[1-(2,2,3,3,3- pentafluoropropyl) pyrrolo[2,3-c]pyridin- 5-yl]pyridin- 1-ium-3-yl] cyclopropane- carbonitrile | 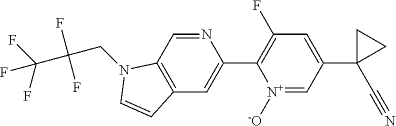 | 0.83 | 427 | 1 | 220 |
| I20 | tert-butyl N-[5-fluoro- 1-oxido-6-[1- (2,2,3,3,3- pentafluoropropyl) pyrrolo[2,3-c] pyridin-5-yl]pyridin- 1-ium-3-yl]-N-methyl- carbamate | 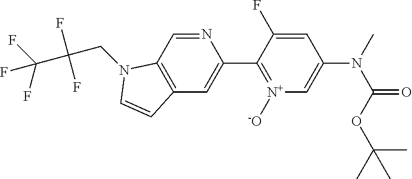 | 0.96 | 491 | 1 | 192- 195 |
| I21 | tert-butyl N-[5- ethylsulfanyl-1-oxido- 6-[1-(2,2,3,3,3- pentafluoropropyl) pyrrolo[2,3-c]pyridin- 5-yl]pyridin-1- ium-3-yl]-N-methyl- carbamate | 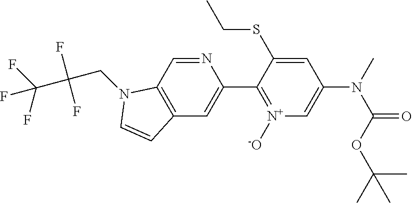 | 0.98 | 534 | 1 | |
| I22 | tert-butyl N-[5- ethylsulfanyl- 6-[1-(2,2,3,3,3- pentafluoropropyl) pyrrolo[2,3-c]pyridin- 5-yl]-3-pyridyl]- N-methyl-carbamate | 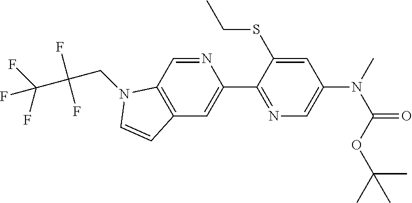 [SMILES: CCSc1cc(N(C)C(=O)OC(C)(C)C)cnc1-c1cc2ccn(CC(F)(F)C(F)(F)F)c2cn1] | 1 .01 | 518 | 1 | |
| I23 | tert-butyl N-[5- ethylsulfonyl- 6-[1-(2,2,3,3,3- pentafluoropropyl) pyrrolo[2,3-c]pyridin- 5-yl]-3-pyridyl]- N-methyl-carbamate | 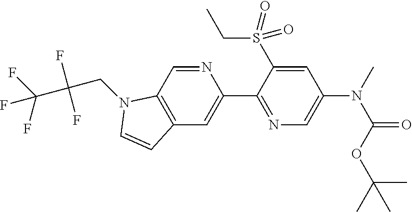 [SMILES: CCS(=O)(=O)c1cc(N(C)C(=O)OC(C)(C)C)cnc1-c1cc2ccn(CC(F)(F)C(F)(F)F)c2cn1] | 1.09 | 550 | 1 | |
| I24 | 3-cyclopropyl-5- fluoro-1-oxido- pyridin-1-ium | 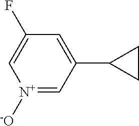 | 0.43 | 154 | 1 | |
| I25 | 2-(5-fluoro-1- oxido-pyridin- 1-ium-3-yl)-2- methyl- propanenitrile | 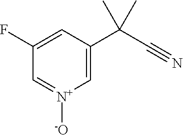 | 0.34 | 181 | 1 | |
| I26 | 1-(5-fluoro-1- oxido-pyridin- 1-ium-3-yl) cyclopropane- carbonitrile | 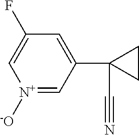 | 0.29 | 179 | 1 | |
| I27 | 3-fluoro-1- oxido-5-(2- pyridyloxy) pyridin-1-ium | 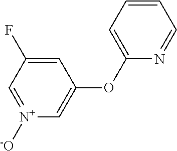 | 0.53 | 207 | 1 | |
| I28 | 6-(3-fluoro-1- oxido-pyridin- 1-ium-2-yl)-3- (2,2,3,3,3- pentafluoropropyl) imidazo[4,5-c] pyridine | 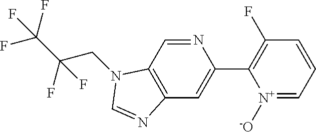 | 0.69 | 363 | 1 | |
| I29 | 6-(5-cyclopropyl- 3-fluoro-1-oxido- pyridin-1-ium-2-yl)- 3-(2,2,3,3,3- pentafluoropropyl) imidazo [4,5-c]pyridine | 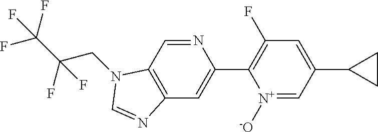 | 0.80 | 403 | 1 | |
| I30 | 2-[5-fluoro-1- oxido-6-[3- pentafluoropropyl) imidazo[4,5-c] pyridin-6-yl]pyridin- 1-ium-3-yl]-2-methyl- propanenitrile | 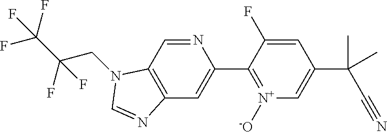 | 0.80 | 430 | 1 | |
| I31 | 1-[5-fluoro-1- oxido-6-[3- pentafluoropropyl) imidazo[4,5-c]pyridin- 6-yl]pyridin-1- ium-3-yl] cyclopropane- carbonitrile | 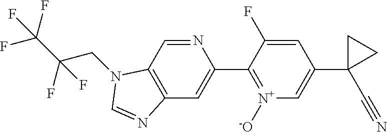 | 0.78 | 428 | 1 | |
| I32 | 5-[3-fluoro-1- oxido-5-(2- pyridyloxy)pyridin- 1-ium-2- pentafluoropropyl) pyrrolo[2,3-c]pyridine | 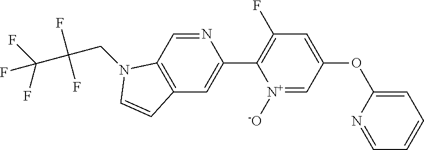 | 0.85 | 455 | 1 | |
| I33 | 5-[3-fluoro-1- oxido-5-(2- pyridyloxy) pyridin-1-ium-2- yl]-1-(2,2,3,3,3- pentafluoropropyl) pyrazolo [3,4-c]pyridine | 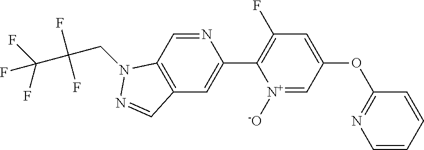 | 0.92 | 456 | 5 | |
| I34 | 2-(5-fluoro-3- pyridyl)-2- methyl- propanenitrile | 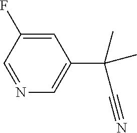 | 0.72 | 165 | 1 | |
| I35 | 1-(5-fluoro-3- pyridyl) cyclopropane- carbonitrile | 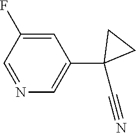 [SMILES: N#CC1(c2cncc(F)c2)CC1] | 0.67 | 163 | 1 | |
| I36 | 3-fluoro-5-(2- pyridyloxy) pyridine | 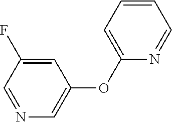 [SMILES: Fc1cncc(Oc2ccccn2)c1] | 0.78 | 191 | 1 | 53- 56 |

The activity of the compositions according to the invention can be broadened considerably, and adapted to prevailing circumstances, by adding other insecticidally, acaricidally and/or fungicidally active ingredients. The mixtures of the compounds of formula I with other insecticidally, acaricidally and/or fungicidally active ingredients may also have further surprising advantages which can also be described, in a wider sense, as synergistic activity. For example, better tolerance by plants, reduced phytotoxicity, insects can be controlled in their different development stages or better behaviour during their production, for example during grinding or mixing, during their storage or during their use. Suitable additions to active ingredients here are, for example, representatives of the following classes of active ingredients: organophosphorus compounds, nitrophenol derivatives, thioureas, juvenile hormones, formamidines, benzophenone derivatives, ureas, pyrrole derivatives, carbamates, pyrethroids, chlorinated hydrocarbons, acylureas, pyridylmethyleneamino derivatives, macrolides, neonicotinoids and Bacillus thuringiensis preparations.

The following mixtures of the compounds of formula I with active ingredients are preferred (the abbreviation “TX” means “one compound selected from the group consisting of the compounds described in Tables A-1 to A-81, Tables B-1 to B-54, Tables C-1 to C-81, Tables D-1 to D-54, Tables E-1 to E-81 and Tables F-1 to F-54, and Table P of the present invention”):

an adjuvant selected from the group of substances consisting of petroleum oils (alternative name) (628)+TX,
an adjuvant selected from the group of substances consisting of petroleum oils (alternative name) (628)+TX,
an acaricide selected from the group of substances consisting of 1,1-bis(4-chlorophenyl)-2-ethoxyethanol (IUPAC name) (910)+TX, 2,4-dichlorophenyl benzenesulfonate (IUPAC/Chemical Abstracts name) (1059)+TX, 2-fluoro-N-methyl-N-1-naphthylacetamide (IUPAC name) (1295)+TX, 4-chlorophenyl phenyl sulfone (IUPAC name) (981)+TX, abamectin (1)+TX, acequinocyl (3)+TX, acetoprole [CCN]+TX, acrinathrin (9)+TX, aldicarb (16)+TX, aldoxycarb (863)+TX, alpha-cypermethrin (202)+TX, amidithion (870)+TX, amidoflumet [CCN]+TX, amidothioate (872)+TX, amiton (875)+TX, amiton hydrogen oxalate (875)+TX, amitraz (24)+TX, aramite (881)+TX, arsenous oxide (882)+TX, AVI 382 (compound code)+TX, AZ 60541 (compound code)+TX, azinphos-ethyl (44)+TX, azinphos-methyl (45)+TX, azobenzene (IUPAC name) (888)+TX, azocyclotin (46)+TX, azothoate (889)+TX, benomyl (62)+TX, benoxafos (alternative name) [CCN]+TX, benzoximate (71)+TX, benzyl benzoate (IUPAC name) [CCN]+TX, bifenazate (74)+TX, bifenthrin (76)+TX, binapacryl (907)+TX, brofenvalerate (alternative name)+TX, bromo-cyclen (918)+TX, bromophos (920)+TX, bromophos-ethyl (921)+TX, bromopropylate (94)+TX, buprofezin (99)+TX, butocarboxim (103)+TX, butoxycarboxim (104)+TX, butylpyridaben (alternative name)+TX, calcium polysulfide (IUPAC name) (111)+TX, camphechlor (941)+TX, carbanolate (943)+TX, carbaryl (115)+TX, carbofuran (118)+TX, carbophenothion (947)+TX, CGA 50′439 (development code) (125)+TX, chinomethionat (126)+TX, chlorbenside (959)+TX, chlordimeform (964)+TX, chlordimeform hydrochloride (964)+TX, chlorfenapyr (130)+TX, chlorfenethol (968)+TX, chlorfenson (970)+TX, chlorfensulfide (971)+TX, chlorfenvinphos (131)+TX, chlorobenzilate (975)+TX, chloromebuform (977)+TX, chloromethiuron (978)+TX, chloropropylate (983)+TX, chlorpyrifos (145)+TX, chlorpyrifos-methyl (146)+TX, chlorthiophos (994)+TX, cinerin I (696)+TX, cinerin II (696)+TX, cinerins (696)+TX, clofentezine (158)+TX, closantel (alternative name) [CCN]+TX, coumaphos (174)+TX, crotamiton (alternative name) [CCN]+TX, crotoxyphos (1010)+TX, cufraneb (1013)+TX, cyanthoate (1020)+TX, cyflumetofen (CAS Reg. No.: 400882-07-7)+TX, cyhalothrin (196)+TX, cyhexatin (199)+TX, cypermetrin (201)+TX, DCPM (1032)+TX, DDT (219)+TX, demephion (1037)+TX, demephion-O (1037)+TX, demephion-S (1037)+TX, demeton (1038)+TX, demeton-methyl (224)+TX, demeton-O (1038)+TX, demeton-O-methyl (224)+TX, demeton-S (1038)+TX, demeton-S-methyl (224)+TX, demeton-S-methylsulfon (1039)+TX, diafenthiuron (226)+TX, dialifos (1042)+TX, diazinon (227)+TX, dichlofluanid (230)+TX, dichlorvos (236)+TX, dicliphos (alternative name)+TX, dicofol (242)+TX, dicrotophos (243)+TX, dienochlor (1071)+TX, dimefox (1081)+TX, dimethoate (262)+TX, dinactin (alternative name) (653)+TX, dinex (1089)+TX, dinex-diclexine (1089)+TX, dinobuton (269)+TX, dinocap (270)+TX, dinocap-4 [CCN]+TX, dinocap-6 [CCN]+TX, dinocton (1090)+TX, dinopenton (1092)+TX, dinosulfon (1097)+TX, dinoterbon (1098)+TX, dioxathion (1102)+TX, diphenyl sulfone (IUPAC name) (1103)+TX, disulfiram (alternative name) [CCN]+TX, disulfoton (278)+TX, DNOC (282)+TX, dofenapyn (1113)+TX, doramectin (alternative name) [CCN]+TX, endosulfan (294)+TX, endothion (1121)+TX, EPN (297)+TX, eprinomectin (alternative name) [CCN]+TX, ethion (309)+TX, ethoate-methyl (1134)+TX, etoxazole (320)+TX, etrimfos (1142)+TX, fenazaflor (1147)+TX, fenazaquin (328)+TX, fenbutatin oxide (330)+TX, fenothiocarb (337)+TX, fenpropathrin (342)+TX, fenpyrad (alternative name)+TX, fenpyroximate (345)+TX, fenson (1157)+TX, fentrifanil (1161)+TX, fenvalerate (349)+TX, fipronil (354)+TX, fluacrypyrim (360)+TX, fluazuron (1166)+TX, flubenzimine (1167)+TX, flucycloxuron (366)+TX, flucythrinate (367)+TX, fluenetil (1169)+TX, flufenoxuron (370)+TX, flumethrin (372)+TX, fluorbenside (1174)+TX, fluvalinate (1184)+TX, FMC 1137 (development code) (1185)+TX, formetanate (405)+TX, formetanate hydrochloride (405)+TX, formothion (1192)+TX, formparanate (1193)+TX, gamma-HCH (430)+TX, glyodin (1205)+TX, halfenprox (424)+TX, heptenophos (432)+TX, hexadecyl cyclopropanecarboxylate (IUPAC/Chemical Abstracts name) (1216)+TX, hexythiazox (441)+TX, iodomethane (IUPAC name) (542)+TX, isocarbophos (alternative name) (473)+TX, isopropyl O-(methoxyaminothiophosphoryl)salicylate (IUPAC name) (473)+TX, ivermectin (alternative name) [CCN]+TX, jasmolin 1 (696)+TX, jasmolin II (696)+TX, jodfenphos (1248)+TX, lindane (430)+TX, lufenuron (490)+TX, malathion (492)+TX, malonoben (1254)+TX, mecarbam (502)+TX, mephosfolan (1261)+TX, mesulfen (alternative name) [CCN]+TX, methacrifos (1266)+TX, methamidophos (527)+TX, methidathion (529)+TX, methiocarb (530)+TX, methomyl (531)+TX, methyl bromide (537)+TX, metolcarb (550)+TX, mevinphos (556)+TX, mexacarbate (1290)+TX, milbemectin (557)+TX, milbemycin oxime (alternative name) [CCN]+TX, mipafox (1293)+TX, monocrotophos (561)+TX, morphothion (1300)+TX, moxidectin (alternative name) [CCN]+TX, naled (567)+TX, NC-184 (compound code)+TX, NC-512 (compound code)+TX, nifluridide (1309)+TX, nikkomycins (alternative name) [CCN]+TX, nitrilacarb (1313)+TX, nitrilacarb 1:1 zinc chloride complex (1313)+TX, NNI-0101 (compound code)+TX, NNI-0250 (compound code)+TX, omethoate (594)+TX, oxamyl (602)+TX, oxydeprofos (1324)+TX, oxydisulfoton (1325)+TX, pp′-DDT (219)+TX, parathion (615)+TX, permethrin (626)+TX, petroleum oils (alternative name) (628)+TX, phenkapton (1330)+TX, phenthoate (631)+TX, phorate (636)+TX, phosalone (637)+TX, phosfolan (1338)+TX, phosmet (638)+TX, phosphamidon (639)+TX, phoxim (642)+TX, pirimiphos-methyl (652)+TX, polychloroterpenes (traditional name) (1347)+TX, polynactins (alternative name) (653)+TX, proclonol (1350)+TX, profenofos (662)+TX, promacyl (1354)+TX, propargite (671)+TX, propetamphos (673)+TX, propoxur (678)+TX, prothidathion (1360)+TX, prothoate (1362)+TX, pyrethrin I (696)+TX, pyrethrin II (696)+TX, pyrethrins (696)+TX, pyridaben (699)+TX, pyridaphenthion (701)+TX, pyrimidifen (706)+TX, pyrimitate (1370)+TX, quinalphos (711)+TX, quintiofos (1381)+TX, R-1492 (development code) (1382)+TX, RA-17 (development code) (1383)+TX, rotenone (722)+TX, schradan (1389)+TX, sebufos (alternative name)+TX, selamectin (alternative name) [CCN]+TX, SI-0009 (compound code)+TX, sophamide (1402)+TX, spirodiclofen (738)+TX, spiromesifen (739)+TX, SSI-121 (development code) (1404)+TX, sulfiram (alternative name) [CCN]+TX, sulfluramid (750)+TX, sulfotep (753)+TX, sulfur (754)+TX, SZI-121 (development code) (757)+TX, tau-fluvalinate (398)+TX, tebufenpyrad (763)+TX, TEPP (1417)+TX, terbam (alternative name)+TX, tetrachlorvinphos (777)+TX, tetradifon (786)+TX, tetranactin (alternative name) (653)+TX, tetrasul (1425)+TX, thiafenox (alternative name)+TX, thiocarboxime (1431)+TX, thiofanox (800)+TX, thiometon (801)+TX, thioquinox (1436)+TX, thuringiensin (alternative name) [CCN]+TX, triamiphos (1441)+TX, triarathene (1443)+TX, triazophos (820)+TX, triazuron (alternative name)+TX, trichlorfon (824)+TX, trifenofos (1455)+TX, trinactin (alternative name) (653)+TX, vamidothion (847)+TX, vaniliprole [CCN] and YI-5302 (compound code)+TX,
an algicide selected from the group of substances consisting of bethoxazin [CCN]+TX, copper dioctanoate (IUPAC name) (170)+TX, copper sulfate (172)+TX, cybutryne [CCN]+TX, dichlone (1052)+TX, dichlorophen (232)+TX, endothal (295)+TX, fentin (347)+TX, hydrated lime [CCN]+TX, nabam (566)+TX, quinoclamine (714)+TX, quinonamid (1379)+TX, simazine (730)+TX, triphenyltin acetate (IUPAC name) (347) and triphenyltin hydroxide (IUPAC name) (347)+TX,
an anthelmintic selected from the group of substances consisting of abamectin (1)+TX, crufomate (1011)+TX, doramectin (alternative name) [CCN]+TX, emamectin (291)+TX, emamectin benzoate (291)+TX, eprinomectin (alternative name) [CCN]+TX, ivermectin (alternative name) [CCN]+TX, milbemycin oxime (alternative name) [CCN]+TX, moxidectin (alternative name) [CCN]+TX, piperazine [CCN]+TX, selamectin (alternative name) [CCN]+TX, spinosad (737) and thiophanate (1435)+TX,
an avicide selected from the group of substances consisting of chloralose (127)+TX, endrin (1122)+TX, fenthion (346)+TX, pyridin-4-amine (IUPAC name) (23) and strychnine (745)+TX, a bactericide selected from the group of substances consisting of 1-hydroxy-1H-pyridine-2-thione (IUPAC name) (1222)+TX, 4-(quinoxalin-2-ylamino)benzenesulfonamide (IUPAC name) (748)+TX, 8-hydroxyquinoline sulfate (446)+TX, bronopol (97)+TX, copper dioctanoate (IUPAC name) (170)+TX, copper hydroxide (IUPAC name) (169)+TX, cresol [CCN]+TX, dichlorophen (232)+TX, dipyrithione (1105)+TX, dodicin (1112)+TX, fenaminosulf (1144)+TX, formaldehyde (404)+TX, hydrargaphen (alternative name) [CCN]+TX, kasugamycin (483)+TX, kasugamycin hydrochloride hydrate (483)+TX, nickel bis(dimethyldithiocarbamate) (IUPAC name) (1308)+TX, nitrapyrin (580)+TX, octhilinone (590)+TX, oxolinic acid (606)+TX, oxytetracycline (611)+TX, potassium hydroxyquinoline sulfate (446)+TX, probenazole (658)+TX, streptomycin (744)+TX, streptomycin sesquisulfate (744)+TX, tecloftalam (766)+TX, and thiomersal (alternative name) [CCN]+TX,
a biological agent selected from the group of substances consisting of Adoxophyes orana GV (alternative name) (12)+TX, Agrobacterium radiobacter (alternative name) (13)+TX, Amblyseius spp. (alternative name) (19)+TX, Anagrapha falcifera NPV (alternative name) (28)+TX, Anagrus atomus (alternative name) (29)+TX, Aphelinus abdominalis (alternative name) (33)+TX, Aphidius colemani (alternative name) (34)+TX, Aphidoletes aphidimyza (alternative name) (35)+TX, Autographa californica NPV (alternative name) (38)+TX, Bacillus firmus (alternative name) (48)+TX, Bacillus sphaericus Neide (scientific name) (49)+TX, Bacillus thuringiensis Berliner (scientific name) (51)+TX, Bacillus thuringiensis subsp. aizawai (scientific name) (51)+TX, Bacillus thuringiensis subsp. israelensis (scientific name) (51)+TX, Bacillus thuringiensis subsp. japonensis (scientific name) (51)+TX, Bacillus thuringiensis subsp. kurstaki (scientific name) (51)+TX, Bacillus thuringiensis subsp. tenebrionis (scientific name) (51)+TX, Beauveria bassiana (alternative name) (53)+TX, Beauveria brongniartii (alternative name) (54)+TX, Chrysoperla carnea (alternative name) (151)+TX, Cryptolaemus montrouzieri (alternative name) (178)+TX, Cydia pomonella GV (alternative name) (191)+TX, Dacnusa sibirica (alternative name) (212)+TX, Diglyphus isaea (alternative name) (254)+TX, Encarsia formosa (scientific name) (293)+TX, Eretmocerus eremicus (alternative name) (300)+TX, Helicoverpa zea NPV (alternative name) (431)+TX, Heterorhabditis bacteriophora and H. megidis (alternative name) (433)+TX, Hippodamia convergens (alternative name) (442)+TX, Leptomastix dactylopii (alternative name) (488)+TX, Macrolophus caliginosus (alternative name) (491)+TX, Mamestra brassicae NPV (alternative name) (494)+TX, Metaphycus helvolus (alternative name) (522)+TX, Metarhizium anisopliae var. acridum (scientific name) (523)+TX, Metarhizium anisopliae var. anisopliae (scientific name) (523)+TX, Neodiprion sertifer NPV and N. lecontei NPV (alternative name) (575)+TX, Orius spp. (alternative name) (596)+TX, Paecilomyces fumosoroseus (alternative name) (613)+TX, Phytoseiulus persimilis (alternative name) (644)+TX, Spodoptera exigua multicapsid nuclear polyhedrosis virus (scientific name) (741)+TX, Steinernema bibionis (alternative name) (742)+TX, Steinernema carpocapsae (alternative name) (742)+TX, Steinernema feltiae (alternative name) (742)+TX, Steinernema glaseri (alternative name) (742)+TX, Steinernema riobrave (alternative name) (742)+TX, Steinernema riobravis (alternative name) (742)+TX, Steinernema scapterisci (alternative name) (742)+TX, Steinernema spp. (alternative name) (742)+TX, Trichogramma spp. (alternative name) (826)+TX, Typhlodromus occidentalis (alternative name) (844) and Verticillium lecanii (alternative name) (848)+TX,
a soil sterilant selected from the group of substances consisting of iodomethane (IUPAC name) (542) and methyl bromide (537)+TX,
a chemosterilant selected from the group of substances consisting of apholate [CCN]+TX, bisazir (alternative name) [CCN]+TX, busulfan (alternative name) [CCN]+TX, diflubenzuron (250)+TX, dimatif (alternative name) [CCN]+TX, hemel [CCN]+TX, hempa [CCN]+TX, metepa [CCN]+TX, methiotepa [CCN]+TX, methyl apholate [CCN]+TX, morzid [CCN]+TX, penfluron (alternative name) [CCN]+TX, tepa [CCN]+TX, thiohempa (alternative name) [CCN]+TX, thiotepa (alternative name) [CCN]+TX, tretamine (alternative name) [CCN] and uredepa (alternative name) [CCN]+TX,
an insect pheromone selected from the group of substances consisting of (E)-dec-5-en-1-yl acetate with (E)-dec-5-en-1-ol (IUPAC name) (222)+TX, (E)-tridec-4-en-1-yl acetate (IUPAC name) (829)+TX, (E)-6-methylhept-2-en-4-ol (IUPAC name) (541)+TX, (E,Z)-tetradeca-4,10-dien-1-yl acetate (IUPAC name) (779)+TX, (Z)-dodec-7-en-1-yl acetate (IUPAC name) (285)+TX, (Z)-hexadec-11-enal (IUPAC name) (436)+TX, (Z)-hexadec-11-en-1-yl acetate (IUPAC name) (437)+TX, (Z)-hexadec-13-en-11-yn-1-yl acetate (IUPAC name) (438)+TX, (Z)-icos-13-en-10-one (IUPAC name) (448)+TX, (Z)-tetradec-7-en-1-al (IUPAC name) (782)+TX, (Z)-tetradec-9-en-1-ol (IUPAC name) (783)+TX, (Z)-tetradec-9-en-1-yl acetate (IUPAC name) (784)+TX, (7E,9Z)-dodeca-7,9-dien-1-yl acetate (IUPAC name) (283)+TX, (9Z,11E)-tetradeca-9,11-dien-1-yl acetate (IUPAC name) (780)+TX, (9Z,12E)-tetradeca-9,12-dien-1-yl acetate (IUPAC name) (781)+TX, 14-methyloctadec-1-ene (IUPAC name) (545)+TX, 4-methylnonan-5-ol with 4-methylnonan-5-one (IUPAC name) (544)+TX, alpha-multistriatin (alternative name) [CCN]+TX, brevicomin (alternative name) [CCN]+TX, codlelure (alternative name) [CCN]+TX, codlemone (alternative name) (167)+TX, cuelure (alternative name) (179)+TX, disparlure (277)+TX, dodec-8-en-1-yl acetate (IUPAC name) (286)+TX, dodec-9-en-1-yl acetate (IUPAC name) (287)+TX, dodeca-8+TX, 10-dien-1-yl acetate (IUPAC name) (284)+TX, dominicalure (alternative name) [CCN]+TX, ethyl 4-methyloctanoate (IUPAC name) (317)+TX, eugenol (alternative name) [CCN]+TX, frontalin (alternative name) [CCN]+TX, gossyplure (alternative name) (420)+TX, grandlure (421)+TX, grandlure I (alternative name) (421)+TX, grandlure II (alternative name) (421)+TX, grandlure III (alternative name) (421)+TX, grandlure IV (alternative name) (421)+TX, hexalure [CCN]+TX, ipsdienol (alternative name) [CCN]+TX, ipsenol (alternative name) [CCN]+TX, japonilure (alternative name) (481)+TX, lineatin (alternative name) [CCN]+TX, litlure (alternative name) [CCN]+TX, looplure (alternative name) [CCN]+TX, medlure [CCN]+TX, megatomoic acid (alternative name) [CCN]+TX, methyl eugenol (alternative name) (540)+TX, muscalure (563)+TX, octadeca-2,13-dien-1-yl acetate (IUPAC name) (588)+TX, octadeca-3,13-dien-1-yl acetate (IUPAC name) (589)+TX, orfralure (alternative name) [CCN]+TX, oryctalure (alternative name) (317)+TX, ostramone (alternative name) [CCN]+TX, siglure [CCN]+TX, sordidin (alternative name) (736)+TX, sulcatol (alternative name) [CCN]+TX, tetradec-11-en-1-yl acetate (IUPAC name) (785)+TX, trimedlure (839)+TX, trimedlure A (alternative name) (839)+TX, trimedlure B₁ (alternative name) (839)+TX, trimedlure B₂ (alternative name) (839)+TX, trimedlure C (alternative name) (839) and trunc-call (alternative name) [CCN]+TX,
an insect repellent selected from the group of substances consisting of 2-(octylthio)ethanol (IUPAC name) (591)+TX, butopyronoxyl (933)+TX, butoxy(polypropylene glycol) (936)+TX, dibutyl adipate (IUPAC name) (1046)+TX, dibutyl phthalate (1047)+TX, dibutyl succinate (IUPAC name) (1048)+TX, diethyltoluamide [CCN]+TX, dimethyl carbate [CCN]+TX, dimethyl phthalate [CCN]+TX, ethyl hexanediol (1137)+TX, hexamide [CCN]+TX, methoquin-butyl (1276)+TX, methylneodecanamide [CCN]+TX, oxamate [CCN] and picaridin [CCN]+TX, an insecticide selected from the group of substances consisting of 1-dichloro-1-nitroethane (IUPAC/Chemical Abstracts name) (1058)+TX, 1,1-dichloro-2,2-bis(4-ethylphenyl)ethane (IUPAC name) (1056), +TX, 1,2-dichloropropane (IUPAC/Chemical Abstracts name) (1062)+TX, 1,2-dichloropropane with 1,3-dichloropropene (IUPAC name) (1063)+TX, 1-bromo-2-chloroethane (IUPAC/Chemical Abstracts name) (916)+TX, 2,2,2-trichloro-1-(3,4-dichlorophenyl)ethyl acetate (IUPAC name) (1451)+TX, 2,2-dichlorovinyl 2-ethylsulfinylethyl methyl phosphate (IUPAC name) (1066)+TX, 2-(1,3-dithiolan-2-yl)phenyl dimethylcarbamate (IUPAC/Chemical Abstracts name) (1109)+TX, 2-(2-butoxyethoxy)ethyl thiocyanate (IUPAC/Chemical Abstracts name) (935)+TX, 2-(4,5-dimethyl-1,3-dioxolan-2-yl)phenyl methylcarbamate (IUPAC/Chemical Abstracts name) (1084)+TX, 2-(4-chloro-3,5-xylyloxy)ethanol (IUPAC name) (986)+TX, 2-chlorovinyl diethyl phosphate (IUPAC name) (984)+TX, 2-imidazolidone (IUPAC name) (1225)+TX, 2-isovalerylindan-1,3-dione (IUPAC name) (1246)+TX, 2-methyl(prop-2-ynyl)aminophenyl methylcarbamate (IUPAC name) (1284)+TX, 2-thiocyanatoethyl laurate (IUPAC name) (1433)+TX, 3-bromo-1-chloroprop-1-ene (IUPAC name) (917)+TX, 3-methyl-1-phenylpyrazol-5-yl dimethylcarbamate (IUPAC name) (1283)+TX, 4-methyl(prop-2-ynyl)amino-3,5-xylyl methylcarbamate (IUPAC name) (1285)+TX, 5,5-dimethyl-3-oxocyclohex-1-enyl dimethylcarbamate (IUPAC name) (1085)+TX, abamectin (1)+TX, acephate (2)+TX, acetamiprid (4)+TX, acethion (alternative name) [CCN]+TX, acetoprole [CCN]+TX, acrinathrin (9)+TX, acrylonitrile (IUPAC name) (861)+TX, alanycarb (15)+TX, aldicarb (16)+TX, aldoxycarb (863)+TX, aldrin (864)+TX, allethrin (17)+TX, allosamidin (alternative name) [CCN]+TX, allyxycarb (866)+TX, alpha-cypermethrin (202)+TX, alpha-ecdysone (alternative name) [CCN]+TX, aluminium phosphide (640)+TX, amidithion (870)+TX, amidothioate (872)+TX, aminocarb (873)+TX, amiton (875)+TX, amiton hydrogen oxalate (875)+TX, amitraz (24)+TX, anabasine (877)+TX, athidathion (883)+TX, AVI 382 (compound code)+TX, AZ 60541 (compound code)+TX, azadirachtin (alternative name) (41)+TX, azamethiphos (42)+TX, azinphos-ethyl (44)+TX, azinphos-methyl (45)+TX, azothoate (889)+TX, Bacillus thuringiensis delta endotoxins (alternative name) (52)+TX, barium hexafluorosilicate (alternative name) [CCN]+TX, barium polysulfide (IUPAC/Chemical Abstracts name) (892)+TX, barthrin [CCN]+TX, Bayer 22/190 (development code) (893)+TX, Bayer 22408 (development code) (894)+TX, bendiocarb (58)+TX, benfuracarb (60)+TX, bensultap (66)+TX, beta-cyfluthrin (194)+TX, beta-cypermethrin (203)+TX, bifenthrin (76)+TX, bioallethrin (78)+TX, bioallethrin S-cyclopentenyl isomer (alternative name) (79)+TX, bioethanomethrin [CCN]+TX, biopermethrin (908)+TX, bioresmethrin (80)+TX, bis(2-chloroethyl) ether (IUPAC name) (909)+TX, bistrifluron (83)+TX, borax (86)+TX, brofenvalerate (alternative name)+TX, bromfenvinfos (914)+TX, bromocyclen (918)+TX, bromo-DDT (alternative name) [CCN]+TX, bromophos (920)+TX, bromophos-ethyl (921)+TX, bufencarb (924)+TX, buprofezin (99)+TX, butacarb (926)+TX, butathiofos (927)+TX, butocarboxim (103)+TX, butonate (932)+TX, butoxycarboxim (104)+TX, butylpyridaben (alternative name)+TX, cadusafos (109)+TX, calcium arsenate [CCN]+TX, calcium cyanide (444)+TX, calcium polysulfide (IUPAC name) (111)+TX, camphechlor (941)+TX, carbanolate (943)+TX, carbaryl (115)+TX, carbofuran (118)+TX, carbon disulfide (IUPAC/Chemical Abstracts name) (945)+TX, carbon tetrachloride (IUPAC name) (946)+TX, carbophenothion (947)+TX, carbosulfan (119)+TX, cartap (123)+TX, cartap hydrochloride (123)+TX, cevadine (alternative name) (725)+TX, chlorbicyclen (960)+TX, chlordane (128)+TX, chlordecone (963)+TX, chlordimeform (964)+TX, chlordimeform hydrochloride (964)+TX, chlorethoxyfos (129)+TX, chlorfenapyr (130)+TX, chlorfenvinphos (131)+TX, chlorfluazuron (132)+TX, chlormephos (136)+TX, chloroform [CCN]+TX, chloropicrin (141)+TX, chlorphoxim (989)+TX, chlorprazophos (990)+TX, chlorpyrifos (145)+TX, chlorpyrifos-methyl (146)+TX, chlorthiophos (994)+TX, chromafenozide (150)+TX, cinerin I (696)+TX, cinerin II (696)+TX, cinerins (696)+TX, cis-resmethrin (alternative name)+TX, cismethrin (80)+TX, clocythrin (alternative name)+TX, cloethocarb (999)+TX, closantel (alternative name) [CCN]+TX, clothianidin (165)+TX, copper acetoarsenite [CCN]+TX, copper arsenate [CCN]+TX, copper oleate [CCN]+TX, coumaphos (174)+TX, coumithoate (1006)+TX, crotamiton (alternative name) [CCN]+TX, crotoxyphos (1010)+TX, crufomate (1011)+TX, cryolite (alternative name) (177)+TX, CS 708 (development code) (1012)+TX, cyanofenphos (1019)+TX, cyanophos (184)+TX, cyanthoate (1020)+TX, cyclethrin [CCN]+TX, cycloprothrin (188)+TX, cyfluthrin (193)+TX, cyhalothrin (196)+TX, cypermethrin (201)+TX, cyphenothrin (206)+TX, cyromazine (209)+TX, cythioate (alternative name) [CCN]+TX, d-limonene (alternative name) [CCN]+TX, d-tetramethrin (alternative name) (788)+TX, DAEP (1031)+TX, dazomet (216)+TX, DDT (219)+TX, decarbofuran (1034)+TX, deltamethrin (223)+TX, demephion (1037)+TX, demephion-O (1037)+TX, demephion-S (1037)+TX, demeton (1038)+TX, demeton-methyl (224)+TX, demeton-O (1038)+TX, demeton-O-methyl (224)+TX, demeton-S (1038)+TX, demeton-S-methyl (224)+TX, demeton-S-methylsulphon (1039)+TX, diafenthiuron (226)+TX, dimpropyridaz+TX, dialifos (1042)+TX, diamidafos (1044)+TX, diazinon (227)+TX, dicapthon (1050)+TX, dichlofenthion (1051)+TX, dichlorvos (236)+TX, dicliphos (alternative name)+TX, dicresyl (alternative name) [CCN]+TX, dicrotophos (243)+TX, dicyclanil (244)+TX, dieldrin (1070)+TX, diethyl 5-methylpyrazol-3-yl phosphate (IUPAC name) (1076)+TX, diflubenzuron (250)+TX, dilor (alternative name) [CCN]+TX, dimefluthrin [CCN]+TX, dimefox (1081)+TX, dimetan (1085)+TX, dimethoate (262)+TX, dimethrin (1083)+TX, dimethylvinphos (265)+TX, dimetilan (1086)+TX, dinex (1089)+TX, dinex-diclexine (1089)+TX, dinoprop (1093)+TX, dinosam (1094)+TX, dinoseb (1095)+TX, dinotefuran (271)+TX, diofenolan (1099)+TX, dioxabenzofos (1100)+TX, dioxacarb (1101)+TX, dioxathion (1102)+TX, disulfoton (278)+TX, dithicrofos (1108)+TX, DNOC (282)+TX, doramectin (alternative name) [CCN]+TX, DSP (1115)+TX, ecdysterone (alternative name) [CCN]+TX, EI 1642 (development code) (1118)+TX, emamectin (291)+TX, emamectin benzoate (291)+TX, EMPC (1120)+TX, empenthrin (292)+TX, endosulfan (294)+TX, endothion (1121)+TX, endrin (1122)+TX, EPBP (1123)+TX, EPN (297)+TX, epofenonane (1124)+TX, eprinomectin (alternative name) [CCN]+TX, esfenvalerate (302)+TX, etaphos (alternative name) [CCN]+TX, ethiofencarb (308)+TX, ethion (309)+TX, ethiprole (310)+TX, ethoate-methyl (1134)+TX, ethoprophos (312)+TX, ethyl formate (IUPAC name) [CCN]+TX, ethyl-DDD (alternative name) (1056)+TX, ethylene dibromide (316)+TX, ethylene dichloride (chemical name) (1136)+TX, ethylene oxide [CCN]+TX, etofenprox (319)+TX, etrimfos (1142)+TX, EXD (1143)+TX, famphur (323)+TX, fenamiphos (326)+TX, fenazaflor (1147)+TX, fenchlorphos (1148)+TX, fenethacarb (1149)+TX, fenfluthrin (1150)+TX, fenitrothion (335)+TX, fenobucarb (336)+TX, fenoxacrim (1153)+TX, fenoxycarb (340)+TX, fenpirithrin (1155)+TX, fenpropathrin (342)+TX, fenpyrad (alternative name)+TX, fensulfothion (1158)+TX, fenthion (346)+TX, fenthion-ethyl [CCN]+TX, fenvalerate (349)+TX, fipronil (354)+TX, flonicamid (358)+TX, flubendiamide (CAS. Reg. No.: 272451-65-7)+TX, flucofuron (1168)+TX, flucycloxuron (366)+TX, flucythrinate (367)+TX, fluenetil (1169)+TX, flufenerim [CCN]+TX, flufenoxuron (370)+TX, flufenprox (1171)+TX, flumethrin (372)+TX, fluvalinate (1184)+TX, FMC 1137 (development code) (1185)+TX, fonofos (1191)+TX, formetanate (405)+TX, formetanate hydrochloride (405)+TX, formothion (1192)+TX, formparanate (1193)+TX, fosmethilan (1194)+TX, fospirate (1195)+TX, fosthiazate (408)+TX, fosthietan (1196)+TX, furathiocarb (412)+TX, furethrin (1200)+TX, gamma-cyhalothrin (197)+TX, gamma-HCH (430)+TX, guazatine (422)+TX, guazatine acetates (422)+TX, GY-81 (development code) (423)+TX, halfenprox (424)+TX, halofenozide (425)+TX, HCH (430)+TX, HEOD (1070)+TX, heptachlor (1211)+TX, heptenophos (432)+TX, heterophos [CCN]+TX, hexaflumuron (439)+TX, HHDN (864)+TX, hydramethylnon (443)+TX, hydrogen cyanide (444)+TX, hydroprene (445)+TX, hyquincarb (1223)+TX, imidacloprid (458)+TX, imiprothrin (460)+TX, indoxacarb (465)+TX, iodomethane (IUPAC name) (542)+TX, IPSP (1229)+TX, isazofos (1231)+TX, isobenzan (1232)+TX, isocarbophos (alternative name) (473)+TX, isodrin (1235)+TX, isofenphos (1236)+TX, isolane (1237)+TX, isoprocarb (472)+TX, isopropyl O-(methoxy-aminothiophosphoryl)salicylate (IUPAC name) (473)+TX, isoprothiolane (474)+TX, isothioate (1244)+TX, isoxathion (480)+TX, ivermectin (alternative name) [CCN]+TX, jasmolin I (696)+TX, jasmolin II (696)+TX, jodfenphos (1248)+TX, juvenile hormone I (alternative name) [CCN]+TX, juvenile hormone II (alternative name) [CCN]+TX, juvenile hormone III (alternative name) [CCN]+TX, kelevan (1249)+TX, kinoprene (484)+TX, lambda-cyhalothrin (198)+TX, lead arsenate [CCN]+TX, lepimectin (CCN)+TX, leptophos (1250)+TX, lindane (430)+TX, lirimfos (1251)+TX, lufenuron (490)+TX, lythidathion (1253)+TX, m-cumenyl methylcarbamate (IUPAC name) (1014)+TX, magnesium phosphide (IUPAC name) (640)+TX, malathion (492)+TX, malonoben (1254)+TX, mazidox (1255)+TX, mecarbam (502)+TX, mecarphon (1258)+TX, menazon (1260)+TX, mephosfolan (1261)+TX, mercurous chloride (513)+TX, mesulfenfos (1263)+TX, metaflumizone (CCN)+TX, metam (519)+TX, metam-potassium (alternative name) (519)+TX, metam-sodium (519)+TX, methacrifos (1266)+TX, methamidophos (527)+TX, methanesulfonyl fluoride (IUPAC/Chemical Abstracts name) (1268)+TX, methidathion (529)+TX, methiocarb (530)+TX, methocrotophos (1273)+TX, methomyl (531)+TX, methoprene (532)+TX, methoquin-butyl (1276)+TX, methothrin (alternative name) (533)+TX, methoxychlor (534)+TX, methoxyfenozide (535)+TX, methyl bromide (537)+TX, methyl isothiocyanate (543)+TX, methylchloroform (alternative name) [CCN]+TX, methylene chloride [CCN]+TX, metofluthrin [CCN]+TX, metolcarb (550)+TX, metoxadiazone (1288)+TX, mevinphos (556)+TX, mexacarbate (1290)+TX, milbemectin (557)+TX, milbemycin oxime (alternative name) [CCN]+TX, mipafox (1293)+TX, mirex (1294)+TX, monocrotophos (561)+TX, morphothion (1300)+TX, moxidectin (alternative name) [CCN]+TX, naftalofos (alternative name) [CCN]+TX, naled (567)+TX, naphthalene (IUPAC/Chemical Abstracts name) (1303)+TX, NC-170 (development code) (1306)+TX, NC-184 (compound code)+TX, nicotine (578)+TX, nicotine sulfate (578)+TX, nifluridide (1309)+TX, nitenpyram (579)+TX, nithiazine (1311)+TX, nitrilacarb (1313)+TX, nitrilacarb 1:1 zinc chloride complex (1313)+TX, NNI-0101 (compound code)+TX, NNI-0250 (compound code)+TX, nornicotine (traditional name) (1319)+TX, novaluron (585)+TX, noviflumuron (586)+TX, O-5-dichloro-4-iodophenyl O-ethyl ethylphosphonothioate (IUPAC name) (1057)+TX, O,O-diethyl O-4-methyl-2-oxo-2H-chromen-7-yl phosphorothioate (IUPAC name) (1074)+TX, O,O-diethyl O-6-methyl-2-propylpyrimidin-4-yl phosphorothioate (IUPAC name) (1075)+TX, O,O,O′,O′-tetrapropyl dithiopyrophosphate (IUPAC name) (1424)+TX, oleic acid (IUPAC name) (593)+TX, omethoate (594)+TX, oxamyl (602)+TX, oxydemeton-methyl (609)+TX, oxydeprofos (1324)+TX, oxydisulfoton (1325)+TX, pp′-DDT (219)+TX, para-dichlorobenzene [CCN]+TX, parathion (615)+TX, parathion-methyl (616)+TX, penfluron (alternative name) [CCN]+TX, pentachlorophenol (623)+TX, pentachlorophenyl laurate (IUPAC name) (623)+TX, permethrin (626)+TX, petroleum oils (alternative name) (628)+TX, PH 60-38 (development code) (1328)+TX, phenkapton (1330)+TX, phenothrin (630)+TX, phenthoate (631)+TX, phorate (636)+TX, phosalone (637)+TX, phosfolan (1338)+TX, phosmet (638)+TX, phosnichlor (1339)+TX, phosphamidon (639)+TX, phosphine (IUPAC name) (640)+TX, phoxim (642)+TX, phoxim-methyl (1340)+TX, pirimetaphos (1344)+TX, pirimicarb (651)+TX, pirimiphos-ethyl (1345)+TX, pirimiphos-methyl (652)+TX, polychlorodicyclopentadiene isomers (IUPAC name) (1346)+TX, polychloroterpenes (traditional name) (1347)+TX, potassium arsenite [CCN]+TX, potassium thiocyanate [CCN]+TX, prallethrin (655)+TX, precocene I (alternative name) [CCN]+TX, precocene II (alternative name) [CCN]+TX, precocene III (alternative name) [CCN]+TX, primidophos (1349)+TX, profenofos (662)+TX, profluthrin [CCN]+TX, promacyl (1354)+TX, promecarb (1355)+TX, propaphos (1356)+TX, propetamphos (673)+TX, propoxur (678)+TX, prothidathion (1360)+TX, prothiofos (686)+TX, prothoate (1362)+TX, protrifenbute [CCN]+TX, pymetrozine (688)+TX, pyraclofos (689)+TX, pyrazophos (693)+TX, pyresmethrin (1367)+TX, pyrethrin I (696)+TX, pyrethrin II (696)+TX, pyrethrins (696)+TX, pyridaben (699)+TX, pyridalyl (700)+TX, pyridaphenthion (701)+TX, pyrimidifen (706)+TX, pyrimitate (1370)+TX, pyriproxyfen (708)+TX, quassia (alternative name) [CCN]+TX, quinalphos (711)+TX, quinalphos-methyl (1376)+TX, quinothion (1380)+TX, quintiofos (1381)+TX, R-1492 (development code) (1382)+TX, rafoxanide (alternative name) [CCN]+TX, resmethrin (719)+TX, rotenone (722)+TX, RU 15525 (development code) (723)+TX, RU 25475 (development code) (1386)+TX, ryania (alternative name) (1387)+TX, ryanodine (traditional name) (1387)+TX, sabadilla (alternative name) (725)+TX, schradan (1389)+TX, sebufos (alternative name)+TX, selamectin (alternative name) [CCN]+TX, SI-0009 (compound code)+TX, SI-0205 (compound code)+TX, SI-0404 (compound code)+TX, SI-0405 (compound code)+TX, silafluofen (728)+TX, SN 72129 (development code) (1397)+TX, sodium arsenite [CCN]+TX, sodium cyanide (444)+TX, sodium fluoride (IUPAC/Chemical Abstracts name) (1399)+TX, sodium hexafluorosilicate (1400)+TX, sodium pentachlorophenoxide (623)+TX, sodium selenate (IUPAC name) (1401)+TX, sodium thiocyanate [CCN]+TX, sophamide (1402)+TX, spinosad (737)+TX, spiromesifen (739)+TX, spirotetrmat (CCN)+TX, sulcofuron (746)+TX, sulcofuron-sodium (746)+TX, sulfluramid (750)+TX, sulfotep (753)+TX, sulfuryl fluoride (756)+TX, sulprofos (1408)+TX, tar oils (alternative name) (758)+TX, tau-fluvalinate (398)+TX, tazimcarb (1412)+TX, TDE (1414)+TX, tebufenozide (762)+TX, tebufenpyrad (763)+TX, tebupirimfos (764)+TX, teflubenzuron (768)+TX, tefluthrin (769)+TX, temephos (770)+TX, TEPP (1417)+TX, terallethrin (1418)+TX, terbam (alternative name)+TX, terbufos (773)+TX, tetrachloroethane [CCN]+TX, tetrachlorvinphos (777)+TX, tetramethrin (787)+TX, theta-cypermethrin (204)+TX, thiacloprid (791)+TX, thiafenox (alternative name)+TX, thiamethoxam (792)+TX, thicrofos (1428)+TX, thiocarboxime (1431)+TX, thiocyclam (798)+TX, thiocyclam hydrogen oxalate (798)+TX, thiodicarb (799)+TX, thiofanox (800)+TX, thiometon (801)+TX, thionazin (1434)+TX, thiosultap (803)+TX, thiosultap-sodium (803)+TX, thuringiensin (alternative name) [CCN]+TX, tolfenpyrad (809)+TX, tralomethrin (812)+TX, transfluthrin (813)+TX, transpermethrin (1440)+TX, triamiphos (1441)+TX, triazamate (818)+TX, triazophos (820)+TX, triazuron (alternative name)+TX, trichlorfon (824)+TX, trichlormetaphos-3 (alternative name) [CCN]+TX, trichloronat (1452)+TX, trifenofos (1455)+TX, triflumuron (835)+TX, trimethacarb (840)+TX, triprene (1459)+TX, vamidothion (847)+TX, vaniliprole [CCN]+TX, veratridine (alternative name) (725)+TX, veratrine (alternative name) (725)+TX, XMC (853)+TX, xylylcarb (854)+TX, YI-5302 (compound code)+TX, zeta-cypermethrin (205)+TX, zetamethrin (alternative name)+TX, zinc phosphide (640)+TX, zolaprofos (1469) and ZXI 8901 (development code) (858)+TX, cyantraniliprole [736994-63-19+TX, chlorantraniliprole [500008-45-7]+TX, cyenopyrafen [560121-52-0]+TX, cyflumetofen [400882-07-7]+TX, pyrifluquinazon [337458-27-2]+TX, spinetoram [187166-40-1+187166-15-0]+TX, spirotetramat [203313-25-1]+TX, sulfoxaflor [946578-00-3]+TX, flufiprole [704886-18-0]+TX, meperfluthrin [915288-13-0]+TX, tetramethylfluthrin [84937-88-2]+TX, triflumezopyrim (disclosed in WO 2012/092115)+TX, fluxametamide (WO 2007/026965)+TX, epsilon-metofluthrin [240494-71-7]+TX, epsilon-momfluorothrin [1065124-65-3]+TX, fluazaindolizine [1254304-22-7]+TX, chloroprallethrin [399572-87-3]+TX, fluxametamide [928783-29-3]+TX, cyhalodiamide [1262605-53-7]+TX, tioxazafen [330459-31-9]+TX, broflanilide [1207727-04-5]+TX, flufiprole [704886-18-0]+TX, cyclaniliprole [1031756-98-5]+TX, tetraniliprole [1229654-66-3]+TX, guadipyr (described in WO2010/060231)+TX, cycloxaprid (described in WO 2005/077934)+TX, spiropidion+TX, Afidopyropen+TX, flupyrimin+TX, Momfluorothrin+TX, kappa-bifenthrin+TX, kappa-tefluthrin+TX, Dichloromezotiaz+TX, Tetrachloraniliprole+TX, benzpyrimoxan+TX
a molluscicide selected from the group of substances consisting of bis(tributyltin) oxide (IUPAC name) (913)+TX, bromoacetamide [CCN]+TX, calcium arsenate [CCN]+TX, cloethocarb (999)+TX, copper acetoarsenite [CCN]+TX, copper sulfate (172)+TX, fentin (347)+TX, ferric phosphate (IUPAC name) (352)+TX, metaldehyde (518)+TX, methiocarb (530)+TX, niclosamide (576)+TX, niclosamide-olamine (576)+TX, pentachlorophenol (623)+TX, sodium pentachlorophenoxide (623)+TX, tazimcarb (1412)+TX, thiodicarb (799)+TX, tributyltin oxide (913)+TX, trifenmorph (1454)+TX, trimethacarb (840)+TX, triphenyltin acetate (IUPAC name) (347) and triphenyltin hydroxide (IUPAC name) (347)+TX, pyriprole [394730-71-3]+TX, a nematicide selected from the group of substances consisting of AKD-3088 (compound code)+TX, 1,2-dibromo-3-chloropropane (IUPAC/Chemical Abstracts name) (1045)+TX, 1,2-dichloropropane (IUPAC/Chemical Abstracts name) (1062)+TX, 1,2-dichloropropane with 1,3-dichloropropene (IUPAC name) (1063)+TX, 1,3-dichloropropene (233)+TX, 3,4-dichlorotetrahydrothiophene 1,1-dioxide (IUPAC/Chemical Abstracts name) (1065)+TX, 3-(4-chlorophenyl)-5-methylrhodanine (IUPAC name) (980)+TX, 5-methyl-6-thioxo-1,3,5-thiadiazinan-3-ylacetic acid (IUPAC name) (1286)+TX, 6-isopentenylaminopurine (alternative name) (210)+TX, abamectin (1)+TX, acetoprole [CCN]+TX, alanycarb (15)+TX, aldicarb (16)+TX, aldoxycarb (863)+TX, AZ 60541 (compound code)+TX, benclothiaz [CCN]+TX, benomyl (62)+TX, butylpyridaben (alternative name)+TX, cadusafos (109)+TX, carbofuran (118)+TX, carbon disulfide (945)+TX, carbosulfan (119)+TX, chloropicrin (141)+TX, chlorpyrifos (145)+TX, cloethocarb (999)+TX, cytokinins (alternative name) (210)+TX, dazomet (216)+TX, DBCP (1045)+TX, DCIP (218)+TX, diamidafos (1044)+TX, dichlofenthion (1051)+TX, dicliphos (alternative name)+TX, dimethoate (262)+TX, doramectin (alternative name) [CCN]+TX, emamectin (291)+TX, emamectin benzoate (291)+TX, eprinomectin (alternative name) [CCN]+TX, ethoprophos (312)+TX, ethylene dibromide (316)+TX, fenamiphos (326)+TX, fenpyrad (alternative name)+TX, fensulfothion (1158)+TX, fosthiazate (408)+TX, fosthietan (1196)+TX, furfural (alternative name) [CCN]+TX, GY-81 (development code) (423)+TX, heterophos [CCN]+TX, iodomethane (IUPAC name) (542)+TX, isamidofos (1230)+TX, isazofos (1231)+TX, ivermectin (alternative name) [CCN]+TX, kinetin (alternative name) (210)+TX, mecarphon (1258)+TX, metam (519)+TX, metam-potassium (alternative name) (519)+TX, metam-sodium (519)+TX, methyl bromide (537)+TX, methyl isothiocyanate (543)+TX, milbemycin oxime (alternative name) [CCN]+TX, moxidectin (alternative name) [CCN]+TX, Myrothecium verrucaria composition (alternative name) (565)+TX, NC-184 (compound code)+TX, oxamyl (602)+TX, phorate (636)+TX, phosphamidon (639)+TX, phosphocarb [CCN]+TX, sebufos (alternative name)+TX, selamectin (alternative name) [CCN]+TX, spinosad (737)+TX, terbam (alternative name)+TX, terbufos (773)+TX, tetrachlorothiophene (IUPAC/Chemical Abstracts name) (1422)+TX, thiafenox (alternative name)+TX, thionazin (1434)+TX, triazophos (820)+TX, triazuron (alternative name)+TX, xylenols [CCN]+TX, YI-5302 (compound code) and zeatin (alternative name) (210)+TX, fluensulfone [318290-98-1]+TX, fluopyram+TX,
a nitrification inhibitor selected from the group of substances consisting of potassium ethylxanthate [CCN] and nitrapyrin (580)+TX,
a plant activator selected from the group of substances consisting of acibenzolar (6)+TX, acibenzolar-S-methyl (6)+TX, probenazole (658) and Reynoutria sachalinensis extract (alternative name) (720)+TX,
a rodenticide selected from the group of substances consisting of 2-isovalerylindan-1,3-dione (IUPAC name) (1246)+TX, 4-(quinoxalin-2-ylamino)benzenesulfonamide (IUPAC name) (748)+TX, alpha-chlorohydrin [CCN]+TX, aluminium phosphide (640)+TX, antu (880)+TX, arsenous oxide (882)+TX, barium carbonate (891)+TX, bisthiosemi (912)+TX, brodifacoum (89)+TX, bromadiolone (91)+TX, bromethalin (92)+TX, calcium cyanide (444)+TX, chloralose (127)+TX, chlorophacinone (140)+TX, cholecalciferol (alternative name) (850)+TX, coumachlor (1004)+TX, coumafuryl (1005)+TX, coumatetralyl (175)+TX, crimidine (1009)+TX, difenacoum (246)+TX, difethialone (249)+TX, diphacinone (273)+TX, ergocalciferol (301)+TX, flocoumafen (357)+TX, fluoroacetamide (379)+TX, flupropadine (1183)+TX, flupropadine hydrochloride (1183)+TX, gamma-HCH (430)+TX, HCH (430)+TX, hydrogen cyanide (444)+TX, iodomethane (IUPAC name) (542)+TX, lindane (430)+TX, magnesium phosphide (IUPAC name) (640)+TX, methyl bromide (537)+TX, norbormide (1318)+TX, phosacetim (1336)+TX, phosphine (IUPAC name) (640)+TX, phosphorus [CCN]+TX, pindone (1341)+TX, potassium arsenite [CCN]+TX, pyrinuron (1371)+TX, scilliroside (1390)+TX, sodium arsenite [CCN]+TX, sodium cyanide (444)+TX, sodium fluoroacetate (735)+TX, strychnine (745)+TX, thallium sulfate [CCN]+TX, warfarin (851) and zinc phosphide (640)+TX,
a synergist selected from the group of substances consisting of 2-(2-butoxyethoxy)ethyl piperonylate (IUPAC name) (934)+TX, 5-(1,3-benzodioxol-5-yl)-3-hexylcyclohex-2-enone (IUPAC name) (903)+TX, farnesol with nerolidol (alternative name) (324)+TX, MB-599 (development code) (498)+TX, MGK 264 (development code) (296)+TX, piperonyl butoxide (649)+TX, piprotal (1343)+TX, propyl isomer (1358)+TX, S421 (development code) (724)+TX, sesamex (1393)+TX, sesasmolin (1394) and sulfoxide (1406)+TX,
an animal repellent selected from the group of substances consisting of anthraquinone (32)+TX, chloralose (127)+TX, copper naphthenate [CCN]+TX, copper oxychloride (171)+TX, diazinon (227)+TX, dicyclopentadiene (chemical name) (1069)+TX, guazatine (422)+TX, guazatine acetates (422)+TX, methiocarb (530)+TX, pyridin-4-amine (IUPAC name) (23)+TX, thiram (804)+TX, trimethacarb (840)+TX, zinc naphthenate [CCN] and ziram (856)+TX, a virucide selected from the group of substances consisting of imanin (alternative name) [CCN] and ribavirin (alternative name) [CCN]+TX,
a wound protectant selected from the group of substances consisting of mercuric oxide (512)+TX, octhilinone (590) and thiophanate-methyl (802)+TX,
and biologically active compounds selected from the group consisting of azaconazole (60207-31-0]+TX, bitertanol [70585-36-3]+TX, bromuconazole [116255-48-2]+TX, cyproconazole [94361-06-5]+TX, difenoconazole [119446-68-3]+TX, diniconazole [83657-24-3]+TX, epoxiconazole [106325-08-0]+TX, fenbuconazole [114369-43-6]+TX, fluquinconazole [136426-54-5]+TX, flusilazole [85509-19-9]+TX, flutriafol [76674-21-0]+TX, hexaconazole [79983-71-4]+TX, imazalil [35554-44-0]+TX, imibenconazole [86598-92-7]+TX, ipconazole [125225-28-7]+TX, metconazole [125116-23-6]+TX, myclobutanil [88671-89-0]+TX, pefurazoate [101903-30-4]+TX, penconazole [66246-88-6]+TX, prothioconazole [178928-70-6]+TX, pyrifenox [88283-41-4]+TX, prochloraz [67747-09-5]+TX, propiconazole [60207-90-1]+TX, simeconazole [149508-90-7]+TX, tebuconazole [107534-96-3]+TX, tetraconazole [112281-77-3]+TX, triadimefon [43121-43-3]+TX, triadimenol [55219-65-3]+TX, triflumizole [99387-89-0]+TX, triticonazole [131983-72-7]+TX, ancymidol [12771-68-5]+TX, fenarimol [60168-88-9]+TX, nuarimol [63284-71-9]+TX, bupirimate [41483-43-6]+TX, dimethirimol [5221-53-4]+TX, ethirimol [23947-60-6]+TX, dodemorph [1593-77-7]+TX, fenpropidine [67306-00-7]+TX, fenpropimorph [67564-91-4]+TX, spiroxamine [118134-30-8]+TX, tridemorph [81412-43-3]+TX, cyprodinil [121552-61-2]+TX, mepanipyrim [110235-47-7]+TX, pyrimethanil [53112-28-0]+TX, fenpiclonil [74738-17-3]+TX, fludioxonil [131341-86-1]+TX, benalaxyl [71626-11-4]+TX, furalaxyl [57646-30-7]+TX, meta-laxyl [57837-19-1]+TX, R-metalaxyl [70630-17-0]+TX, ofurace [58810-48-3]+TX, oxadixyl [77732-09-3]+TX, benomyl [17804-35-2]+TX, carbendazim [10605-21-7]+TX, debacarb [62732-91-6]+TX, fuberidazole [3878-19-1]+TX, thiabendazole [148-79-8]+TX, chlozolinate [84332-86-5]+TX, dichlozoline [24201-58-9]+TX, iprodione [36734-19-7]+TX, myclozoline [54864-61-8]+TX, procymidone [32809-16-8]+TX, vinclozoline [50471-44-8]+TX, boscalid [188425-85-6]+TX, carboxin [5234-68-4]+TX, fenfuram [24691-80-3]+TX, flutolanil [66332-96-5]+TX, mepronil [55814-41-0]+TX, oxycarboxin [5259-88-1]+TX, penthiopyrad [183675-82-3]+TX, thifluzamide [130000-40-7]+TX, guazatine [108173-90-6]+TX, dodine [2439-10-3] [112-65-2] (free base)+TX, iminoctadine [13516-27-3]+TX, azoxystrobin [131860-33-8]+TX, dimoxystrobin [149961-52-4]+TX, enestroburin {Proc. BCPC, Int. Congr., Glasgow, 2003, 1, 93}+TX, fluoxastrobin [361377-29-9]+TX, kresoxim-methyl [143390-89-0]+TX, metominostrobin [133408-50-1]+TX, trifloxystrobin [141517-21-7]+TX, orysastrobin [248593-16-0]+TX, picoxystrobin [117428-22-5]+TX, pyraclostrobin [175013-18-0]+TX, ferbam [14484-64-1]+TX, mancozeb [8018-01-7]+TX, maneb [12427-38-2]+TX, metiram [9006-42-2]+TX, propineb [12071-83-9]+TX, thiram [137-26-8]+TX, zineb [12122-67-7]+TX, ziram [137-30-4]+TX, captafol [2425-06-1]+TX, captan [133-06-2]+TX, dichlofluanid [1085-98-9]+TX, fluoroimide [41205-21-4]+TX, folpet [133-07-3]+TX, tolylfluanid [731-27-1]+TX, bordeaux mixture [8011-63-0]+TX, copperhydroxid [20427-59-2]+TX, copperoxychlorid [1332-40-7]+TX, coppersulfat [7758-98-7]+TX, copperoxid [1317-39-1]+TX, mancopper [53988-93-5]+TX, oxine-copper [10380-28-6]+TX, dinocap [131-72-6]+TX, nitrothal-isopropyl [10552-74-6]+TX, edifenphos [17109-49-8]+TX, iprobenphos [26087-47-8]+TX, isoprothiolane [50512-35-1]+TX, phosdiphen [36519-00-3]+TX, pyrazophos [13457-18-6]+TX, tolclofos-methyl [57018-04-9]+TX, acibenzo-lar-S-methyl [135158-54-2]+TX, anilazine [101-05-3]+TX, benthiavalicarb [413615-35-7]+TX, blasticidin-S [2079-00-7]+TX, chinomethionat [2439-01-2]+TX, chloroneb [2675-77-6]+TX, chlorothalonil [1897-45-6]+TX, cyflufenamid [180409-60-3]+TX, cymoxanil [57966-95-7]+TX, dichlone [117-80-6]+TX, diclocymet [139920-32-4]+TX, diclomezine [62865-36-5]+TX, dicloran [99-30-9]+TX, diethofencarb [87130-20-9]+TX, dimethomorph [110488-70-5]+TX, SYP-LI90 (Flumorph) [211867-47-9]+TX, dithianon [3347-22-6]+TX, ethaboxam [162650-77-3]+TX, etridiazole [2593-15-9]+TX, famoxadone [131807-57-3]+TX, fenamidone [161326-34-7]+TX, fenoxanil [115852-48-7]+TX, fentin [668-34-8]+TX, ferimzone [89269-64-7]+TX, fluazinam [79622-59-6]+TX, fluopicolide [239110-15-7]+TX, flusulfamide [106917-52-6]+TX, fenhexamid [126833-17-8]+TX, fosetyl-aluminium [39148-24-8]+TX, hymexazol [10004-44-1]+TX, iprovalicarb [140923-17-7]+TX, IKF-916 (Cyazofamid) [120116-88-3]+TX, kasugamycin [6980-18-3]+TX, methasulfocarb [66952-49-6]+TX, metrafenone [220899-03-6]+TX, pencycuron [66063-05-6]+TX, phthalide [27355-22-2]+TX, polyoxins [11113-80-7]+TX, probenazole [27605-76-1]+TX, propamocarb [25606-41-1]+TX, proquinazid [189278-12-4]+TX, pyroquilon [57369-32-1]+TX, quinoxyfen [124495-18-7]+TX, quintozene [82-68-8]+TX, sulfur [7704-34-9]+TX, tiadinil [223580-51-6]+TX, triazoxide [72459-58-6]+TX, tricyclazole [41814-78-2]+TX, triforine [26644-46-2]+TX, validamycin [37248-47-8]+TX, zoxamide (RH7281) [156052-68-5]+TX, mandipropamid [374726-62-2]+TX, isopyrazam [881685-58-1]+TX, sedaxane [874967-67-6]+TX, 3-difluoromethyl-1-methyl-1H-pyrazole-4-carboxylic acid (9-dichloromethylene-1,2,3,4-tetrahydro-1,4-methano-naphthalen-5-yl)-amide (disclosed in WO 2007/048556)+TX, 3-difluoromethyl-1-methyl-1H-pyrazole-4-carboxylic acid (3′,4′,5′-trifluoro-biphenyl-2-yl)-amide (disclosed in WO 2006/087343)+TX, [(3S,4R,4aR,6S,6aS,12R,12aS,12bS)-3-[(cyclopropylcarbonyl)oxy]-1,3,4,4a,5,6,6a,12,12a,12b-decahydro-6,12-dihydroxy-4,6a,12b-trimethyl-11-oxo-9-(3-pyridinyl)-2H,11Hnaphtho[2,1-b]pyrano[3,4-e]pyran-4-yl]methyl-cyclopropanecarboxylate [915972-17-7]+TX and 1,3,5-trimethyl-N-(2-methyl-1-oxopropyl)-N-[3-(2-methylpropyl)-4-[2,2,2-trifluoro-1-methoxy-1-(trifluoromethyl)ethyl]phenyl]-1H-pyrazole-4-carboxamide [926914-55-8]+TX; lancotrione [1486617-21-3]+TX, florpyrauxifen [943832-81-3]]+TX, ipfentrifluconazole[1417782-08-1]+TX, mefentrifluconazole [1417782-03-6]+TX, quinofumelin [861647-84-9]+TX, chloroprallethrin [399572-87-3]+TX, cyhalodiamide [1262605-53-7]]+TX, fluazaindolizine [1254304-22-7]+TX, fluxametamide [928783-29-3]+TX, epsilon-metofluthrin [240494-71-7]+TX, epsilon-momfluorothrin [1065124-65-3]+TX, pydiflumetofen [1228284-64-7]+TX, kappa-bifenthrin [439680-76-9]+TX, broflanilide [1207727-04-5]+TX, dicloromezotiaz [1263629-39-5]+TX, dipymetitrone [16114-35-5]+TX, pyraziflumid [942515-63-1]+TX, kappa-tefluthrin [391634-71-2]+TX, fenpicoxamid [517875-34-2]+TX; fluindapyr [1383809-87-7]+TX; alpha-bromadiolone [28772-56-7]+TX; flupyrimin [1689566-03-7]+TX; benzpyrimoxan [1449021-97-9]+TX; acynonapyr [1332838-17-1]+TX; inpyrfluxam [1352994-67-2]+TX, isoflucypram [1255734-28-1]+TX; rescalure [64309-03-1]+TX; aminopyrifen [1531626-08-0]+TX; tyclopyrazoflor [1477919-27-9]+TX; and spiropidion [1229023-00-0]+TX; and microbials including: Acinetobacter lwoffii+TX, Acremonium alternatum+TX+TX, Acremonium cephalosporium+TX+TX, Acremonium diospyri+TX, Acremonium obclavatum+TX, Adoxophyes orana granulovirus (AdoxGV) (Capex®)+TX, Agrobacterium radiobacter strain K84 (Galltrol-A®)+TX, Alternaria alternate+TX, Alternaria cassia+TX, Alternaria destruens (Smolder®)+TX, Ampelomyces quisqualis (AQ10®)+TX, Aspergillus flavus AF36 (AF36®)+TX, Aspergillus flavus NRRL 21882 (Aflaguard®)+TX, Aspergillus spp.+TX, Aureobasidium pullulans+TX, Azospirillum+TX, (MicroAZ®+TX, TAZO B®)+TX, Azotobacter+TX, Azotobacter chroocuccum (Azotomeal®)+TX, Azotobacter cysts (Bionatural Blooming Blossoms®)+TX, Bacillus amyloliquefaciens+TX, Bacillus cereus+TX, Bacillus chitinosporus strain CM-1+TX, Bacillus chitinosporus strain AQ746+TX, Bacillus licheniformis strain HB-2 (Biostart™ Rhizoboost®)+TX, Bacillus licheniformis strain 3086 (EcoGuard®+TX, Green Releaf®)+TX, Bacillus circulans+TX, Bacillus firmus (BioSafe®+TX, BioNem-WP®+TX, VOTiVO®)+TX, Bacillus firmus strain 1-1582+TX, Bacillus macerans+TX, Bacillus marismortui+TX, Bacillus megaterium+TX, Bacillus mycoides strain AQ726+TX, Bacillus papillae (Milky Spore Powder®)+TX, Bacillus pumilus spp.+TX, Bacillus pumilus strain GB34 (Yield Shield®)+TX, Bacillus pumilus strain AQ717+TX, Bacillus pumilus strain QST 2808 (Sonata®+TX, Ballad Plus®)+TX, Bacillus spahericus (VectoLex®)+TX, Bacillus spp.+TX, Bacillus spp. strain AQ175+TX, Bacillus spp. strain AQ177+TX, Bacillus spp. strain AQ178+TX, Bacillus subtilis strain QST 713 (CEASE®+TX, Serenade®+TX, Rhapsody®)+TX, Bacillus subtilis strain QST 714 (JAZZ®)+TX, Bacillus subtilis strain AQ153+TX, Bacillus subtilis strain AQ743+TX, Bacillus subtilis strain QST3002+TX, Bacillus subtilis strain QST3004+TX, Bacillus subtilis var. amyloliquefaciens strain FZB24 (Taegro®+TX, Rhizopro®)+TX, Bacillus thuringiensis Cry 2Ae+TX, Bacillus thuringiensis Cry1Ab+TX, Bacillus thuringiensis aizawai GC 91 (Agree®)+TX, Bacillus thuringiensis israelensis (BMP123®+TX, Aquabac®+TX, VectoBac®)+TX, Bacillus thuringiensis kurstaki (Javelin®+TX, Deliver®+TX, CryMax®+TX, Bonide®+TX, Scutella WP®+TX, Turilav WP®+TX, Astuto®+TX, Dipel WP®+TX, Biobit®+TX, Foray®)+TX, Bacillus thuringiensis kurstaki BMP 123 (Baritone®)+TX, Bacillus thuringiensis kurstaki HD-1 (Bioprotec-CAF/3P®)+TX, Bacillus thuringiensis strain BD #32+TX, Bacillus thuringiensis strain AQ52+TX, Bacillus thuringiensis var. aizawai (XenTari®+TX, DiPel®)+TX, bacteria spp. (GROWMEND®+TX, GROWSWEET®+TX, Shootup®)+TX, bacteriophage of Clavipacter michiganensis (AgriPhage®)+TX, Bakflor®+TX, Beauveria bassiana (Beaugenic®+TX, Brocaril WP®)+TX, Beauveria bassiana GHA (Mycotrol ES®+TX, Mycotrol O®+TX, BotaniGuard®)+TX, Beauveria brongniartii (Engerlingspilz®+TX, Schweizer Beauveria®+TX, Melocont®)+TX, Beauveria spp.+TX, Botrytis cineria+TX, Bradyrhizobium japonicum (TerraMax®)+TX, Brevibacillus brevis+TX, Bacillus thuringiensis tenebrionis (Novodor®)+TX, BtBooster+TX, Burkholderia cepacia (Deny®+TX, Intercept®+TX, Blue Circle®)+TX, Burkholderia gladii+TX, Burkholderia gladioli+TX, Burkholderia spp.+TX, Canadian thistle fungus (CBH Canadian Bioherbicide®)+TX, Candida butyri+TX, Candida famata+TX, Candida fructus+TX, Candida glabrata+TX, Candida guilliermondii+TX, Candida melibiosica+TX, Candida oleophila strain O+TX, Candida parapsilosis+TX, Candida pelliculosa+TX, Candida pulcherrima+TX, Candida reukaufii+TX, Candida saitoana (Bio-Coat®+TX, Biocure®)+TX, Candida sake+TX, Candida spp.+TX, Candida tenius+TX, Cedecea dravisae+TX, Cellulomonas flavigena+TX, Chaetomium cochliodes (Nova-Cide®)+TX, Chaetomium globosum (Nova-Cide®)+TX, Chromobacterium subtsugae strain PRAA4-1T (Grandevo®)+TX, Cladosporium cladosporioides+TX, Cladosporium oxysporum+TX, Cladosporium chlorocephalum+TX, Cladosporium spp.+TX, Cladosporium tenuissimum+TX, Clonostachys rosea (EndoFine®)+TX, Colletotrichum acutatum+TX, Coniothyrium minitans (Cotans WG®)+TX, Coniothyrium spp.+TX, Cryptococcus albidus (YIELDPLUS®)+TX, Cryptococcus humicola+TX, Cryptococcus infirmo-miniatus+TX, Cryptococcus laurentii+TX, Cryptophlebia leucotreta granulovirus (Cryptex®)+TX, Cupriavidus campinensis+TX, Cydia pomonella granulovirus (CYD-X®)+TX, Cydia pomonella granulovirus (Madex®+TX, Madex Plus®+TX, Madex Max/Carpovirusine®)+TX, Cylindrobasidium laeve (Stumpout®)+TX, Cylindrocladium+TX, Debaryomyces hansenii+TX, Drechslera hawaiinensis+TX, Enterobacter cloacae+TX, Enterobacteriaceae+TX, Entomophtora virulenta (Vektor®)+TX, Epicoccum nigrum+TX, Epicoccum purpurascens+TX, Epicoccum spp.+TX, Filobasidium floriforme+TX, Fusarium acuminatum+TX, Fusarium chlamydosporum+TX, Fusarium oxysporum (Fusaclean®/Biofox C®)+TX, Fusarium proliferatum+TX, Fusarium spp.+TX, Galactomyces geotrichum+TX, Gliocladium catenulatum (Primastop®+TX, Prestop®)+TX, Gliocladium roseum+TX, Gliocladium spp. (SoilGard®)+TX, Gliocladium virens (Soilgard®)+TX, Granulovirus (Granupom®)+TX, Halobacillus halophilus+TX, Halobacillus litoralis+TX, Halobacillus trueperi+TX, Halomonas spp.+TX, Halomonas subglaciescola+TX, Halovibrio variabilis+TX, Hanseniaspora uvarum+TX, Helicoverpa armigera nucleopolyhedrovirus (Helicovex®)+TX, Helicoverpa zea nuclear polyhedrosis virus (Gemstar®)+TX, Isoflavone—formononetin (Myconate®)+TX, Kloeckera apiculata+TX, Kloeckera spp.+TX, Lagenidium giganteum (Laginex®)+TX, Lecanicillium longisporum (Vertiblast®)+TX, Lecanicillium muscarium (Vertikil®)+TX, Lymantria dispar nucleopolyhedrosis virus (Disparvirus®)+TX, Marinococcus halophilus+TX, Meira geulakonigii+TX, Metarhizium anisopliae (Met52®)+TX, Metarhizium anisopliae (Destruxin WP®)+TX, Metschnikowia fruticola (Shemer®)+TX, Metschnikowia pulcherrima+TX, Microdochium dimerum (Antibot®)+TX, Micromonospora coerulea+TX, Microsphaeropsis ochracea+TX, Muscodor albus 620 (Muscudor®)+TX, Muscodor roseus strain A3-5+TX, Mycorrhizae spp. (AMykor®+TX, Root Maximizer®)+TX, Myrothecium verrucaria strain AARC-0255 (DiTera®)+TX, BROS PLUS®+TX, Ophiostoma piliferum strain D97 (Sylvanex®)+TX, Paecilomyces farinosus+TX, Paecilomyces fumosoroseus (PFR-97®+TX, PreFeRal®)+TX, Paecilomyces linacinus (Biostat WP®)+TX, Paecilomyces lilacinus strain 251 (MeloCon WG®)+TX, Paenibacillus polymyxa+TX, Pantoea agglomerans (BlightBan C9-1®)+TX, Pantoea spp.+TX, Pasteuria spp. (Econem®)+TX, Pasteuria nishizawae+TX, Penicillium aurantiogriseum+TX, Penicillium billai (Jumpstart®+TX, TagTeam®)+TX, Penicillium brevicompactum+TX, Penicillium frequentans+TX, Penicillium griseofulvum+TX, Penicillium purpurogenum+TX, Penicillium spp.+TX, Penicillium viridicatum+TX, Phlebiopsis gigantean (Rotstop®)+TX, phosphate solubilizing bacteria (Phosphomeal®)+TX, Phytophthora cryptogea+TX, Phytophthora palmivora (Devine®)+TX, Pichia anomala+TX, Pichia guilermondii+TX, Pichia membranaefaciens+TX, Pichia onychis+TX, Pichia stipites+TX, Pseudomonas aeruginosa+TX, Pseudomonas aureofasciens (Spot-Less Biofungicide®)+TX, Pseudomonas cepacia+TX, Pseudomonas chlororaphis (AtEze®)+TX, Pseudomonas corrugate+TX, Pseudomonas fluorescens strain A506 (BlightBan A506®)+TX, Pseudomonas putida+TX, Pseudomonas reactans+TX, Pseudomonas spp.+TX, Pseudomonas syringae (Bio-Save®)+TX, Pseudomonas viridiflava+TX, Pseudomons fluorescens (Zequanox®)+TX, Pseudozyma flocculosa strain PF-A22 UL (Sporodex L®)+TX, Puccinia canaliculata+TX, Puccinia thlaspeos (Wood Warrior®)+TX, Pythium paroecandrum+TX, Pythium oligandrum (Polygandron®+TX, Polyversum®)+TX, Pythium periplocum+TX, Rhanella aquatilis+TX, Rhanella spp.+TX, Rhizobia (Dormal®+TX, Vault®)+TX, Rhizoctonia+TX, Rhodococcus globerulus strain AQ719+TX, Rhodosporidium diobovatum+TX, Rhodosporidium toruloides+TX, Rhodotorula spp.+TX, Rhodotorula glutinis+TX, Rhodotorula graminis+TX, Rhodotorula mucilagnosa+TX, Rhodotorula rubra+TX, Saccharomyces cerevisiae+TX, Salinococcus roseus+TX, Sclerotinia minor+TX, Sclerotinia minor (SARRITOR®)+TX, Scytalidium spp.+TX, Scytalidium uredinicola+TX, Spodoptera exigua nuclear polyhedrosis virus (Spod-X®+TX, Spexit®)+TX, Serratia marcescens+TX, Serratia plymuthica+TX, Serratia spp.+TX, Sordaria fimicola+TX, Spodoptera littoralis nucleopolyhedrovirus (Littovir®)+TX, Sporobolomyces roseus+TX, Stenotrophomonas maltophilia+TX, Streptomyces ahygroscopicus+TX, Streptomyces albaduncus+TX, Streptomyces exfoliates+TX, Streptomyces galbus+TX, Streptomyces griseoplanus+TX, Streptomyces griseoviridis (Mycostop®)+TX, Streptomyces lydicus (Actinovate®)+TX, Streptomyces lydicus WYEC-108 (ActinoGrow®)+TX, Streptomyces violaceus+TX, Tilletiopsis minor+TX, Tilletiopsis spp.+TX, Trichoderma asperellum (T34 Biocontrol®)+TX, Trichoderma gamsii (Tenet®)+TX, Trichoderma atroviride (Plantmate®)+TX, Trichoderma hamatum TH 382+TX, Trichoderma harzianum rifai (Mycostar®)+TX, Trichoderma harzianum T-22 (Trianum-P®+TX, PlantShield HCO+TX, RootShield®+TX, Trianum-G®)+TX, Trichoderma harzianum T-39 (Trichodex®)+TX, Trichoderma inhamatum+TX, Trichoderma koningii+TX, Trichoderma spp. LC 52 (Sentinel®)+TX, Trichoderma lignorum+TX, Trichoderma longibrachiatum+TX, Trichoderma polysporum (Binab T®)+TX, Trichoderma taxi+TX, Trichoderma virens+TX, Trichoderma virens (formerly Gliocladium virens GL-21) (SoilGuard®)+TX, Trichoderma viride+TX, Trichoderma viride strain ICC 080 (Remedier®)+TX, Trichosporon pullulans+TX, Trichosporon spp.+TX, Trichothecium spp.+TX, Trichothecium roseum+TX, Typhula phacorrhiza strain 94670+TX, Typhula phacorrhiza strain 94671+TX, Ulocladium atrum+TX, Ulocladium oudemansii (Botry-Zen®)+TX, Ustilago maydis+TX, various bacteria and supplementary micronutrients (Natural II®)+TX, various fungi (Millennium Microbes®)+TX, Verticillium chlamydosporium+TX, Verticillium lecanii (Mycotal®+TX, Vertalec®)+TX, Vip3Aa20 (VIPtera®)+TX, Virgibaclillus marismortui+TX, Xanthomonas campestris pv. Poae (Camperico®)+TX, Xenorhabdus bovienii+TX, Xenorhabdus nematophilus; and
Plant extracts including: pine oil (Retenol®)+TX, azadirachtin (Plasma Neem Oil®+TX, AzaGuard®+TX, MeemAzal®+TX, Molt-X®+TX, Botanical IGR (Neemazad®+TX, Neemix®)+TX, canola oil (Lilly Miller Vegol®)+TX, Chenopodium ambrosioides near ambrosioides (Requiem®)+TX, Chrysanthemum extract (Crisant®)+TX, extract of neem oil (Trilogy®)+TX, essentials oils of Labiatae (Botania®)+TX, extracts of clove rosemary peppermint and thyme oil (Garden insect Killer®)+TX, Glycinebetaine (Greenstim®)+TX, garlic+TX, lemongrass oil (GreenMatch®)+TX, neem oil+TX, Nepeta cataria (Catnip oil)+TX, Nepeta catarina+TX, nicotine+TX, oregano oil (MossBuster®)+TX, Pedaliaceae oil (Nematon®)+TX, pyrethrum+TX, Quillaja saponaria (NemaQ®)+TX, Reynoutria sachalinensis (Regalia®+TX, Sakalia®)+TX, rotenone (Eco Roten®)+TX, Rutaceae plant extract (Soleo®)+TX, soybean oil (Ortho Ecosense®)+TX, tea tree oil (Timorex Gold®)+TX, thymus oil+TX, AGNIQUE® MMF+TX, BugOil®+TX, mixture of rosemary sesame peppermint thyme and cinnamon extracts (EF 300®)+TX, mixture of clove rosemary and peppermint extract (EF 400®)+TX, mixture of clove peppermint garlic oil and mint (Soil Shot®)+TX, kaolin (Screen®)+TX, storage glucam of brown algae (Laminarin®); and
pheromones including: blackheaded fireworm pheromone (3M Sprayable Blackheaded Fireworm Pheromone®)+TX, Codling Moth Pheromone (Paramount dispenser-(CM)/Isomate C-Plus®)+TX, Grape Berry Moth Pheromone (3M MEC-GBM Sprayable Pheromone®)+TX, Leafroller pheromone (3M MEC—LR Sprayable Pheromone®)+TX, Muscamone (Snip7 Fly Bait®+TX, Starbar Premium Fly Bait®)+TX, Oriental Fruit Moth Pheromone (3M oriental fruit moth sprayable Pheromone®)+TX, Peachtree Borer Pheromone (Isomate-P®)+TX, Tomato Pinworm Pheromone (3M Sprayable Pheromone®)+TX, Entostat powder (extract from palm tree) (Exosex CM®)+TX, (E+TX,Z+TX,Z)-3+TX,8+TX,11 Tetradecatrienyl acetate+TX, (Z+TX,Z+TX,E)-7+TX,11+TX,13-Hexadecatrienal+TX, (E+TX,Z)-7+TX,9-Dodecadien-1-yl acetate+TX, 2-Methyl-1-butanol+TX, Calcium acetate+TX, Scenturion®+TX, Biolure®+TX, Check-Mate®+TX, Lavandulyl senecioate; and
Macrobials including: Aphelinus abdominalis+TX, Aphidius ervi (Aphelinus-System®)+TX, Acerophagus papaya+TX, Adalia bipunctata (Adalia-System®)+TX, Adalia bipunctata (Adaline®)+TX, Adalia bipunctata (Aphidalia®)+TX, Ageniaspis citricola+TX, Ageniaspis fuscicollis+TX, Amblyseius andersoni (Anderline®+TX, Andersoni-System®)+TX, Amblyseius californicus (Amblyline®+TX, Spical®)+TX, Amblyseius cucumeris (Thripex®+TX, Bugline Cucumeris®)+TX, Amblyseius fallacis (Fallacis®)+TX, Amblyseius swirskii (Bugline Swirskii®+TX, Swirskii-Mite®)+TX, Amblyseius womersleyi (WomerMite®)+TX, Amitus hesperidum+TX, Anagrus atomus+TX, Anagyrus fusciventris+TX, Anagyrus kamali+TX, Anagyrus loecki+TX, Anagyrus pseudococci (Citripar®)+TX, Anicetus benefices+TX, Anisopteromalus calandrae+TX, Anthocoris nemoralis (Anthocoris-System®)+TX, Aphelinus abdominalis (Apheline®+TX, Aphiline®)+TX, Aphelinus asychis+TX, Aphidius colemani (Aphipar®)+TX, Aphidius ervi (Ervipar®)+TX, Aphidius gifuensis+TX, Aphidius matricariae (Aphipar-M®)+TX, Aphidoletes aphidimyza (Aphidend®)+TX, Aphidoletes aphidimyza (Aphidoline®)+TX, Aphytis lingnanensis+TX, Aphytis melinus+TX, Aprostocetus hagenowii+TX, Atheta coriaria (Staphyline®)+TX, Bombus spp.+TX, Bombus terrestris (Natupol Beehive®)+TX, Bombus terrestris (Beeline®+TX, Tripol®)+TX, Cephalonomia stephanoderis+TX, Chilocorus nigritus+TX, Chrysoperla carnea (Chrysoline®)+TX, Chrysoperla carnea (Chrysopa®)+TX, Chrysoperla rufilabris+TX, Cirrospilus ingenuus+TX, Cirrospilus quadristriatus+TX, Citrostichus phyllocnistoides+TX, Closterocerus chamaeleon+TX, Closterocerus spp.+TX, Coccidoxenoides perminutus (Planopar®)+TX, Coccophagus cowperi+TX, Coccophagus lycimnia+TX, Cotesia flavipes+TX, Cotesia plutellae+TX, Cryptolaemus montrouzieri (Cryptobug®+TX, Cryptoline®)+TX, Cybocephalus nipponicus+TX, Dacnusa sibirica+TX, Dacnusa sibirica (Minusa®)+TX, Diglyphus isaea (Diminex®)+TX, Delphastus catalinae (Delphastus®)+TX, Delphastus pusillus+TX, Diachasmimorpha krausii+TX, Diachasmimorpha longicaudata+TX, Diaparsis jucunda+TX, Diaphorencyrtus aligarhensis+TX, Diglyphus isaea+TX, Diglyphus isaea (Miglyphus®+TX, Digline®)+TX, Dacnusa sibirica (DacDigline®+TX, Minex®)+TX, Diversinervus spp.+TX, Encarsia citrina+TX, Encarsia formosa (Encarsia Max®+TX, Encarline®+TX, En-Strip®)+TX, Eretmocerus eremicus (Enermix®)+TX, Encarsia guadeloupae+TX, Encarsia haitiensis+TX, Episyrphus balteatus (Syrphidend®)+TX, Eretmoceris siphonini+TX, Eretmocerus californicus+TX, Eretmocerus eremicus (Ercal®+TX, Eretline E®)+TX, Eretmocerus eremicus (Bemimix®)+TX, Eretmocerus hayati+TX, Eretmocerus mundus (Bemipar®+TX, Eretline M®)+TX, Eretmocerus siphonini+TX, Exochomus quadripustulatus+TX, Feltiella acarisuga (Spidend®)+TX, Feltiella acarisuga (Feltiline®)+TX, Fopius arisanus+TX, Fopius ceratitivorus+TX, Formononetin (Wirless Beehome®)+TX, Franklinothrips vespiformis (Vespop®)+TX, Galendromus occidentalis+TX, Goniozus legneri+TX, Habrobracon hebetor+TX, Harmonia axyridis (HarmoBeetle®)+TX, Heterorhabditis spp. (Lawn Patrol®)+TX, Heterorhabditis bacteriophora (NemaShield HB®+TX, Nemaseek®+TX, Terranem-Nam®+TX, Terranem®+TX, Larvanem®+TX, B-Green®+TX, NemAttack®+TX, Nematop®)+TX, Heterorhabditis megidis (Nemasys H®+TX, BioNem H®+TX, Exhibitline Hm®+TX, Larvanem-M®)+TX, Hippodamia convergens+TX, Hypoaspis aculeifer (Aculeifer-System®+TX, Entomite-A®)+TX, Hypoaspis miles (Hypoline M®+TX, Entomite-M®)+TX, Lbalia leucospoides+TX, Lecanoideus floccissimus+TX, Lemophagus errabundus+TX, Leptomastidea abnormis+TX, Leptomastix dactylopii (Leptopar®)+TX, Leptomastix epona+TX, Lindorus lophanthae+TX, Lipolexis oregmae+TX, Lucilia caesar (Natufly®)+TX, Lysiphlebus testaceipes+TX, Macrolophus caliginosus (Mirical-N®+TX, Macroline C®+TX, Mirical®)+TX, Mesoseiulus longipes+TX, Metaphycus flavus+TX, Metaphycus lounsburyi+TX, Micromus angulatus (Milacewing®)+TX, Microterys flavus+TX, Muscidifurax raptorellus and Spalangia cameroni (Biopar®)+TX, Neodryinus typhlocybae+TX, Neoseiulus californicus+TX, Neoseiulus cucumeris (THRYPEX®)+TX, Neoseiulus fallacis+TX, Nesideocoris tenuis (NesidioBug®+TX, Nesibug®)+TX, Ophyra aenescens (Biofly®)+TX, Orius insidiosus (Thripor-I®+TX, Oriline I®)+TX, Orius laevigatus (Thripor-L®+TX, Oriline I®)+TX, Orius majusculus (Oriline M®)+TX, Orius strigicollis (Thripor-S®)+TX, Pauesia juniperorum+TX, Pediobius foveolatus+TX, Phasmarhabditis hermaphrodita (Nemaslug®)+TX, Phymastichus coffea+TX, Phytoseiulus macropilus+TX, Phytoseiulus persimilis (Spidex®+TX, Phytoline P®)+TX, Podisus maculiventris (Podisus®)+TX, Pseudacteon curvatus+TX, Pseudacteon obtusus+TX, Pseudacteon tricuspis+TX, Pseudaphycus maculipennis+TX, Pseudleptomastix mexicana+TX, Psyllaephagus pilosus+TX, Psyttalia concolor (complex)+TX, Quadrastichus spp.+TX, Rhyzobius lophanthae+TX, Rodolia cardinalis+TX, Rumina decollate+TX, Semielacher petiolatus+TX, Sitobion avenae (Ervibank®)+TX, Steinernema carpocapsae (Nematac C®+TX, Millenium®+TX, BioNem C®+TX, NemAttack®+TX, Nemastar®+TX, Capsanem®)+TX, Steinernema feltiae (NemaShield®+TX, Nemasys F®+TX, BioNem F®+TX, Steinernema-System®+TX, NemAttack®+TX, Nemaplus®+TX, Exhibitline Sf®+TX, Scia-Rid®+TX, Entonem®)+TX, Steinernema kraussei (Nemasys L®+TX, BioNem L®+TX, Exhibitline Srb®)+TX, Steinernema riobrave (BioVector®+TX, BioVektor®)+TX, Steinernema scapterisci (Nematac S®)+TX, Steinernema spp.+TX, Steinernematid spp. (Guardian Nematodes®)+TX, Stethorus punctillum (Stethorus®)+TX, Tamarixia radiate+TX, Tetrastichus setifer+TX, Thripobius semiluteus+TX, Torymus sinensis+TX, Trichogramma brassicae (Tricholine B®)+TX, Trichogramma brassicae (Tricho-Strip®)+TX, Trichogramma evanescens+TX, Trichogramma minutum+TX, Trichogramma ostriniae+TX, Trichogramma platneri+TX, Trichogramma pretiosum+TX, Xanthopimpla stemmator; and
other biologicals including: abscisic acid+TX, bioSea®+TX, Chondrostereum purpureum (Chontrol Paste®)+TX, Colletotrichum gloeosporioides (Collego®)+TX, Copper Octanoate (Cueva®)+TX, Delta traps (Trapline D®)+TX, Erwinia amylovora (Harpin) (ProAct®+TX, Ni-HIBIT Gold CST®)+TX, Ferri-phosphate (Ferramol®)+TX, Funnel traps (Trapline Y®)+TX, Gallex®+TX, Grower's Secret®+TX, Homo-brassonolide+TX, Iron Phosphate (Lilly Miller Worry Free Ferramol Slug & Snail Bait®)+TX, MCP hail trap (Trapline F®)+TX, Microctonus hyperodae+TX, Mycoleptodiscus terrestris (Des-X®)+TX, BioGain®+TX, Aminomite®+TX, Zenox®+TX, Pheromone trap (Thripline Ams®)+TX, potassium bicarbonate (MilStop®)+TX, potassium salts of fatty acids (Sanova®)+TX, potassium silicate solution (Sil-Matrix®)+TX, potassium iodide+potassiumthiocyanate (Enzicur®)+TX, SuffOil-X®+TX, Spider venom+TX, Nosema locustae (Semaspore Organic Grasshopper Control®)+TX, Sticky traps (Trapline YF®+TX, Rebell Amarillo®)+TX and Traps (Takitrapline y+B®)+TX,
or a biologically active compound or agent selected from: Brofluthrinate+TX, Diflovidazine+TX, Flometoquin+TX, Fluhexafon+TX, Plutella xylostella Granulosis virus+TX, Cydia pomonella Granulosis virus+TX, Imicyafos+TX, Heliothis virescens Nucleopolyhedrovirus+TX, Heliothis punctigera Nucleopolyhedrovirus+TX, Helicoverpa zea Nucleopolyhedrovirus+TX, Spodoptera frugiperda Nucleopolyhedrovirus+TX, Plutella xylostella Nucleopolyhedrovirus+TX, p-cymene+TX, Pyflubumide+TX, Pyrafluprole+TX, QRD 420+TX, QRD 452+TX, QRD 460+TX, Terpenoid blends+TX, Terpenoids+TX, Tetraniliprole+TX, and α-terpinene+TX; or an active substance referenced by a code+TX, such as code AE 1887196 (BSC-BX60309)+TX, code NNI-0745 GR+TX, code IKI-3106+TX, code JT-L001+TX, code ZNQ-08056+TX, code IPPA152201+TX, code HNPC-A9908 (CAS: [660411-21-2])+TX, code HNPC-A2005 (CAS: [860028-12-2])+TX, code JS118+TX, code ZJ0967+TX, code ZJ2242+TX, code JS7119 (CAS: [929545-74-4])+TX, code SN-1172+TX, code HNPC-A9835+TX, code HNPC-A9955+TX, code HNPC-A3061+TX, code Chuanhua 89-1+TX, code IPP-10+TX, code ZJ3265+TX, code JS9117+TX, code ZJ3757+TX, code ZJ4042+TX, code ZJ4014+TX, code ITM-121+TX, code DPX—RAB55 (DKI-2301)+TX, code NA-89+TX, code MIE-1209+TX, code MCI-8007+TX, code BCS-CL73507+TX, code S-1871+TX, code DPX—RDS63+TX, Quinofumelin+TX, mefentrifluconazol+TX, fenpicoxamid+TX, fluindapyr+TX, flufenpyrrolidone+TX, inpyrfluxam+TX or indiflumetpyr+TX, isoflucypram+TX, isocycloseram+TX, pyrapropoyne+TX, florylpicoxamid+TX, metyltetraprole+TX, ipflufenoquin+TX, pyridachlometyl+TX or chlopyridiflu+TX, tetrachlorantraniliprole+TX, tetrachloraniliprole+TX, Tetflupyrolimet+TX, Triflufenpyrrolidone+TX, Tyclopyrazoflor+TX, flupyrimin+TX or pyrifluramide+TX, benzpyrimoxan+TX, beflubutamid-M+TX, Benzosufyl+TX or oxazosulfyl+TX, etpyrafen+TX, acynonapyr+TX or pyrinonafen+TX, oxotrione+TX, bixlozone+TX or clofendizone+TX or dicloroxizone+TX, cyclopyranil+TX or pyrazocyclonil+TX or cyclopyrazonil+TX, alpha-bromadiolone+TX, code AKD-1193+TX, Oxathiapiprolin+TX, Fluopyram+TX, Penflufen+TX, Fluoxopyrosad+TX, fluoxapiprolin+TX, Flupyradifurone+TX, cyetpyrafen+TX, cyclobutrifluram+TX, dimesulfazet+TX, flubeneteram+TX, and flupentiofenox+TX.

The references in brackets behind the active ingredients, e.g. [3878-19-1] refer to the Chemical Abstracts Registry number. The above described mixing partners are known. Where the active ingredients are included in “The Pesticide Manual” [The Pesticide Manual—A World Compendium; Thirteenth Edition; Editor: C. D. S. TomLin; The British Crop Protection Council], they are described therein under the entry number given in round brackets hereinabove for the particular compound; for example, the compound “abamectin” is described under entry number (1). Where “[CCN]” is added hereinabove to the particular compound, the compound in question is included in the “Compendium of Pesticide Common Names”, which is accessible on the internet [A. Wood; Compendium of Pesticide Common Names, Copyright © 1995-2004]; for example, the compound “acetoprole” is described under the internet address http://www.alanwood.net/pesticides/acetoprole.html.

Most of the active ingredients described above are referred to hereinabove by a so-called “common name”, the relevant “ISO common name” or another “common name” being used in individual cases. If the designation is not a “common name”, the nature of the designation used instead is given in round brackets for the particular compound; in that case, the IUPAC name, the IUPAC/Chemical Abstracts name, a “chemical name”, a “traditional name”, a “compound name” or a “development code” is used or, if neither one of those designations nor a “common name” is used, an “alternative name” is employed. “CAS Reg. No” means the Chemical Abstracts Registry Number.

The active ingredient mixture of the compounds of formula I selected from Tables A-1 to A-81, Tables B-1 to B-54, Tables C-1 to C-81, Tables D-1 to D-54, Tables E-1 to E-81 and Tables F-1 to F-54, and Table P with active ingredients described above comprises a compound selected from Tables A-1 to A-81, Tables B-1 to B-54, Tables C-1 to C-81, Tables D-1 to D-54, Tables E-1 to E-81 and Tables F-1 to F-54, and Table P and an active ingredient as described above preferably in a mixing ratio of from 100:1 to 1:6000, especially from 50:1 to 1:50, more especially in a ratio of from 20:1 to 1:20, even more especially from 10:1 to 1:10, very especially from 5:1 and 1:5, special preference being given to a ratio of from 2:1 to 1:2, and a ratio of from 4:1 to 2:1 being likewise preferred, above all in a ratio of 1:1, or 5:1, or 5:2, or 5:3, or 5:4, or 4:1, or 4:2, or 4:3, or 3:1, or 3:2, or 2:1, or 1:5, or 2:5, or 3:5, or 4:5, or 1:4, or 2:4, or 3:4, or 1:3, or 2:3, or 1:2, or 1:600, or 1:300, or 1:150, or 1:35, or 2:35, or 4:35, or 1:75, or 2:75, or 4:75, or 1:6000, or 1:3000, or 1:1500, or 1:350, or 2:350, or 4:350, or 1:750, or 2:750, or 4:750. Those mixing ratios are by weight.

The mixtures as described above can be used in a method for controlling pests, which comprises applying a composition comprising a mixture as described above to the pests or their environment, with the exception of a method for treatment of the human or animal body by surgery or therapy and diagnostic methods practised on the human or animal body.

The mixtures comprising a compound of formula I selected from Tables A-1 to A-81, Tables B-1 to B-54, Tables C-1 to C-81, Tables D-1 to D-54, Tables E-1 to E-81 and Tables F-1 to F-54, and Table P and one or more active ingredients as described above can be applied, for example, in a single “ready-mix” form, in a combined spray mixture composed from separate formulations of the single active ingredient components, such as a “tank-mix”, and in a combined use of the single active ingredients when applied in a sequential manner, i.e. one after the other with a reasonably short period, such as a few hours or days. The order of applying the compounds of formula I selected from Tables A-1 to A-81, Tables B-1 to B-54, Tables C-1 to C-81, Tables D-1 to D-54, Tables E-1 to E-81 and Tables F-1 to F-54, and Table P and the active ingredients as described above is not essential for working the present invention.

The compositions according to the invention can also comprise further solid or liquid auxiliaries, such as stabilizers, for example unepoxidized or epoxidized vegetable oils (for example epoxidized coconut oil, rapeseed oil or soya oil), antifoams, for example silicone oil, preservatives, viscosity regulators, binders and/or tackifiers, fertilizers or other active ingredients for achieving specific effects, for example bactericides, fungicides, nematocides, plant activators, molluscicides or herbicides.

The compositions according to the invention are prepared in a manner known per se, in the absence of auxiliaries for example by grinding, screening and/or compressing a solid active ingredient and in the presence of at least one auxiliary for example by intimately mixing and/or grinding the active ingredient with the auxiliary (auxiliaries). These processes for the preparation of the compositions and the use of the compounds I for the preparation of these compositions are also a subject of the invention.

The application methods for the compositions, that is the methods of controlling pests of the abovementioned type, such as spraying, atomizing, dusting, brushing on, dressing, scattering or pouring—which are to be selected to suit the intended aims of the prevailing circumstances—and the use of the compositions for controlling pests of the abovementioned type are other subjects of the invention. Typical rates of concentration are between 0.1 and 1000 ppm, preferably between 0.1 and 500 ppm, of active ingredient. The rate of application per hectare is generally 1 to 2000 g of active ingredient per hectare, in particular 10 to 1000 g/ha, preferably 10 to 600 g/ha.

A preferred method of application in the field of crop protection is application to the foliage of the plants (foliar application), it being possible to select frequency and rate of application to match the danger of infestation with the pest in question. Alternatively, the active ingredient can reach the plants via the root system (systemic action), by drenching the locus of the plants with a liquid composition or by incorporating the active ingredient in solid form into the locus of the plants, for example into the soil, for example in the form of granules (soil application). In the case of paddy rice crops, such granules can be metered into the flooded paddy-field.

The compounds of the invention and compositions thereof are also be suitable for the protection of plant propagation material, for example seeds, such as fruit, tubers or kernels, or nursery plants, against pests of the abovementioned type. The propagation material can be treated with the compound prior to planting, for example seed can be treated prior to sowing. Alternatively, the compound can be applied to seed kernels (coating), either by soaking the kernels in a liquid composition or by applying a layer of a solid composition. It is also possible to apply the compositions when the propagation material is planted to the site of application, for example into the seed furrow during drilling. These treatment methods for plant propagation material and the plant propagation material thus treated are further subjects of the invention. Typical treatment rates would depend on the plant and pest/fungi to be controlled and are generally between 1 to 200 grams per 100 kg of seeds, preferably between 5 to 150 grams per 100 kg of seeds, such as between 10 to 100 grams per 100 kg of seeds.

The term seed embraces seeds and plant propagules of all kinds including but not limited to true seeds, seed pieces, suckers, corns, bulbs, fruit, tubers, grains, rhizomes, cuttings, cut shoots and the like and means in a preferred embodiment true seeds.

The present invention also comprises seeds coated or treated with or containing a compound of formula I. The term “coated or treated with and/or containing” generally signifies that the active ingredient is for the most part on the surface of the seed at the time of application, although a greater or lesser part of the ingredient may penetrate into the seed material, depending on the method of application. When the said seed product is (re)planted, it may absorb the active ingredient. In an embodiment, the present invention makes available a plant propagation material adhered thereto with a compound of formula (I). Further, it is hereby made available, a composition comprising a plant propagation material treated with a compound of formula (I).

Seed treatment comprises all suitable seed treatment techniques known in the art, such as seed dressing, seed coating, seed dusting, seed soaking and seed pelleting. The seed treatment application of the compound formula (I) can be carried out by any known methods, such as spraying or by dusting the seeds before sowing or during the sowing/planting of the seeds.

### BIOLOGICAL EXAMPLES

The Examples which follow serve to illustrate the invention. Certain compounds of the invention can be distinguished from known compounds by virtue of greater efficacy at low application rates, which can be verified by the person skilled in the art using the experimental procedures outlined in the Examples, using lower application rates if necessary, for example 50 ppm, 12.5 ppm, δ ppm, 3 ppm, 1.5 ppm, 0.8 ppm or 0.2 ppm.

### Example B1: Activity Against Diabrotica balteata (Corn Root Worm)

Maize sprouts placed onto an agar layer in 24-well microtiter plates were treated with aqueous test solutions prepared from 10′000 ppm DMSO stock solutions by spraying. After drying, the plates were infested with L2 larvae (6 to 10 per well). The samples were assessed for mortality and growth inhibition in comparison to untreated samples 4 days after infestation.

The following compounds gave an effect of at least 80% in at least one of the two categories (mortality or growth inhibition) at an application rate of 200 ppm: P2, P4, P5, P6, P7, P8, P9, P10, P11, P12, P14, P15, P16, P17, P18, P19, P20, P21, P22, P23, P24, P25, P26, P28, P29, P30, P31, P32, P33, P34, P35, P36, P37, P38, P39, P40, P42, P45, P46, P47, P49, P50, P51, P52.

### Example B2: Activity Against Euschistus heros (Neotropical Brown Stink Bug)

Soybean leaves on agar in 24-well microtiter plates were sprayed with aqueous test solutions prepared from 10′000 ppm DMSO stock solutions. After drying the leaves were infested with N2 nymphs. The samples were assessed for mortality and growth inhibition in comparison to untreated samples 5 days after infestation.

The following compounds gave an effect of at least 80% in at least one of the two categories (mortality or growth inhibition) at an application rate of 200 ppm: P2, P4, P5, P6, P8, P10, P11, P12, P14, P15, P17, P18, P19, P20, P21, P22, P23, P24, P25, P26, P28, P30, P31, P32, P33, P34, P38, P40, P42, P45, P46, P47, P49, P52, PN4 and PN8.

### Example B3: Activity Against Plutella xylostella (Diamond Back Moth)

24-well microtiter plates with artificial diet were treated with aqueous test solutions prepared from 10′000 ppm DMSO stock solutions by pipetting. After drying, Plutella eggs were pipetted through a plastic stencil onto a gel blotting paper and the plate was closed with it. The samples were assessed for mortality and growth inhibition in comparison to untreated samples 8 days after infestation. The following compounds gave an effect of at least 80% in at least one of the two categories (mortality or growth inhibition) at an application rate of 200 ppm: P2, P4, P5, P6, P8, P9, P10, P11, P12, P14, P15, P16, P17, P18, P19, P20, P21, P22, P23, P24, P25, P26, P27, P28, P29, P30, P31, P32, P33, P34, P35, P36, P38, P39, P40, P42, P45, P46, P47, P49, P50, P51, P52.

### Example B4: Activity Against Myzus persicae (Green Peach Aphid) Feeding/Contact Activity

Sunflower leaf discs were placed onto agar in a 24-well microtiter plate and sprayed with aqueous test solutions prepared from 10′000 ppm DMSO stock solutions. After drying, the leaf discs were infested with an aphid population of mixed ages. The samples were assessed for mortality 6 days after infestation.

The following compounds resulted in at least 80% mortality at an application rate of 200 ppm: P2, P4, P5, P6, P7, P8, P11, P12, P14, P15, P16, P17, P21, P22, P23, P24, P25, P26, P27, P32, P33, P34, P40, P42, P44, P46 and PN5.

### Example B5: Activity Against Myzus persicae (Green Peach Aphid) Systemic Activity

Roots of pea seedlings infested with an aphid population of mixed ages were placed directly into aqueous test solutions prepared from 10′000 DMSO stock solutions. The samples were assessed for mortality 6 days after placing seedlings into test solutions.

The following compounds resulted in at least 80% mortality at a test rate of 24 ppm: P2, P4, P5, P12, P16, P25, P34, P40, P42, P44, P52.

### Example B6: Activity Against Spodoptera littoralis (Egyptian Cotton Leaf Worm)

Cotton leaf discs were placed onto agar in 24-well microtiter plates and sprayed with aqueous test solutions prepared from 10′000 ppm DMSO stock solutions. After drying the leaf discs were infested with five L1 larvae. The samples were assessed for mortality, anti-feeding effect, and growth inhibition in comparison to untreated samples 3 days after infestation. Control of Spodoptera littoralis by a test sample is given when at least one of the categories mortality, anti-feedant effect, and growth inhibition is higher than the untreated sample.

The following compounds resulted in at least 80% control at an application rate of 200 ppm: P2, P4, P5, P6, P8, P10, P11, P12, P14, P15, P16, P17, P18, P19, P20, P21, P22, P23, P24, P25, P26, P27, P28, P30, P31, P33, P35, P36, P37, P38, P39, P40, P45, P46, P49, P50, P51, P52.

### Example B7: Activity Against Bemisia tabaci (Cotton White Fly)

Cotton leaf discs were placed on agar in 24-well microtiter plates and sprayed with aqueous test solutions prepared from 10′000 ppm DMSO stock solutions. After drying the leaf discs were infested with adult white flies. The samples were checked for mortality 6 days after incubation.

The following compounds resulted in at least 80% mortality at an application rate of 200 ppm: P5, P6, P11, P31.

### Example B8: Activity Against Frankliniella occidentalis (Western Flower Thrips)

Sunflower leaf discs were placed on agar in 24-well microtiter plates and sprayed with aqueous test solutions prepared from 10′000 DMSO stock solutions. After drying the leaf discs were infested with a Frankliniella population of mixed ages. The samples were assessed for mortality 7 days after infestation.

The following compounds resulted in at least 80% mortality at an application rate of 200 ppm: P6, P10, P11, P25, P26, P47.

### Example B9: Activity Against Tetranychus urticae (Two-Spotted Spider Mite)

Bean leaf discs on agar in 24-well microtiter plates were sprayed with aqueous test solutions prepared from 10′000 ppm DMSO stock solutions. After drying the leaf discs were infested with a mite population of mixed ages. The samples were assessed for mortality on mixed population (mobile stages) 8 days after infestation.

The following compounds resulted in at least 80% mortality at an application rate of 200 ppm: P26 and PN6.

1. A compound of formula (I) 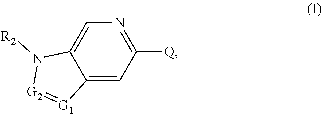
wherein
G₁ and G₂ are, independently from each other, CH or N;
R₂ is C₁-C₆haloalkyl;
Q is a radical selected from the group consisting of formula Qa and Qb 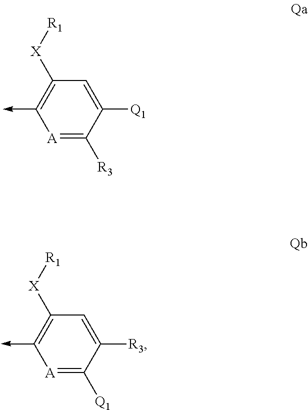
wherein the arrow denotes the point of attachment to the carbon atom of the bicyclic ring;
and wherein A represents CH or N;
X is S, SO or SO₂;
R₁ is C₁-C₄alkyl or C₃-C₆cycloalkyl-C₁-C₄alkyl;
Q₁ is hydrogen, halogen, C₁-C₆haloalkyl, C₃-C₆cycloalkyl, C₃-C₆cycloalkyl monosubstituted by cyano, C₁-C₆cyanoalkyl, C₁-C₆cyanoalkoxy, C₁-C₆haloalkoxy, —N(R₄)₂, —N(R₄)COR₅, —N(R₄)CON(R₄)₂, (oxazolidin-2-one)-3-yl or 2-pyridyloxy; or
Q₁ is a five- to six-membered aromatic ring system linked via a ring carbon atom to the ring which contains the substituent A, said ring system is unsubstituted or is mono- or polysubstituted by substituents selected from the group consisting of halogen, cyano, C₁-C₄alkyl, C₁-C₄haloalkyl, C₁-C₄alkoxy, C₁-C₄haloalkoxy, C₁-C₄alkylsulfanyl, C₁-C₄alkylsulfinyl and C₁-C₄alkylsulfonyl; and said ring system can contain 1, 2 or 3 ring heteroatoms selected from the group consisting of nitrogen, oxygen and sulphur, where said ring system may not contain more than one ring oxygen atom and not more than one ring sulfur atom; or
Q₁ is a five-membered aromatic ring system linked via a ring nitrogen atom to the ring which contains the substituent A, said ring system is unsubstituted or is mono- or polysubstituted by substituents selected from the group consisting of halogen, cyano, C₁-C₄alkyl, C₁-C₄haloalkyl, C₁-C₄alkoxy, C₁-C₄haloalkoxy, C₁-C₄alkylsulfanyl, C₁-C₄alkylsulfinyl and C₁-C₄alkylsulfonyl; and said ring system contains 1, 2 or 3 ring heteroatoms selected from the group consisting of nitrogen, oxygen and sulphur, where said ring system contains at least one ring nitrogen atom and may not contain more than one ring oxygen atom and not more than one ring sulfur atom;
R₃ is hydrogen, halogen or C₁-C₄alkyl;
each R₄ is independently hydrogen, C₁-C₄alkyl or C₃-C₆cycloalkyl; and
R₅ is C₁-C₆alkyl, C₁-C₆haloalkyl or C₃-C₆cycloalkyl;
or an agrochemically acceptable salt, stereoisomer, enantiomer, tautomer or N-oxide of a compound of formula I.

2. A compound of formula I according to claim 1, represented by the compounds of formula (I-1) 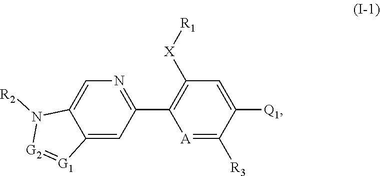
wherein A, X, R₁, R₂, G₁ and G₂ are as defined under formula I in claim 1, and wherein
Q₁ is hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂, —N(R₄)COR₅, or —N(R₄)CON(R₄)₂, (oxazolidin-2-one)-3-yl or 2-pyridyloxy; or
Q₁ is a five- to six-membered aromatic ring system linked via a ring carbon atom to the ring which contains the substituent A, said ring system is unsubstituted or is mono-substituted by a substituent selected from the group consisting of halogen, cyano and C₁-C₄haloalkyl; and said ring system can contain 1 or 2 ring nitrogen atoms; or
Q₁ is a five-membered aromatic ring system linked via a ring nitrogen atom to the ring which contains the substituent A, said ring system is unsubstituted or is mono-substituted by a substituent selected from the group consisting of halogen, cyano and C₁-C₄haloalkyl; and said ring system contains 2 or 3 ring nitrogen atoms;
R₃ is hydrogen or C₁-C₄alkyl;
each R₄ is independently hydrogen or C₁-C₄alkyl; and
R₅ is C₁-C₆alkyl or C₃-C₆cycloalkyl;
or an agrochemically acceptable salt, stereoisomer, enantiomer, tautomer or N-oxide of a compound of formula I-1.

3. A compound of formula I according to claim 1, represented by the compounds of formula (I-5) 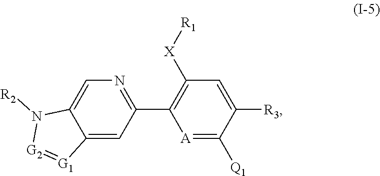
wherein A, X, R₁, R₂, G₁ and G₂ are as defined under formula I in claim 1, and wherein
Q₁ is hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂, —N(R₄)COR₅, or —N(R₄)CON(R₄)₂, (oxazolidin-2-one)-3-yl or 2-pyridyloxy; or
Q₁ is a five- to six-membered aromatic ring system linked via a ring carbon atom to the ring which contains the substituent A, said ring system is unsubstituted or is mono-substituted by a substituent selected from the group consisting of halogen, cyano and C₁-C₄haloalkyl; and said ring system can contain 1 or 2 ring nitrogen atoms; or
Q₁ is a five-membered aromatic ring system linked via a ring nitrogen atom to the ring which contains the substituent A, said ring system is unsubstituted or is mono-substituted by a substituent selected from the group consisting of halogen, cyano and C₁-C₄haloalkyl; and said ring system contains 2 or 3 ring nitrogen atoms;
R₃ is hydrogen or C₁-C₄alkyl;
each R₄ is independently hydrogen or C₁-C₄alkyl; and
R₅ is C₁-C₆alkyl or C₃-C₆cycloalkyl;
or an agrochemically acceptable salt, stereoisomer, enantiomer, tautomer or N-oxide of a compound of formula I-5.

4. A compound of formula I according to claim 1, represented by the compounds of formula (I-2) 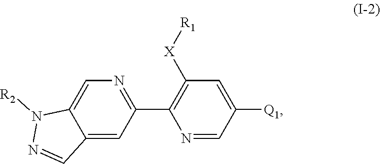
wherein X, R₁ and R₂ are as defined under formula I in claim 1, and wherein
Q₁ is hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂, —N(R₄)COR₅, or —N(R₄)CON(R₄)₂, (oxazolidin-2-one)-3-yl or 2-pyridyloxy; or
Q₁ is a five- to six-membered aromatic ring system linked via a ring carbon atom to the pyridyl substituted by X—R₁, said ring system is unsubstituted or is mono-substituted by a substituent selected from the group consisting of halogen, cyano and C₁-C₄haloalkyl; and said ring system can contain 1 or 2 ring nitrogen atoms; or
Q₁ is a five-membered aromatic ring system linked via a ring nitrogen atom to the pyridyl ring substituted by X—R₁, said ring system is unsubstituted or is mono-substituted by a substituent selected from the group consisting of halogen, cyano and C₁-C₄haloalkyl; and said ring system contains 2 or 3 ring nitrogen atoms;
each R₄ is independently hydrogen or C₁-C₄alkyl; and
R₅ is C₁-C₆alkyl or C₃-C₆cycloalkyl; preferably R₅ is methyl or cyclopropyl;
or an agrochemically acceptable salt, stereoisomer, enantiomer, tautomer or N-oxide of a compound of formula I-2.

5. A compound of formula I according to claim 1, represented by the compounds of formula (I-6) 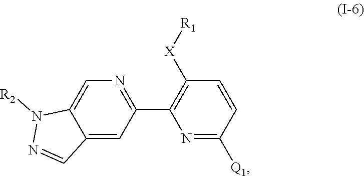
wherein X, R₁ and R₂ are as defined under formula I in claim 1, and wherein
Q₁ is hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂, —N(R₄)COR₅, or —N(R₄)CON(R₄)₂, (oxazolidin-2-one)-3-yl or 2-pyridyloxy; or
Q₁ is a five- to six-membered aromatic ring system linked via a ring carbon atom to the ring which contains the substituent A, said ring system is unsubstituted or is mono-substituted by a substituent selected from the group consisting of halogen, cyano and C₁-C₄haloalkyl; and said ring system can contain 1 or 2 ring nitrogen atoms; or
Q₁ is a five-membered aromatic ring system linked via a ring nitrogen atom to the ring which contains the substituent A, said ring system is unsubstituted or is mono-substituted by a substituent selected from the group consisting of halogen, cyano and C₁-C₄haloalkyl; and said ring system contains 2 or 3 ring nitrogen atoms;
each R₄ is independently hydrogen or C₁-C₄alkyl; and
R₅ is C₁-C₆alkyl or C₃-C₆cycloalkyl; preferably R₅ is methyl or cyclopropyl;
or an agrochemically acceptable salt, stereoisomer, enantiomer, tautomer or N-oxide of a compound of formula I-6.

6. A compound of formula I according to claim 1, represented by the compounds of formula (I-9) 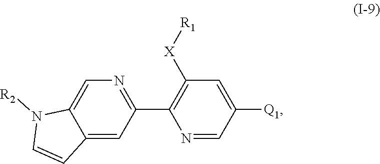
wherein X, R₁ and R₂ are as defined as defined under formula I in claim 1, and wherein
Q₁ is hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂, —N(R₄)COR₅, or —N(R₄)CON(R₄)₂, (oxazolidin-2-one)-3-yl or 2-pyridyloxy; or
Q₁ is a five- to six-membered aromatic ring system linked via a ring carbon atom to the ring which contains the substituent A, said ring system is unsubstituted or is mono-substituted by a substituent selected from the group consisting of halogen, cyano and C₁-C₄haloalkyl; and said ring system can contain 1 or 2 ring nitrogen atoms; or
Q₁ is a five-membered aromatic ring system linked via a ring nitrogen atom to the ring which contains the substituent A, said ring system is unsubstituted or is mono-substituted by a substituent selected from the group consisting of halogen, cyano and C₁-C₄haloalkyl; and said ring system contains 2 or 3 ring nitrogen atoms;
each R₄ is independently hydrogen or C₁-C₄alkyl; and
R₅ is C₁-C₆alkyl or C₃-C₆cycloalkyl; preferably R₅ is methyl or cyclopropyl;
or an agrochemically acceptable salt, stereoisomer, enantiomer, tautomer or N-oxide of a compound of formula I-9.

7. A compound of formula I according to claim 1, represented by the compounds of formula (I-12) 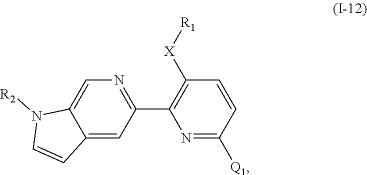
wherein X, R₁ and R₂ are as defined under formula I in claim 1, and wherein
Q₁ is hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂, —N(R₄)COR₅, or —N(R₄)CON(R₄)₂, (oxazolidin-2-one)-3-yl or 2-pyridyloxy; or
Q₁ is a five- to six-membered aromatic ring system linked via a ring carbon atom to the ring which contains the substituent A, said ring system is unsubstituted or is mono-substituted by a substituent selected from the group consisting of halogen, cyano and C₁-C₄haloalkyl; and said ring system can contain 1 or 2 ring nitrogen atoms; or
Q₁ is a five-membered aromatic ring system linked via a ring nitrogen atom to the ring which contains the substituent A, said ring system is unsubstituted or is mono-substituted by a substituent selected from the group consisting of halogen, cyano or C₁-C₄haloalkyl; and said ring system contains 2 or 3 nitrogen atoms;
each R₄ is independently hydrogen or C₁-C₄alkyl; and
R₅ is C₁-C₆alkyl or C₃-C₆cycloalkyl; preferably R₅ is methyl or cyclopropyl;
or an agrochemically acceptable salt, stereoisomer, enantiomer, tautomer or N-oxide of a compound of formula I-12.

8. A compound of formula I according to claim 1, represented by the compounds of formula (I-15) 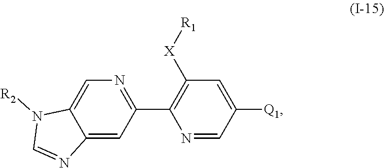
wherein X, R₁ and R₂ are as defined under formula I in claim 1, and wherein
Q₁ is hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂, —N(R₄)COR₅, or —N(R₄)CON(R₄)₂, (oxazolidin-2-one)-3-yl or 2-pyridyloxy; or
Q₁ is a five- to six-membered aromatic ring system linked via a ring carbon atom to the ring which contains the substituent A, said ring system is unsubstituted or is mono-substituted by a substituent selected from the group consisting of halogen, cyano and C₁-C₄haloalkyl; and said ring system can contain 1 or 2 ring nitrogen atoms; or
Q₁ is a five-membered aromatic ring system linked via a ring nitrogen atom to the ring which contains the substituent A, said ring system is unsubstituted or is mono-substituted by a substituent selected from the group consisting of halogen, cyano and C₁-C₄haloalkyl; and said ring system contains 2 or 3 ring nitrogen atoms;
each R₄ is independently hydrogen or C₁-C₄alkyl; and
R₅ is C₁-C₆alkyl or C₃-C₆cycloalkyl; preferably R₅ is methyl or cyclopropyl;
or an agrochemically acceptable salt, stereoisomer, enantiomer, tautomer or N-oxide of a compound of formula I-15.

9. A compound of formula I according to claim 1, represented by the compounds of formula (I-18) 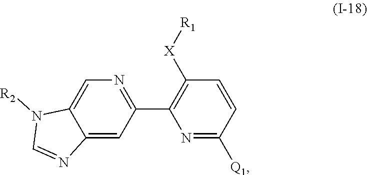
wherein X, R₁ and R₂ are as defined under formula I in claim 1, and wherein
Q₁ is hydrogen, halogen, C₁-C₆haloalkyl, C₃-C₆cycloalkyl, C₃-C₆cycloalkyl monosubstituted by cyano, C₁-C₆cyanoalkyl, C₁-C₆haloalkoxy, —N(R₄)₂, —N(R₄)COR₅, or —N(R₄)CON(R₄)₂, (oxazolidin-2-one)-3-yl or 2-pyridyloxy; or
Q₁ is a five- to six-membered aromatic ring system linked via a ring carbon atom to the ring which contains the substituent A, said ring system is unsubstituted or is mono-substituted by a substituent selected from the group consisting of halogen, cyano and C₁-C₄haloalkyl; and said ring system can contain 1 or 2 ring nitrogen atoms; or
Q₁ is a five-membered aromatic ring system linked via a ring nitrogen atom to the ring which contains the substituent A, said ring system is unsubstituted or is mono-substituted by a substituent selected from the group consisting of halogen, cyano and C₁-C₄haloalkyl; and said ring system contains 2 or 3 ring nitrogen atoms;
each R₄ is independently hydrogen or C₁-C₄alkyl; and
R₅ is C₁-C₆alkyl or C₃-C₆cycloalkyl; preferably R₅ is methyl or cyclopropyl;
or an agrochemically acceptable salt, stereoisomer, enantiomer, tautomer or N-oxide of a compound of formula I-18.

10. A compound according to claim 1, wherein
X is S or SO₂; preferably X is SO₂; and.
R₁ is C₁-C₄alkyl or cyclopropyl-C₁-C₄alkyl; preferably R₁ is ethyl or cyclopropylmethyl.

11. A compound according to claim 1, wherein Q₁ is hydrogen, halogen, C₁-C₆haloalkyl, C₃-C₆cycloalkyl, C₃-C₆cycloalkyl monosubstituted by cyano, C₁-C₆cyanoalkyl, —N(R₄)₂, —N(R₄)COR₅ or —N(R₄)CON(R₄)₂, in each of which R₄ is independently either hydrogen or C₁-C₄alkyl (preferably hydrogen or methyl) and R₅ is C₁-C₆alkyl or C₃-C₆cycloalkyl (preferably methyl or cyclopropyl); or Q₁ is (oxazolidin-2-one)-3-yl, 2-pyridyloxy, N-linked pyrazolyl which is mono-substituted by chloro or trifluoromethyl; or Q₁ is C-linked pyrimidinyl or N-linked triazolyl; preferably Q₁ is hydrogen, chlorine, trifluoromethyl, cyclopropyl, 1-cyanocyclopropyl, 1-cyano-1-methyl-ethyl, —NH(CH₃), —N(CH₃)COCH₃, —N(CH₃)CO(cyclopropyl), —N(CH₃)CONH(CH₃), (oxazolidin-2-one)-3-yl, 2-pyridyloxy, 3-chloro-pyrazol-1-yl, 3-trifluoromethyl-pyrazol-1-yl, pyrimidin-2-yl or 1,2,4-triazol-1-yl.

12. A compound according to claim 1, wherein R₂ is C₁-C₆fluoroalkyl; preferably R₂ is —CH₂CF₂CHF₂ or —CH₂CF₂CF₃.

13. A compound according to claim 1, wherein Q₁ is hydrogen, chlorine, bromine, trifluoromethyl, cyclopropyl, 1-cyanocyclopropyl, 1-cyano-1-methyl-ethyl, 2,2,2-trifluoroethoxy, —NH(CH₃), —N(CH₃)COCH₃, —N(CH₃)CO(cyclopropyl), —N(H)CONH(CH₃), —N(CH₃)CONH(CH₃), (oxazolidin-2-one)-3-yl, 2-pyridyloxy, pyrazol-1-yl, 3-chloro-pyrazol-1-yl, 3-cyano-pyrazol-1-yl, 3-trifluoromethyl-pyrazol-1-yl, 1,2,4-triazol-1-yl or pyrimidin-2-yl.

14. A compound of formula I as claimed in claim 1, selected from the group consisting of:
5-ethylsulfonyl-N-methyl-6-[1-(2,2,3,3,3-pentafluoropropyl) pyrazolo[3,4-c]pyridin-5-yl]pyridin-3-amine (compound P1);
N-[5-ethylsulfonyl-6-[1-(2,2,3,3,3-pentafluoropropyl) pyrazolo[3,4-c]pyridin-5-yl]-3-pyridyl]-N-methyl-acetamide (compound P2);
5-(3-ethylsulfanyl-2-pyridyl)-1-(2,2,3,3,3-pentafluoropropyl) pyrazolo[3,4-c]pyridine (compound P3);
5-(3-ethylsulfanyl-2-pyridyl)-1-(2,2,3,3,3-pentafluoropropyl) pyrazolo[3,4-c]pyridine (compound P4);
2-[5-ethylsulfonyl-6-[1-(2,2,3,3,3-pentafluoropropyl)pyrazolo[3,4-c]pyridin-5-yl]-3-pyridyl]-2-methyl-propanenitrile (compound P5);
2-[5-ethylsulfanyl-6-[1-(2,2,3,3,3-pentafluoropropyl)pyrazolo[3,4-c]pyridin-5-yl]-3-pyridyl]-2-methyl-propanenitrile (compound P6);
5-(3-ethylsulfinyl-2-pyridyl)-1-(2,2,3,3,3-pentafluoropropyl)pyrrolo[2,3-c]pyridine (compound P7);
5-(3-ethylsulfonyl-2-pyridyl)-1-(2,2,3,3,3-pentafluoropropyl)pyrrolo[2,3-c]pyridine (compound P8);
6-(5-cyclopropyl-3-ethylsulfanyl-2-pyridyl)-3-(2,2,3,3,3-pentafluoropropyl)imidazo[4,5-c]pyridine (compound P9);
1-[5-ethylsulfanyl-6-[1-(2,2,3,3,3-pentafluoropropyl)pyrazolo[3,4-c]pyridin-5-yl]-3-pyridyl]cyclopropanecarbonitrile (compound P10);
1-[5-ethylsulfonyl-6-[1-(2,2,3,3,3-pentafluoropropyl)pyrazolo[3,4-c]pyridin-5-yl]-3-pyridyl]cyclopropanecarbonitrile (compound P11);
N-[5-ethylsulfonyl-6-[1-(2,2,3,3,3-pentafluoropropyl)pyrazolo[3,4-c]pyridin-5-yl]-2-pyridyl]-N-methyl-acetamide (compound P12);
1-[5-ethylsulfonyl-6-[1-(2,2,3,3,3-pentafluoropropyl)pyrrolo[2,3-c]pyridin-5-yl]-2-pyridyl]-1,3-dimethyl-urea (compound P13);
5-ethylsulfonyl-N-methyl-6-[1-(2,2,3,3,3-pentafluoropropyl)pyrazolo[3,4-c]pyridin-5-yl]pyridin-2-amine (compound P14);
N-[5-ethylsulfonyl-6-[1-(2,2,3,3,3-pentafluoropropyl)pyrazolo[3,4-c]pyridin-5-yl]-2-pyridyl]-N-methyl-cyclopropanecarboxamide (compound P15);
N-[5-ethylsulfonyl-6-[1-(2,2,3,3,3-pentafluoropropyl)pyrrolo[2,3-c]pyridin-5-yl]-3-pyridyl]-N-methyl-acetamide (compound P16);
5-[3-ethylsulfonyl-5-(2-pyridyloxy)-2-pyridyl]-1-(2,2,3,3,3-pentafluoropropyl)pyrrolo[2,3-c]pyridine (compound P17);
5-ethylsulfonyl-N-methyl-6-[1-(2,2,3,3,3-pentafluoropropyl)pyrrolo[2,3-c]pyridin-5-yl]pyridin-3-amine (compound P18);
2-[5-ethylsulfonyl-6-[1-(2,2,3,3,3-pentafluoropropyl)pyrrolo[2,3-c]pyridin-5-yl]-3-pyridyl]-2-methyl-propanenitrile (compound P19);
5-[3-ethylsulfanyl-5-(2-pyridyloxy)-2-pyridyl]-1-(2,2,3,3,3-pentafluoropropyl)pyrazolo[3,4-c]pyridine (compound P20);
5-[3-ethylsulfonyl-5-(2-pyridyloxy)-2-pyridyl]-1-(2,2,3,3,3-pentafluoropropyl)pyrazolo[3,4-c]pyridine (compound P21);
5-[3-ethylsulfonyl-6-(1,2,4-triazol-1-yl)-2-pyridyl]-1-(2,2,3,3,3-pentafluoropropyl)pyrazolo[3,4-c]pyridine (compound P22);
1-[5-ethylsulfonyl-6-[1-(2,2,3,3,3-pentafluoropropyl)pyrrolo[2,3-c]pyridin-5-yl]-3-pyridyl]cyclopropanecarbonitrile (compound P23);
5-ethylsulfonyl-N-methyl-6-[1-(2,2,3,3,3-pentafluoropropyl)pyrrolo[2,3-c]pyridin-5-yl]pyridin-2-amine (compound P24);
5-(3-ethylsulfonyl-6-pyrimidin-2-yl-2-pyridyl)-1-(2,2,3,3,3-pentafluoropropyl)pyrazolo[3,4-c]pyridine (compound P25);
5-(6-cyclopropyl-3-ethylsulfonyl-2-pyridyl)-1-(2,2,3,3,3-pentafluoropropyl)pyrazolo[3,4-c]pyridine (compound P26);
3-[5-ethylsulfonyl-6-[1-(2,2,3,3,3-pentafluoropropyl)pyrazolo[3,4-c]pyridin-5-yl]-2-pyridyl]oxazolidin-2-one (compound P27);
5-(5-cyclopropyl-3-ethylsulfonyl-2-pyridyl)-1-(2,2,3,3,3-pentafluoropropyl)pyrazolo[3,4-c]pyridine (compound P28);
2-[5-ethylsulfanyl-6-[1-(2,2,3,3,3-pentafluoropropyl)pyrrolo[2,3-c]pyridin-5-yl]-3-pyridyl]-2-methyl-propanenitrile (compound P29);
5-[3-ethylsulfanyl-5-(trifluoromethyl)-2-pyridyl]-1-(2,2,3,3,3-pentafluoropropyl)pyrazolo[3,4-c]pyridine (compound P30);
5-[3-ethylsulfonyl-5-(trifluoromethyl)-2-pyridyl]-1-(2,2,3,3,3-pentafluoropropyl)pyrazolo[3,4-c]pyridine (compound P31);
6-[3-ethylsulfonyl-6-(1,2,4-triazol-1-yl)-2-pyridyl]-3-(2,2,3,3,3-pentafluoropropyl)imidazo[4,5-c]pyridine (compound P32);
1-[5-ethylsulfonyl-6-[1-(2,2,3,3,3-pentafluoropropyl)pyrazolo[3,4-c]pyridin-5-yl]-2-pyridyl]-1,3-dimethyl-urea (compound P33);
1-[5-ethylsulfonyl-6-[3-(2,2,3,3,3-pentafluoropropyl)imidazo[4,5-c]pyridin-6-yl]-3-pyridyl]cyclopropanecarbonitrile (compound P34);
5-[3-ethylsulfonyl-6-(1,2,4-triazol-1-yl)-2-pyridyl]-1-(2,2,3,3,3-pentafluoropropyl)pyrrolo[2,3-c]pyridine (compound P35);
5-(6-chloro-3-ethylsulfonyl-2-pyridyl)-1-(2,2,3,3,3-pentafluoropropyl)pyrrolo[2,3-c]pyridine (compound P36);
1-[5-ethylsulfanyl-6-[1-(2,2,3,3,3-pentafluoropropyl)pyrrolo[2,3-c]pyridin-5-yl]-3-pyridyl]cyclopropanecarbonitrile (compound P37);
1-[5-ethylsulfanyl-6-[3-(2,2,3,3,3-pentafluoropropyl)imidazo[4,5-c]pyridin-6-yl]-3-pyridyl]cyclopropanecarbonitrile (compound P38);
5-[5-(3-chloropyrazol-1-yl)-3-ethylsulfanyl-2-pyridyl]-1-(2,2,3,3,3-pentafluoropropyl)pyrazolo[3,4-c]pyridine (compound P39);
2-[5-ethylsulfonyl-6-[3-(2,2,3,3,3-pentafluoropropyl)imidazo[4,5-c]pyridin-6-yl]-3-pyridyl]-2-methyl-propanenitrile (compound P40);
6-(6-chloro-3-ethylsulfonyl-2-pyridyl)-3-(2,2,3,3,3-pentafluoropropyl)imidazo[4,5-c]pyridine (compound P41);
6-(3-ethylsulfonyl-2-pyridyl)-3-(2,2,3,3,3-pentafluoropropyl)imidazo[4,5-c]pyridine (compound P42);
6-(6-chloro-3-ethylsulfanyl-2-pyridyl)-3-(2,2,3,3,3-pentafluoropropyl)imidazo[4,5-c]pyridine (compound P43);
6-(3-ethylsulfanyl-2-pyridyl)-3-(2,2,3,3,3-pentafluoropropyl)imidazo[4,5-c]pyridine (compound P44);
5-[3-ethylsulfonyl-5-[3-(trifluoromethyl)pyrazol-1-yl]-2-pyridyl]-1-(2,2,3,3,3-pentafluoropropyl)pyrazolo[3,4-c]pyridine (compound P45);
5-[5-(3-chloropyrazol-1-yl)-3-ethylsulfonyl-2-pyridyl]-1-(2,2,3,3,3-pentafluoropropyl)pyrazolo[3,4-c]pyridine (compound P46);
2-[5-ethylsulfanyl-6-[3-(2,2,3,3,3-pentafluoropropyl)imidazo[4,5-c]pyridin-6-yl]-3-pyridyl]-2-methyl-propanenitrile (compound P47);
5-(6-chloro-3-ethylsulfanyl-2-pyridyl)-1-(2,2,3,3,3-pentafluoropropyl)pyrrolo[2,3-c]pyridine (compound P48);
5-(6-chloro-3-ethylsulfonyl-2-pyridyl)-1-(2,2,3,3,3-pentafluoropropyl)pyrazolo[3,4-c]pyridine (compound P49);
5-[3-ethylsulfanyl-5-[3-(trifluoromethyl)pyrazol-1-yl]-2-pyridyl]-1-(2,2,3,3,3-pentafluoropropyl)pyrazolo[3,4-c]pyridine (compound P50);
5-(5-cyclopropyl-3-ethylsulfonyl-2-pyridyl)-1-(2,2,3,3,3-pentafluoropropyl)pyrrolo[2,3-c]pyridine (compound P51);
6-(5-cyclopropyl-3-ethylsulfonyl-2-pyridyl)-3-(2,2,3,3,3-pentafluoropropyl)imidazo[4,5-c]pyridine (compound P52);
5-(6-chloro-3-ethylsulfanyl-2-pyridyl)-1-(2,2,3,3,3-pentafluoropropyl)pyrazolo[3,4-c]pyridine (compound P53); and
5-[3-ethylsulfanyl-5-(2-pyridyloxy)-2-pyridyl]-1-(2,2,3,3,3-pentafluoropropyl)pyrrolo[2,3-c]pyridine (compound P54).

15. A compound of formula I as claimed in claim 1, selected from the group consisting of:
5-(3-ethylsulfanyl-1-oxido-pyridin-1-ium-2-yl)-1-(2,2,3,3,3-pentafluoropropyl)pyrazolo[3,4-c]pyridine (compound PN1);
5-(3-ethylsulfanyl-1-oxido-pyridin-1-ium-2-yl)-1-(2,2,3,3,3-pentafluoropropyl)pyrrolo[2,3-c]pyridine (compound PN2);
6-(3-ethylsulfanyl-1-oxido-pyridin-1-ium-2-yl)-3-(2,2,3,3,3-pentafluoropropyl)imidazo[4,5-c]pyridine (compound PN3);
6-(5-cyclopropyl-3-ethylsulfanyl-1-oxido-pyridin-1-ium-2-yl)-3-(2,2,3,3,3-pentafluoropropyl)imidazo[4,5-c]pyridine (compound PN4);
5-(5-cyclopropyl-3-ethylsulfanyl-1-oxido-pyridin-1-ium-2-yl)-1-(2,2,3,3,3-pentafluoropropyl)pyrrolo[2,3-c]pyridine (compound PN5);
2-[5-ethylsulfanyl-1-oxido-6-[1-(2,2,3,3,3-pentafluoropropyl)pyrrolo[2,3-c]pyridin-5-yl]pyridin-1-ium-3-yl]-2-methyl-propanenitrile (compound PN6);
2-[5-ethylsulfanyl-1-oxido-6-[3-(2,2,3,3,3-pentafluoropropyl)imidazo[4,5-c]pyridin-6-yl]pyridin-1-ium-3-yl]-2-methyl-propanenitrile (compound PN7);
1-[5-ethylsulfanyl-1-oxido-6-[3-(2,2,3,3,3-pentafluoropropyl)imidazo[4,5-c]pyridin-6-yl]pyridin-1-ium-3-yl]cyclopropanecarbonitrile (compound PN8);
1-[5-ethylsulfanyl-1-oxido-6-[1-(2,2,3,3,3-pentafluoropropyl)pyrrolo[2,3-c]pyridin-5-yl]pyridin-1-ium-3-yl]cyclopropanecarbonitrile (compound PN9);
5-[3-ethylsulfanyl-1-oxido-5-(2-pyridyloxy)pyridin-1-ium-2-yl]-1-(2,2,3,3,3-pentafluoropropyl)pyrrolo[2,3-c]pyridine (compound PN10); and
5-[3-ethylsulfanyl-1-oxido-5-(2-pyridyloxy)pyridin-1-ium-2-yl]-1-(2,2,3,3,3-pentafluoropropyl)pyrazolo[3,4-c]pyridine (compound PN11).

16. A composition comprising an insecticidally, acaricidally, nematicidally or molluscicidally effective amount of a compound of formula (I), or an agrochemically acceptable salt, stereoisomer, enantiomer, tautomer or N-oxide thereof, as defined in claim 1.

17. A method of combating and controlling insects, acarines, nematodes or molluscs which comprises applying to a pest, to a locus of a pest, or to a plant susceptible to attack by a pest an insecticidally, acaricidally, nematicidally or molluscicidally effective amount of a compound of formula (I), or an agrochemically acceptable salt, stereoisomer, enantiomer, tautomer or N-oxide thereof, as defined in claim 1.

18. A method for the protection of plant propagation material from the attack by insects, acarines, nematodes or molluscs, which comprises treating the propagation material or the site, where the propagation material is planted, with a composition according to claim 16.

19. A compound of formula IXa-1-SP 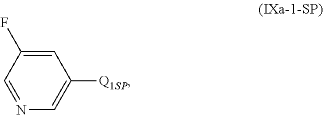
wherein
Q_{1SP} is 1-cyanocyclopropyl, 1-cyano-1-methyl-ethyl or 2-pyridyloxy.

20. A compound of formula VIIa-1-SP′ 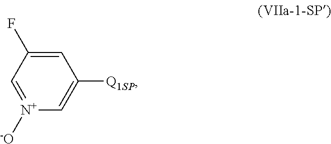
wherein
Q_{1SP′} is cyclopropyl, 1-cyanocyclopropyl, 1-cyano-1-methyl-ethyl or 2-pyridyloxy.

21. A compound of formula VI 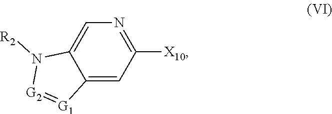
wherein
G₁, G₂ and R₂ are as defined under formula I above; and
X₁₀ is a halogen or a pseudo-halogen leaving group.

22. A compound of formula II-Qa 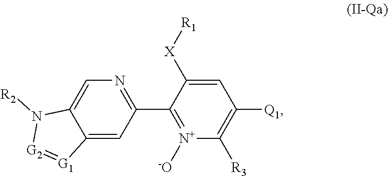
wherein
X is S; and
G₁, G₂, R₂, Q₁, R₃ and R₁ are as defined under formula I in claim 1.

23. A compound of formula III-Qa 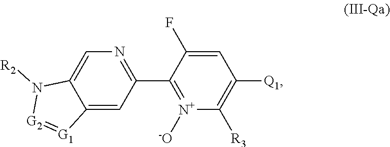
wherein
G₁, G₂, R₂, Q₁ and R₃ are as defined under formula I in claim 1.

24. A compound of formula IV-Qa 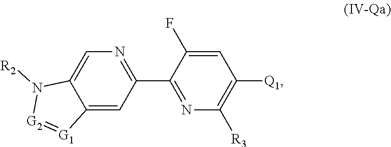
wherein
G₁, G₂, R₂, Q₁ and R₃ are as defined under formula I in claim 1.

25. A composition comprising an insecticidally, acaricidally, nematicidally or molluscicidally effective amount of a compound of formula (I), or an agrochemically acceptable salt, stereoisomer, enantiomer, tautomer or N-oxide thereof, as defined in an auxiliary or diluent.

26. A method of combating and controlling insects, acarines, nematodes or molluscs which comprises applying to a pest, to a locus of a pest, or to a plant susceptible to attack by a pest an insecticidally, acaricidally, nematicidally or molluscicidally effective amount of a compound of formula (I), or an agrochemically acceptable salt, stereoisomer, enantiomer, tautomer or N-oxide thereof, with a composition as defined claim 16.
